(19) United States
(12) Patent Application Publication
(10) Pub. No.: US 2026/0284176 A1
(43) Pub. Date: Sep. 24, 2026

(54) HIV VACCINE

(71) Applicants: BioNTech SE, Mainz (DE); THE GENERAL HOSPITAL CORPORATION, Boston, MA (US)

(72) Inventors: Valentin LE DOUCE, Mainz (DE); Ekaterina ESAULOVA, Cambridge, MA (US); Daphne KAXIRAS, Cambridge, MA (US); Michael Steven ROONEY, Cambridge, MA (US); Theresa ADDONA, Cambridge, MA (US); Ugur SAHIN, Mainz (DE); Gaurav GAIHA, Boston, MA (US)

(21) Appl. No.: 19/474,552

(22) PCT Filed: Apr. 12, 2024
(86) PCT No.: PCT/US2024/024503

§ 371 Date: Oct. 10, 2025

Related U.S. Application Data
(60) Provisional application No. 63/459,449, filed on Apr. 14, 2023.
(60) Provisional application No. 63/547,796, filed on Nov. 8, 2023.
(60) Provisional application No. 63/549,262, filed on Feb. 2, 2024.

(51) Int. Cl. A61K 39/21; A61K 9/00; A61K 31/7115; A61K 39/00; A61K 39/295; A61P 31/18; C07K 14/16; C12N 15/113

(52) U.S. Cl. CPC A61K 39/21; A61K 9/0019; A61K 31/7115; A61K 39/295; A61P 31/18; C07K 14/16; C12N 15/113; A61K 2039/53; A61K 2039/54; A61K 2039/545; A61K 2039/55544; A61K 2039/55555; A61K 2039/70; C12N 2310/532; C12N 2830/50

(57) ABSTRACT

The present invention concerns compositions comprising an RNA molecule, wherein the RNA molecule comprises an expression cassette encoding an immunogenic peptide, which peptide comprises at least two fragments, wherein each fragment comprises at least one epitope, wherein the epitope is derived from an amino acid sequence encoded by the human immunodeficiency virus (HIV) and medical preparations comprising such compositions. Furthermore, the invention concerns methods for preventing or treating HIV.

### CROSS REFERENCE TO RELATED APPLICATIONS

The present application claims priority to United States Provisional Patent Application Nos. 63/459,449, filed Apr. 14, 2023, 63/547,796, filed Nov. 8, 2023, and 63/549,262, filed Feb. 2, 2024, each of which are hereby incorporated by reference in its entirety.

### TECHNICAL FIELD

The present invention concerns compositions comprising an RNA molecule, wherein the RNA molecule comprises an expression cassette encoding an immunogenic peptide, which peptide comprises at least two fragments, wherein each fragment comprises at least one epitope, wherein the epitope is derived from an amino acid sequence encoded by the human immunodeficiency virus (HIV) and medical preparations comprising such compositions. Furthermore, the invention concerns methods for preventing or treating HIV.

### BACKGROUND

HIV remains a global health threat with ~38 million infected individuals worldwide (WHO, Global progress report on HIV, viral hepatitis and sexually transmitted infections, 2021). Recent developments in successful combinatorial anti-retroviral therapy (cART) have dramatically decreased the HIV burden. However, a prophylactic HIV vaccine remains elusive and cART is unable to facilitate complete viral clearance in patients. Hence, HIV remains as a lifelong chronic condition for over 21 million people with access to therapeutics. Increasing cART global availability, decreasing the cost of treatment regimens, and addressing HIV drug resistance are critical goals to create a “functional cure”.

Cure therapy is the holy grail of HIV therapeutics and remains a tremendous challenge in the HIV field. Current cure strategies aim to elicit strong HIV-specific CD8+ T cell responses and/or use of bNAbs as a therapeutic agent to prevent (re)infection and promote elimination of infected cells. Key HIV immunotherapy challenges are i) creation of drug products with broad applicability; ii) high diversity of circulating HIV strains; iii) viral repertoire-mediated immune escape within an infected host over time; iv) the presence of immunodominant non-protective epitopes; and (v) compromised host immune function (Barouch, Nature 2008; 455(7213):613-619; Fischer et al., Nat Med. 2007; 13(1):100-106).

Rapidly emerging insights into HIV immune control mechanisms coupled with the growing spectrum of well-characterized bNAbs provide an exciting opportunity to develop novel drug products for HIV prophylactics and therapeutic treatments.

Hence, there remains an urgent need to improve HIV vaccines and/or treatments.

The present invention fulfills such needs. The present invention has the particular advantages that it provides vaccines that elicit an HIV-specific CD8+ T cell response for control or prevention of HIV infection, in particular by providing a vaccine that supports the generation of CD8+ T cells that support and/or reproduce naturally observed treatment-free viremic control.

### SUMMARY

One aspect provided by the present disclosure is a composition comprising an RNA molecule, wherein the RNA molecule comprises an expression cassette encoding an immunogenic peptide, which peptide comprises at least two fragments, wherein each fragment comprises at least one epitope, wherein the epitope is derived from an amino acid sequence encoded by the human immunodeficiency virus (HIV) and wherein the epitope is comprised within or comprises a sequence selected from any one of SEQ ID NOs: 1 to 148 and 198 to 202 or a variant thereof.

In an embodiment, at least one of the fragments can comprise at least 2, 3, 4, 5, 6, 7, 8, 9, or 10 epitopes. In an embodiment, the epitopes in one fragment can be different from the epitopes in at least one other fragment or in all other fragments. In an embodiment, at least 60%, 65%, 70%, 75%, 80%, 85%, 90%, 95%, 97%, 98%, 99% or all of the fragments do not comprise any epitope that is comprised in another fragment.

In an embodiment, the sequences of the epitopes overlap in at least one of the fragments and/or the sequences of the epitopes do not overlap in at least one of the fragments.

In an embodiment, the immunogenic peptide comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 35 or 40 fragments in total. In an embodiment, the immunogenic peptide can comprise at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 66, 67, 68, 69, or 70 epitopes of those depicted in SEQ ID NOs: 1 to 148 and 198 to 202 in total. In an embodiment, each fragment can comprise the same or different number of epitopes.

In an embodiment, the epitopes can be combined within the expression cassette so as to minimize the length of the RNA molecule and/or can be ordered within the expression cassette so as to minimize HLA-II epitope of HIV.

In an embodiment, the epitopes can be derived from the gp41, gp120, p31, p24, nef, p51, protease, tat, rev, vif, vpr, vpx, or vpu protein of HIV. Preferably, at least one or all epitopes can be derived from the gp41, gp120, p31, p24, nef, p51, or protease protein of HIV. Preferably, at least one or all epitopes can be derived from the gp41, gp120 or nef protein of HIV, in particular at least one or all epitopes can be derived from the nef protein of HIV. In an embodiment, the peptide can comprise at least one epitope derived from each of the gp41, gp120 or nef proteins of HIV.

In an embodiment, at least one amino acid can separate the sequences of the non-overlapping epitopes in the peptide and/or in the fragment, and/or wherein at least one amino acid separates at least two fragments. Preferably, a linker can separate the sequences of at least two fragments.

In an embodiment, the two or more non-overlapping epitopes comprised within a fragment or two or more fragments are not flanked 5′ and 3′ by the consensus flanking amino acid sequence of the HIV amino acid sequence for the clade from which the epitopes were derived.

The term “clade” means in the context of the present invention a group of organisms, in particular viruses, such as HIV, that are monophyletic, i.e., composed of a common ancestor and all its lineal descendants on a phylogenetic tree. The phylogenetic tree can be constructed from the genomic sequences, e.g., by an alignment of the genomes of viruses and employing the neighbor-joining or maximum parsimony methods known in the art.

In an embodiment, the variant of an epitope can be a polymorph of the epitope, wherein the epitope is a consensus sequence from at least two different clones of HIV.

The term “polymorph” means in the context of the present invention a sequence variant of a consensus sequence of a part of a genome, for example, from a virus, which differs from the consensus sequence by at least one different nucleotide. For example, if the sequence atgacc is a consensus sequence, and at the second position a “g” is also known to occur, aggacc is a polymorph of the consensus sequence. Preferably, a polymorph will have at least 50%, 60%, 70%, 80%, 90%, 95% sequence identity to the consensus sequence.

In an embodiment, the variant can differ from the epitope in one, two, three, four or five amino acids.

In an embodiment, the composition can further comprise a second RNA molecule, wherein the second RNA molecule comprises a second expression cassette encoding a second immunogenic peptide, which peptide comprises at least two fragments, wherein each fragment comprises at least one epitope, wherein the epitope is derived from an amino acid sequence encoded by the human immunodeficiency virus (HIV) and wherein the epitope is comprised within or comprises a sequence selected from any one of SEQ ID NOs: 1 to 148 and 198 to 202 or a variant thereof.

In an embodiment, the immunogenic peptide comprises epitopes derived from one clade of HIV and the second immunogenic peptide comprises epitopes derived from another clade of HIV, preferably wherein the one clade is clade B and the another clade is clade C. In an embodiment, each immunogenic peptide comprises epitopes which are identical in both clades. In an embodiment, each immunogenic peptide comprises polymorphic epitopes from each respective clade.

In an embodiment, the epitope can be a T cell epitope and/or the epitope can be CD8 minimal epitope.

In an embodiment, the epitope can be 9 to 21 amino acids in length. Preferably, the epitope can be 9 or more amino acids in length. Preferably, the epitope can be 10 or more amino acids in length. Preferably, the epitope can be 11 or more amino acids in length. In some embodiments, the epitope can be 9 to 14 amino acids in length. Preferably, the epitope can be 9 to 13 amino acids in length. Preferably, the epitope can be 9 to 12 amino acids in length. Preferably, the epitope can be 9 to 11 amino acids in length. Preferably, the epitope can be 9 or 10 amino acids in length. Preferably, the epitope can be 9 amino acids in length. In an embodiment, the fragments can range from 9 to 21 amino acids in length.

In an embodiment, the immunogenic peptide can comprise 2, 3, 4, 5, 6, 7, 8, 9, 10, 11, 12, 13, 14, 15, 16, 17, 18, 19, or 20 or more fragments having an amino acid sequence selected from the group of amino acid sequences of SEQ ID NOs: 60, 103, 98, 149, 2, 150, 142, 86, 1, 141, 151, 152, 130, 153, 154, 155, 156, 157, 158, 4, 159, 160, 161, 162, 35, 28, 163, 164, 165, 166, 87, 167, 85, 125, 29, 168, 169, 57, 3, 170, 171, 172, 99, 131, 173, 174, 175, 104, 176, 61, 177, 178, 179, 5, 180, 181, 182, 140, 199, 203 and 204.

In an embodiment, the immunogenic peptide can comprise the amino acid sequence of a) SEQ ID NOs: 60, 103, 98, 149, 2, 150, 142, 86, 1, 141, 151, 152, 130, 153, 154, 155, 156, 157, 158, 4, 159, 160, 161, 162, 35, 28, 163, 164, 165, 166, 87, 167, and 85 or b) SEQ ID NOs: 60, 103, 98, 149, 2, 150, 142, 86, 1, 141, 151, 152, 199, 153, 154, 155, 156, 157, 158, 4, 159, 160, 161, 162, 35, 28, 203, 164, 165, 166, 87, 167, and 85. In an embodiment, the immunogenic peptide can comprise the amino acid sequence of a) SEQ ID NOs: 98, 125, 29, 85, 168, 35, 150, 149, 169, 87, 57, 3, 170, 171, 172, 99, 131, 173, 174, 175, 104, 176, 61, 177, 178, 179, 5, 180, 181, 1, 182, and 140 or b) SEQ ID NOs: 98, 125, 29, 85, 168, 35, 150, 149, 169, 87, 57, 3, 170, 171, 172, 99, 131, 173, 174, 175, 104, 176, 61, 177, 204, 179, 5, 180, 181, 1, 182, and 140. In an embodiment, the immunogenic peptide can comprise the amino acid sequence of SEQ ID NO: 183, 185, 187, 189, 205 or 208. The 5′ to 3′ order of the fragments in the peptides can be the order given or can be different from the order given.

In an embodiment, the first immunogenic peptide can comprise the amino acid sequence of a) SEQ ID NOs: 60, 103, 98, 149, 2, 150, 142, 86, 1, 141, 151, 152, 130, 153, 154, 155, 156, 157, 158, 4, 159, 160, 161, 162, 35, 28, 163, 164, 165, 166, 87, 167, and 85 or b) SEQ ID NOs: 60, 103, 98, 149, 2, 150, 142, 86, 1, 141, 151, 152, 199, 153, 154, 155, 156, 157, 158, 4, 159, 160, 161, 162, 35, 28, 203, 164, 165, 166, 87, 167, and 85, and the second immunogenic peptide can comprise the amino acid sequence of c) SEQ ID NOs: 98, 125, 29, 85, 168, 35, 150, 149, 169, 87, 57, 3, 170, 171, 172, 99, 131, 173, 174, 175, 104, 176, 61, 177, 178, 179, 5, 180, 181, 1, 182, and 140 or d) SEQ ID NOs: 98, 125, 29, 85, 168, 35, 150, 149, 169, 87, 57, 3, 170, 171, 172, 99, 131, 173, 174, 175, 104, 176, 61, 177, 204, 179, 5, 180, 181, 1, 182, and 140. In an embodiment, a) the first immunogenic peptide can comprise the amino acid sequence of SEQ ID NO: 183 or 205 and the second immunogenic peptide comprises the amino acid sequence of 185 or 208, b) the first immunogenic peptide comprises the amino acid sequence of SEQ ID NO: 187 and the second immunogenic peptide can comprise the amino acid sequence of SEQ ID NO: 189. In an embodiment, the first immunogenic peptide comprises the amino acid of SEQ ID 205 and the second immunogenic peptide comprises the amino acid of SEQ ID NO: 208.

In an embodiment, the RNA molecule can comprise the nucleotide sequence of SEQ ID NO: 184, 186, 188, 190, 206, 207, 209, 210 or 211 to 218.

In an embodiment, the epitope can be a non-protective epitope which can lead to CD8+ T cell protection collapse. Preferably, protection collapse can be T cell escape or T cell exhaustion or loss of CD4+ helper T cells.

In an embodiment, the peptide can further comprise sequences which enhance epitope presentation on the surface of a cell. Preferably, the cell can be an immune cell. Preferably, the immune cell can be an antigen presenting cell (APC). In some embodiments, the peptide can further comprise a signal peptide. In some embodiments, the peptide can further comprise a MHC class I trafficking signal (MITD).

In an embodiment, the peptide can further comprise a non-HIV HLA-II helper epitope. Preferably, the helper epitope can be the P2 and/or P16 amino acid sequences derived from the tetanus toxoid (TT) of Clostridium tetani.

The RNA molecule(s) can be linear or circular and/or can comprise a 5′ cap. Preferably, the 5′ cap can be a modified or artificial cap or a cap analog.

In an embodiment, the expression cassette(s) can further comprise a 5′ untranslated region (5′ UTR) and/or a 3′ untranslated region (3′ UTR). Preferably, the 5′ UTR can comprise the nucleotide sequence of SEQ ID NO: 71, or a nucleotide sequence having at least 99%, 98%, 97%, 96%, 95%, 90%, 85%, or 80% identity to the nucleotide sequence of SEQ ID NO: 71. Preferably, the 3′ UTR can comprise the nucleotide sequence of SEQ ID NO: 72, or a nucleotide sequence having at least 99%, 98%, 97%, 96%, 95%, 90%, 85%, or 80% identity to the nucleotide sequence of SEQ ID NO: 72.

In an embodiment, the expression cassette(s) can further comprise a poly A structure. Preferably, the poly A structure can be an interrupted poly A structure. Preferably, the poly A structure can comprise the nucleotide sequence of SEQ ID NO: 73, or a nucleotide sequence having at least 99%, 98%, 97%, 96%, 95%, 90%, 85%, or 80% identity to the nucleotide sequence of SEQ ID NO: 73.

In an embodiment, the RNA molecule(s) can be a replicable RNA molecule. Preferably, the replicable RNA molecule can further encode an RNA-dependent RNA polymerase (replicase) which is able to replicate the replicable RNA molecule or the replicable RNA molecule does not encode an RNA-dependent RNA polymerase (replicase).

Preferably, the composition can further comprise a non-replicable RNA molecule that encodes an RNA-dependent RNA polymerase (replicase) which is able to replicate the replicable RNA molecule.

In an embodiment, the RNA molecule(s) can be non-immunogenic. Preferably, the RNA molecule(s) can be made non-immunogenic by removal of double-stranded RNA.

In an embodiment, the RNA molecule(s) can comprise a nucleotide modification. Preferably, the modification can be the substitution of one or more U residues with pseudouridine, N1-methylpseudoruridine or 5-methyluridine.

Preferably, the one or more substituted U residues can be N1-methylpseudouridine. Preferably, at least 50%, at least 70%, at least 90% at least 99% or 100% of the U residues in the RNA molecule can be substituted.

In an embodiment, the RNA molecule(s) can be formulated in the composition with at least one lipid. Preferably, the RNA molecule(s) and at least one lipid can form particles. Preferably, the particles can be lipid nanoparticles (LNP), or lipoplexes (LPX) or liposomes.

In an embodiment, the particles can be nanoparticles, in which:
(i) the number of positive charges in the nanoparticles does not exceed the number of negative charges in the nanoparticles, and/or
(ii) the nanoparticles have a net negative charge, and/or
(iii) the zeta potential of the nanoparticles is 0 or less. Preferably, the charge ratio of positive charges to negative charges in the nanoparticles can be between 1:1 and 1:8, preferably between 1:1 and 1:4.

In an embodiment, the at least one lipid can be a cationic lipid. Preferably, the lipid can comprise a cationic head group. In some embodiments, the lipid can be a pH responsive lipid. In some embodiments, the at least one lipid can be a PEGylated-lipid. In some embodiments, the composition can further comprise at least one helper lipid.

Preferably, the helper lipid can be a neutral lipid.

In an embodiment, the at least one cationic lipid can comprise 1,2-di-O-octadecenyl-3-trimethylammonium propane (DOTMA), 1,2-dioleyloxy-3-dimethylaminopropane (DODMA), and/or 1,2-dioleoyl-3-trimethylammonium-propane (DOTAP). In some embodiments, the at least one helper lipid can comprise 1,2-di-(9Z-octadecenoyl)-sn-glycero-3-phosphoethanolamine (DOPE), cholesterol (Chol), 1,2-dioleoyl-sn-glycero-3-phosphocholine (DOPC), and/or 1,2-distearoyl-sn-glycero-3-phosphocholine (DSPC).

In an embodiment, the molar ratio of the at least one cationic lipid to the at least one helper lipid can be from 10:0 to 3:7, preferably 9:1 to 3:7, 4:1 to 1:2, 4:1 to 2:3, 7:3 to 1:1, or 2:1 to 1:1, preferably about 1:1.

In some embodiments, the nanoparticles can be lipoplexes comprising DODMA and DOPE in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2.

In some embodiments, the nanoparticles can be lipoplexes comprising DODMA and Cholesterol in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2; or the nanoparticles can be lipoplexes comprising DODMA and DSPC in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2; or the nanoparticles can be lipoplexes comprising DODMA:Cholesterol:DOPE:PEGcerC16 in a molar ratio of 40:48:10:2; or the nanoparticles can be lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2; or the nanoparticles can be lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2; or the nanoparticles can be lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In an embodiment, the particles can be LNPs, which are complexed with and/or encapsulate the RNA molecule. In an embodiment, the particles can be vesicles encapsulating the RNA molecule, preferably unilamellar liposomes. In some embodiments, the RNA molecule(s) can be formulated in a composition comprising a polyalkyleneimine, preferably is a polyalkyleneimine. Preferably, the molar ratio of the number of nitrogen atoms (N) in the polyalkyleneimine to the number of phosphor atoms (P) in the RNA molecules (N:P ratio) can be 2.0 to 15.0, preferably 6.0 to 12.0.

In an embodiment, the ionic strength of the composition can be 50 mM or less, preferably wherein the concentration of monovalent cationic ions can be 25 mM or less and the concentration of divalent cationic ions can be 20 μM or less.

In an embodiment, the particles formed can be polyplexes.

In an embodiment, the polyalkyleneimine can comprise the following general formula (I):

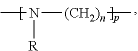

wherein
R is H, an acyl group or a group comprising the following general formula (II):

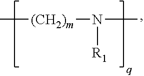

wherein R1 is H or a group comprising the following general formula (III):

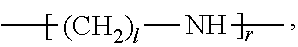

n, m, and l are independently selected from integers from 2 to 10; and
p, q, and r are integers, wherein the sum of p, q, and r is such that the average molecular weight of the polymer is 1.5-102 to 107 Da, preferably 5000 to 105 Da, more preferably 10000 to 40000 Da, more preferably 15000 to 30000 Da, even more preferably 20000 to 25000 Da.

In an embodiment, the polyalkyleneimine can comprise polyethylenimine and/or polypropylenimine, preferably polyethyleneimine. In some embodiments, at least 92% of the N atoms in the polyalkyleneimine can be protonatable.

In an embodiment, the composition can be a pharmaceutical composition. Preferably, the composition can further comprise a pharmaceutically acceptable carrier or excipient.

In an embodiment, the composition can be in the form of a dry powder; or the composition can be lyophilized; or the composition can be frozen. Preferably, the composition can have a temperature of −20° C. or lower.

In an embodiment, the composition can further comprise one or more additives, wherein the additives optionally are selected from the group consisting of buffering substances, saccharides, stabilizers, cryoprotectants, lyoprotectants, and chelating agents. Preferably, the buffering substances can comprise at least one selected from the group consisting of 4-(2-hydroxyethyl)-1-piperazineethanesulfonic acid (HEPES), 2-(N-morpholino)ethanesulfonic acid (MES), 3-morpholino-2-hydroxypropanesulfonic acid (MOPSO), acetic acid, acetate buffers and analogues, phosphoric acid and phosphate buffers, and citric acid and citrate buffers. Preferably, the saccharides can comprise at least one selected from the group consisting of monosaccharides, disaccharides, trisaccharides, oligosaccharides, and polysaccharides preferably from glucose, trehalose, and saccharose. Preferably, the cryoprotectants can comprise at least one selected from the group consisting of glycols, such as ethylene glycol, propylene glycol, and glycerol. Preferably, the chelating agent can comprise EDTA.

In an embodiment, the composition can be a vaccine.

A further aspect of the present disclosure is a medical preparation comprising a composition disclosed herein.

A further aspect of the present disclosure is a kit comprising one or more RNA molecules defined herein. Preferably, the RNA molecule(s) can be in the form of a dry powder composition. Preferably, the RNA molecule(s) can be lyophilized. Preferably, the kit can further comprise instructions for administering the RNA molecule(s).

A further aspect of the present disclosure is a method for preventing HIV infection in a subject, said method comprising administering a composition disclosed herein to the subject.

A further aspect of the present disclosure is a method for preventing HIV infection in a subject, said method comprising dissolving a dry powder composition disclosed herein into a suitable liquid pharmaceutical solution forming a solution for administration and administering the solution for administration to the subject.

A further aspect of the present disclosure is a method for treating HIV infection in a HIV-positive subject, said method comprising administering a composition disclosed herein to the subject.

A further aspect of the present disclosure is a method for treating HIV infection in a HIV-positive subject, said method comprising dissolving a dry powder composition disclosed herein into a suitable liquid pharmaceutical solution forming a solution for administration and administering the solution for administration to the subject.

In an embodiment of the methods disclosed herein, the severity of one or more symptoms of the HIV infection can be reduced. In an embodiment, the method can involve only a single administration of the composition or can comprise multiple administrations of the composition.

In an embodiment of the methods disclosed herein, the method can further comprise administering a booster dose of the composition.

In an embodiment of the methods disclosed herein, administering the composition can comprise intradermal, subcutaneous, or intramuscular administration, such as by intradermal, subcutaneous or intramuscular injection.

Preferably, the injection can be by use of a needle or is by use of a needleless injection device. In a embodiment of the methods disclosed herein, administering can comprise administration by intramuscular injection, preferably with a needle.

A further aspect of the present disclosure is a composition described herein for use in a method for preventing or treating HIV infection in a subject, said method comprising administering the composition to the subject. In an embodiment, the subject can be HIV-positive.

A further aspect of the present disclosure is a composition described herein for use in the manufacture of a medicament to prevent or treat HIV infection in a subject.

In an embodiment of the aspects of the disclosure regarding treatment or prevention, the method can comprise administering a first composition comprising a first RNA molecule, which first molecule comprises an expression cassette encoding an immunogenic peptide described herein, and wherein the method further comprises administering a second composition comprising a second RNA molecule, which second molecule comprises an expression cassette encoding a second immunogenic peptide described herein, which second immunogenic peptide is different from the first immunogenic peptide. The compositions can be administered concurrently or at different time points and by the same or different mode of administration. The method can further comprise additional rounds of administering the compositions.

In an embodiment of the aspects of the disclosure regarding treatment or prevention, the method can comprise administering a first RNA molecule and a second RNA molecule to a subject, wherein the first RNA molecule can comprise a nucleotide sequence encoding a first peptide, which first peptide comprises the amino acid sequences of a) SEQ ID NOs: 60, 103, 98, 149, 2, 150, 142, 86, 1, 141, 151, 152, 130, 153, 154, 155, 156, 157, 158, 4, 159, 160, 161, 162, 35, 28, 163, 164, 165, 166, 87, 167, and 85 or b) SEQ ID NOs: 60, 103, 98, 149, 2, 150, 142, 86, 1, 141, 151, 152, 199, 153, 154, 155, 156, 157, 158, 4, 159, 160, 161, 162, 35, 28, 203, 164, 165, 166, 87, 167, and 85; and wherein the second RNA molecule can comprise a nucleotide sequence encoding a second peptide, which second peptide comprises the amino acid sequences of c) SEQ ID NOs: 98, 125, 29, 85, 168, 35, 150, 149, 169, 87, 57, 3, 170, 171, 172, 99, 131, 173, 174, 175, 104, 176, 61, 177, 178, 179, 5, 180, 181, 1, 182, and 140 or d) SEQ ID NOs: 98, 125, 29, 85, 168, 35, 150, 149, 169, 87, 57, 3, 170, 171, 172, 99, 131, 173, 174, 175, 104, 176, 61, 177, 204, 179, 5, 180, 181, 1, 182, and 140. In an embodiment, the first RNA molecule can comprise the nucleotide sequence of SEQ ID NO: 184, 211, 212, 206 or 207 and the second RNA molecule comprises the nucleotide sequence of SEQ ID NO: 186, 213, 214, 209 or 210, or b) the first RNA molecule comprises the nucleotide sequence of SEQ ID NO: 188, 215 or 216, and the second RNA molecule can comprise the nucleotide sequence of SEQ ID NO: 190, 217 or 218. In an embodiment, the first RNA molecule comprises the nucleotide sequence of SEQ ID NO: 206 or 207, and the second RNA molecule comprises the nucleotide sequence of SEQ ID NO: 209 or 210. In an embodiment, the first and second RNA molecules can be administered at least 2 weeks apart. In an embodiment, the 5′ to 3′ order of the amino acid sequences in the first and/or second peptide can be the order given or the 5′ to 3′ order of the amino acid sequences in the first and/or second peptides can be different from the order given.

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, 1. Sambrook et al. eds., Cold Spring Harbor Laboratory Press, Cold Spring Harbor 1989).

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise.

The term “about” means approximately or nearly, and in the context of a numerical value or range set forth herein preferably means +/−10% of the numerical value or range recited or claimed.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless expressly specified otherwise, the term “comprising” is used in the context of the present document to indicate that further members may optionally be present in addition to the members of the list introduced by “comprising”. It is, however, contemplated as a specific embodiment of the present invention that the term “comprising” encompasses the possibility of no further members being present, i.e., for the purpose of this embodiment “comprising” is to be understood as having the meaning of “consisting of”.

Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present invention was not entitled to antedate such disclosure.

Terms such as “reduce” or “inhibit” as used herein means the ability to cause an overall decrease, preferably of 5% or greater, 10% or greater, 20% or greater, more preferably of 50% or greater, and most preferably 75% or greater, in the level. The term “inhibit” or similar phrases includes a complete or essentially complete inhibition, i.e., a reduction to zero or essentially to zero.

Terms such as “increase” or “enhance” preferably relate to an increase or enhancement by about at least 10%, preferably at least 20%, preferably at least 30%, more preferably at least 40%, more preferably at least 50%, even more preferably at least 80%, and most preferably at least 100%.

The term “net charge” refers to the charge on a whole object, such as a compound or particle.

An ion having an overall net positive charge is a cation, while an ion having an overall net negative charge is an anion. Thus, according to the invention, an anion is an ion with more electrons than protons, giving it a net negative charge; and a cation is an ion with fewer electrons than protons, giving it a net positive charge.

Terms as “charged”, “net charge”, “negatively charged” or “positively charged”, with reference to a given compound or particle, refer to the electric net charge of the given compound or particle when dissolved or suspended in water at pH 7.0.

The term “nucleic acid” according to the invention also comprises a chemical derivatization of a nucleic acid on a nucleotide base, on the sugar or on the phosphate, and nucleic acids containing non-natural nucleotides and nucleotide analogs. In some embodiments, the nucleic acid is a deoxyribonucleic acid (DNA) or a ribonucleic acid (RNA). In general, a nucleic acid molecule or a nucleic acid sequence refers to a nucleic acid which is preferably deoxyribonucleic acid (DNA) or ribonucleic acid (RNA). According to the invention, nucleic acids comprise genomic DNA, cDNA, mRNA, viral RNA, recombinantly prepared and chemically synthesized molecules. According to the invention, a nucleic acid may be in the form of a single-stranded or double-stranded and linear or covalently closed circular molecule.

According to the invention “nucleic acid sequence” refers to the sequence of nucleotides in a nucleic acid, e.g.; a ribonucleic acid (RNA) or a deoxyribonucleic acid (DNA). The term may refer to an entire nucleic acid molecule (such as to the single strand of an entire nucleic acid molecule) or to a part (e.g. a fragment) thereof.

According to the present invention, the term “RNA” or “RNA molecule” relates to a molecule which comprises ribonucleotide residues and which is preferably entirely or substantially composed of ribonucleotide residues. The term “ribonucleotide” relates to a nucleotide with a hydroxyl group at the 2′-position of a P-D-ribofuranosyl group. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of an RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally occurring RNAs.

According to the invention, RNA may be single-stranded or double-stranded. In some embodiments of the present invention, single-stranded RNA is preferred. The term “single-stranded RNA” generally refers to an RNA molecule to which no complementary nucleic acid molecule (typically no complementary RNA molecule) is associated. Single-stranded RNA may contain self-complementary sequences that allow parts of the RNA to fold back and to form secondary structure motifs including without limitation base pairs, stems, stem loops and bulges. Single-stranded RNA can exist as minus strand [(−) strand] or as plus strand [(+) strand]. The (+) strand is the strand that comprises or encodes genetic information. The genetic information may be for example a polynucleotide sequence encoding a protein. When the (+) strand RNA encodes a protein, the (+) strand may serve directly as template for translation (protein synthesis). The (−) strand is the complement of the (+) strand. In the case of double-stranded RNA, (+) strand and (−) strand are two separate RNA molecules, and both these RNA molecules associate with each other to form a double-stranded RNA (“duplex RNA”).

The term “stability” of RNA relates to the “half-life” of RNA. “Half-life” relates to the period of time which is needed to eliminate half of the activity, amount, or number of molecules. In the context of the present invention, the half-life of an RNA is indicative for the stability of said RNA. The half-life of RNA may influence the “duration of expression” of the RNA. It can be expected that RNA having a long half-life will be expressed for an extended time period.

The term “translation efficiency” relates to the amount of translation product provided by an RNA molecule within a particular period of time.

“Fragment”, with reference to a nucleic acid sequence, relates to a part of a nucleic acid sequence, i.e.; a sequence which represents the nucleic acid sequence shortened at the 5′- and/or 3′-end(s). Preferably, a fragment of a nucleic acid sequence comprises at least 80%, preferably at least 90%, 95%, 96%, 97%, 98%, or 99% of the nucleotide residues from said nucleic acid sequence. In the present invention those fragments of RNA molecules are preferred which retain RNA stability and/or translational efficiency.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises e.g. at least 1%, at least 2%, at least 3%, at least 4%, at least 5%, at least 10%, at least 20%, at least 30%, at least 40%, at least 50%, at least 60%, at least 70%, at least 80%, at least 90% of the amino acid residues from an amino acid sequence. As used herein with regard to the immunogenic peptide, a fragment is a sub-part of the peptide comprising one or more epitopes.

The term “variant” with respect to, for example, nucleic acid and amino acid sequences, according to the invention includes any variants, in particular mutants, viral strain variants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. With respect to nucleic acid molecules, the term “variant” includes degenerate nucleic acid sequences, wherein a degenerate nucleic acid according to the invention is a nucleic acid that differs from a reference nucleic acid in codon sequence due to the degeneracy of the genetic code. A species homolog is a nucleic acid or amino acid sequence with a different species of origin from that of a given nucleic acid or amino acid sequence. A virus homolog is a nucleic acid or amino acid sequence with a different virus of origin from that of a given nucleic acid or amino acid sequence.

Nucleic acid variants include single or multiple nucleotide deletions, additions, mutations, substitutions and/or insertions in comparison with the reference nucleic acid. Deletions include removal of one or more nucleotides from the reference nucleic acid. Addition variants comprise 5′- and/or 3-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations include abasic sites, crosslinked sites, and chemically altered or modified bases. Insertions include the addition of at least one nucleotide into the reference nucleic acid.

According to the invention, “nucleotide change” can refer to single or multiple nucleotide deletions, additions, mutations, substitutions and/or insertions in comparison with the reference nucleic acid. In some embodiments, a “nucleotide change” is selected from the group consisting of a deletion of a single nucleotide, the addition of a single nucleotide, the mutation of a single nucleotide, the substitution of a single nucleotide and/or the insertion of a single nucleotide, in comparison with the reference nucleic acid. According to the invention, a nucleic acid variant can comprise one or more nucleotide changes in comparison with the reference nucleic acid.

Variants of specific nucleic acid sequences preferably have at least one functional property of said specific sequences and preferably are functionally equivalent to said specific sequences, e.g., nucleic acid sequences exhibiting properties identical or similar to those of the specific nucleic acid sequences.

As described below, some embodiments of the present invention are characterized, inter alia, by nucleic acid sequences that are homologous to other nucleic acid sequences. These homologous sequences are variants of other nucleic acid sequences.

Preferably the degree of identity between a given nucleic acid sequence and a nucleic acid sequence which is a variant of said given nucleic acid sequence or between a given amino acid sequence and an amino acid sequence which is a variant of said given amino acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic or amino acid sequence.

“Sequence similarity” indicates the percentage of amino acids that either are identical or that represent conservative amino acid substitutions. “Sequence identity” between two polypeptide or nucleic acid sequences indicates the percentage of amino acids or nucleotides that are identical between the sequences.

The term “% identical” is intended to refer, in particular, to a percentage of nucleotides or amino acids which are identical in an optimal alignment between two sequences to be compared, with said percentage being purely statistical, and the differences between the two sequences may be randomly distributed over the entire length of the sequence and the sequence to be compared may comprise additions or deletions in comparison with the reference sequence, in order to obtain optimal alignment between two sequences. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2:482, with the aid of the local homology algorithm by Needleman and Wunsch, 1970, 1. Mol. Biol. 48:443, and with the aid of the similarity search algorithm by Pearson and Lipman, 1988, Proc. Natl Acad. Sci. USA 85:2444 or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions in which the sequences to be compared correspond, dividing this number by the number of positions compared and multiplying this result by 100.

For example, the BLAST program “BLAST 2 sequences” which is available on the website http://www.ncbi.nlm.nih.gov/blast/bl2seq/wblast2.cgi may be used.

A nucleic acid is “capable of hybridizing” or “hybridizes” to another nucleic acid if the two sequences are complementary with one another. A nucleic acid is “complementary” to another nucleic acid if the two sequences are capable of forming a stable duplex with one another. According to the invention, hybridization is preferably carried out under conditions which allow specific hybridization between polynucleotides (stringent conditions). Stringent conditions are described, for example, in Molecular Cloning: A Laboratory Manual, J. Sambrook et al., Editors, 2nd Edition, Cold Spring Harbor Laboratory press, Cold Spring Harbor, New York, 1989 or Current Protocols in Molecular Biology, F. M. Ausubel et al., Editors, John Wiley & Sons, Inc., New York and refer, for example, to hybridization at 65° C. in hybridization buffer (3.5×SSC, 0.02% Ficoll, 0.02% polyvinylpyrrolidone, 0.02% bovine serum albumin, 2.5 mM NaH2PO4 (pH 7), 0.5% SDS, 2 mM EDTA). SSC is 0.15 M sodium chloride/0.15 M sodium citrate, pH 7. After hybridization, the membrane to which the DNA has been transferred is washed, for example, in 2×SSC at room temperature and then in 0.1-0.5×SSC/0.1×SDS at temperatures of up to 68° C.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or “fully complementary” means that all the contiguous residues of a nucleic acid sequence will hydrogen bond with the same number of contiguous residues in a second nucleic acid sequence. Preferably, the degree of complementarity according to the invention is at least 70%, preferably at least 75%, preferably at least 80%, more preferably at least 85%, even more preferably at least 90% or most preferably at least 95%, 96%, 97%, 98% or 99%. Most preferably, the degree of complementarity according to the invention is 100%.

The term “derivative” comprises any chemical derivatization of a nucleic acid on a nucleotide base, on the sugar or on the phosphate. The term “derivative” also comprises nucleic acids which contain nucleotides and nucleotide analogs not occurring naturally. Preferably, a derivatization of a nucleic acid increases its stability.

A “nucleic acid sequence which is derived from a nucleic acid sequence” refers to a nucleic acid which is a variant of the nucleic acid from which it is derived. Preferably, a sequence which is a variant with respect to a specific sequence, when it replaces the specific sequence in an RNA molecule retains RNA stability and/or translational efficiency.

“nt” is an abbreviation for nucleotide; or for nucleotides, preferably consecutive nucleotides in a nucleic acid molecule.

According to the invention, the term “codon” refers to a base triplet in a coding nucleic acid that specifies which amino acid will be added next during protein synthesis at the ribosome.

The terms “transcription” and “transcribing” relate to a process during which a nucleic acid molecule with a particular nucleic acid sequence (the “nucleic acid template”) is read by an RNA polymerase so that the RNA polymerase produces a single-stranded RNA molecule. During transcription, the genetic information in a nucleic acid template is transcribed. The nucleic acid template may be DNA; however, e.g.; in the case of transcription from an alphaviral nucleic acid template, the template is typically RNA. Subsequently, the transcribed RNA may be translated into protein. According to the present invention, the term “transcription” comprises “in vitro transcription”, wherein the term “in vitro transcription” relates to a process wherein RNA, in particular mRNA, is in vitro synthesized in a cell-free system. Preferably, cloning vectors are applied for the generation of transcripts. These cloning vectors are generally designated as transcription vectors and are according to the present invention encompassed by the term “vector”. The cloning vectors are preferably plasmids. According to the present invention, RNA preferably is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

The single-stranded nucleic acid molecule produced during transcription typically has a nucleic acid sequence that is the complementary sequence of the template.

According to the invention, the terms “template” or “nucleic acid template” or “template nucleic acid” generally refer to a nucleic acid sequence that may be replicated or transcribed.

“Nucleic acid sequence transcribed from a nucleic acid sequence” and similar terms refer to a nucleic acid sequence, where appropriate as part of a complete RNA molecule, which is a transcription product of a template nucleic acid sequence. Typically, the transcribed nucleic acid sequence is a single-stranded RNA molecule.

“3′ end of a nucleic acid” refers according to the invention to that end which has a free hydroxy group. In a diagrammatic representation of double-stranded nucleic acids, in particular DNA, the 3′ end is always on the right-hand side. “5′ end of a nucleic acid” refers according to the invention to that end which has a free phosphate group. In a diagrammatic representation of double-strand nucleic acids, in particular DNA, the 5′ end is always on the left-hand side.

| | | |
|---|---|---|
| 5′ end | 5′--P-NNNNNNN-OH-3′ | 3′ end |
| | 3′-HO-NNNNNNN-P--5′ | |

“Upstream” describes the relative positioning of a first element of a nucleic acid molecule with respect to a second element of that nucleic acid molecule, wherein both elements are comprised in the same nucleic acid molecule, and wherein the first element is located nearer to the 5′ end of the nucleic acid molecule than the second element of that nucleic acid molecule. The second element is then said to be “downstream” of the first element of that nucleic acid molecule. An element that is located “upstream” of a second element can be synonymously referred to as being located “5′” of that second element. For a double-stranded nucleic acid molecule, indications like “upstream” and “downstream” are given with respect to the (+) strand.

According to the invention, “functional linkage” or “functionally linked” relates to a connection within a functional relationship. A nucleic acid is “functionally linked” if it is functionally related to another nucleic acid sequence. For example, a promoter is functionally linked to a coding sequence if it influences transcription of said coding sequence. Functionally linked nucleic acids are typically adjacent to one another, where appropriate separated by further nucleic acid sequences, and, in particular embodiments, are transcribed by RNA polymerase to give a single RNA molecule (common transcript).

In particular embodiments, a nucleic acid is functionally linked according to the invention to expression control sequences which may be homologous or heterologous with respect to the nucleic acid.

The term “expression control sequence” comprises according to the invention promoters, ribosome-binding sequences and other control elements which control transcription of a gene or translation of the derived RNA. In particular embodiments of the invention, the expression control sequences can be regulated. The precise structure of expression control sequences may vary depending on the species or cell type but usually includes 5′-untranscribed and 5′- and 3′-untranslated sequences involved in initiating transcription and translation, respectively. More specifically, 5′-untranscribed expression control sequences include a promoter region which encompasses a promoter sequence for transcription control of the functionally linked gene. Expression control sequences may also include enhancer sequences or upstream activator sequences. An expression control sequence of a DNA molecule usually includes 5′-untranscribed and 5′- and 3′-untranslated sequences such as TATA box, capping sequence, CAAT sequence and the like. An expression control sequence of alphaviral RNA may include a subgenomic promoter and/or one or more conserved sequence element(s). A specific expression control sequence according to the present invention is a subgenomic promoter of an alphavirus, as described herein.

The nucleic acid sequences specified herein, in particular transcribable and coding nucleic acid sequences, may be combined with any expression control sequences, in particular promoters, which may be homologous or heterologous to said nucleic acid sequences, with the term “homologous” referring to the fact that a nucleic acid sequence is also functionally linked naturally to the expression control sequence, and the term “heterologous” referring to the fact that a nucleic acid sequence is not naturally functionally linked to the expression control sequence.

A transcribable nucleic acid sequence, in particular a nucleic acid sequence coding for a peptide or protein, and an expression control sequence are “functionally” linked to one another, if they are covalently linked to one another in such a way that transcription or expression of the transcribable and in particular coding nucleic acid sequence is under the control or under the influence of the expression control sequence. If the nucleic acid sequence is to be translated into a functional peptide or protein, induction of an expression control sequence functionally linked to the coding sequence results in transcription of said coding sequence, without causing a frame shift in the coding sequence or the coding sequence being unable to be translated into the desired peptide or protein.

The term “promoter” or “promoter region” refers to a nucleic acid sequence which controls synthesis of a transcript, e.g. a transcript comprising a coding sequence, by providing a recognition and binding site for RNA polymerase. The promoter region may include further recognition or binding sites for further factors involved in regulating transcription of said gene. A promoter may control transcription of a prokaryotic or eukaryotic gene. A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor. A specific promoter according to the present invention is a subgenomic promoter, e.g., of an alphavirus, as described herein. Other specific promoters are genomic plus-strand or negative-strand promoters, e.g., of an alphavirus.

The term “core promoter” refers to a nucleic acid sequence that is comprised by the promoter. The core promoter is typically the minimal portion of the promoter required to properly initiate transcription. The core promoter typically includes the transcription start site and a binding site for RNA polymerase.

A “polymerase” generally refers to a molecular entity capable of catalyzing the synthesis of a polymeric molecule from monomeric building blocks. An “RNA polymerase” is a molecular entity capable of catalyzing the synthesis of an RNA molecule from ribonucleotide building blocks. A “DNA polymerase” is a molecular entity capable of catalyzing the synthesis of a DNA molecule from deoxy ribonucleotide building blocks. For the case of DNA polymerases and RNA polymerases, the molecular entity is typically a protein or an assembly or complex of multiple proteins. Typically, a DNA polymerase synthesizes a DNA molecule based on a template nucleic acid, which is typically a DNA molecule. Typically, an RNA polymerase synthesizes an RNA molecule based on a template nucleic acid, which is either a DNA molecule (in that case the RNA polymerase is a DNA-dependent RNA polymerase, DdRP), or is an RNA molecule (in that case the RNA polymerase is an RNA-dependent RNA polymerase, RdRP).

An “RNA-dependent RNA polymerase” or “RdRP”, is an enzyme that catalyzes the transcription of RNA from an RNA template. In the case of alphaviral RNA-dependent RNA polymerase, sequential synthesis of (−) strand complement of genomic RNA and of (+) strand genomic RNA leads to RNA replication. RNA-dependent RNA polymerase is thus synonymously referred to as “RNA replicase” or simply “replicase”. In nature, RNA-dependent RNA polymerases are typically encoded by all RNA viruses except retroviruses. Typical representatives of viruses encoding an RNA-dependent RNA polymerase are alphaviruses.

According to the present invention, “RNA replication” generally refers to an RNA molecule synthesized based on the nucleotide sequence of a given RNA molecule (template RNA molecule). The RNA molecule that is synthesized may be, e.g., identical or complementary to the template RNA molecule. In general, RNA replication may occur via synthesis of a DNA intermediate, or may occur directly by RNA-dependent RNA replication mediated by an RNA-dependent RNA polymerase (RdRP). In the case of alphaviruses, RNA replication does not occur via a DNA intermediate, but is mediated by a RNA-dependent RNA polymerase (RdRP): a template RNA strand (first RNA strand)—or a part thereof—serves as template for the synthesis of a second RNA strand that is complementary to the first RNA strand or to a part thereof. The second RNA strand—or a part thereof—may in turn optionally serve as a template for synthesis of a third RNA strand that is complementary to the second RNA strand or to a part thereof. Thereby, the third RNA strand is identical to the first RNA strand or to a part thereof. Thus, RNA-dependent RNA polymerase is capable of directly synthesizing a complementary RNA strand of a template, and of indirectly synthesizing an identical RNA strand (via a complementary intermediate strand).

According to the invention, the term “template RNA” refers to RNA that can be transcribed or replicated by an RNA-dependent RNA polymerase.

According to the invention, the term “gene” refers to a particular nucleic acid sequence which is responsible for producing one or more cellular products and/or for achieving one or more intercellular or intracellular functions. More specifically, said term relates to a nucleic acid section (typically DNA; but RNA in the case of RNA viruses) which comprises a nucleic acid coding for a specific protein or a functional or structural RNA molecule.

An “isolated molecule” as used herein, is intended to refer to a molecule which is substantially free of other molecules such as other cellular material. The term “isolated nucleic acid” means according to the invention that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant techniques.

The term “vector” is used here in its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, virus genomes, and fractions thereof.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the invention, the term “expression” is used in its most general meaning and comprises production of RNA and/or protein. It also comprises partial expression of nucleic acids. Furthermore, expression may be transient or stable. With respect to RNA, the term “expression” or “translation” relates to the process in the ribosomes of a cell by which a strand of coding RNA (e.g. messenger RNA) directs the assembly of a sequence of amino acids to make a peptide or protein.

According to the invention, the term “mRNA” means “messenger-RNA” and relates to a transcript which is typically generated by using a DNA template and encodes a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to the skilled person. For example, there is a variety of in vitro transcription kits commercially available. According to the invention, mRNA may be modified by stabilizing modifications and capping.

According to the invention, the terms “poly(A) sequence” or “poly(A) tail” refer to an uninterrupted or interrupted sequence of adenylate residues which is typically located at the 3′ end of an RNA molecule. An uninterrupted sequence is characterized by consecutive adenylate residues. In nature, an uninterrupted poly(A) sequence is typical. While a poly(A) sequence is normally not encoded in eukaryotic DNA, but is attached during eukaryotic transcription in the cell nucleus to the free 3′ end of the RNA by a template-independent RNA polymerase after transcription, the present invention encompasses poly(A) sequences encoded by DNA.

According to the invention, the term “primary structure”, with reference to a nucleic acid molecule, refers to the linear sequence of nucleotide monomers.

According to the invention, the term “secondary structure”, with reference to a nucleic acid molecule, refers to a two-dimensional representation of a nucleic acid molecule that reflects base pairings; e.g.; in the case of a single-stranded RNA molecule particularly intramolecular base pairings. Although each RNA molecule has only a single polynucleotide chain, the molecule is typically characterized by regions of (intramolecular) base pairs. According to the invention, the term “secondary structure” comprises structural motifs including without limitation base pairs, stems, stem loops, bulges, loops such as interior loops and multi-branch loops. The secondary structure of a nucleic acid molecule can be represented by a two-dimensional drawing (planar graph), showing base pairings (for further details on secondary structure of RNA molecules, see Auber et al., 2006; J. Graph Algorithms Appl. 10:329-351). As described herein, the secondary structure of certain RNA molecules is relevant in the context of the present invention.

According to the invention, secondary structure of a nucleic acid molecule, particularly of a single-stranded RNA molecule, is determined by prediction using the web server for RNA secondary structure prediction (http://rna.urmc.rochester.edu/RNAstructureWeb/Servers/Predictl/Predictl.html). Preferably, according to the invention, “secondary structure”, with reference to a nucleic acid molecule, specifically refers to the secondary structure determined by said prediction. The prediction may also be performed or confirmed using MFOLD structure prediction (http://unafold.rna.albany.edu/?q=mfold).

According to the invention, a “base pair” is a structural motif of a secondary structure wherein two nucleotide bases associate with each other through hydrogen bonds between donor and acceptor sites on the bases. The complementary bases, A:U and G:C, form stable base pairs through hydrogen bonds between donor and acceptor sites on the bases; the A:U and G:C base pairs are called Watson-Crick base pairs. A weaker base pair (called Wobble base pair) is formed by the bases G and U (G:U). The base pairs A:U and G:C are called canonical base pairs. Other base pairs like G:U (which occurs fairly often in RNA) and other rare base-pairs (e.g. A:C; U:U) are called non-canonical base pairs.

According to the invention, “nucleotide pairing” refers to two nucleotides that associate with each other so that their bases form a base pair (canonical or non-canonical base pair, preferably canonical base pair, most preferably Watson-Crick base pair).

According to the invention, the terms “stem loop” or “hairpin” or “hairpin loop”, with reference to a nucleic acid molecule, all interchangeably refer to a particular secondary structure of a nucleic acid molecule, typically a single-stranded nucleic acid molecule, such as single-stranded RNA. The particular secondary structure represented by the stem loop consists of a consecutive nucleic acid sequence comprising a stem and a (terminal) loop, also called hairpin loop, wherein the stem is formed by two neighbored entirely or partially complementary sequence elements; which are separated by a short sequence (e.g. 3-10 nucleotides), which forms the loop of the stem-loop structure. The two neighbored entirely or partially complementary sequences may be defined as, e.g., stem loop elements stem 1 and stem 2. The stem loop is formed when these two neighbored entirely or partially reverse complementary sequences, e.g. stem loop elements stem 1 and stem 2, form base-pairs with each other, leading to a double stranded nucleic acid sequence comprising an unpaired loop at its terminal ending formed by the short sequence located between stem loop elements stem 1 and stem 2. Thus, a stem loop comprises two stems (stem 1 and stem 2), which—at the level of secondary structure of the nucleic acid molecule—form base pairs with each other, and which—at the level of the primary structure of the nucleic acid molecule—are separated by a short sequence that is not part of stem 1 or stem 2. For illustration, a two-dimensional representation of the stem loop resembles a lollipop-shaped structure. The formation of a stem-loop structure requires the presence of a sequence that can fold back on itself to form a paired double strand; the paired double strand is formed by stem 1 and stem 2. The stability of paired stem loop elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). According to the present invention, the optimal loop length is 3-10 nucleotides, more preferably 4 to 7, nucleotides, such as 4 nucleotides, 5 nucleotides, 6 nucleotides or 7 nucleotides. If a given nucleic acid sequence is characterized by a stem loop, the respective complementary nucleic acid sequence is typically also characterized by a stem loop. A stem loop is typically formed by single-stranded RNA molecules. For example, several stem loops are present in the 5′ replication recognition sequence of alphaviral genomic RNA.

According to the invention, “disruption” or “disrupt”, with reference to a specific secondary structure of a nucleic acid molecule (e.g., a stem loop) means that the specific secondary structure is absent or altered. Typically, a secondary structure may be disrupted as a consequence of a change of at least one nucleotide that is part of the secondary structure. For example, a stem loop may be disrupted by change of one or more nucleotides that form the stem, so that nucleotide pairing is not possible.

According to the invention, the term “tertiary structure”, with reference to a nucleic acid molecule, refers to the three-dimensional structure of a nucleic acid molecule, as defined by the atomic coordinates.

In some embodiments of the present disclosure, an RNA molecule is “replicon RNA” or “replicon RNA molecule” or simply a “replicon”, in particular “self-replicating RNA” or “self-amplifying RNA” or “replicable RNA molecule”. A replicon RNA molecule is an RNA that is able to be replicated by an RNA-dependent RNA polymerase (replicase) by virtue of comprising nucleotide sequences that can be recognized by the replicase such that the RNA is replicated. The replicon does not necessarily encode the replicase, such that replicons can be replicated in cis (by the encoded replicase; also called a “cis-replicon”) or in trans (by a replicase provided in another manner, e.g., a separate replicase encoding nucleic acid, such as an mRNA; also called “trans-replicon”).

In certain embodiments, the replicon or self-replicating RNA is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Jose et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

According to the invention, a nucleic acid such as RNA, e.g., rRNA, may encode a peptide or protein. Accordingly, a transcribable nucleic acid sequence or a transcript thereof may contain an open reading frame (ORF) encoding a peptide or protein.

According to the invention, the term “nucleic acid encoding a peptide or protein” means that the nucleic acid, if present in the appropriate environment, preferably within a cell, can direct the assembly of amino acids to produce the peptide or protein during the process of translation. Preferably, coding RNA according to the invention is able to interact with the cellular translation machinery allowing translation of the coding RNA to yield a peptide or protein.

According to the invention, the term “peptide” comprises oligo- and polypeptides and refers to substances which comprise two or more, preferably 3 or more, preferably 4 or more, preferably 6 or more, preferably 8 or more, preferably 10 or more, preferably 13 or more, preferably 16 or more, preferably 20 or more, and up to preferably 50, preferably 100 or preferably 150, consecutive amino acids linked to one another via peptide bonds. The term “protein” refers to large peptides, preferably peptides having at least 151 amino acids, but the terms “peptide” and “protein” are used herein usually as synonyms.

The terms “peptide” and “protein” comprise, according to the invention, substances which contain not only amino acid components but also non-amino acid components such as sugars and phosphate structures, and also comprise substances containing bonds such as ester, thioether or disulfide bonds.

According to the invention, the terms “initiation codon” and “start codon” synonymously refer to a codon (base triplet) of an RNA molecule that is potentially the first codon that is translated by a ribosome. Such codon typically encodes the amino acid methionine in eukaryotes and a modified methionine in prokaryotes. The most common initiation codon in eukaryotes and prokaryotes is AUG. Unless specifically stated herein that an initiation codon other than AUG is meant, the terms “initiation codon” and “start codon”, with reference to an RNA molecule, refer to the codon AUG. According to the invention, the terms “initiation codon” and “start codon” are also used to refer to a corresponding base triplet of a deoxyribonucleic acid, namely the base triplet encoding the initiation codon of an RNA. If the initiation codon of messenger RNA is AUG, the base triplet encoding the AUG is ATG. According to the invention, the terms “initiation codon” and “start codon” preferably refer to a functional initiation codon or start codon, i.e., to an initiation codon or start codon that is used or would be used as a codon by a ribosome to start translation. There may be AUG codons in an RNA molecule that are not used as codons by a ribosome to start translation, e.g., due to a short distance of the codons to the cap. These codons are not encompassed by the term functional initiation codon or start codon.

The following provides specific and/or preferred variants of the individual features of the invention. The present invention also contemplates as particularly preferred embodiments those embodiments, which are generated by combining two or more of the specific and/or preferred variants described for two or more of the features of the present invention.

“Isolated” means altered or removed from the natural state. For example, a cell, a nucleic acid or a peptide naturally present in a living animal is not “isolated”, but the same cell, nucleic acid or peptide partially or completely separated from the coexisting materials of its natural state is “isolated”. Preferably an isolated cell, nucleic acid or peptide exists in a purified or substantially purified state. An isolated cell or cell population preferably does exist without cells of a different cell type, e.g., an isolated T cell exists without other blood cells such as dendritic cells. Preferably an isolated cell does exist only with isogeneic cells of the same cell type.

The term “isogeneic” is used to describe a cell that has the same genetic information as another cell or cell population.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues.

The term “heterologous” is used to describe something consisting of multiple different elements. As an example, the transfer of one individual's bone marrow into a different individual constitutes a heterologous transplant. A heterologous gene is a gene derived from a source other than the subject.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “immune effector cell” or “immunoreactive cell” in the context of the present invention relates to a cell which exerts effector functions during an immune reaction.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis.

Preferably, the effector functions in the context of the present invention are T cell mediated effector functions. Such functions comprise in the case of a helper T cell (CD4⁺ T cell) the release of cytokines and/or the activation of CD8+ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

An “immune effector cell” in one embodiment is capable of binding an antigen such as an antigen presented in the context of MHC (the term MHC includes HLA) on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4+ and/or CD8+ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

Preferably, an “immune effector cell” recognizes an antigen with some degree of specificity, in particular if presented in the context of MHC or present on the surface of diseased cells such as cancer cells. Preferably, said recognition enables the cell that recognizes an antigen to be responsive or reactive. If the cell is a helper T cell (CD4⁺ T cell) such responsiveness or reactivity may involve the release of cytokines and/or the activation of CD8+ lymphocytes (CTLs) and/or B cells. If the cell is a CTL such responsiveness or reactivity may involve the elimination of cells, i.e., cells characterized by expression of an antigen, for example, via apoptosis or perforin-mediated cell lysis. According to the invention, CTL responsiveness may include sustained calcium flux, cell division, production of cytokines such as IFN-γ and TNF-α, up-regulation of activation markers such as CD44 and CD69, and specific cytolytic killing of antigen expressing target cells. CTL responsiveness may also be determined using an artificial reporter that accurately indicates CTL responsiveness. Such CTL that recognizes an antigen and are responsive or reactive are also termed “antigen-responsive CTL” herein.

A “lymphoid cell” is a cell which, optionally after suitable modification, e.g., after transfer of an antigen receptor such as a TCR or a CAR, is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell which can be modified to express an antigen receptor on the cell surface. In one embodiment, the lymphoid cell lacks endogenous expression of a T cell receptor.

The terms “T cell” and “T lymphocyte” are used interchangeably herein and include T helper cells (CD4⁺ T cells) and cytotoxic T cells (CTLs, CD8⁺ T cells) which comprise cytolytic T cells. The term “antigen-specific T cell” or similar terms relate to a T cell which recognizes the antigen to which the T cell is targeted and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptors (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4⁺ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8⁺ T cells since they express the CD8 glycoprotein on their surface. These cells recognize their targets by binding to antigen associated with MHC class I, which is present on the surface of nearly every cell of the body.

“Regulatory T cells” or “Tregs” are a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FoxP3, and CD25.

As used herein, the term “naïve T cell” refers to mature T cells that, unlike activated or memory T cells, have not encountered their cognate antigen within the periphery. Naïve T cells are commonly characterized by the surface expression of L-selectin (CD62L), the absence of the activation markers CD25, CD44 or CD69 and the absence of the memory CD45RO isoform.

As used herein, the term “memory T cells” refers to a subgroup or subpopulation of T cells that have previously encountered and responded to their cognate antigen. At a second encounter with the antigen, memory T cells can reproduce to mount a faster and stronger immune response than the first time the immune system responded to the antigen. Memory T cells may be either CD4⁺ or CD8⁺ and usually express CD45RO.

According to the invention, the term “T cell” also includes a cell which can mature into a T cell with suitable stimulation.

A majority of T cells have a T cell receptor (TCR) existing as a complex of several proteins. The actual T cell receptor is composed of two separate peptide chains, which are produced from the independent T cell receptor alpha and beta (TCRα and TCRβ) genes and are called α- and β-TCR chains. γδ T cells (gamma delta T cells) represent a small subset of T cells that possess a distinct T cell receptor (TCR) on their surface. However, in γδ T cells, the TCR is made up of one γ-chain and one δ-chain. This group of T cells is much less common (2% of total T cells) than the αβ T cells.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development they become double-positive thymocytes (CD4⁺CD8⁺), and finally mature to single-positive (CD4⁺CD8⁻ or CD4⁻CD8⁺) thymocytes that are then released from the thymus to peripheral tissues.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

The following provides specific and/or preferred variants of the individual features of the invention. The present invention also contemplates as particularly preferred embodiments those embodiments, which are generated by combining two or more of the specific and/or preferred variants described for two or more of the features of the present invention.

### Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In some embodiments, at least one or all of the first, second, third, and fourth RNA molecule is a modified RNA molecule.

### Modified RNA

In some embodiments, the RNA or RNA molecules described herein are modified RNA. In some embodiments, the modified RNA contains at least one functional analog of A, C, G and/or U.

In an embodiment, the RNA described herein may have modified nucleotides/nucleosides/backbone modifications. The term “RNA modification” as used herein may refer to chemical modifications comprising backbone modifications as well as sugar modifications or base modifications.

In this context, a modified RNA molecule as defined herein may contain nucleotide analogues/modifications, e.g., backbone modifications, sugar modifications or base modifications. A backbone modification in connection with the present disclosure is a modification, in which phosphates of the backbone of the nucleotides contained in an RNA molecule as defined herein are chemically modified. A sugar modification in connection with the present disclosure is a chemical modification of the sugar of the nucleotides of the RNA molecule as defined herein. Furthermore, a base modification in connection with the present disclosure is a chemical modification of the base moiety of the nucleotides of the RNA molecule. In this context, nucleotide analogues or modifications are preferably selected from nucleotide analogues, which are applicable for transcription and/or translation.

Sugar Modifications: The modified nucleosides and nucleotides, which may be incorporated into a modified RNA molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R=H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

Backbone Modifications: The phosphate backbone may further be modified in the modified nucleosides and nucleotides, which may be incorporated into a modified RNA molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene—phosphonates).

Base Modifications: The modified nucleosides and nucleotides, which may be incorporated into a modified RNA molecule as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particular embodiments of the present disclosure, the nucleotide analogues/modifications are selected from base modifications, which are preferably selected from 2-amino-6-chloropurineriboside-5′-triphosphate, 2-aminopurine-riboside-5′-triphosphate; 2-aminoadenosine-5′-triphosphate, 2′-amino-2′-deoxy-cytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thio-uridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-tri-phosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deaza-adenosine-5′-triphosphate, 7-deazaguanosine-5′-triphosphate, 8-azaadenosine-5′-triphosphate, 8-azidoadenosine-5′-tri phosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, N6-methylguanosine-5′-triphosphate, pseudo-uridine-5′-triphosphate, or puromycin-5′-triphosphate, xanthosine-5′-triphosphate. Particular preference may be given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thiouridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydro-pseudouridine, 2-thio-dihydrouridine, 2-thio-dihydropseudouridine, 2-methoxy-uridine, 2-methoxy-4-thio-uridine, 4-methoxy-pseudouridine, and 4-methoxy-2-thio-pseudouridine. In a preferred embodiment the functional analog replacing uridine is N1-methyl-pseudouridine (m1ψ).

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methyl-thio-N6-threonylcarbamoyladenosine, N6,N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In further embodiments, a modified RNA may comprise nucleoside modifications selected from 6-aza-cytidine, 2-thio-cytidine, a-thio-cytidine, pseudo-iso-cytidine, 5-aminoallyl-uridine, 5-iodo-uridine, Nl-methyl-pseudouridine, 5,6-dihydrouridine, a-thio-uridine, 4-thio-uridine, 6-aza-uridine, 5-hydroxy-uridine, deoxy-thymidine, 5-methyl-uridine, pyrrolo-cytidine, inosine, a-thio-guanosine, 6-methyl-guanosine, 5-methyl-cytidine, 8-oxo-guanosine, 7-deaza-guanosine, Nl-methyl-adenosine, 2-amino-6-chloro-purine, N6-methyl-2-amino-purine, pseudo-iso-cytidine, 6-chloro-purine, N6-methyl-adenosine, a-thio-adenosine, 8-azido-adenosine, 7-deaza-adenosine.

In certain preferred embodiments, the RNA comprises a modified nucleoside in place of at least one (e.g., every) uridine.

The term “uracil,” as used herein, describes one of the nucleobases that can occur in the nucleic acid of RNA. The structure of uracil is:

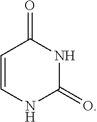

The term “uridine,” as used herein, describes one of the nucleosides that can occur in RNA. The structure of uridine is:

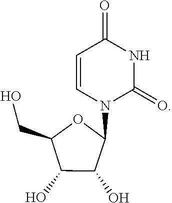 [SMILES: O=c1ccn([C@@H]2O[C@H](CO)[C@@H](O)[C@H]2O)c(=O)[nH]1]

UTP (uridine 5′-triphosphate) has the following structure:

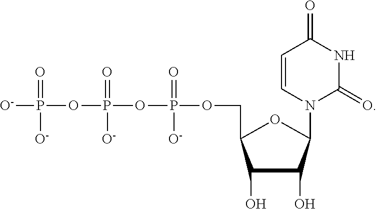

Pseudo-UTP (pseudouridine 5′-triphosphate) has the following structure:

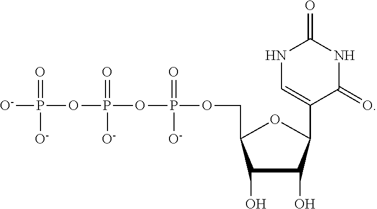

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

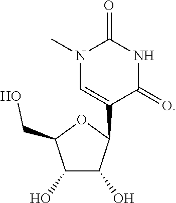 [SMILES: Cn1cc([C@@H]2O[C@H](CO)[C@@H](O)[C@H]2O)c(=O)[nH]c1=O]

N1-methyl-pseudo-UTP has the following structure:

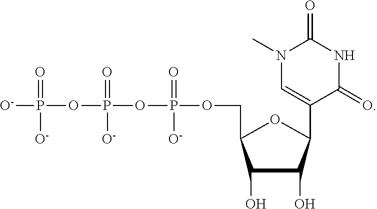

Another exemplary modified nucleoside is 5-methyl-uridine (m5U), which has the structure:

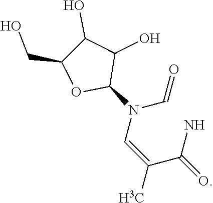 [SMILES: [3CH3]/C(=C/N(C=O)[C@H]1O[C@@H](CO)C(O)C1O)C(N)=O]

In certain preferred embodiments, one or more uridine in the RNA described herein is replaced by a modified nucleoside. In some embodiments, the modified nucleoside is a modified uridine.

In certain preferred embodiments, RNA comprises a modified nucleoside in place of at least one uridine. In some embodiments, RNA comprises a modified nucleoside in place of each uridine.

In certain preferred embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In certain preferred embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm^{S}U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm^{S}U), 5-methoxycarbonylmethyl-2-thio-uridine (mcm⁵s²U), 5-aminomethyl-2-thio-uridine (τm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψn), 2-thio-2′-O-methyl-uridine (s²Um), 5-methoxycarbonylmethyl-2′-O-methyl-uridine (mcm^{S}Um), 5-carbamoylmethyl-2′-O-methyl-uridine (ncm^{S}Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In an embodiment, the RNA comprises other modified nucleosides or comprises further modified nucleosides, e.g., modified cytidine such as those described above. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In an embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

### Cap

In some embodiments, an RNA or RNA molecule described herein may optionally comprise a 5′ cap, 5′ UTR, a coding sequence, a 3′ UTR and/or a poly-(A) tail. In some embodiments the coding sequence or open reading frame may be optimized with respect to the codon usage.

“RNA which comprises a 5′-cap” or “RNA which is provided with a 5′-cap” or “RNA which is modified with a 5′-cap” or “capped RNA” refers to RNA which comprises a 5′-cap. For example, providing an RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In the present disclosure, a natural occurring cap is typically selected from the group consisting of a non-methylated cap dinucleotide (G(5′)ppp(5′)N; also termed GpppN) and a methylated cap dinucleotide ((^{m7}G(5′)ppp(5′)N; also termed m⁷GpppN). m⁷GpppN (wherein N is G) is represented by the following formula:

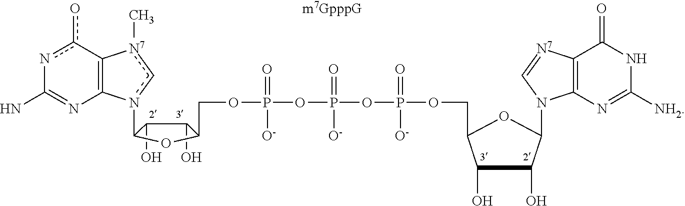

Capped RNA of the present disclosure can be prepared in vitro, and therefore, does not depend on a capping machinery in a host cell. Co-transcriptional capping works by transcribing in vitro a DNA template with either a bacterial or bacteriophage nucleic acid polymerase in the presence of all four ribonucleoside triphosphates or functional analogs thereof and a capping reagent such as m⁷G(5′)ppp(5′)G (also called m⁷GpppG). The nucleic acid polymerase initiates transcription with a nucleophilic attack by the 3′-OH of the guanosine moiety of m⁷GpppG on the α-phosphate of the next templated nucleoside triphosphate (pppN), resulting in the intermediate m⁷GpppGpN (wherein N is the second base of the RNA molecule).

In preferred embodiments of the present disclosure, the RNA molecule comprises a 5′-cap analog. Cap analogs have been initially described to facilitate large scale synthesis of RNA transcripts by means of in vitro transcription.

For messenger RNA, some cap analogs (also called synthetic caps) have been generally described to date, and they can all be used in the context of the present disclosure. Ideally, a cap analog is selected that is associated with higher translation efficiency and/or increased resistance to in vivo degradation and/or increased resistance to in vitro degradation.

Preferably, a cap analog is used that can only be incorporated into an RNA chain in one orientation. Pasquinelli et al., 1995, RNA J. 1:957-967) demonstrated that during in vitro transcription, bacteriophage RNA polymerases use the 7-methylguanosine unit for initiation of transcription, whereby around 40-50% of the transcripts with cap possess the cap dinucleotide in a reverse orientation (i.e., the initial reaction product is Gpppm⁷GpN). Compared to the RNAs with a correct cap, RNAs with a reverse cap are not functional with respect to translation of a nucleic acid sequence into protein. Thus, it is desirable to incorporate the cap in the correct orientation, i.e., resulting in an RNA with a structure essentially corresponding to m⁷GpppGpN etc. It has been shown that the reverse integration of the cap-dinucleotide is inhibited by the substitution of either the 2′- or the 3′—OH group of the methylated guanosine unit (Stepinski et al., 2001, RNA J. 7:1486-1495; Peng et al., 2002, Org. Lett. 24:161-164). RNAs which are synthesized in presence of such “anti reverse cap analogs” are translated more efficiently than RNAs which are in vitro transcribed in presence of the conventional 5′-cap m⁷GpppG. To that end, one cap analog in which the 3′ OH group of the methylated guanosine unit is replaced by OCH₃ is described, e.g., by Holtkamp et al., 2006, Blood 108:4009-4017 (7-methyl(3′-O-methyl)GpppG; anti-reverse cap analog (ARCA)). ARCA is a suitable cap dinucleotide according to the present disclosure:

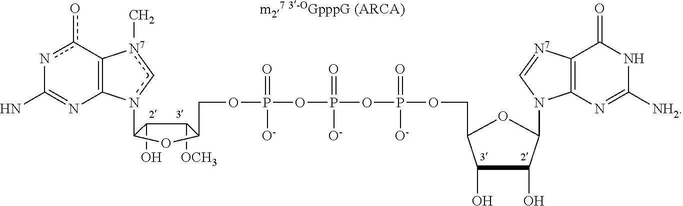

In an embodiment, the cap is having the effect that RNA with such a cap is essentially not susceptible to decapping. This is important because, in general, the amount of protein produced from synthetic mRNAs introduced into cultured mammalian cells is limited by the natural degradation of mRNA. One in vivo pathway for mRNA degradation begins with the removal of the mRNA cap. This removal is catalyzed by a heterodimeric pyrophosphatase, which contains a regulatory subunit (Dcp1) and a catalytic subunit (Dcp2). The catalytic subunit cleaves between the α and β phosphate groups of the triphosphate bridge. In the present disclosure, a cap may be selected that is not susceptible, or less susceptible, to that type of cleavage. A suitable cap analog for this purpose may be selected from a cap dinucleotide according to formula (I):

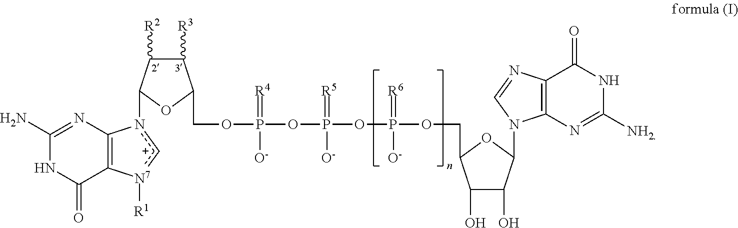

wherein R¹ is selected from the group consisting of optionally substituted alkyl, optionally substituted alkenyl, optionally substituted alkynyl, optionally substituted cycloalkyl, optionally substituted heterocyclyl, optionally substituted aryl, and optionally substituted heteroaryl,
R² and R³ are independently selected from the group consisting of H, halo, OH, and optionally substituted alkoxy, or R² and R³ together form O—X—O, wherein X is selected from the group consisting of optionally substituted CH₂, CH₂CH₂, CH₂CH₂CH₂, CH₂CH(CH₃), and
C(CH₃)₂, or R² is combined with the hydrogen atom at position 4′ of the ring to which R² is attached to form —O—CH₂— or —CH₂—O—,
R⁵ is selected from the group consisting of S, Se, and BH₃,
R⁴ and R⁶ are independently selected from the group consisting of O, S, Se, and BH₃.
n is 1, 2, or 3.

Preferred embodiments for R¹, R², R3, R⁴, R⁵, R⁶ are disclosed in WO 2011/015347 A1 and may be selected accordingly in the present disclosure.

For example, in an embodiment, the RNA molecules of the present disclosure comprise a phosphorothioate-cap-analog. Phosphorothioate-cap-analogs are specific cap analogs in which one of the three non-bridging 0 atoms in the triphosphate chain is replaced with an S atom, i.e., one of R⁴, R⁵ or R⁶ in Formula (I) is S. Phosphorothioate-cap-analogs have been described by Kowalska et al., 2008, RNA, 14:1119-1131, as a solution to the undesired decapping process, and thus to increase the stability of RNA in vivo. In particular, the substitution of an oxygen atom for a sulphur atom at the beta-phosphate group of the 5′-cap results in stabilization against Dcp2. In that embodiment, which is preferred in the present disclosure, R⁵ in Formula (I) is S; and R⁴ and R⁶ are 0.

In a further embodiment, the RNA molecules of the present disclosure comprise a phosphorothioate-cap-analog wherein the phosphorothioate modification of the RNA 5′-cap is combined with an “anti-reverse cap analog” (ARCA) modification. Respective ARCA-phosphorothioate-cap-analogs are described in WO 2008/157688 A2, and they can all be used in the RNA of the present disclosure. In that embodiment, at least one of R² or R³ in Formula (I) is not OH, preferably one among R² and R³ is methoxy (OCH₃), and the other one among R² and R³ is preferably OH. In a preferred embodiment, an oxygen atom is substituted for a sulphur atom at the beta-phosphate group (so that R⁵ in Formula (I) is S; and R⁴ and R⁶ are O). It is believed that the phosphorothioate modification of the ARCA ensures that the α, β, and γ phosphorothioate groups are precisely positioned within the active sites of cap-binding proteins in both the translational and decapping machinery. At least some of these analogs are essentially resistant to pyrophosphatase Dcp1/Dcp2. Phosphorothioate-modified ARCAs were described to have a much higher affinity for eIF4E than the corresponding ARCAs lacking a phosphorothioate group.

A respective cap that is particularly preferred in the present disclosure, i.e., m₂^{7,2′O}GppₛpG, is termed beta-S-ARCA (WO 2008/157688 A2; Kuhn et al., 2010, Gene Ther. 17:961-971). Thus, in one embodiment of the present disclosure, the RNA of the present disclosure is modified with beta-S-ARCA. beta-S-ARCA is represented by the following structure:

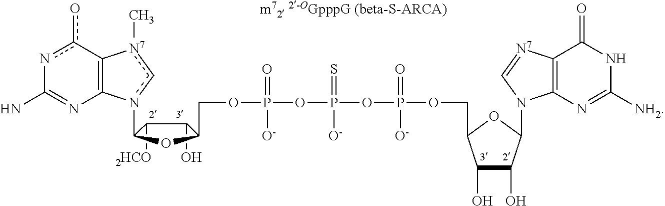

In general, the replacement of an oxygen atom for a sulphur atom at a bridging phosphate results in phosphorothioate diastereomers which are designated D1 and D2, based on their elution pattern in HPLC. Briefly, the D1 diastereomer of beta-S-ARCA” or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time. Determination of the stereochemical configuration by HPLC is described in WO 2011/015347 A1.

In a first particularly preferred embodiment of the present disclosure, RNA of the present disclosure is modified with the beta-S-ARCA(D2) diastereomer. The two diastereomers of beta-S-ARCA differ in sensitivity against nucleases. It has been shown that RNA carrying the D2 diastereomer of beta-S-ARCA is almost fully resistant against Dcp2 cleavage (only 6% cleavage compared to RNA which has been synthesized in presence of the unmodified ARCA 5-cap), whereas RNA with the beta-S-ARCA(D1) 5′-cap exhibits an intermediary sensitivity to Dcp2 cleavage (71% cleavage). It has further been shown that the increased stability against Dcp2 cleavage correlates with increased protein expression in mammalian cells. In particular, it has been shown that RNAs carrying the beta-S-ARCA(D2) cap are more efficiently translated in mammalian cells than RNAs carrying the beta-S-ARCA(D1) cap. Therefore, in one embodiment of the present disclosure, RNA of the present disclosure is modified with a cap analog according to Formula (I), characterized by a stereochemical configuration at the P atom comprising the substituent R⁵ in Formula (I) that corresponds to that at the P_{β} atom of the D2 diastereomer of beta-S-ARCA. In that embodiment, R⁵ in Formula (I) is S; and R⁴ and R⁶ are 0. Additionally, at least one of R² or R³ in Formula (I) is preferably not OH, preferably one among R² and R³ is methoxy (OCH₃), and the other one among R² and R³ is preferably OH.

In a second particularly preferred embodiment, RNA of the present disclosure is modified with the beta-S-ARCA(D1) diastereomer. This embodiment is particularly suitable for transfer of capped RNA into immature antigen presenting cells, such as for vaccination purposes. It has been demonstrated that the beta-S-ARCA(D1) diastereomer, upon transfer of respectively capped RNA into immature antigen presenting cells, is particularly suitable for increasing the stability of the RNA, increasing translation efficiency of the RNA, prolonging translation of the RNA, increasing total protein expression of the RNA, and/or increasing the immune response against an antigen or antigen peptide encoded by said RNA (Kuhn et al., 2010, Gene Ther. 17:961-971). Therefore, in an alternative embodiment of the present disclosure, RNA of the present disclosure is modified with a cap analog according to Formula (I), characterized by a stereochemical configuration at the P atom comprising the substituent R⁵ in Formula (I) that corresponds to that at the P_{β} atom of the D1 diastereomer of beta-S-ARCA. Respective cap analogs and embodiments thereof are described in WO 2011/015347 A1 and Kuhn et al., 2010, Gene Ther. 17:961-971. Any cap analog described in WO 2011/015347 A1, wherein the stereochemical configuration at the P atom comprising the substituent R⁵ corresponds to that at the P_{β} atom of the D1 diastereomer of beta-S-ARCA, may be used in the present disclosure. Preferably, R⁵ in Formula (I) is S; and R⁴ and R⁶ are 0. Additionally, at least one of R² or R³ in Formula (I) is preferably not OH, preferably one among R² and R³ is methoxy (OCH₃), and the other one among R² and R³ is preferably OH.

In one embodiment, RNA of the present disclosure is modified with a 5′-cap structure according to Formula (I), wherein any one phosphate group is replaced by a boranophosphate group or a phosphoroselenoate group. Such caps have increased stability both in vitro and in vivo. Optionally, the respective compound has a 2′-O— or 3′-O-alkyl group (wherein alkyl is preferably methyl); respective cap analogs are termed BH₃-ARCAs or Se-ARCAs. Compounds that are particularly suitable for capping of mRNA include the β-BH₃-ARCAs and β-Se-ARCAs, as described in WO 2009/149253 A2. For these compounds, a stereochemical configuration at the P atom comprising the substituent R⁵ in Formula (I) that corresponds to that at the P_{β} atom of the D1 diastereomer of beta-S-ARCA is preferred.

In some embodiments, the RNA comprises a cap which may be suitable in the context of the present disclosure is a cap0 (methylation of the first nucleobase, e.g. ^{m7}GpppN), cap1 (additional methylation of the ribose of the adjacent nucleotide of ^{m7}GpppN), cap2 (additional methylation of the ribose of the 2nd nucleotide downstream of the ^{m7}GpppN), cap3 (additional methylation of the ribose of the 3rd nucleotide downstream of the ^{m7}GpppN), cap4 (additional methylation of the ribose of the 4th nucleotide downstream of the ^{m7}GpppN), ARCA (anti-reverse cap analogue), modified ARCA (e.g. phosphothioate modified ARCA, e.g., beta-S-ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the RNA comprises a cap that is a Cap-0 (also referred herein as “Cap0”), a Cap-1 (also referred herein as “Cap1”), or Cap-2 (also referred herein as “Cap2”). See, e.g., FIG. 1 of Ramanathan A et al., and FIG. 1 of Decroly E et al.

In some embodiments, a Cap0 comprises a guanosine nucleoside methylated at the 7-position of guanine (^{m7}G). In some embodiments, a Cap0 is connected to an RNA via a 5′- to 5-triphosphate linkage and is also referred to herein as ^{m7}Gppp or ^{m7}G(5′)ppp(5′).

In some embodiments, a Cap1 comprises a guanosine nucleoside methylated at the 7-position of guanine (^{m}7G or ^{7m}G) and a 2′O methylated first nucleotide in an RNA (^{2′OMe}N₁ or N₁2′OMe or N₁^{2′OMe}). In some embodiments, a Cap1 is connected to an RNA via a 5′- to 5-triphosphate linkage; in some embodiments, a Cap1 may be represented as ^{m7}Gppp(N₁^{2′OMe}) or ^{m7}G(5′)ppp(5′)(N₁^{2′OMe}) or ^{7m}G(5′)ppp(5′)N₁^{2′-OMe}). In some embodiments, N₁ is chosen from A, C, G, or U. In some embodiments, N₁ is A. In some embodiments, N₁ is C. In some embodiments, N₁ is G. In some embodiments, N₁ is U.

In some embodiments, a ^{m7}G(5′)ppp(5′)(N₁^{2′OMe}) Cap1 comprises a second nucleotide, N₂ which is a cap proximal A, G, C, or U at position +2. In some embodiments, such Cap1's are represented as (^{m7}G(5′)ppp(5′)(N₁^{2′OMe})pN₂). In some embodiments, N₂ is A. In some embodiments, N₂ is C. In some embodiments, N₂ is G. In some embodiments, N₂ is U.

In some embodiments, a Cap1 is or comprises ^{m7}G(5′)ppp(5′)(A₁^{2′OMe})pG₂ wherein A₁ is a cap proximal A at position +1 and G₂ is a cap proximal G at position +2, and has the following structure:

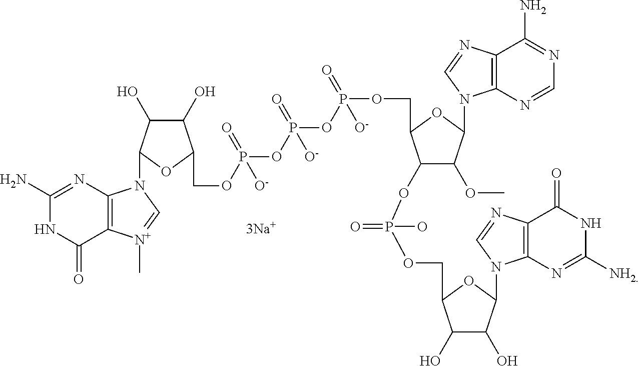 [SMILES: COC1C(OP(=O)(O)OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In some embodiments, a Cap1 is or comprises m7G(5′)ppp(5′)(A₁^{2′OMe})pU₂ wherein A₁ is a cap proximal A at position +1 and U₂ is a cap proximal U at position +2, and has the following structure:

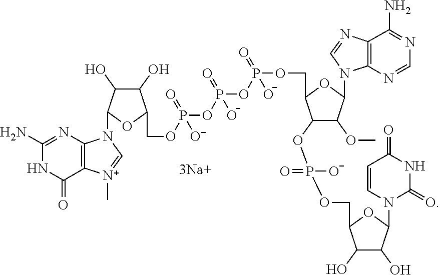 [SMILES: COC1C(OP(=O)([O-])OCC2OC(n3ccc(=O)[nH]c3=O)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

In some embodiments, a Cap1 is or comprises ^{m7}G(5′)ppp(5′)(G₁^{2′OMe})pG₂ wherein G₁ is a cap proximal G at position +1 and G₂ is a cap proximal G at position +2, and has the following structure:

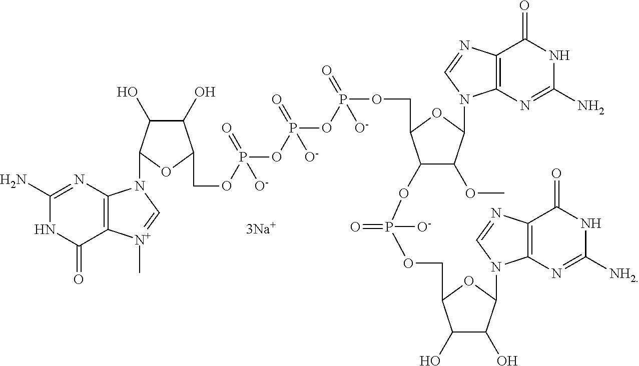 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(=O)[nH]c(N)nc21]

In some embodiments, a Cap1 comprises a guanosine nucleoside methylated at the 7-position of guanine (m7G) and one or more additional modifications, e.g., methylation on a ribose, and a 2′O methylated first nucleotide in an RNA. In some embodiments, a Cap1 comprises a guanosine nucleoside methylated at the 7-position of guanine and a 3′O methylation at a ribose (m7G3′OMe or 7mG^{3′OMe}); and a 2′O methylated first nucleotide in an RNA (N₁^{2′OMe}). In some embodiments, a Cap1 is connected to an RNA via a 5′- to 5′-triphosphate linkage and is also referred to herein as (m7G3′OMe)ppp(2′OMeN₁) or (^{m7}G^{3′OMe})(5′)ppp(5′)(^{2′OMe}N₁). In some embodiments, N₁ is chosen from A, C, G, or U. In some embodiments, N₁ is A. In some embodiments, N₁ is C. In some embodiments, N₁ is G. In some embodiments, N₁ is U.

In some embodiments, a (^{m7}G^{3′OMe})(5′)ppp(5′)(N₁^{2′OMe}) Cap1 comprises a second nucleotide, N₂ which is a cap proximal nucleotide at position 2 and is chosen from A, G, C, or U (^{m7}G^{3′OMe})(5′)ppp(5′)(N₁^{2′OMe})pN₂). In some embodiments, N₂ is A. In some embodiments, N₂ is C. In some embodiments, N₂ is G. In some embodiments, N₂ is U.

In some embodiments, a Cap1 is or comprises (^{m7}G^{3′OMe})(5′)ppp(5′)(A₁^{2′OMe})pG₂ wherein A₁ is a cap proximal A at position +1 and G₂ is a cap proximal G at position +2, and has the following structure:

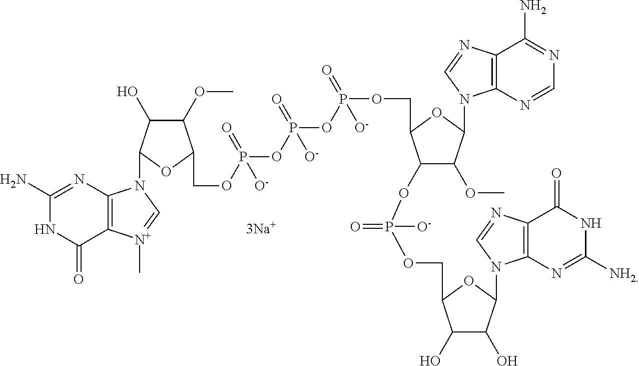 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

In some embodiments, a Cap1 is or comprises (^{m7}G^{3′OMe})(5′)ppp(5′)(G₁^{2′OMe})pG₂ wherein G₁ is a cap proximal G at position +1 and G₂ is a cap proximal G at position +2, and has the following structure:

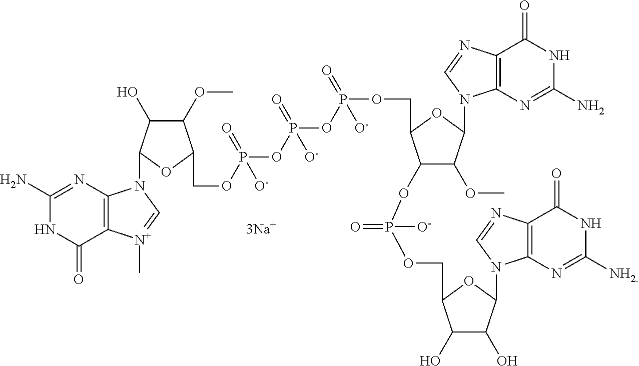 [SMILES: COC1C(COP(=O)([O-])OP(=O)([O-])OP(=O)([O-])OCC2OC(n3cnc4c(=O)[nH]c(N)nc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

In some embodiments, a second nucleotide in a Cap1 can comprise one or more modifications, e.g., methylation. In some embodiments, a Cap1 comprising a second nucleotide comprising a 2′O methylation is a Cap2 structure.

In some embodiments, an RNA polynucleotide comprising a Cap1 has increased translation efficiency, increased translation rate and/or increased expression of an encoded payload relative to an appropriate reference comparator. In some embodiments, an RNA polynucleotide comprising a Cap1 having (^{m7}G^{3′OMe})(5′)ppp(5′)(A₁^{2′OMe})pG₂ wherein Aₕ is a cap proximal nucleotide at position +1 and G₂ is a cap proximal nucleotide at position +2 has increased translation efficiency relative to an RNA polynucleotide comprising a Cap1 having (^{m7}G^{3′OMe})(5′)ppp(5′)(G₁^{2′OMe})pG₂ wherein G₁ is a cap proximal nucleotide at position 1 and G₂ is a cap proximal nucleotide at position 2. In some embodiments, increased translation efficiency is assessed upon administration of an RNA polynucleotide to a cell or an organism.

In some embodiments, a cap analog used in an RNA polynucleotide is ^{m7}G^{3′OMe}Gppp(m1^{2′-OMe})ApG (also sometimes referred to as m2^{7,3′-OMe}G(5)ppp(5)m^{2′-OMe}ApG or (^{m7}G^{3′OMe})(5′)ppp(5′)(A^{2′OMe})pG), which has the following structure:

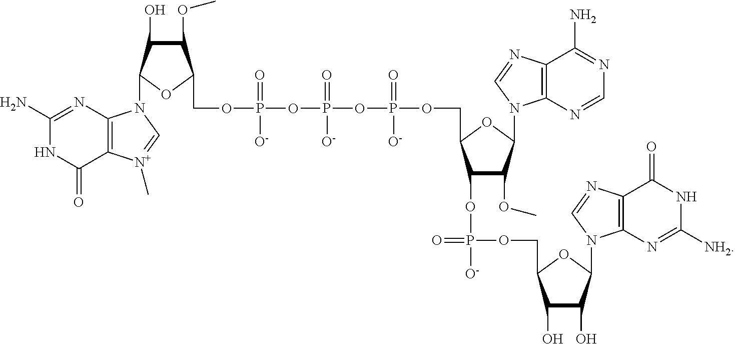

Below is an exemplary Cap1 RNA, which comprises RNA and m2^{7,3′OMe}G(5′)ppp(5)m^{2′-oMe}ApG:

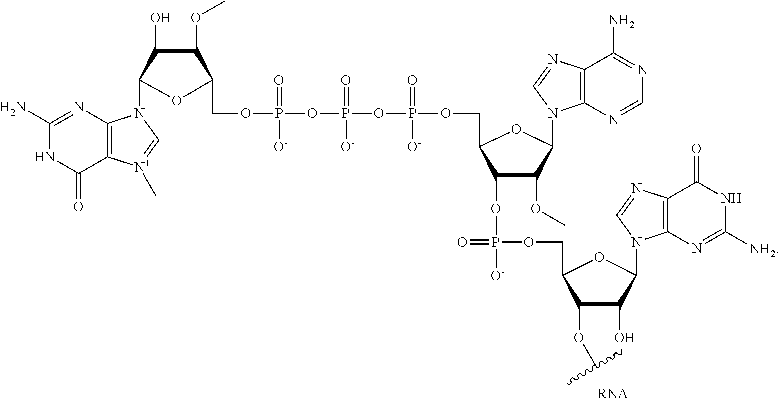

Below is another exemplary Cap1 RNA:

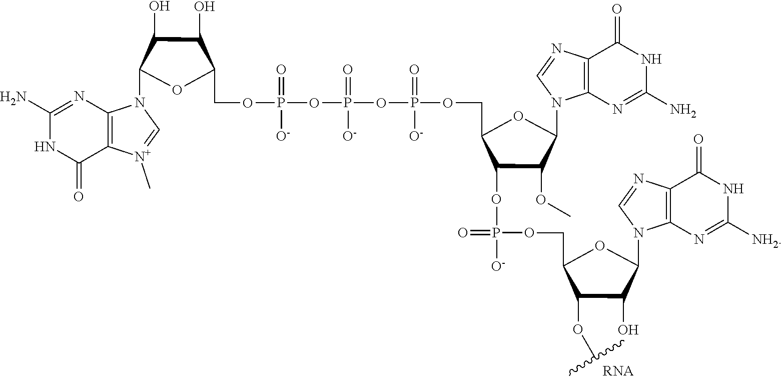

### UTR

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR).

A 3′-UTR, if present, is located at the 3′ end of a gene, downstream of the termination codon of a protein-encoding region, but the term “3′-UTR” does preferably not include the poly(A) tail. Thus, the 3′-UTR is upstream of the poly(A) tail (if present), e.g. directly adjacent to the poly(A) tail.

A 5′-UTR, if present, is located at the 5′ end of a gene, upstream of the start codon of a protein-encoding region. A 5′-UTR is downstream of the 5′-cap, e.g. directly adjacent to the 5′-cap.

5′- and/or 3′-untranslated regions may, according to the disclosure, be functionally linked to an open reading frame, so as for these regions to be associated with the open reading frame in such a way that the stability and/or translation efficiency of the RNA comprising said open reading frame are increased.

In some embodiments, the RNA molecules according to the present disclosure comprise a 5′-UTR and/or a 3′-UTR.

UTRs are implicated in stability and translation efficiency of RNA. Both can be improved, besides structural modifications concerning the 5′-cap and/or the 3′ poly(A)-tail as described herein, by selecting specific 5′ and/or 3′ untranslated regions (UTRs). Sequence elements within the UTRs are generally understood to influence translational efficiency (mainly 5′-UTR) and RNA stability (mainly 3′-UTR). It is preferable that a 5′-UTR is present that is active in order to increase the translation efficiency and/or stability of the RNA. Independently or additionally, it is preferable that a 3′-UTR is present that is active in order to increase the translation efficiency and/or stability of the RNA molecule.

The terms “active in order to increase the translation efficiency” and/or “active in order to increase the stability”, with reference to a first nucleic acid sequence (e.g. a UTR), means that the first nucleic acid sequence is capable of modifying, in a common transcript with a second nucleic acid sequence, the translation efficiency and/or stability of said second nucleic acid sequence in such a way that said translation efficiency and/or stability is increased in comparison with the translation efficiency and/or stability of said second nucleic acid sequence in the absence of said first nucleic acid sequence.

A 5′-UTR according to the present disclosure can comprise any combination of more than one nucleic acid sequence, optionally separated by a linker. A 3′-UTR according to the present disclosure can comprise any combination of more than one nucleic acid sequence, optionally separated by a linker.

The term “linker” according to the disclosure relates to a nucleic acid sequence added between two nucleic acid sequences to connect said two nucleic acid sequences. There is no particular limitation regarding the linker sequence.

A 3′-UTR typically has a length of 200 to 2000 nucleotides, e.g. 500 to 1500 nucleotides. The 3′-untranslated regions of immunoglobulin mRNAs are relatively short (fewer than about 300 nucleotides), while the 3′-untranslated regions of other genes are relatively long. For example, the 3-untranslated region of tPA is about 800 nucleotides in length, that of factor VIII is about 1800 nucleotides in length and that of erythropoietin is about 560 nucleotides in length. The 3-untranslated regions of mammalian mRNA typically have a homology region known as the AAUAAA hexanucleotide sequence. This sequence is presumably the poly(A) attachment signal and is frequently located from 10 to 30 bases upstream of the poly(A) attachment site. 3-untranslated regions may contain one or more inverted repeats which can fold to give stem-loop structures which act as barriers for exoribonucleases or interact with proteins known to increase RNA stability (e.g. RNA-binding proteins).

The human beta-globin 3′-UTR, particularly two consecutive identical copies of the human beta-globin 3′-UTR, contributes to high transcript stability and translational efficiency (Holtkamp et al., 2006, Blood 108:4009-4017). Thus, in one embodiment, the RNA molecule according to the present disclosure comprises two consecutive identical copies of the human beta-globin 3′-UTR. Thus, it comprises in the 5′-3′ direction: (a) optionally a 5′-UTR; (b) an open reading frame; (c) a 3′-UTR; said 3′-UTR comprising two consecutive identical copies of the human beta-globin 3′-UTR, a fragment thereof, or a variant of the human beta-globin 3′-UTR or fragment thereof.

In an embodiment, the RNA molecules according to the present disclosure comprise a 3′-UTR which is active in order to increase translation efficiency and/or stability, but which is not the human beta-globin 3′-UTR, a fragment thereof, or a variant of the human beta-globin 3′-UTR or fragment thereof.

In an embodiment, the RNA molecules according to the present disclosure comprise a 5′-UTR which is active in order to increase translation efficiency and/or stability.

### Poly(A) Sequence

In some embodiments, the RNA molecules according to the present disclosure comprise a 3′-poly(A) sequence.

According to the disclosure, in one embodiment, a poly(A) sequence comprises or essentially consists of or consists of at least 20, preferably at least 26, preferably at least 40, preferably at least 80, preferably at least 100 and preferably up to 500, preferably up to 400, preferably up to 300, preferably up to 200, and in particular up to 150 A nucleotides, and in particular about 120 A nucleotides. In this context “essentially consists of” means that most nucleotides in the poly(A) sequence, typically at least 50%, and preferably at least 75% by number of nucleotides in the “poly(A) sequence”, are A nucleotides (adenylate), but permits that remaining nucleotides are nucleotides other than A nucleotides, such as U nucleotides (uridylate), G nucleotides (guanylate), C nucleotides (cytidylate). In this context “consists of” means that all nucleotides in the poly(A) sequence, i.e., 100% by number of nucleotides in the poly(A) sequence, are A nucleotides. The term “A nucleotide” or “A” refers to adenylate.

Indeed, it has been demonstrated that a 3′-poly(A) sequence of about 120 A nucleotides has a beneficial influence on the levels of RNA in transfected eukaryotic cells, as well as on the levels of protein that is translated from an open reading frame that is present upstream (5′) of the 3′-poly(A) sequence (Holtkamp et al., 2006, Blood, vol. 108, pp. 4009-4017).

The present disclosure provides for a 3′-poly(A) sequence to be attached during RNA transcription, i.e. during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In some embodiments of the present disclosure, the 3′-poly(A) cassette present in the coding strand of DNA template molecules essentially consists of dA nucleotides, but is interrupted by a random sequence having an equal distribution of the four nucleotides (dA, dC, dG, dT). Such random sequence may be 5 to 50, preferably 10 to 30, more preferably 10 to 20 nucleotides in length. Such a cassette is disclosed in WO 2016/005004 A1. Any poly(A) cassette disclosed in WO 2016/005004 A1 may be used in the present disclosure. A poly(A) cassette that essentially consists of dA nucleotides, but is interrupted by a random sequence having an equal distribution of the four nucleotides (dA, dC, dG, dT) and having a length of, e.g., 5 to 50 nucleotides shows, on DNA level, constant propagation of plasmid DNA in E. coli and is still associated, on RNA level, with the beneficial properties with respect to supporting RNA stability and translational efficiency.

Consequently, in some embodiments of the present disclosure, the 3′-poly(A) sequence contained in an RNA molecule described herein essentially consists of A nucleotides, but is interrupted by a random sequence having an equal distribution of the four nucleotides (A, C, G, U). Such random sequence may be 5 to 50, preferably 10 to 30, more preferably 10 to 20 nucleotides in length.

### Codon Usage

In general, the degeneracy of the genetic code will allow the substitution of certain codons (base triplets coding for an amino acid) that are present in an RNA sequence by other codons (base triplets), while maintaining the same coding capacity (so that the replacing codon encodes the same amino acid as the replaced codon). In some embodiments of the present disclosure, at least one codon of an open reading frame comprised by an RNA molecule differs from the respective codon in the respective open reading frame in the species from which the open reading frame originates. In that embodiment, the coding sequence of the open reading frame is said to be “adapted” or “modified”. The coding sequence of an open reading frame comprised by the RNA molecule may be adapted.

For example, when the coding sequence of an open reading frame is adapted, frequently used codons may be selected: WO 2009/024567 A1 describes the adaptation of a coding sequence of a nucleic acid molecule, involving the substitution of rare codons by more frequently used codons. Since the frequency of codon usage depends on the host cell or host organism, that type of adaptation is suitable to fit a nucleic acid sequence to expression in a particular host cell or host organism. Generally, speaking, more frequently used codons are typically translated more efficiently in a host cell or host organism, although adaptation of all codons of an open reading frame is not always required.

For example, when the coding sequence of an open reading frame is adapted, the content of G (guanylate) residues and C (cytidylate) residues may be altered by selecting codons with the highest GC-rich content for each amino acid. RNA molecules with GC-rich open reading frames were reported to have the potential to reduce immune activation and to improve translation and half-life of RNA (Thess et al., 2015, Mol. Ther. 23:1457-1465).

### Particle

To overcome the barriers to safe and effective nucleic acid delivery, nucleic acids may be administered with one or more delivery vehicles that protect the nucleic acids from degradation, maximize delivery to on-target cells and minimize exposure to off-target cells. Such nucleic acid delivery vehicles may complex or encapsulate nucleic acids and include a range of materials, including polymers and lipids. In some embodiments, such nucleic acid delivery vehicles may form particles with nucleic acids, preferably RNA.

RNA, in particular mRNA, described herein may be present in particles comprising (i) the RNA, and (ii) at least one cationic or cationically ionizable compound such as a polymer or lipid complexing the RNA. Electrostatic interactions between positively charged molecules such as polymers and lipids and negatively charged RNA are involved in particle formation. This results in complexation and spontaneous formation of nucleic acid, in particular RNA, particles.

In some embodiments, the compositions described herein comprise the RNA molecule or molecules in a particle.

Different types of nucleic acid containing particles have been described previously to be suitable for delivery of RNA in particulate form (cf., e.g., Kaczmarek, J. C. et al., 2017, Genome Medicine 9, 60). For non-viral RNA delivery vehicles, nanoparticle encapsulation of nucleic acids physically protects the nucleic acids from degradation and, depending on the specific chemistry, can aid in cellular uptake and endosomal escape.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids) optionally in combination with additional substances (e.g., additional lipids) which do not have to be amphiphilic. In some embodiments, the term “particle” relates to a micro- or nano-sized structure, such as a micro- or nano-sized compact structure. According to the present disclosure, the term “particle” includes nanoparticles.

A “DNA particle”, “RNA particle” or “DNA and RNA particle” can be used to deliver DNA and/or RNA to a target site of interest (e.g., cell, tissue, organ, and the like). A DNA and/or RNA particle may be formed from lipids comprising at least one cationic or cationically ionizable lipid. Without intending to be bound by any theory, it is believed that the cationic or cationically ionizable lipid combines together with the nucleic acids to form aggregates, and this aggregation results in colloidally stable particles.

RNA particles described herein include lipid nanoparticle (LNP)-based and lipoplex (LPX)-based formulations.

A lipoplex (LPX) described herein is obtainable from mixing two aqueous phases, namely a phase comprising RNA and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that the lipids in the mixture as required are able to form lamellar (bilayer) phases in the applied aqueous environment.

In some embodiments, liposomes comprise unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of RNA are amphiphilic in nature and consist of a positively charged (cationic) amine head group linked to a hydrocarbon chain or cholesterol derivative via glycerol.

In some embodiments, lipoplexes are multilamellar liposome-based formulations that form upon electrostatic interaction of cationic liposomes with nucleic acids. In some embodiments, formed lipoplexes possess distinct internal arrangements of molecules that arise due to the transformation from liposomal structure into compact RNA-lipoplexes.

In some embodiments, an LPX particle comprises an amphiphilic lipid, in particular cationic or cationically ionizable amphiphilic lipid, and RNA (especially mRNA) as described herein. In some embodiments, electrostatic interactions between positively charged liposomes (made from one or more amphiphilic lipids, in particular cationic or cationically ionizable amphiphilic lipids) and negatively charged RNA (especially mRNA) results in complexation and spontaneous formation of RNA lipoplex particles. Positively charged liposomes may be generally synthesized using a cationic or cationically ionizable amphiphilic lipid, such as DOTMA and/or DODMA, and optionally additional lipids, such as DOPE or DSPC.

In general, a lipid nanoparticle (LNP) is typically obtainable from direct mixing of RNA in an aqueous phase with lipids in a phase comprising an organic solvent, such as ethanol. In that case, lipids or lipid mixtures can be used for particle formation, which do not form lamellar (bilayer) phases in water.

In some embodiments, LNPs comprise or consist of a cationic/cationically ionizable lipid and helper lipids such as phospholipids, cholesterol, and/or polymer-conjugated lipids (e.g., polyethylene glycol (PEG) lipids). In some embodiments, in the RNA LNPs described herein the RNA (in particular, mRNA) is bound by cationically ionizable lipid that occupies the central core of the LNP. In some embodiments, polymer-conjugated lipid forms the surface of the LNP, along with phospholipids. In some embodiments, cholesterol and cationically ionizable lipid in charged and uncharged forms can be distributed throughout the LNP.

In some embodiments, RNA (e.g., mRNA) described herein may be noncovalently associated with a particle as described herein. In embodiments, the RNA (especially mRNA) may be adhered to the outer surface of the particle (surface RNA (especially surface mRNA)) and/or may be contained in the particle (encapsulated RNA (especially encapsulated mRNA)).

In some embodiments, the particles (e.g., LNPs and LPXs) described herein have a size (such as a diameter) in the range of about 10 to about 2000 nm, such as at least about 15 nm (e.g., at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, or at least about 100 nm) and/or at most about 1900 nm (e.g., at most about 1800 nm, at most about 1700 nm, at most about 1600 nm, at most about 1500 nm, at most about 1400 nm, at most about 1300 nm, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 nm, at most about 600 nm, at most about 550 nm, or at most about 500 nm), such as in the range of about 20 to about 1500 nm, such as about 30 to about 1200 nm, about 40 to about 1100 nm, about 50 to about 1000 nm, about 60 to about 900 nm, about 70 to about 800 nm, about 80 to about 700 nm, about 90 to about 600 nm, or about 50 to about 500 nm or about 100 to about 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 nm, 70 to 150 nm, or 80 to 150 nm. In some embodiments, the particles (e.g., LNPs and LPXs) described herein have a size (such as a diameter) in the range of from about 40 nm to about 200 nm, such as from about 50 nm to about 180 nm, from about 60 nm to about 160 nm, from about 80 nm to about 150 nm or from about 80 nm to about 120 nm.

In some embodiments, the particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, from about 200 nm to about 250 nm, or from about 80 to about 150 nm. In some embodiments, the particles (e.g., LNPs and LPXs) described herein have an average diameter that in some embodiments ranges from about 40 nm to about 200 nm, such as from about 50 nm to about 180 nm, from about 60 nm to about 160 nm, from about 80 nm to about 150 nm or from about 80 nm to about 120 nm.

RNA particles (especially mRNA particles) described herein may exhibit a polydispersity index (PDI) less than about 0.5, less than about 0.4, less than about 0.3, less than about 0.2, less than about 0.1, or less than about 0.05. By way of example, the RNA particles can exhibit a polydispersity index in a range of about 0.01 to about 0.4 or about 0.1 to about 0.3.

The N/P ratio gives the ratio of the nitrogen groups in the lipid to the number of phosphate groups in the nucleic acid. It is correlated to the charge ratio, as the nitrogen atoms (depending on the pH) are usually positively charged and the phosphate groups are negatively charged. The N/P ratio, where a charge equilibrium exists, depends on the pH. Lipid formulations may be formed at N/P ratios larger than four up to twelve, because positively charged nanoparticles can be favorable for transfection. In that case, RNA is considered to be completely bound to nanoparticles.

The present disclosure describes compositions comprising RNA (especially mRNA) and at least one cationic or cationically ionizable lipid which associates with the RNA to form RNA particles and formulations comprising such particles. The RNA particles may comprise RNA which is complexed in different forms by non-covalent interactions to the particle. The particles described herein are not viral particles, in particular infectious viral particles, i.e., they are not able to virally infect cells.

Suitable cationic or cationically ionizable lipids are those that form RNA particles and are included by the term “particle forming components” or “particle forming agents”. The term “particle forming components” or “particle forming agents” relates to any components which associate with RNA to form or RNA particles. Such components include any component which can be part of RNA particles.

In some embodiments, RNA particles (especially mRNA particles) comprise more than one type of RNA molecules, where the molecular parameters of the RNA molecules may be similar or different from each other, like with respect to molar mass or fundamental structural elements such as molecular architecture, capping, coding regions or other features,

In particulate formulation, it is possible that each RNA species is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the present disclosure are obtainable by forming, separately, individual particulate formulations, followed by a step of mixing of the individual particulate formulations. By the step of mixing, a formulation comprising a mixed population of RNA-containing particles is obtainable. Individual particulate populations may be together in one container, comprising a mixed population of individual particulate formulations. Alternatively, it is possible that all RNA species of the pharmaceutical composition are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Polymers

Given their high degree of chemical flexibility, polymers are commonly used materials for nanoparticle-based delivery. Typically, cationic polymers are used to electrostatically condense the negatively charged RNA into particles, in particular nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(β-amino esters), in particular, have gained widespread use in nucleic acid delivery owing to their ease of synthesis and biodegradability. Such synthetic polymers are also suitable as cationic polymers herein.

A “polymer,” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties.

If more than one type of repeat unit is present within the polymer, then the polymer is said to be a “copolymer.” It is to be understood that the polymer being employed herein can be a copolymer. The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, the polymer is biocompatible. Biocompatible polymers are polymers that typically do not result in significant cell death at moderate concentrations. In certain embodiments, the biocompatible polymer is biodegradable, i.e., the polymer is able to degrade, chemically and/or biologically, within a physiological environment, such as within the body.

In certain embodiments, polymer may be protamine or polyalkyleneimine.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

In one embodiment, the polyalkyleneimine comprises polyethylenimine and/or polypropylenimine, preferably polyethyleneimine. A preferred polyalkyleneimine is polyethyleneimine (PEI). The average molecular weight of PEI is preferably 0.75-102 to 107 Da, preferably 1000 to 105 Da, more preferably 10000 to 40000 Da, more preferably 15000 to 30000 Da, even more preferably 20000 to 25000 Da.

Preferred according to the disclosure is linear polyalkyleneimine such as linear polyethyleneimine (PEI).

Cationic polymers (including polycationic polymers) contemplated for use herein include any cationic polymers which are able to electrostatically bind nucleic acid. In one embodiment, cationic polymers contemplated for use herein include any cationic polymers with which nucleic acid can be associated, e.g. by forming complexes with the nucleic acid or forming vesicles in which the nucleic acid is enclosed or encapsulated.

Particles described herein may also comprise polymers other than cationic polymers, i.e., non-cationic polymers and/or anionic polymers. Collectively, anionic and neutral polymers are referred to herein as non-cationic polymers.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The monovalent radical of a hydrocarbon is referred to as hydrocarbyl herein. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes non-cyclic, e.g., linear (straight) or branched, hydrocarbyl groups, such as alkyl, alkenyl, or alkynyl as defined herein. It should be appreciated that one or more of the hydrogen atoms in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

The term “alkyl” refers to a saturated linear or branched monovalent hydrocarbon moiety which may have one to thirty, typically one to twenty, often six to eighteen carbon atoms. Exemplary nonpolar alkyl groups include, but are not limited to, methyl, ethyl, propyl, isopropyl, hexyl, decyl, dodecyl, tetradecyl, hexadecyl, octadecyl, and the like.

The term “alkenyl” refers to a linear or branched monovalent hydrocarbon moiety having at least one carbon-carbon double bond in which the total carbon atoms may be six to thirty, typically six to twenty often six to eighteen. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds.

The term “alkynyl” refers to a linear or branched monovalent hydrocarbon moiety having at least one carbon-carbon triple bond in which the total carbon atoms may be six to thirty, typically six to twenty, often six to eighteen. Alkynyl groups can optionally have one or more carbon-carbon double bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds.

The term “alkylene” refers to a saturated linear or branched divalent hydrocarbon moiety which may have one to thirty, typically two to twenty, often four to twelve carbon atoms. Exemplary nonpolar alkylene groups include, but are not limited to, methylene, ethylene, trimethylene, hexamethylene, decamethylene, dodecamethylene, tetradecamethylene, hexadecamethylene, octadecmethylene, and the like.

The term “alkenylene” refers to a linear or branched divalent hydrocarbon moiety having at least one carbon-carbon double bond in which the total carbon atoms may be two to thirty, typically two to twenty, often four to twelve. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds.

The term “cycloalkyl” represents cyclic non-aromatic versions of “alkyl” and “alkenyl” with preferably 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The cycloalkyl group may consist of one ring (monocyclic), two rings (bicyclic), or more than two rings (polycyclic).

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure. Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic).

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term includes molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids comprising two long alkyl chains and a polar head group are generally cylindrical. The area occupied by the two alkyl chains is similar to the area occupied by the polar head group. Such lipids have low solubility as monomers and tend to aggregate into planar bilayers that are water insoluble. Traditional surfactant monomers comprising only one linear alkyl chain and a hydrophilic head group are generally cone shaped. The hydrophilic head group tends to occupy more molecular space than the linear alkyl chain. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more cis double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic/Cationically Ionizable Lipids

In some embodiments, the RNA compositions and formulations and nucleic acid particles described herein comprise at least one cationic or cationically ionizable lipid as particle forming agent. Cationic or cationically ionizable lipids contemplated for use herein include any cationic or cationically ionizable lipids (including lipid-like materials) which are able to electrostatically bind nucleic acid. In some embodiments, cationic or cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” refers to a lipid or lipid-like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In some embodiments, a cationic lipid has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., which is not permanently cationic. Thus, depending on the pH of the composition in which the cationically ionizable lipid is solved, the cationically ionizable lipid is either positively charged or neutral. For purposes of the present disclosure, cationically ionizable lipids are covered by the term “cationic lipid” unless contradicted by the circumstances.

In some embodiments, the cationic or cationically ionizable lipid comprises a head group which includes at least one nitrogen atom (N) which is positive charged or capable of being protonated, e.g., under physiological conditions.

Examples of cationic or cationically ionizable lipids include, but are not limited to N,N-dimethyl-2,3-dioleyloxypropylamine (DODMA), 1,2-dioleoyl-3-trimethylammonium propane (DOTAP); 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-l-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethyl amino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In some embodiments, the cationic or cationically ionizable lipid is DOTMA. In some embodiments, the cationic or cationically ionizable lipid is DODMA.

DOTMA is a cationic lipid with a quaternary amine headgroup. The structure of DOTMA may be represented as follows:

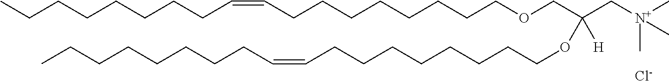 [SMILES: CCCCCCCC/C=C\CCCCCCCCOCC(C[N+](C)(C)C)OCCCCCCCC/C=C\CCCCCCCC ; [Cl-]]

DODMA is an ionizable cationic lipid with a tertiary amine headgroup. The structure of DODMA may be represented as follows:

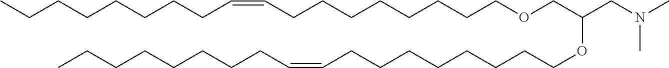 [SMILES: CCCCCCCC/C=C\CCCCCCCCOCC(CN(C)C)OCCCCCCCC/C=C\CCCCCCCC]

In some embodiments, the cationic or cationically ionizable lipid may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the particle.

### Additional Lipids

The RNA compositions and formulations and RNA particles described herein may also comprise lipids (including lipid-like materials) other than cationic or cationically ionizable lipids (also collectively referred to herein as cationic lipids), i.e., non-cationic lipids (including non-cationic or non-cationically ionizable lipids or lipid-like materials). Collectively, anionic and neutral lipids or lipid-like materials are referred to herein as non-cationic lipids. Optimizing the formulation of RNA particles by addition of other hydrophobic moieties, such as cholesterol and lipids, in addition to a cationic or cationically ionizable lipid may enhance particle stability and efficacy of nucleic acid delivery.

One or more additional lipids may or may not affect the overall charge of the RNA particles. In some embodiments, the or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In some embodiments, the RNA compositions and formulations and RNA particles described herein comprise a cationic or cationically ionizable lipid and one or more additional lipids.

Without wishing to be bound by theory, the amount of the cationic or cationically ionizable lipid compared to the amount of the one or more additional lipids may affect important RNA particle characteristics, such as charge, particle size, stability, tissue selectivity, and bioactivity of the RNA. Accordingly, in some embodiments, the molar ratio of the cationic or cationically ionizable lipid to the one or more additional lipids is from about 10:0 to about 1:9, about 4:1 to about 1:2, about 4:1 to about 1:1, about 3:1 to about 1:1, or about 3:1 to about 2:1.

In some embodiments, the one or more additional lipids comprised in the RNA compositions and formulations and RNA particles described herein comprise one or more of the following: neutral lipids, steroids, and combinations thereof.

In some embodiments, the one or more additional lipids comprise a neutral lipid which is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC. In some embodiments, the neutral lipid is DOPE.

In some embodiments, the additional lipid comprises one of the following: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Thus, in some embodiments, the RNA compositions and formulations and RNA particles described herein comprise (1) a cationic or cationically ionizable lipid, and a phospholipid such as DSPC or DOPE or (2) a cationic or cationically ionizable lipid and a phospholipid such as DSPC or DOPE and cholesterol.

In some embodiments, the RNA particles (especially the particles comprising mRNA) described herein comprise (1) DOTMA and DOPE, (2) DOTMA, DOPE and cholesterol, (3) DODMA and DOPE or (4) DODMA, DOPE and cholesterol.

DSPC is a neutral phospholipid. The structure of DSPC may be represented as follows:

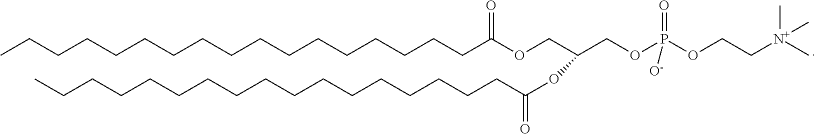 [SMILES: CCCCCCCCCCCCCCCCCC(=O)OC[C@H](COP(=O)([O-])OCC[N+](C)(C)C)OC(=O)CCCCCCCCCCCCCCCCC]

DOPE is a neutral phospholipid. The structure of DOPE may be represented as follows:

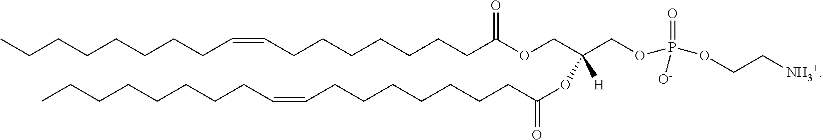

The structure of cholesterol may be represented as follows:

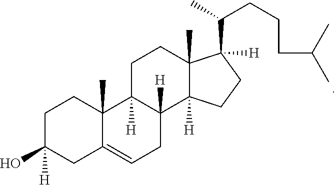

In some embodiments, RNA compositions and formulations and RNA particles described herein do not include a polymer conjugated lipid such as a pegylated lipid. The term “pegylated lipid” refers to a molecule comprising both a lipid portion and a polyethylene glycol portion. Pegylated lipids are known in the art.

In some embodiments, the additional lipid (e.g., one or more phospholipids and/or cholesterol) may comprise from about 0 mol % to about 90 mol %, from about 0 mol % to about 80 mol %, from about 2 mol % to about 80 mol %, from about 5 mol % to about 80 mol %, from about 5 mol % to about 60 mol %, from about 5 mol % to about 50 mol %, from about 7.5 mol % to about 50 mol %, or from about 10 mol % to about 40 mol % of the total lipid present in the particle. In some embodiments, the additional lipid (e.g., one or more phospholipids and/or cholesterol) comprises about 10 mol %, about 15 mol %, or about 20 mol % of the total lipid present in the particle.

In some embodiments, the additional lipid comprises a mixture of: (i) a phospholipid such as DOPE; and (ii) cholesterol or a derivative thereof. In some embodiments, the molar ratio of the phospholipid such as DOPE to the cholesterol or a derivative thereof is from about 9:0 to about 1:10, about 2:1 to about 1:4, about 1:1 to about 1:4, or about 1:1 to about 1:3.

### Polymer-Conjugated Lipids

In some embodiments, RNA compositions and formulations and RNA particles described herein may comprise at least one polymer-conjugated lipid. A polymer-conjugated lipid is typically a molecule comprising a lipid portion and a polymer portion conjugated thereto. In some embodiments, a polymer-conjugated lipid is a PEG-conjugated lipid, also referred to herein as pegylated lipid or PEG-lipid. The term “pegylated lipid” refers to a molecule comprising both a lipid portion and a polyethylene glycol portion. Pegylated lipids are known in the art. In some embodiments, a polymer-conjugated lipid is a polysarcosine-conjugated lipid, also referred to herein as sarcosinylated lipid or pSar-lipid. The term “sarcosinylated lipid” refers to a molecule comprising both a lipid portion and a polysarcosine portion.

In some embodiments, a polymer-conjugated lipid is designed to sterically stabilize a lipid particle by forming a protective hydrophilic layer that shields the hydrophobic lipid layer. In some embodiments, a polymer-conjugated lipid can reduce its association with serum proteins and/or the resulting uptake by the reticuloendothelial system when such lipid particles are administered in vivo.

### Polethyleneglycol (PEG)-Conjugated Lipids

In some embodiments, RNA compositions/formulations and RNA particles described herein comprise a PEG-conjugated lipid.

In some embodiments, the PEG-conjugated lipid (pegylated lipid) is a lipid having the structure of the following general formula:

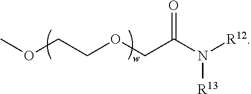

or a pharmaceutically acceptable salt, tautomer or stereoisomer thereof, wherein:
each of R12 and R13 is each independently a straight or branched, alkyl or alkenyl chain containing from 10 to 30 carbon atoms, wherein the alkyl/alkenyl chain is optionally interrupted by one or more ester bonds; and w has a mean value ranging from 30 to 60.

In some embodiments of this formula, each of R12 and R13 is independently a straight alkyl chain containing from 10 to 18 carbon atoms, preferably from 12 to 16 carbon atoms.

In some embodiments of this formula, R12 and R13 are identical. In some embodiments, each of R12 and R13 is a straight alkyl chain containing 12 carbon atoms. In some embodiments, each of R12 and R13 is a straight alkyl chain containing 14 carbon atoms. In some embodiments, each of R12 and R13 is a straight alkyl chain containing 16 carbon atoms.

In some embodiments of this formula, R12 and R13 are different. In some embodiments, one of R12 and R13 is a straight alkyl chain containing 12 carbon atoms and the other of R12 and R13 is a straight alkyl chain containing 14 carbon atoms.

In some embodiments of this formula, w has a mean value ranging from 40 to 50, such as a mean value of 45.

In some embodiments of this formula, w is within a range such that the PEG portion of the pegylated lipid has an average molecular weight of from about 400 to about 6000 g/mol, such as from about 1000 to about 5000 g/mol, from about 1500 to about 4000 g/mol, or from about 2000 to about 3000 g/mol. In some embodiments, each of R12 and R13 is a straight alkyl chain containing 14 carbon atoms and w has a mean value of 45.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω methoxy(polyethoxy)ethyl)carbamate, and the like.

In some embodiments, the PEG-conjugated lipid (pegylated lipid) is or comprises 2-[(polyethylene glycol)-2000]-N,N-ditetradecylacetamide. In some embodiments, the pegylated lipid has the following structure:

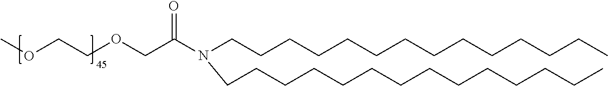

In some embodiments, the PEG-conjugated lipid (pegylated lipid) is DMG-PEG 2000, e.g., having the following structure:

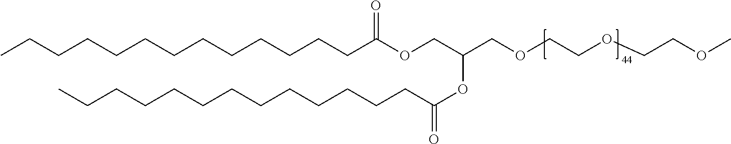

In some embodiments, the PEG-conjugated lipid (pegylated lipid) has the following structure:

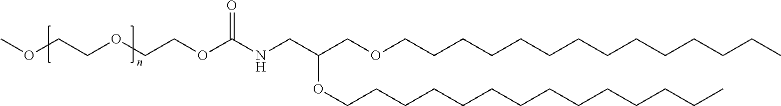

wherein n has a mean value ranging from 30 to 60, such as about 50. In one embodiment, the PEG-conjugated lipid (pegylated lipid) is PEG2000-C-DMA which preferably refers to 3-N-[(ω-methoxy poly(ethylene glycol)2000)carbamoyl]-1,2-dimyristyloxy-propylamine (MPEG-(2 kDa)-C-DMA) or methoxy-polyethylene glycol-2,3-bis(tetradecyloxy)propylcarbamate (2000).

In some embodiments, RNA compositions/formulations described herein may comprise one or more PEG-conjugated lipids or pegylated lipids as described in WO 2017/075531 and WO 2018/081480, the entire contents of each of which are incorporated herein by reference for the purposes described herein.

In some embodiments, the pegylated lipid comprises from about 1 mol % to about 10 mol %, preferably from about 1 mol % to about 5 mol %, more preferably from about 1 mol % to about 2.5 mol % of the total lipid present in the RNA compositions/formulations and RNA particles described herein.

### Embodiments of Lipoplex Particles

In some embodiments of the present disclosure, the RNA described herein may be present in RNA lipoplex particles.

Lipoplexes (LPX) are electrostatic complexes which are generally formed by mixing preformed cationic lipid liposomes with anionic nucleic acids. Formed lipoplexes possess distinct internal arrangements of molecules that arise due to the transformation from liposomal structure into compact RNA-lipoplexes.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in some embodiments ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In some embodiments, the RNA lipoplex particles have an average diameter that ranges from about 250 nm to about 700 nm. In some embodiments, the RNA lipoplex particles have an average diameter that ranges from about 300 nm to about 500 nm. In an exemplary embodiment, the RNA lipoplex particles have an average diameter of about 400 nm.

The RNA lipoplex particles and compositions comprising RNA lipoplex particles described herein are useful for delivery of RNA to a target tissue after parenteral administration, in particular after intravenous administration.

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In some embodiments, after administration of the DNA and/or RNA lipoplex particles, RNA accumulation and/or RNA expression in antigen presenting cells, such as professional antigen presenting cells in the spleen occurs. Thus, RNA lipoplex particles of the disclosure may be used for targeting RNA, e.g., RNA encoding an antigen or at least one epitope, to the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen. In some embodiments, the target cell is a spleen cell. In some embodiments, the target cell is an antigen presenting cell such as a professional antigen presenting cell in the spleen. In some embodiments, the target cell is a dendritic cell in the spleen.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)]. The concentration of RNA and the at least one cationic lipid amount can be determined using routine methods by one skilled in the art.

In some embodiments, at physiological pH the charge ratio of positive charges to negative charges in the RNA lipoplex particles is from about 1.6:2 to about 1:2, or about 1.6:2 to about 1.1:2. In specific embodiments, the charge ratio of positive charges to negative charges in the RNA lipoplex particles at physiological pH is about 1.6:2.0, about 1.5:2.0, about 1.4:2.0, about 1.3:2.0, about 1.2:2.0, about 1.1:2.0, or about 1:2.0.

### Embodiments of Lipid Nanoparticles (LNPs)

In some embodiments, RNA described herein is present in the form of lipid nanoparticles (LNPs). LNPs typically comprise four components: cationically ionizable lipid, neutral lipids such as phospholipids, a steroid such as cholesterol, and a polymer-conjugated lipid such as PEG-lipid. LNPs may be prepared by mixing lipids dissolved in ethanol with RNA in an aqueous buffer.

In some embodiments, in the RNA LNPs described herein the RNA is bound by cationically ionizable lipid that occupies the central core of the LNP. Polymer-conjugated lipid forms the surface of the LNP, along with phospholipids. In some embodiments, cholesterol and cationically ionizable lipid can be distributed throughout the LNP.

In some embodiments, the LNP comprises one or more cationically ionizable lipids, and one or more stabilizing lipids. Stabilizing lipids include neutral lipids and polymer-conjugated lipids.

In some embodiments, the LNP comprises a cationically ionizable lipid, a neutral lipid, a steroid, a polymer-conjugated lipid; and the RNA, encapsulated within or associated with the lipid nanoparticle.

In some embodiments, the LNP comprises from 35 to 65 mol percent, 40 to 60 mol percent, 40 to 55 mol percent, from 45 to 55 mol percent, or from 45 to 50 mol percent of the cationically ionizable lipid.

In some embodiments, the neutral lipid is present in a concentration ranging from 5 to 15 mol percent, from 7 to 13 mol percent, or from 9 to 11 mol percent.

In some embodiments, the steroid is present in a concentration ranging from 30 to 50 mol percent, from 30 to 45 mol percent, from 35 to 45 mol percent or from 35 to 43 mol percent.

In some embodiments, the LNP comprises from 1 to 10 mol percent, from 1 to 5 mol percent, or from 1 to 2.5 mol percent of the polymer-conjugated lipid.

In some embodiments, the LNP comprises from 45 to 55 mol percent of a cationically ionizable lipid; from 5 to 15 mol percent of a neutral lipid; from 30 to 45 mol percent of a steroid; from 1 to 5 mol percent of a polymer-conjugated lipid; and the RNA, encapsulated within or associated with the lipid nanoparticle.

In some embodiments, the mol percent is determined based on total mol of lipid present in the lipid nanoparticle. In some embodiments, the mol percent is determined based on total mol of cationically ionizable lipid, neutral lipid, steroid and polymer-conjugated lipid present in the lipid nanoparticle.

In some embodiments, the neutral lipid is selected from the group consisting of DSPC, DPPC, DMPC, DOPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

In some embodiments, the steroid is cholesterol.

In some embodiments, the polymer conjugated lipid is a pegylated lipid, e.g., a pegylated lipid as described above.

In some embodiments, the cationically ionizable lipid component of the LNPs has the structure of Formula (III):

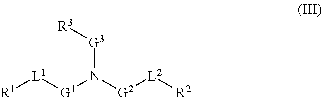

or a pharmaceutically acceptable salt, tautomer, prodrug or stereoisomer thereof, wherein:
one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa- or —NRaC(═O)O—, and the other of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, NRaC(═O)NRa—, —OC(═O)NRa- or —NRaC(═O)O— or a direct bond;
G1 and G2 are each independently unsubstituted C1-C12 alkylene or C1-C12 alkenylene;
G3 is C1-C24 alkylene, C1-C24 alkenylene, C3-C8 cycloalkylene, C3-C8 cycloalkenylene;
Ra is H or C1-C12 alkyl;
R1 and R2 are each independently C6-C24 alkyl or C6-C24 alkenyl;
R3 is H, OR5, CN, —C(═O)OR4, —OC(═O)R4 or —NR5C(═O)R4;
R4 is C1-C12 alkyl;
R5 is H or C1-C6 alkyl; and
x is 0, 1 or 2.

In some of the foregoing embodiments of Formula (III), the lipid has one of the following structures (IIIA) or (IIIB):

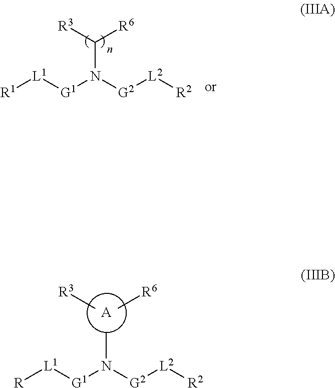

wherein:
A is a 3 to 8-membered cycloalkyl or cycloalkylene ring;
R6 is, at each occurrence, independently H, OH or C1-C24 alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

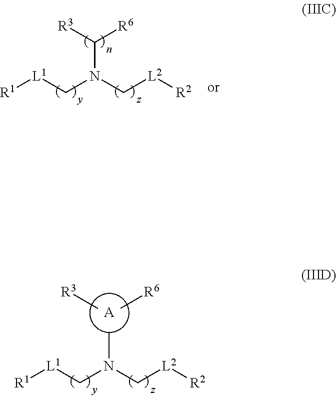

wherein y and z are each independently integers ranging from 1 to 12.

In any of the foregoing embodiments of Formula (III), one of L1 or L2 is —O(C═O)—. For example, in some embodiments each of L1 and L2 are —O(C═O)—. In some different embodiments of any of the foregoing, L1 and L2 are each independently —(C═O)O—or —O(C═O)—. For example, in some embodiments each of L1 and L2 is —(C═O)O—.

In some different embodiments of Formula (III), the lipid has one of the following structures (IIIE) or (IIIF):

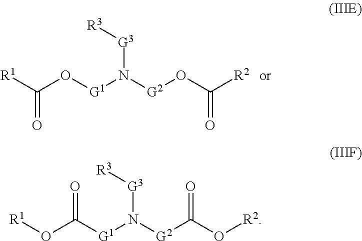

In some of the foregoing embodiments of Formula (III), the lipid has one of the following structures (IIIG), (IIIH), (IIII), or (IIIJ);

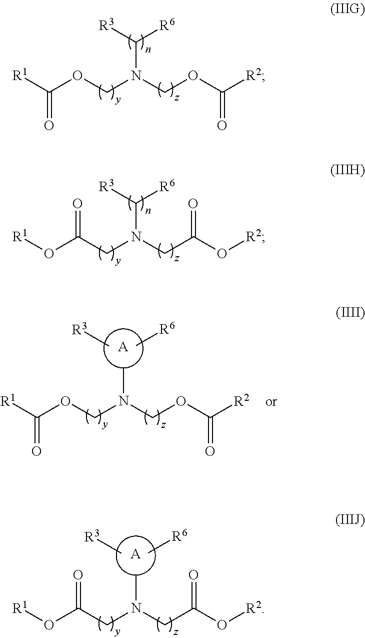

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R6 is H. In other of the foregoing embodiments, R6 is C1-C24 alkyl. In other embodiments, R6 is OH.

In some embodiments of Formula (III), G3 is unsubstituted. In other embodiments, G3 is substituted. In various different embodiments, G3 is linear C1-C24 alkylene or linear C1-C24 alkenylene.

In some other foregoing embodiments of Formula (III), R1 or R2, or both, is C6-C24 alkenyl. For example, in some embodiments, R1 and R2 each, independently have the following structure:

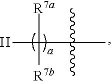

wherein:
R7a and R7b are, at each occurrence, independently H or C1-C12 alkyl; and
a is an integer from 2 to 12,
wherein R7a, R7b and a are each selected such that R1 and R2 each independently comprise from 6 to 20 carbon atoms. For example, in some embodiments a is an integer ranging from 5 to 9 or from 8 to 12.

In some of the foregoing embodiments of Formula (III), at least one occurrence of R7a is H. For example, in some embodiments, R7a is H at each occurrence. In other different embodiments of the foregoing, at least one occurrence of R7b is C1-C8 alkyl. For example, in some embodiments, C1-C8 alkyl is methyl, ethyl, n-propyl, iso-propyl, n-butyl, iso-butyl, tert-butyl, n-hexyl or n-octyl.

In different embodiments of Formula (III), R1 or R2, or both, has one of the following structures:

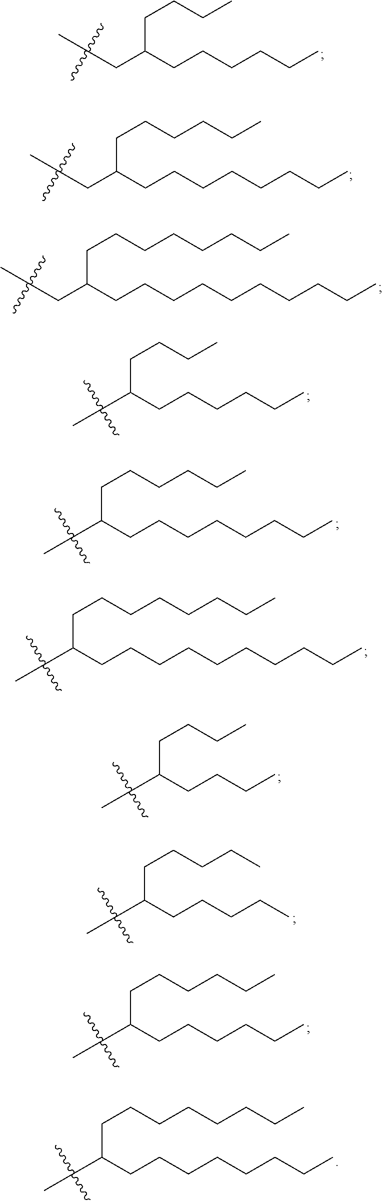

In some of the foregoing embodiments of Formula (III), R3 is OH, CN, —C(═O)OR4, —OC(═O)R4 or —NHC(═O)R4. In some embodiments, R4 is methyl or ethyl.

In various different embodiments, the cationic lipid of Formula (III) has one of the structures set forth in the table below.

Representative Compounds of Formula (III).

| No. | Structure |
|---|---|
| III-1 | 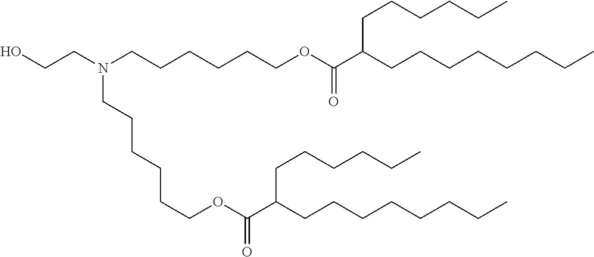 [SMILES: CCCCCCCCC(CCCCCC)C(=O)OCCCCCCN(CCO)CCCCCCOC(=O)C(CCCCCC)CCCCCCCC] |
| III-2 | 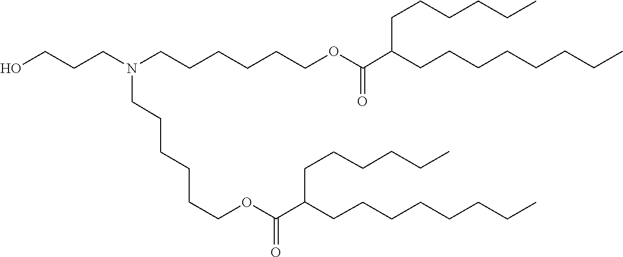 [SMILES: CCCCCCCCC(CCCCCC)C(=O)OCCCCCCN(CCCO)CCCCCCOC(=O)C(CCCCCC)CCCCCCCC] |
| III-3 | 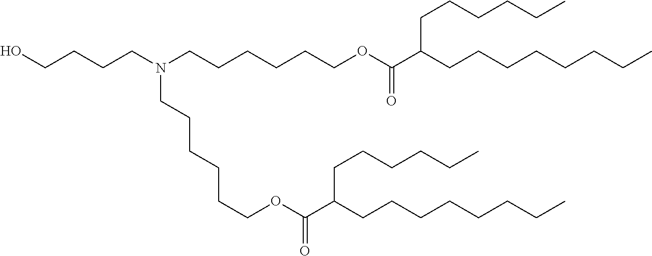 [SMILES: CCCCCCCCC(CCCCCC)C(=O)OCCCCCCN(CCCCO)CCCCCCOC(=O)C(CCCCCC)CCCCCCCC] |
| III-4 | 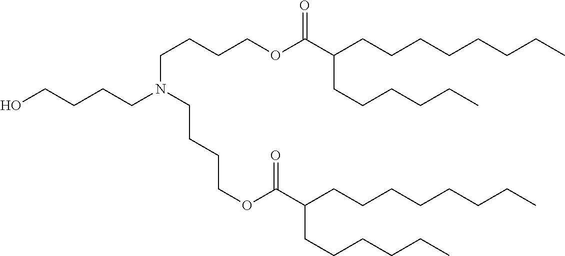 [SMILES: CCCCCCCCC(CCCCCC)C(=O)OCCCCN(CCCCO)CCCCOC(=O)C(CCCCCC)CCCCCCCC] |
| III-5 | 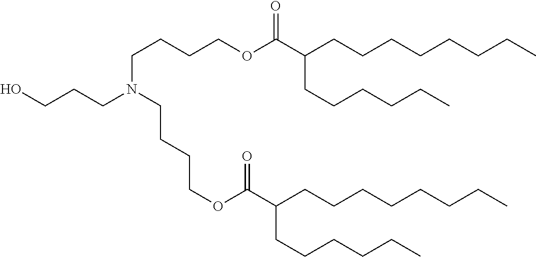 [SMILES: CCCCCCCCC(CCCCCC)C(=O)OCCCCN(CCCO)CCCCOC(=O)C(CCCCCC)CCCCCCCC] |
| III-6 | 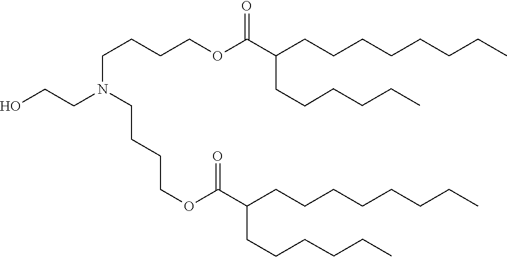 [SMILES: CCCCCCCCC(CCCCCC)C(=O)OCCCCN(CCO)CCCCOC(=O)C(CCCCCC)CCCCCCCC] |
| III-7 | 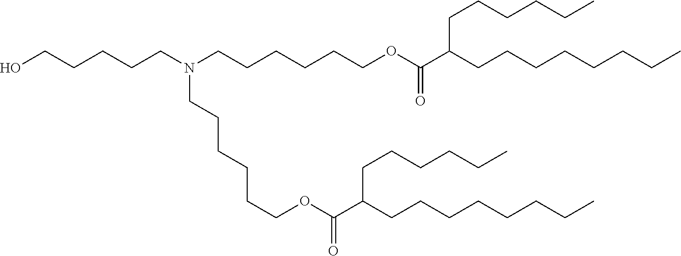 [SMILES: CCCCCCCCC(CCCCCC)C(=O)OCCCCCCN(CCCCCO)CCCCCCOC(=O)C(CCCCCC)CCCCCCCC] |
| III-8 | 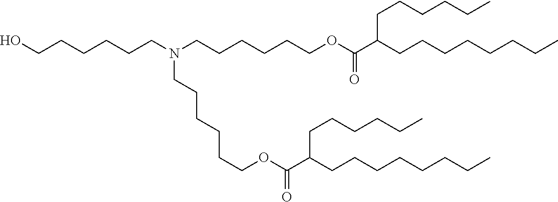 [SMILES: CCCCCCCCC(CCCCCC)C(=O)OCCCCCCN(CCCCCCO)CCCCCCOC(=O)C(CCCCCC)CCCCCCCC] |
| III-9 | 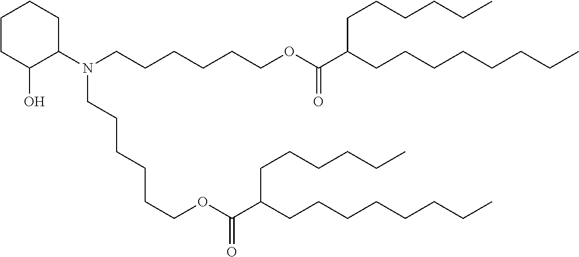 [SMILES: CCCCCCCCC(CCCCCC)C(=O)OCCCCCCN(CCCCCCOC(=O)C(CCCCCC)CCCCCCCC)C1CCCCC1O] |
| III-10 | 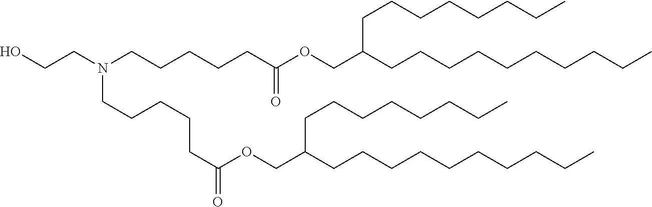 [SMILES: CCCCCCCCCCC(CCCCCCCC)COC(=O)CCCCCN(CCO)CCCCCC(=O)OCC(CCCCCCCC)CCCCCCCCCC] |
| III-11 | 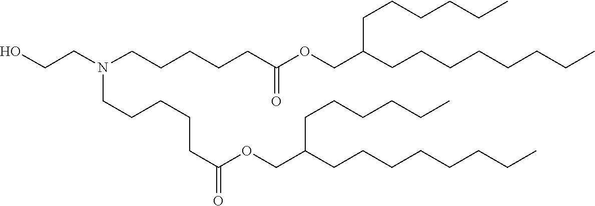 [SMILES: CCCCCCCCC(CCCCCC)COC(=O)CCCCCN(CCO)CCCCCC(=O)OCC(CCCCCC)CCCCCCCC] |
| III-12 | 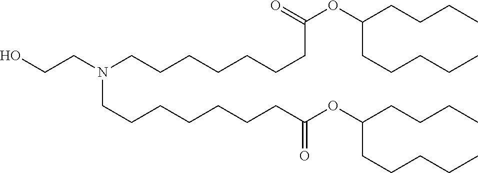 [SMILES: CCCCCC(CCCC)OC(=O)CCCCCCCN(CCO)CCCCCCCC(=O)OC(CCCC)CCCCC] |
| III-13 | 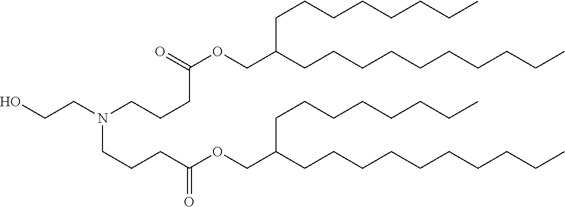 [SMILES: CCCCCCCCCCC(CCCCCCCC)COC(=O)CCCN(CCO)CCCC(=O)OCC(CCCCCCCC)CCCCCCCCCC] |
| III-14 | 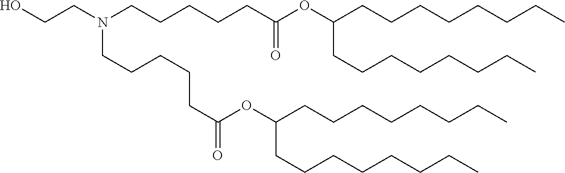 [SMILES: CCCCCCCCC(CCCCCCCC)OC(=O)CCCCCN(CCO)CCCCCC(=O)OC(CCCCCCCC)CCCCCCCC] |
| III-15 | 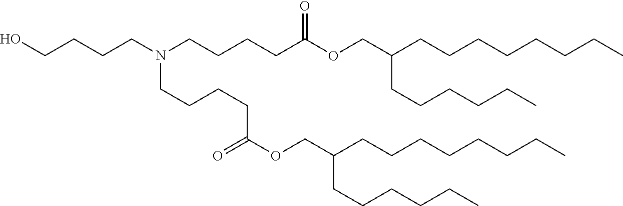 [SMILES: CCCCCCCCC(CCCCCC)COC(=O)CCCCN(CCCCO)CCCCC(=O)OCC(CCCCCC)CCCCCCCC] |
| III-16 | 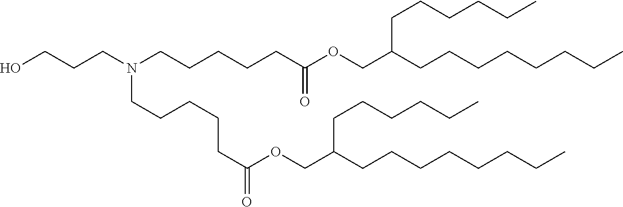 [SMILES: CCCCCCCCC(CCCCCC)COC(=O)CCCCCN(CCCO)CCCCCC(=O)OCC(CCCCCC)CCCCCCCC] |
| III-17 | 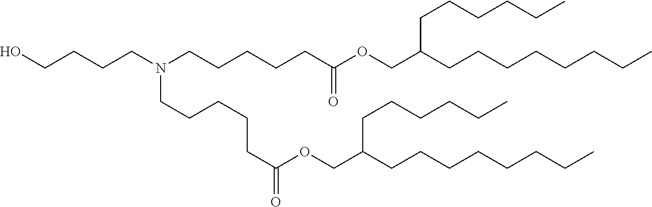 [SMILES: CCCCCCCCC(CCCCCC)COC(=O)CCCCCN(CCCCO)CCCCCC(=O)OCC(CCCCCC)CCCCCCCC] |
| III-18 | 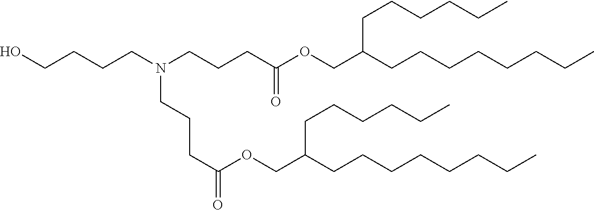 [SMILES: CCCCCCCCC(CCCCCC)COC(=O)CCCN(CCCCO)CCCC(=O)OCC(CCCCCC)CCCCCCCC] |
| III-19 | 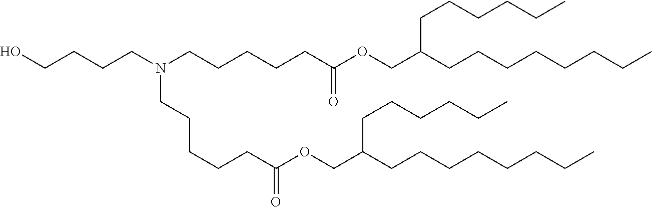 [SMILES: CCCCCCCCC(CCCCCC)COC(=O)CCCCCN(CCCCO)CCCCCC(=O)OCC(CCCCCC)CCCCCCCC] |
| III-20 | 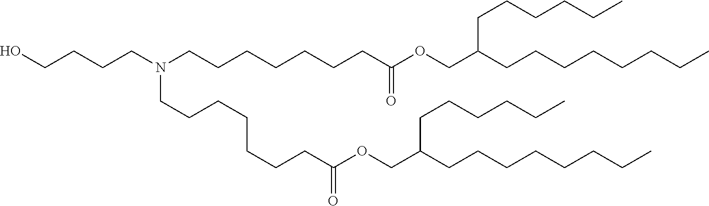 [SMILES: CCCCCCCCC(CCCCCC)COC(=O)CCCCCCCN(CCCCO)CCCCCCCC(=O)OCC(CCCCCC)CCCCCCCC] |
| III-21 | 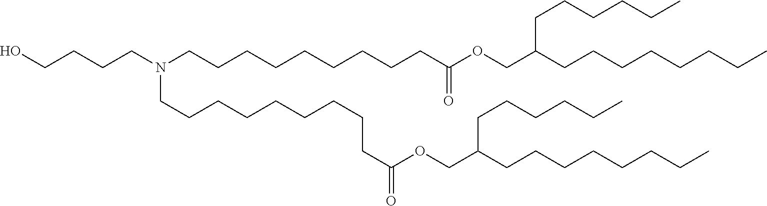 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 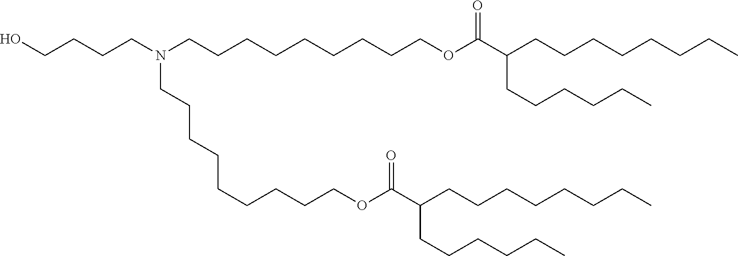 [SMILES: CCCCCCCCC(CCCCCC)C(=O)OCCCCCCCCCN(CCCCO)CCCCCCCCCOC(=O)C(CCCCCC)CCCCCCCC] |
| III-23 | 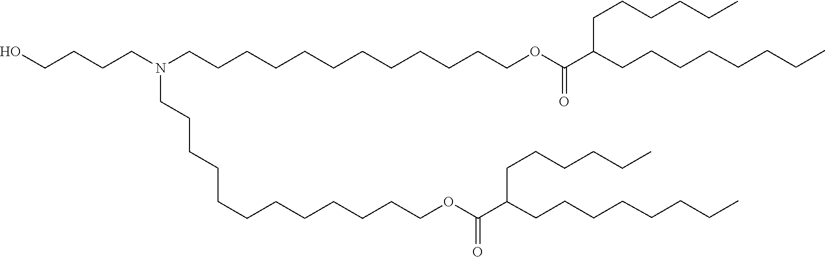 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 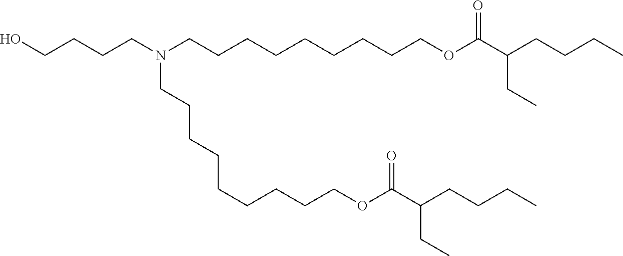 [SMILES: CCCCC(CC)C(=O)OCCCCCCCCCN(CCCCO)CCCCCCCCCOC(=O)C(CC)CCCC] |
| III-25 | 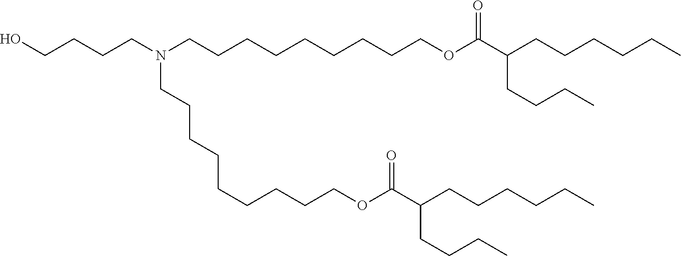 [SMILES: CCCCCCC(CCCC)C(=O)OCCCCCCCCCN(CCCCO)CCCCCCCCCOC(=O)C(CCCC)CCCCCC] |
| III-26 | 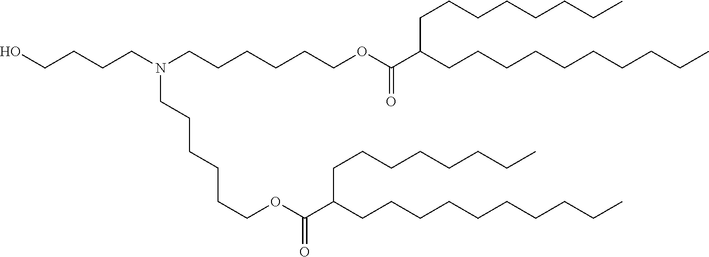 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 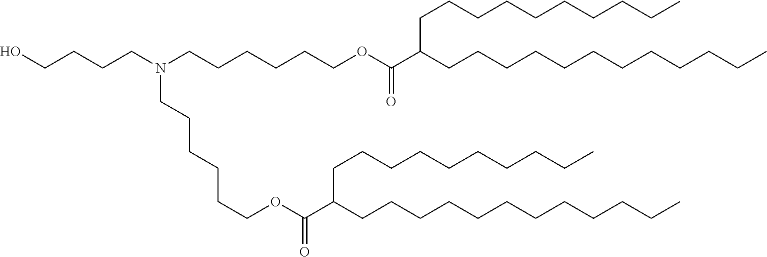 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 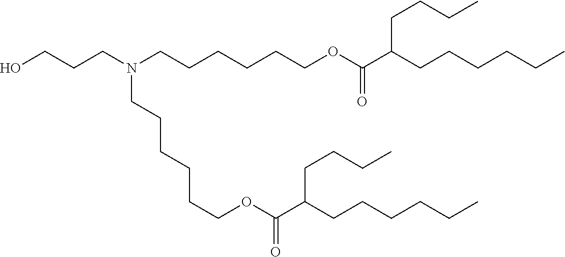 [SMILES: CCCCCCC(CCCC)C(=O)OCCCCCCN(CCCO)CCCCCCOC(=O)C(CCCC)CCCCCC] |
| III-29 | 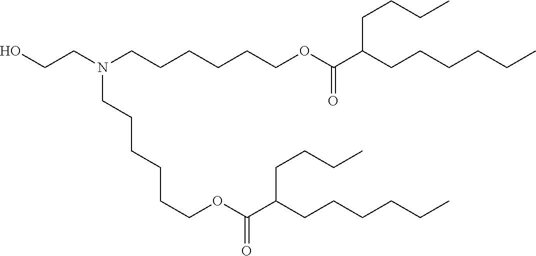 [SMILES: CCCCCCC(CCCC)C(=O)OCCCCCCN(CCO)CCCCCCOC(=O)C(CCCC)CCCCCC] |
| III-30 | 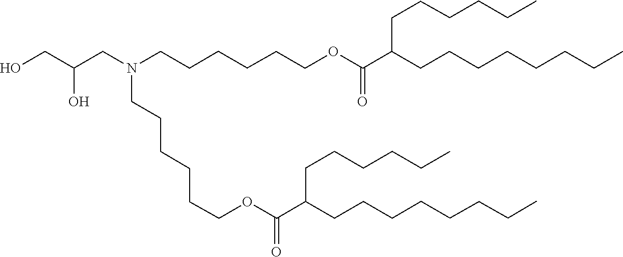 [SMILES: CCCCCCCCC(CCCCCC)C(=O)OCCCCCCN(CCCCCCOC(=O)C(CCCCCC)CCCCCCCC)CC(O)CO] |
| III-31 | 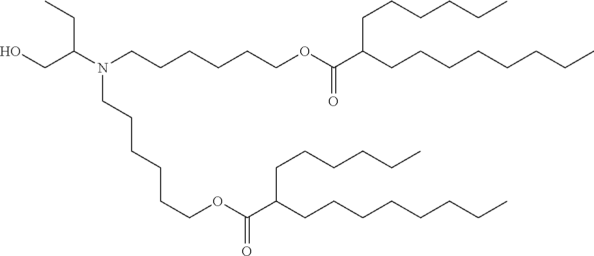 [SMILES: CCCCCCCCC(CCCCCC)C(=O)OCCCCCCN(CCCCCCOC(=O)C(CCCCCC)CCCCCCCC)C(CC)CO] |
| III-32 | 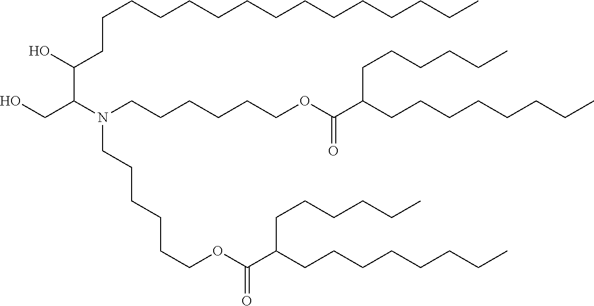 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 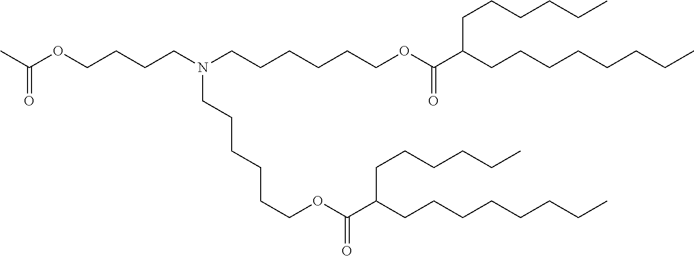 [SMILES: CCCCCCCCC(CCCCCC)C(=O)OCCCCCCN(CCCCCCOC(=O)C(CCCCCC)CCCCCCCC)CCCCOC(C)=O] |
| III-34 | 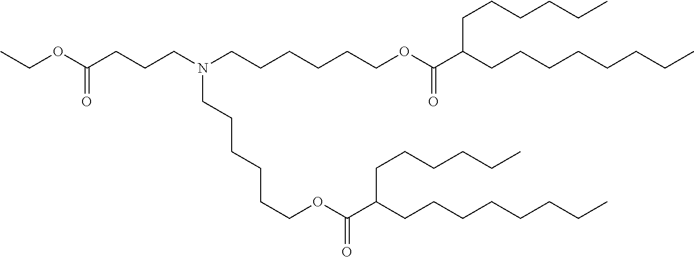 [SMILES: CCCCCCCCC(CCCCCC)C(=O)OCCCCCCN(CCCCCCOC(=O)C(CCCCCC)CCCCCCCC)CCCC(=O)OCC] |
| III-35 | 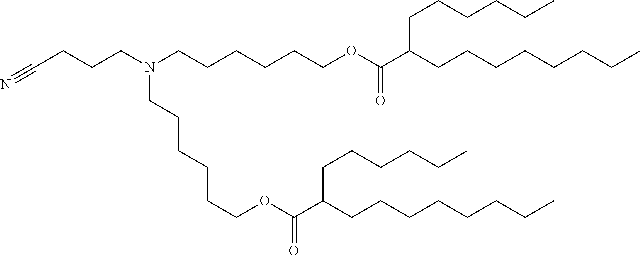 [SMILES: CCCCCCCCC(CCCCCC)C(=O)OCCCCCCN(CCCC#N)CCCCCCOC(=O)C(CCCCCC)CCCCCCCC] |
| III-36 | 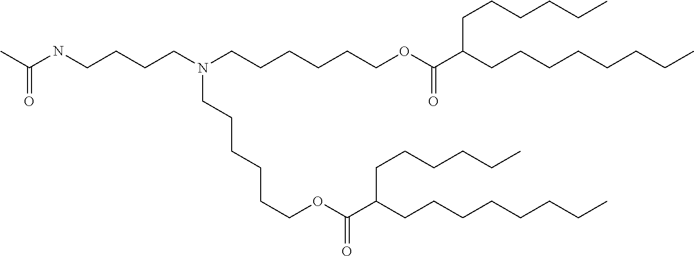 [SMILES: CCCCCCCCC(CCCCCC)C(=O)OCCCCCCN(CCCCCCOC(=O)C(CCCCCC)CCCCCCCC)CCCCNC(C)=O] |
| III-37 | 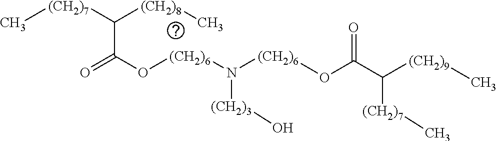 |
| III-38 | 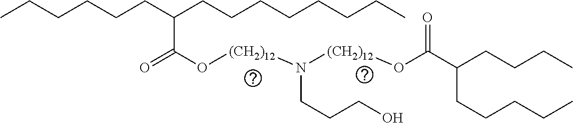 |
| III-39 | 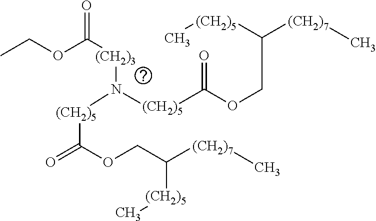 |
| III-40 | 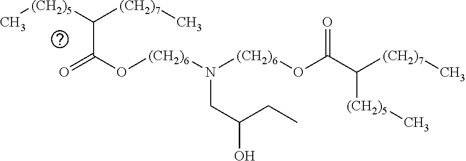 |
| III-41 | 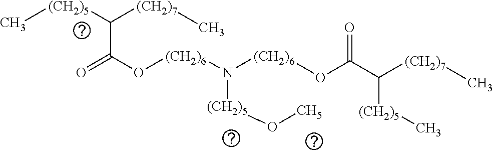 |
| III-42 | 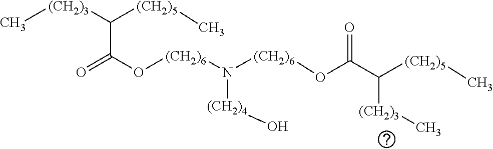 |
| III-43 | 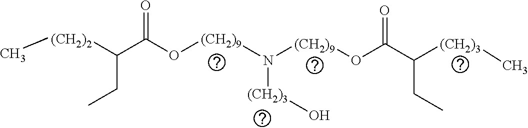 |
| III-44 | 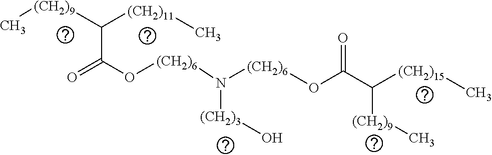 |
| III-45 | 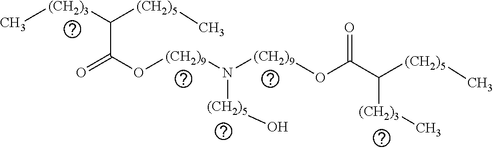 |
| III-46 | 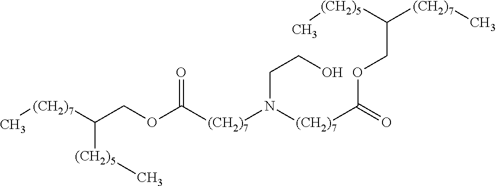 |
| III-47 | 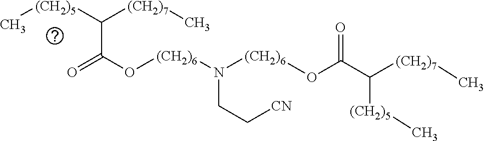 |
| III-48 | 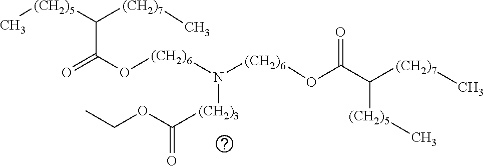 |
| III-49 | 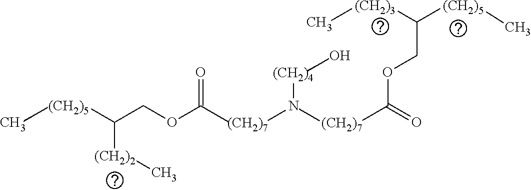 |

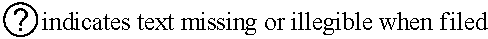

Further representative cationically ionizable lipids are as follows:

| No. | Structure |
|---|---|
| A | 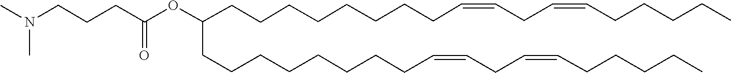 [SMILES: CCCCC/C=C\C/C=C\CCCCCCCCC(CCCCCCCC/C=C\C/C=C\CCCCC)OC(=O)CCCN(C)C] |
| B | 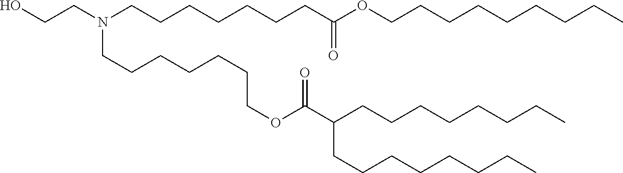 [SMILES: CCCCCCCCCOC(=O)CCCCCCCN(CCO)CCCCCCCOC(=O)C(CCCCCCCC)CCCCCCCC] |
| C | 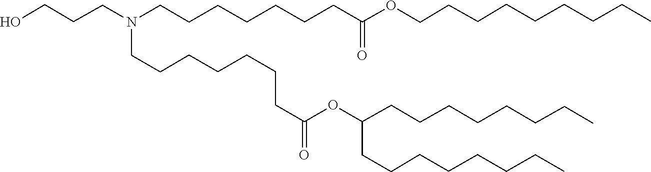 [SMILES: CCCCCCCCCOC(=O)CCCCCCCN(CCCO)CCCCCCCC(=O)OC(CCCCCCCC)CCCCCCCC] |
| D | 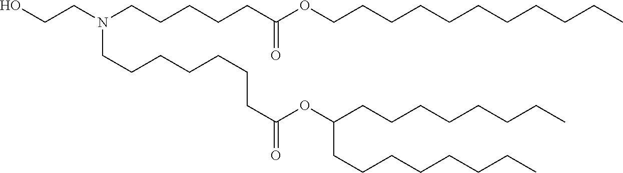 [SMILES: CCCCCCCCCCCOC(=O)CCCCCN(CCO)CCCCCCCC(=O)OC(CCCCCCCC)CCCCCCCC] |
| E | 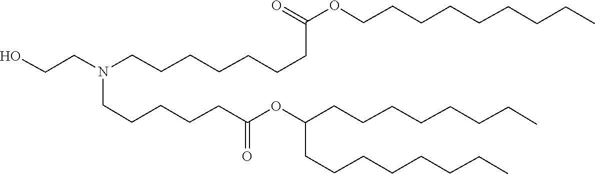 [SMILES: CCCCCCCCCOC(=O)CCCCCCCN(CCO)CCCCCC(=O)OC(CCCCCCCC)CCCCCCCC] |
| F | 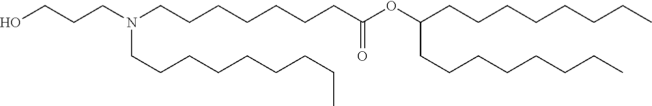 [SMILES: CCCCCCCCCN(CCCO)CCCCCCCC(=O)OC(CCCCCCCC)CCCCCCCC] |

In some embodiments, RNA described herein is formulated in an LNP composition comprising a cationically ionizable lipid, e.g., a cationically ionizable lipid as shown above, a neutral lipid, a steroid, and a polymer conjugated lipid.

In some embodiments, RNA described herein is formulated in an LNP composition comprising a cationically ionizable lipid of Formula III, a neutral lipid, a steroid, and a polymer conjugated lipid.

In some embodiments, RNA described herein is formulated in an LNP composition comprising a cationically ionizable lipid shown in the above tables, a neutral lipid, a steroid, and a polymer conjugated lipid.

In some embodiments, RNA described herein is formulated in an LNP composition comprising 3D-P-DMA, a neutral lipid, a steroid, and a polymer conjugated lipid.

In some embodiments, RNA described herein is formulated in an LNP composition comprising ALC-0366, a neutral lipid, a steroid, and a polymer conjugated lipid.

In some embodiments, RNA described herein is formulated in an LNP composition comprising ALC-0315, a neutral lipid, a steroid, and a polymer conjugated lipid.

In some embodiments, the neutral lipid is DSPC. In some embodiments, the steroid is cholesterol. In some embodiments, the polymer conjugated lipid is a pegylated lipid, e.g., DMG-PEG 2000, PEG2000-C-DMA, or ALC-0159.

In some embodiments, RNA described herein is formulated in an LNP composition comprising a cationically ionizable lipid, e.g., a cationically ionizable lipid as shown above, a neutral lipid, a steroid, and a pegylated lipid.

In some embodiments, RNA described herein is formulated in an LNP composition comprising a cationically ionizable lipid of Formula III, a neutral lipid, a steroid, and a pegylated lipid.

In some embodiments, RNA described herein is formulated in an LNP composition comprising a cationically ionizable lipid shown in the above tables, a neutral lipid, a steroid, and a pegylated lipid.

In some embodiments, RNA described herein is formulated in an LNP composition comprising 3D-P-DMA, a neutral lipid, a steroid, and a pegylated lipid.

In some embodiments, RNA described herein is formulated in an LNP composition comprising ALC-0366, a neutral lipid, a steroid, and a pegylated lipid.

In some embodiments, RNA described herein is formulated in an LNP composition comprising ALC-0315, a neutral lipid, a steroid, and a pegylated lipid.

In some embodiments, the neutral lipid is DSPC. In some embodiments, the steroid is cholesterol. In some embodiments, the pegylated lipid is DMG-PEG 2000, PEG2000-C-DMA, or ALC-0159.

In some embodiments, RNA described herein is formulated in an LNP composition comprising a cationically ionizable lipid, e.g., a cationically ionizable lipid as shown above, DSPC, cholesterol, and a pegylated lipid.

In some embodiments, RNA described herein is formulated in an LNP composition comprising a cationically ionizable lipid of Formula III, DSPC, cholesterol, and a pegylated lipid.

In some embodiments, RNA described herein is formulated in an LNP composition comprising a cationically ionizable lipid shown in the above tables, DSPC, cholesterol, and a pegylated lipid.

In some embodiments, RNA described herein is formulated in an LNP composition comprising 3D-P-DMA, DSPC, cholesterol, and a pegylated lipid.

In some embodiments, RNA described herein is formulated in an LNP composition comprising ALC-0366, DSPC, cholesterol, and a pegylated lipid.

In some embodiments, RNA described herein is formulated in an LNP composition comprising ALC-0315, DSPC, cholesterol, and a pegylated lipid.

In some embodiments, the pegylated lipid is DMG-PEG 2000, PEG2000-C-DMA, or ALC-0159.

In some embodiments, RNA described herein is formulated in an LNP composition comprising a cationically ionizable lipid, e.g., a cationically ionizable lipid as shown above, DSPC, cholesterol, and DMG-PEG 2000.

In some embodiments, RNA described herein is formulated in an LNP composition comprising a cationically ionizable lipid of Formula III, DSPC, cholesterol, and DMG-PEG 2000.

In some embodiments, RNA described herein is formulated in an LNP composition comprising a cationically ionizable lipid shown in the above tables, DSPC, cholesterol, and DMG-PEG 2000.

In some embodiments, RNA described herein is formulated in an LNP composition comprising 3D-P-DMA, DSPC, cholesterol, and DMG-PEG 2000.

In some embodiments, RNA described herein is formulated in an LNP composition comprising ALC-0366, DSPC, cholesterol, and DMG-PEG 2000.

In some embodiments, RNA described herein is formulated in an LNP composition comprising ALC-0315, DSPC, cholesterol, and DMG-PEG 2000.

In some embodiments, RNA described herein is formulated in an LNP composition comprising a cationically ionizable lipid, e.g., a cationically ionizable lipid as shown above, DSPC, cholesterol, and PEG2000-C-DMA.

In some embodiments, RNA described herein is formulated in an LNP composition comprising a cationically ionizable lipid of Formula III, DSPC, cholesterol, and PEG2000-C-DMA.

In some embodiments, RNA described herein is formulated in an LNP composition comprising a cationically ionizable lipid shown in the above tables, DSPC, cholesterol, and PEG2000-C-DMA.

In some embodiments, RNA described herein is formulated in an LNP composition comprising 3D-P-DMA, DSPC, cholesterol, and PEG2000-C-DMA.

In some embodiments, RNA described herein is formulated in an LNP composition comprising ALC-0366, DSPC, cholesterol, and PEG2000-C-DMA.

In some embodiments, RNA described herein is formulated in an LNP composition comprising ALC-0315, DSPC, cholesterol, and PEG2000-C-DMA.

In some embodiments, RNA described herein is formulated in an LNP composition comprising a cationically ionizable lipid, e.g., a cationically ionizable lipid as shown above, DSPC, cholesterol, and ALC-0159.

In some embodiments, RNA described herein is formulated in an LNP composition comprising a cationically ionizable lipid of Formula III, DSPC, cholesterol, and ALC-0159.

In some embodiments, RNA described herein is formulated in an LNP composition comprising a cationically ionizable lipid shown in the above tables, DSPC, cholesterol, and ALC-0159.

In some embodiments, RNA described herein is formulated in an LNP composition comprising 3D-P-DMA, DSPC, cholesterol, and ALC-0159.

In some embodiments, RNA described herein is formulated in an LNP composition comprising ALC-0366, DSPC, cholesterol, and ALC-0159.

In some embodiments, RNA described herein is formulated in an LNP composition comprising ALC-0315, DSPC, cholesterol, and ALC-0159.

### 3D-P-DMA: (6Z,16Z)-12-((Z)-dec-4-en-1-yl)docosa-6,16-dien-11-yl 5-(dimethylamino)pentanoate

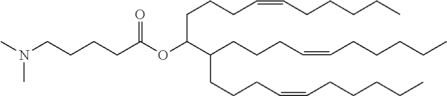 [SMILES: CCCCC/C=C\CCCC(CCC/C=C\CCCCC)C(CCC/C=C\CCCCC)OC(=O)CCCCN(C)C]

### ALC-0366: ((3-hydroxypropyl)azanediyl)bis(nonane-9,1-diyl) bis(2-butyloctanoate)

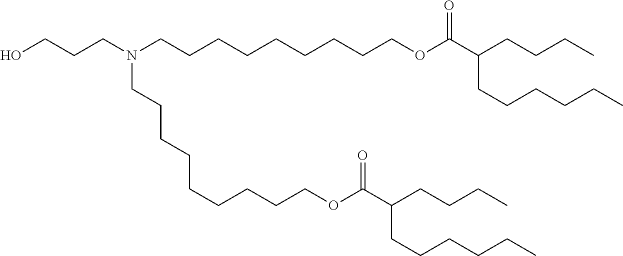 [SMILES: CCCCCCC(CCCC)C(=O)OCCCCCCCCCN(CCCO)CCCCCCCCCOC(=O)C(CCCC)CCCCCC]

### ALC-0315: ((4-hydroxybutyl)azanediyl)bis(hexane-6,1-diyl)bis(2-hexyldecanoate)/6-[N-6-(2-hexyldecanoyloxy)hexyl-N-(4-hydroxybutyl)amino]hexyl 2-hexyldecanoate

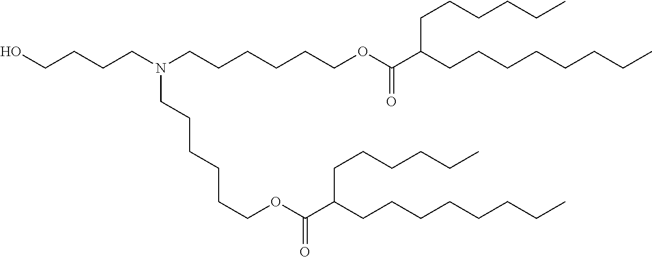 [SMILES: CCCCCCCCC(CCCCCC)C(=O)OCCCCCCN(CCCCO)CCCCCCOC(=O)C(CCCCCC)CCCCCCCC]

### DMG-PEG 2000:

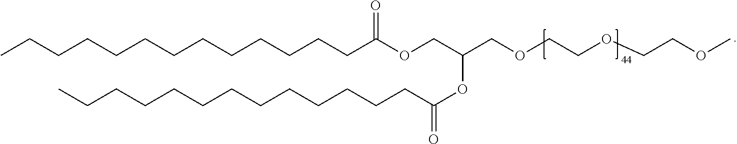

### PEG2000-C-DMA: 3-N-[(ω-Methoxy poly(ethylene glycol)2000) carbamoyl]-1,2-dimyristyloxy-propylamine (MPEG-(2 kDa)-C-DMA or Methoxy-polyethylene glycol-2,3-bis(tetradecyloxy)propylcarbamate (2000))

wherein n has a mean value ranging from 30 to 60, such as about 50.

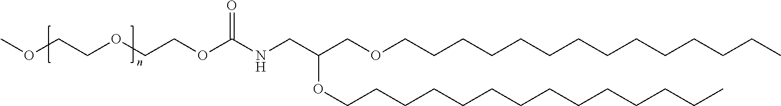

### ALC-0159: 2-[(polyethylene glycol)-2000]-N,N-ditetradecylacetamide/2-[2-(ω-methoxy (polyethyleneglycol2000) ethoxy]-N,N-ditetradecylacetamide

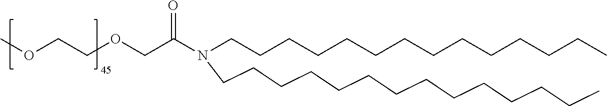

### DSPC: 1,2-Distearoyl-sn-glycero-3-phosphocholine

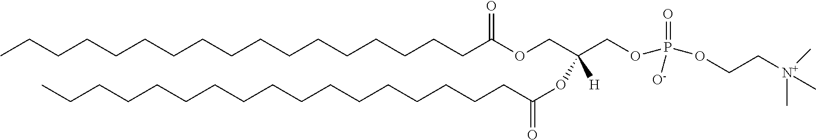 [SMILES: CCCCCCCCCCCCCCCCCC(=O)OC[C@H](COP(=O)([O-])OCC[N+](C)(C)C)OC(=O)CCCCCCCCCCCCCCCCC]

### Cholesterol:

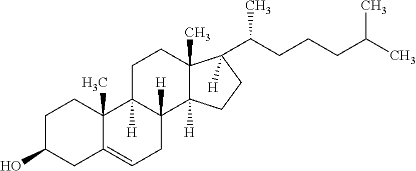

The N/P value is preferably at least about 4. In some embodiments, the N/P value ranges from 4 to 20, 4 to 12, 4 to 10, 4 to 8, or 5 to 7. In some embodiments, the N/P value is about 6.

The term “stealth” is used herein to describe the ability of the particles described herein not to be detected and then sequestered and/or degraded, or to be hardly detected and then sequestered and/or degraded, and/or to be detected and then sequestered and/or degraded late, by the immune system of the host to which they are administered.

Macrophages constitute one of the most important components of the immune system and play a predominant role in eliminating foreign particles, including liposomes and other colloidal particles, from the blood circulation. At the molecular level, the clearance of particles takes place in two steps: opsonization by the depositing of serum proteins (or “opsonins”) at the surface of the particles followed by recognition and capture of the opsonized particles by macrophages.

Modification of the surface of particles with chains of hydrophilic and flexible polymers, e.g., polymers of the poly(ethylene glycol) type, confers them a steric protection by preventing the opsonins reaching the surface of the particles.

In some embodiments, the amphiphilic derivative of a polymer used herein has as a hydrophobic group (e.g., lipid) as specified herein. In some embodiments, the amphiphilic derivative of a polymer used herein has as a hydrophobic group (e.g., lipid) a phospholipid, e.g., a biodegradable phospholipid such as phosphatidylethanolamine. In some embodiments, the phospholipid is selected from the group consisting of DSPE (distearoylphosphatidylethanolamine), DPPE (dipalmitoylphosphatidylethanolamine), DOPE (dioleoylphosphatidylethanolamine), and POPE (palmitoyloleylphosphatidylethanolamine), and mixtures thereof. In some embodiments, as a phospholipid, DSPE will be used for its qualities of stability in the particles described herein. Moreover, as hydrophobic group (e.g., lipid), a compound having at least one alkyl chain providing hydrophobic anchoring to a particle as described herein may be used.

In some embodiments, the polymer for use herein is selected from the group consisting of poly(ethylene glycol) (PEG), polysarcosine (pSar) (poly(N-methylglycine), polyoxazoline (POX), polyoxazine (POZ), and poly-2-(2-(2-aminoethoxy)ethoxy)acetic acid (pAEEA) (including derivatives thereof).

In some embodiments, a polymer is designed to sterically stabilize a particle by forming a protective hydrophilic layer. In some embodiments, a polymer can reduce association of a particle with serum proteins and/or the resulting uptake by the reticuloendothelial system when such particles are administered in vivo.

In some embodiments, the PEG is an optionally substituted linear or branched polymer of ethylene glycol or ethylene oxide. In some embodiments, the PEG is unsubstituted. In some embodiments, the PEG is substituted, e.g., by one or more alkyl, alkoxy, acyl, hydroxy or aryl groups. In some embodiments, the PEG has a molecular weight of from about 130 to about 50,000, in another embodiment about 150 to about 30,000, in another embodiment about 150 to about 20,000, in another embodiment about 150 to about 15,000, in another embodiment about 150 to about 10,000, in another embodiment about 150 to about 6000, in another embodiment about 150 to about 5000, in another embodiment about 150 to about 4000, in another embodiment about 150 to about 3000, in another embodiment about 300 to about 3000, in another embodiment about 1000 to about 3000, and in still another embodiment about 1500 to about 2500.

In some embodiments, the PEG moiety of the amphiphilic derivative of a polymer has a molecular weight of 1000 or more. In some embodiments, the PEG moiety of the amphiphilic derivative of a polymer comprises 10 units or more of formula (0—CH2-CH2)n. In some embodiments, the PEG comprises from 20 to 200 ethylene oxide units, such as about 45 ethylene oxide units.

In some embodiments, the PEG comprises “PEG2k”, also termed “PEG 2000”, which has an average molecular weight of about 2000 Daltons.

In some embodiments, DSPE-PEG2000, DSPE-PEG3000 and DSPE-PEG5000 are used as the amphiphilic derivative of a polymer.

In some embodiments, a pSar comprises between 2 and 200 sarcosine units, such as between 5 and 100 sarcosine units, between 10 and 50 sarcosine units, between 15 and 40 sarcosine units, e.g., about 23 sarcosine units.

In some embodiments, a pSar comprises the structure of the following general formula:

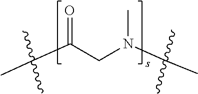

wherein s is the number of sarcosine units.

In some embodiments, the POX and/or POZ polymer comprises between 2 and 200, between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70 POX and/or POZ repeating units.

In some embodiments, the POX and/or POZ polymer comprises the following general formula:

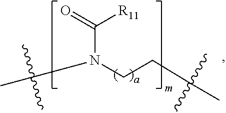

wherein a is an integer between 1 and 2; R11 is alkyl, in particular C1-3 alkyl, such as methyl, ethyl, iso-propyl, or n-propyl, and is independently selected for each repeating unit; and m refers to the number of POX and/or POZ repeating units.

In some embodiments, the POX and/or POZ polymer is a polymer of POX and comprises repeating units of the following general formula:

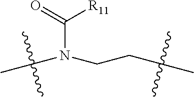

In some embodiments, the POX and/or POZ polymer is a polymer of POZ and comprises repeating units of the following general formula:

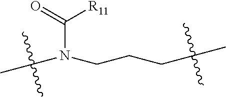

In any of the above embodiments of formulas, m (i.e., the number of repeating units in the polymer) preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments, m is 2 to 180, such as 4 to 160, 6 to 140, 8 to 120 or 10 to 100, e.g., 20 to 80, 30 to 70, or 40 to 50.

In some embodiments, the POX and/or POZ polymer is a copolymer comprising repeating units of the following general formulas:

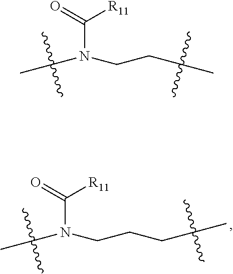

wherein the number of repeating units shown on the left in the copolymer is 1 to 199; the number of repeating units of formula on the right in the copolymer is 1 to 199; and the sum of the number of repeating units of formula on the left and the number of repeating units of formula on the right in the copolymer is 2 to 200.

In some embodiments of the oxazolinylated and/or oxazinylated hydrophobic moiety (e.g., lipid), the number of repeating units of formula on the left in the copolymer is 1 to 179, such as 1 to 159, 1 to 139, 1 to 119 or 1 to 99; the number of repeating units of formula on the right in the copolymer is 1 to 179, such as 1 to 159, 1 to 139, 1 to 119 or 1 to 99; and the sum of the number of repeating units of formula on the left and the number of repeating units of formula on the right in the copolymer is 2 to 180, such as 4 to 160, 6 to 140, 8 to 120 or 10 to 100, e.g., 20 to 80, 30 to 70, or 40 to 50.

In some of the above embodiments, R11 at each occurrence (i.e., in each repeating unit) may be the same alkyl group (e.g., R11 may be methyl in each repeating unit). In some alternative embodiments, R11 in at least one repeating unit differs from R11 in another repeating unit (e.g., for at least one repeating unit R11 is one specific alkyl (such as ethyl), and for at least one different repeating unit R11 is a different specific alkyl (such as methyl)). For example, each R11 may be selected from two different alkyl groups (such as methyl and ethyl) and not all R11 are the same alkyl.

In any of the above embodiments, R11 preferably is methyl or ethyl, more preferably methyl. Thus, in some embodiments, each R11 is methyl or each R11 is ethyl. In some alternative embodiments, R11 is independently selected from methyl and ethyl for each repeating unit, wherein in at least one repeating unit R11 is methyl, and in at least one repeating unit R11 is ethyl.

In some embodiments, the polymer comprises poly-2-(2-(2-aminoethoxy)ethoxy)acetic acid (pAEEA) or poly-2-(2-(2-methylaminoethoxy)ethoxy)acetic acid (pMAEEA), or a derivative thereof.

In some embodiments, the polymer comprises the following general formula:

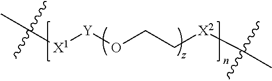

wherein
X2 and X1 taken together are optionally substituted amide, optionally substituted thioamide or ester;
Y is —CH2-, —(CH2)2-, or —(CH2)3-;
z is 2 to 24; and
n is 1 to 100.

In some embodiments,
(i) when X1 is —C(O)— then X2 is —NR1-;
(ii) when X1 is —NR1- then X2 is —C(O)—;
(iii) when X1 is —C(S)— then X2 is —NR1-;
(iv) when X1 is —NR1- then X2 is —C(S)—;
(v) when X1 is —C(O)— then X2 is —O—; or
(vi) when X1 is —O— then X2 is —C(O)—;
wherein R1 is hydrogen or C1-8 alkyl.

In some embodiments, X1 is —C(O)— and X2 is —NR1-, wherein R1 is hydrogen or C1-8 alkyl. In some embodiments, X1 is —C(O)— and X2 is —NR1-, wherein R1 is hydrogen or methyl. In some embodiments, X1 is —C(O)— and X2 is —NR1-, wherein R1 is hydrogen.

In some embodiments, Y is —CH2- or —(CH2)2-. In some embodiments, Y is —CH2-.

In some embodiments, the polymer comprises the following general formula:

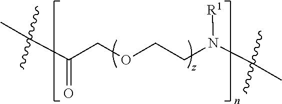

wherein
R1 is hydrogen or C1-8 alkyl;
z is 2 to 24; and
n is 1 to 100.

In some embodiments of the above formulas, z is 2 to 10. In some embodiments, z is 2 to 7. In some embodiments, z is 2 to 5. In some embodiments, z is 2 or 3. In some embodiments, z is 2.

In some embodiments, the polymer comprises the following general formula:

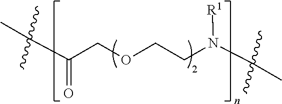

wherein
R1 is hydrogen or C1-8 alkyl; and
n is 1 to 100.

In some embodiments of the above formulas, R1 is hydrogen or methyl. In some embodiments, R1 is hydrogen.

In some embodiments, the polymer comprises the following general formula:

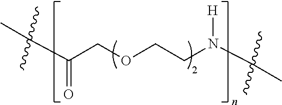

wherein
n is 1 to 100.

In some embodiments of the above formulas, n is 5 to 50. In some embodiments, n is 5 to 25. In some embodiments, n is 7 to 14. In some embodiments, n is 10 to 25. In some embodiments, n is 14 to 17. In some embodiments, n is 8 or 14.

In some embodiments, the molar proportion of the amphiphilic derivative of a polymer integrated into the particles is between 0.5 and 20 mol % of the lipid molecules making up the particle, preferably between 1 and 10 mol %.

### Pharmaceutical Compositions

The particles, or compositions described herein may be administered in pharmaceutical compositions or medicaments and may be administered in the form of any suitable pharmaceutical composition. In some embodiments, the pharmaceutical composition is for therapeutic or prophylactic treatments, e.g., for use in treating or preventing a disease involving an antigen such as a cancer disease or an infectious disease, in particular HIV infection.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease by administration of said pharmaceutical composition to a subject.

The pharmaceutical compositions of the present disclosure may comprise one or more adjuvants or may be administered with one or more adjuvants. In some embodiments, the pharmaceutical composition does not comprise an adjuvant. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminum hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in a storable form (e.g., in a frozen or lyophilized/freeze-dried form) or in a “ready-to-use form” (i.e., in a form which can be immediately administered to a subject, e.g., without any processing such as diluting). Thus, prior to administration of a storable form of a pharmaceutical composition, this storable form has to be processed or transferred into a ready-to-use or administrable form. E.g., a frozen pharmaceutical composition has to be thawed, or a freeze-dried pharmaceutical composition has to be reconstituted, e.g. by using a suitable solvent (e.g., deionized water, such as water for injection) or liquid (e.g., an aqueous solution).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In some embodiments relating to the treatment of a particular disease, the desired reaction may relate to inhibition of the course of the disease. This comprises slowing down the progress of the disease and, in some embodiments, interrupting or reversing the progress of the disease. The desired reaction in a treatment of a disease may also be delay of the onset or a prevention of the onset of said disease or said condition, or symptoms thereof. An effective amount of the pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the pharmaceutical compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition.

A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Methods of Treatment

The agents, compositions and methods described herein can be used to treat a subject with a disease, e.g., a disease characterized by HIV infection. The agents, compositions and methods described herein may be used in the therapeutic or prophylactic treatment of various diseases. In some embodiments, the agents, compositions and methods described herein are useful in a prophylactic and/or therapeutic treatment of a disease involving an antigen.

Such antigen may serve as target for immune effector cells expressing an antigen receptor. For example, if the antigen is derived from a virus, the agents, compositions and methods may be useful in the treatment of a viral disease caused by said virus. If the antigen is a tumor antigen, the agents, compositions and methods may be useful in the treatment of a cancer disease wherein cancer cells express said tumor antigen.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

The term “disease involving an antigen”, “disease involving cells expressing an antigen” or similar terms refer to any disease which implicates an antigen, e.g., a disease which is characterized by the presence of an antigen. The disease involving an antigen can be an infectious disease, or a cancer disease or simply cancer. As mentioned above, the antigen may be a disease-associated antigen, such as a tumor-associated antigen, a viral antigen, or a bacterial antigen. In some embodiments, a disease involving an antigen is a disease involving cells expressing an antigen, preferably on the cell surface.

The term “infectious disease” refers to any disease which can be transmitted from individual to individual or from organism to organism, and is caused by a microbial agent (e.g. HIV). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The methods and agents described herein are, in particular, useful for the treatment of HIV infection.

“Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to cells called T cells or T lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

In some embodiments, the compositions, particles or pharmaceutical compositions described herein may be administered intravenously, intraarterially, subcutaneously, intradermally, dermally, intranodally, intramuscularly, intratumorally, or peritumorally. In some embodiments, the compositions, particles or pharmaceutical compositions described herein may be administered intramuscularly. In some embodiments, the compositions, particles or pharmaceutical compositions is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the compositions, particles or pharmaceutical compositions are formulated for systemic administration. In some embodiments, the systemic administration is by intravenous administration. In some embodiments, the compositions, particles or pharmaceutical compositions are formulated for intramuscular administration.

The present invention is described in detail and is illustrated by the figures and examples, which are used only for illustration purposes and are not meant to be limiting. Owing to the description and the examples, further embodiments which are likewise included in the invention are accessible to the skilled worker.

SEQUENCE LISTING

| | Sequence | |
|---|---|---|
| SEQ ID NO: 1 | KLTPLCVTL | |
| SEQ ID NO: 2 | SFNCGGEFF | |
| SEQ ID NO: 3 | SFNCRGEFF | |
| SEQ ID NO: 4 | RAIEAQQHL | |
| SEQ ID NO: 5 | RAIEAQQHM | |
| SEQ ID NO: 6 | GQMVHQAISP | |
| SEQ ID NO: 7 | GQMVHQAISPR | |
| SEQ ID NO: 8 | VHQAISPRTL | |
| SEQ ID NO: 9 | HQAISPRTL | |
| SEQ ID NO: 10 | QAISPRTLNAW | |
| SEQ ID NO: 11 | ISPRTLNAW | |
| SEQ ID NO: 12 | SPRTLNAWV | |
| SEQ ID NO: 13 | VKVVEEKAF | |
| SEQ ID NO: 14 | VKVIEEKAF | |
| SEQ ID NO: 15 | EEKAFSPEV | |
| SEQ ID NO: 16 | KAFSPEVIPM | |
| SEQ ID NO: 17 | KAFSPEVIPMF | |
| SEQ ID NO: 18 | EVIPMFSAL | |
| SEQ ID NO: 19 | EVIPMFTAL | |
| SEQ ID NO: 20 | VIPMFSAL | |
| SEQ ID NO: 21 | VIPMFTAL | |
| SEQ ID NO: 22 | SALSEGATP | |
| SEQ ID NO: 23 | TALSEGATP | |
| SEQ ID NO: 24 | SEGATPQDL | |
| SEQ ID NO: 25 | ATPQDLNTMLNT | |
| SEQ ID NO: 26 | TPQDLNTML | |
| SEQ ID NO: 27 | GHQAAMQML | |
| SEQ ID NO: 28 | TSTLQEQIGW | |
| SEQ ID NO: 29 | TSTLQEQIAW | |
| SEQ ID NO: 30 | EIYKRWII | |
| SEQ ID NO: 31 | DIYKRWIIL | |
| SEQ ID NO: 32 | EIYKRWIIL | |
| SEQ ID NO: 33 | DIYKRWIIL | |
| SEQ ID NO: 34 | KRWIILGLNK | |
| SEQ ID NO: 35 | GLNKIVRMY | |
| SEQ ID NO: 36 | VRMYSPTSI | |
| SEQ ID NO: 37 | VRMYSPVSI | |
| SEQ ID NO: 38 | RMYSPTSI | |
| SEQ ID NO: 39 | RMYSPVSI | |
| SEQ ID NO: 40 | RMYSPTSIL | |
| SEQ ID NO: 41 | RMYSPVSIL | |
| SEQ ID NO: 42 | YSPTSILDI | |
| SEQ ID NO: 43 | YSPVSILDI | |
| SEQ ID NO: 44 | FRDYVDRFY | |
| SEQ ID NO: 45 | FRDYVDRFF | |
| SEQ ID NO: 46 | RDYVDRFYKTL | |
| SEQ ID NO: 47 | RDYVDRFFKTL | |
| SEQ ID NO: 48 | DYVDRFYKTLR | |
| SEQ ID NO: 49 | DYVDRFFKTLR | |
| SEQ ID NO: 50 | YVDRFYKTL | |
| SEQ ID NO: 51 | YVDRFFKTL | |
| SEQ ID NO: 52 | DRFYKTLRA | |
| SEQ ID NO: 53 | DRFFKTLRA | |
| SEQ ID NO: 54 | AEQASQEVKNW | |
| SEQ ID NO: 55 | AEQATQDVKNW | |
| SEQ ID NO: 56 | AEQASQEVKNWM | |
| SEQ ID NO: 57 | AEQATQDVKNWM | |
| SEQ ID NO: 58 | QASQEVKNW | |
| SEQ ID NO: 59 | QATQDVKNW | |
| SEQ ID NO: 60 | DCKTILKAL | |
| SEQ ID NO: 61 | DCKTILRAL | |
| SEQ ID NO: 62 | ACQGVGGPGHK | |
| SEQ ID NO: 63 | GPGHKARVL | |
| SEQ ID NO: 64 | HTQGYFPDW | |
| SEQ ID NO: 65 | HTQGFFPDW | |
| SEQ ID NO: 66 | TQGYFPDWQNY | |
| SEQ ID NO: 67 | TQGFFPDWQNY | |
| SEQ ID NO: 68 | YFPDWQNY | |
| SEQ ID NO: 69 | FFPDWQNY | |
| SEQ ID NO: 70 | YFPDWQNYT | |
| SEQ ID NO: 71 | FFPDWQNYT | |
| SEQ ID NO: 72 | YTPGPGIRY | |
| SEQ ID NO: 73 | YTPGPGVRY | |
| SEQ ID NO: 74 | TPGPGIRYPL | |
| SEQ ID NO: 75 | TPGPGVRYPL | |
| SEQ ID NO: 76 | IRYPLTFGW | |
| SEQ ID NO: 77 | VRYPLTFGW | |
| SEQ ID NO: 78 | RYPLTFGW | |
| SEQ ID NO: 79 | RYPLTFGWCF | |
| SEQ ID NO: 80 | YPLTFGWCF | |
| SEQ ID NO: 81 | LTFGWCFKL | |
| SEQ ID NO: 82 | LVGPTPVNI | |
| SEQ ID NO: 83 | TPVNIIGRNLL | |
| SEQ ID NO: 84 | TPVNIIGRNML | |
| SEQ ID NO: 85 | GIPHPAGLK | |
| SEQ ID NO: 86 | TAFTIPSI | |
| SEQ ID NO: 87 | TPGIRYQYNVL | |
| SEQ ID NO: 88 | IRYQYNVL | |
| SEQ ID NO: 89 | KQNPDIVIY | |
| SEQ ID NO: 90 | AQNPEIVIY | |
| SEQ ID NO: 91 | VIYQYMDDL | |
| SEQ ID NO: 92 | PIQKETWEAW | |
| SEQ ID NO: 93 | PIQKETWETW | |
| SEQ ID NO: 94 | TWEAWWTEYW | |
| SEQ ID NO: 95 | TWETWWTDYW | |
| SEQ ID NO: 96 | WEAWWTEYW | |
| SEQ ID NO: 97 | WETWWTDYW | |
| SEQ ID NO: 98 | IVTDSQYAL | |
| SEQ ID NO: 99 | FKRKGGIGGY | |
| SEQ ID NO: 100 | KRKGGIGGY | |
| SEQ ID NO: 101 | GERIVDII | |
| SEQ ID NO: 102 | GERIIDII | |
| SEQ ID NO: 103 | GERIVDIIA | |
| SEQ ID NO: 104 | GERIIDIIA | |
| SEQ ID NO: 105 | GQMVHQALSP | |
| SEQ ID NO: 106 | GQMVHQPLSP | |
| SEQ ID NO: 107 | GQMVHQPISP | |
| SEQ ID NO: 108 | GQMVHQALSPR | |
| SEQ ID NO: 109 | GQMVHQPLSPR | |
| SEQ ID NO: 110 | GQMVHQPISPR | |
| SEQ ID NO: 111 | VHQALSPRTL | |
| SEQ ID NO: 112 | VHQPLSPRTL | |
| SEQ ID NO: 113 | VHQPISPRTL | |
| SEQ ID NO: 114 | HQALSPRTL | |
| SEQ ID NO: 115 | HQPLSPRTL | |
| SEQ ID NO: 116 | HQPISPRTL | |
| SEQ ID NO: 117 | QALSPRTLNAW | |
| SEQ ID NO: 118 | QPLSPRTLNAW | |
| SEQ ID NO: 119 | QPISPRTLNAW | |
| SEQ ID NO: 120 | LSPRTLNAW | |
| SEQ ID NO: 121 | LSPRTLNAW | |
| SEQ ID NO: 122 | AEQASQDVKNW | |
| SEQ ID NO: 123 | AEQATQEVKNW | |
| SEQ ID NO: 124 | AEQASQDVKNWM | |
| SEQ ID NO: 125 | AEQATQEVKNWM | |
| SEQ ID NO: 126 | QASQDVKNWM | |
| SEQ ID NO: 127 | QATQEVKNWM | |
| SEQ ID NO: 128 | ACQGVGGPSHK | |
| SEQ ID NO: 129 | GPSHKARVL | |
| SEQ ID NO: 130 | NTQGYYPDW | |
| SEQ ID NO: 131 | NTQGYFPDW | |
| SEQ ID NO: 132 | YTPGPGTRY | |
| SEQ ID NO: 133 | YTPGPGVRF | |
| SEQ ID NO: 134 | TPGPGTRYPL | |
| SEQ ID NO: 135 | TPGPGVRFPL | |
| SEQ ID NO: 136 | TRYPLTFGW | |
| SEQ ID NO: 137 | VRFPLTFGW | |
| SEQ ID NO: 138 | RFPLTFGW | |
| SEQ ID NO: 139 | LIGPTPVNI | |
| SEQ ID NO: 140 | TAFTIPST | |
| SEQ ID NO: 141 | TAFTIPSV | |
| SEQ ID NO: 142 | KQNPEIVIY | |
| SEQ ID NO: 143 | AKNPEIVIY | |
| SEQ ID NO: 144 | PIQKEIWETW | |
| SEQ ID NO: 145 | IWETWWTDYW | |
| SEQ ID NO: 146 | IWETWWTEYW | |
| SEQ ID NO: 147 | WETWWTDYW | |
| SEQ ID NO: 148 | WETWWTEYW | |
| SEQ ID NO: 149 | AEQASQEVKNWM | |
| SEQ ID NO: 150 | GHQAAMQMLKETI | |
| SEQ ID NO: 151 | QNYTPGPGIRYPLTFGWCFKL | |
| SEQ ID NO: 152 | FKRKGGIGGY | |
| SEQ ID NO: 153 | SALSEGATPQDLNTMLNT | |
| SEQ ID NO: 154 | LVGPTPVNIIGRNLL | |
| SEQ ID NO: 155 | VKVVEEKAFSPEVIPMFSAL | |
| SEQ ID NO: 156 | KQNPDIVIYQYMDDL | |
| SEQ ID NO: 157 | FRDYVDRFYKTLRA | |
| SEQ ID NO: 158 | AEQASQDVKNWM | |
| SEQ ID NO: 159 | VRMYSPTSILDI | |
| SEQ ID NO: 160 | PIQKETWEAWWTEYW | |
| SEQ ID NO: 161 | HTQGYFPDWQNYT | |
| SEQ ID NO: 162 | EIYKRWIILGLNK | |
| SEQ ID NO: 163 | PIQKEIWETWWTDYW | |
| SEQ ID NO: 164 | GQMVHQPLSPRTLNAW | |
| SEQ ID NO: 165 | YTPGPGTRYPLTFGW | |
| SEQ ID NO: 166 | GQMVHQAISPRTLNAWVKVV | |
| SEQ ID NO: 167 | ACQGVGGPGHKARVL | |
| SEQ ID NO: 168 | VRMYSPVSILDI | |
| SEQ ID NO: 169 | GQMVHQALSPRTLNAWVKVI | |
| SEQ ID NO: 170 | YTPGPGVRFPLTFGWCF | |
| SEQ ID NO: 171 | AQNPEIVIYQYMDDL | |
| SEQ ID NO: 172 | QNYTPGPGVRYPLTFGWCFKL | |
| SEQ ID NO: 173 | HTQGFFPDWQNYT | |
| SEQ ID NO: 174 | PIQKETWETWWTDYW | |
| SEQ ID NO: 175 | GQMVHQPISPRTLNAW | |
| SEQ ID NO: 176 | LIGPTPVNIIGRNML | |
| SEQ ID NO: 177 | VKVIEEKAFSPEVIPMFTAL | |
| SEQ ID NO: 178 | PIQKEIWETWWTEYW | |
| SEQ ID NO: 179 | FRDYVDRFFKTLRA | |
| SEQ ID NO: 180 | TALSEGATPQDLNTMLNT | |
| SEQ ID NO: 181 | DIYKRWIILGLNK | |
| SEQ ID NO: 182 | ACQGVGGPSHKARVL | |
| SEQ ID NO: 198 | WEIWWTDYW | |
| SEQ ID NO: 199 | NTQGFFPDW | |
| SEQ ID NO: 200 | PIQKETWEIW | |
| SEQ ID NO: 201 | TWEIWWTDYW | |
| SEQ ID NO: 202 | TWETWWTEYW | |
| SEQ ID NO: 203 | PIQKETWEIWWTDYW | |
| SEQ ID NO: 204 | PIQKETWETWWTEYW | |
| SEQ ID NO: | Description | Sequence |
| 183 | String 1 non-GS aa | MDCKTILKALRMYGERIVDIIARIVTDSQYALAEQASQEVKNWMKRNRSFNCG |
| | | GEFFAAAYGHQAAMQMLKETIAKQNPEIVIYRTAFTIPSIRMAKLTPLCVTLSM |
| | | MTAFTIPSVRQNYTPGPGIRYPLTFGWCFKLRKRAFKRKGGIGGYRKYNTQGY |
| | | YPDWKRMYSALSEGATPQDLNTMLNTYYLVGPTPVNIIGRNLLRYYVKVVEEK |
| | | AFSPEVIPMFSALKQNPDIVIYQYMDDLARGFRDYVDRFYKTLRARAEQASQD |
| | | VKNWMSGARAIEAQQHLRRAVRMYSPTSILDIAMAPIQKETWEAWWTEYWG |
| | | HTQGYFPDWQNYTRRYEIYKRWIILGLNKRAAGLNKIVRMYKMRTSTLQEQIG |
| | | WARMKPIQKEIWETWWTDYWGQMVHQPLSPRTLNAWYTPGPGTRYPLTFG |
| | | WAMYGQMVHQAISPRTLNAWVKVVYMATPGIRYQYNVLRKMYACQGVGGP |
| | | GHKARVLMGIPHPAGLKAKKQYIKANSKFIGITELKKLGGGKRGGGKKMTNSV |
| | | DDALINSTKIYSYFPSVISKVNQGAQGKKL |
| 184 | String 1 non-GS | ATGGATTGTAAGACTATTTTAAAAGCATTGCGCATGTACGGGGAAAGAATA |
| | nt1 | GTAGACATAATAGCAAGGATAGTAACAGACTCACAGTATGCATTAGCCGAGC |
| | | AAGCTTCACAGGAGGTAAAAAATTGGATGAAGAGGAACAGGAGTTTTAATT |
| | | GTGGAGGGGAATTTTTCGCTGCCGCGTATGGACATCAAGCAGCCATGCAAA |
| | | TGTTAAAAGAGACCATCGCCAAACAAAATCCAGAGATAGTTATCTATCGGAC |
| | | TGCATTCACCATACCTAGTATAAGAATGGCGAAGTTGACCCCACTCTGTGTC |
| | | ACTTTATCCATGATGACTGCATTCACCATACCTAGTGTACGTCAGAACTACA |
| | | CACCAGGGCCAGGGATCAGATATCCACTGACCTTTGGATGGTGCTTCAAGC |
| | | TAAGAAAACGGGCATTTAAAAGAAAAGGGGGGATTGGGGGGTACAGAAAAT |
| | | ACAACACACAAGGCTACTACCCTGATTGGAAGCGAATGTATTCAGCATTATC |
| | | AGAAGGAGCCACCCCACAAGATTTAAACACCATGCTAAACACATACTACTTA |
| | | GTAGGACCTACACCTGTCAACATAATTGGAAGAAATCTGTTGAGGTATTACG |
| | | TAAAAGTAGTAGAAGAGAAGGCTTTCAGCCCAGAAGTAATACCCATGTTTTC |
| | | AGCATTAAAACAAAATCCAGACATAGTTATCTATCAATACATGGATGATTTG |
| | | GCCAGAGGTTTTAGAGACTATGTAGACCGGTTCTATAAAACTCTAAGAGCCC |
| | | GAGCTGAACAAGCTTCACAAGATGTAAAAAATTGGATGAGCGGCGCAAGGG |
| | | CTATTGAGGCGCAACAGCATCTGCGACGGGCTGTAAGAATGTATAGCCCTA |
| | | CCAGCATTCTGGACATAGCTATGGCACCCATACAAAAAGAAACATGGGAAGC |
| | | ATGGTGGACAGAGTATTGGGGGCACACACAAGGCTACTTCCCTGATTGGCA |
| | | GAACTACACACGGCGCTATGAAATCTATAAAAGATGGATAATCCTGGGATTA |
| | | AATAAACGAGCAGCTGGGTTAAATAAAATAGTAAGAATGTATAAAATGCGTA |
| | | CTAGTACCCTTCAGGAACAAATAGGATGGGCAAGGATGAAACCCATCCAAAA |
| | | AGAAATATGGGAGACATGGTGGACAGACTATTGGGGGCAAATGGTACACCA |
| | | GCCCCTATCACCTAGAACTTTGAATGCATGGTACACACCGGGACCAGGGACT |
| | | AGATATCCACTGACCTTTGGATGGGCGATGTATGGGCAAATGGTACATCAG |
| | | GCCATATCACCTAGAACTTTAAATGCATGGGTAAAAGTAGTATATATGGCAA |
| | | CACCAGGGATTAGATATCAATATAATGTGCTTCGTAAGATGTATGCATGTCA |
| | | GGGAGTGGGAGGACCTGGCCACAAAGCAAGAGTGTTGATGGGAATACCACA |
| | | CCCAGCAGGGTTAAAAGCTAAGAAGCAGTACATCAAGGCCAACAGCAAGTTC |
| | | ATCGGCATCACCGAGCTGAAGAAGCTGGGAGGGGGCAAACGGGGAGGCGG |
| | | CAAAAAGATGACCAACAGCGTGGACGACGCCCTGATCAACAGCACCAAGATC |
| | | TACAGCTACTTCCCCAGCGTGATCAGCAAAGTGAACCAGGGCGCTCAGGGC |
| | | AAGAAACTG |
| 211 | String 1 non-GS | ATGGACTGCAAGACCATCCTGAAGGCCCTGCGCATGTACGGCGAGAGAATC |
| | nt2 | GTGGACATCATTGCCAGGATCGTGACCGATAGCCAGTACGCCCTGGCCGAA |
| | | CAGGCCAGCCAAGAAGTGAAGAACTGGATGAAGCGGAACCGGAGCTTCAAT |
| | | TGTGGCGGCGAGTTCTTCGCTGCCGCGTATGGACATCAGGCCGCTATGCAG |
| | | ATGCTGAAAGAGACAATCGCCAAGCAGAACCCCGAGATCGTGATCTACCGG |
| | | ACCGCCTTCACAATCCCCAGCATCAGAATGGCGAAGCTGACCCCTCTGTGTG |
| | | TGACCCTGTCCATGATGACCGCCTTCACAATCCCTAGCGTGCGTCAGAACTA |
| | | CACACCCGGACCTGGCATCAGATACCCTCTGACCTTCGGCTGGTGCTTCAAG |
| | | CTGAGAAAACGGGCATTCAAGCGGAAAGGCGGCATCGGCGGCTATAGAAAA |
| | | TACAACACCCAGGGCTACTACCCCGATTGGAAGCGAATGTATTCTGCTCTGT |
| | | CTGAAGGCGCCACACCTCAGGACCTGAACACCATGCTGAACACCTACTACCT |
| | | CGTTGGACCCACACCTGTGAACATCATCGGCCGGAATCTGCTGAGGTATTAC |
| | | GTGAAGGTGGTGGAAGAAAAGGCTTTCAGCCCCGAAGTGATCCCCATGTTC |
| | | AGCGCTCTTAAGCAGAACCCCGACATCGTGATCTACCAGTACATGGACGACC |
| | | TGGCCAGAGGTTTCAGAGACTACGTGGACCGGTTCTACAAGACCCTGAGAG |
| | | CCCGAGCCGAACAGGCTTCCCAGGACGTGAAGAACTGGATGAGCGGCGCAA |
| | | GAGCCATTGAAGCCCAGCAGCACCTCCGACGGGCTGTGAGAATGTACAGCC |
| | | CCACCAGCATCCTGGACATCGCTATGGCACCCATCCAGAAAGAGACATGGGA |
| | | AGCCTGGTGGACCGAGTACTGGGGGCACACCCAGGGCTACTTCCCCGACTG |
| | | GCAGAATTACACCCGGCGCTATGAGATCTACAAGCGGTGGATCATCCTGGG |
| | | CCTGAACAAGCGAGCAGCTGGCCTGAACAAGATCGTGCGGATGTACAAAAT |
| | | GCGTACCAGCACACTGCAAGAGCAGATCGGCTGGGCAAGGATGAAACCCAT |
| | | CCAGAAAGAGATCTGGGAAACCTGGTGGACCGACTACTGGGGCCAGATGGT |
| | | TCACCAGCCTCTGAGCCCCAGAACACTGAATGCTTGGTACACACCTGGACCT |
| | | GGCACAAGATACCCTCTGACCTTTGGCTGGGCGATGTATGGACAGATGGTG |
| | | CATCAGGCCATCTCTCCCAGAACACTGAACGCCTGGGTCAAAGTGGTGTATA |
| | | TGGCAACCCCTGGCATCAGATACCAGTACAACGTGCTGCGTAAGATGTATGC |
| | | CTGTCAAGGTGTTGGAGGACCTGGACACAAGGCCAGAGTTCTGATGGGCAT |
| | | TCCTCATCCTGCCGGACTGAAAGCTAAGAAGCAGTACATCAAGGCCAACAGC |
| | | AAGTTCATCGGCATCACCGAGCTGAAGAAGCTGGGAGGGGGCAAACGGGGA |
| | | GGCGGCAAAAAGATGACCAACAGCGTGGACGACGCCCTGATCAACAGCACC |
| | | AAGATCTACAGCTACTTCCCCAGCGTGATCAGCAAAGTGAACCAGGGCGCTC |
| | | AGGGCAAGAAACTGTGATAA |
| 212 | String 1 non-GS | ATGGATTGCAAAACAATCTTAAAAGCTTTACGCATGTACGGAGAAAGAATTG |
| | nt3 | TGGATATTATTGCTAGGATTGTAACAGATTCTCAATATGCTCTGGCTGAACA |
| | | AGCTTCTCAGGAAGTGAAAAATTGGATGAAGCGGAACCGGTCTTTTAATTGT |
| | | GGAGGAGAATTCTTCGCTGCCGCGTATGGACACCAAGCAGCAATGCAAATG |
| | | TTGAAAGAAACCATCGCCAAACAAAATCCTGAAATTGTGATTTACCGGACCG |
| | | CTTTTACCATTCCATCAATTAGAATGGCGAAATTGACCCCTTTGTGTGTGAC |
| | | CTTGTCCATGATGACCGCTTTTACCATTCCAAGTGTTCGTCAGAATTACACC |
| | | CCTGGACCTGGAATCAGATACCCTCTGACCTTTGGATGGTGCTTTAAACTGA |
| | | GAAAACGGGCATTTAAAAGAAAAGGAGGAATTGGAGGATACAGAAAATACA |
| | | ACACCCAAGGATACTACCCTGATTGGAAGCGAATGTATTCTGCATTGTCTGA |
| | | AGGAGCAACCCCTCAAGATTTGAATACCATGTTGAATACCTACTACCTTGTA |
| | | GGACCAACACCTGTAAATATCATCGGAAGAAATCTTCTTAGGTATTACGTGA |
| | | AAGTGGTGGAAGAAAAAGCTTTTTCTCCTGAAGTGATTCCAATGTTTTCTGC |
| | | TCTTAAACAAAATCCTGATATTGTAATTTACCAATACATGGATGATCTGGCC |
| | | AGAGGTTTTAGAGATTACGTGGATAGATTTTACAAAACACTGAGAGCACGA |
| | | GCTGAACAAGCTTCTCAAGACGTTAAAAATTGGATGAGCGGCGCAAGAGCA |
| | | ATTGAAGCACAGCAGCACCTCCGACGGGCTGTGAGAATGTACTCACCAACCT |
| | | CAATTCTGGATATTGCTATGGCACCAATTCAGAAAGAAACATGGGAAGCTTG |
| | | GTGGACAGAATACTGGGGGCACACACAGGGATACTTTCCTGATTGGCAGAA |
| | | TTACACACGGCGCTATGAAATTTACAAAAGATGGATTATTCTGGGACTGAAT |
| | | AAACGAGCAGCTGGATTAAATAAAATTGTAAGAATGTACAAAATGCGTACAT |
| | | CAACACTTCAGGAACAGATTGGATGGGCAAGGATGAAACCAATCCAGAAAG |
| | | AAATCTGGGAAACATGGTGGACAGATTACTGGGGACAGATGGTGCACCAGC |
| | | CATTGTCTCCAAGAACATTGAATGCTTGGTACACACCTGGACCTGGAACAAG |
| | | ATACCCTCTGACATTTGGATGGGCGATGTATGGACAGATGGTTCACCAGGC |
| | | AATTTCTCCAAGAACATTAAATGCATGGGTTAAAGTTGTTTATATGGCAACA |
| | | CCTGGAATAAGATACCAATACAATGTGCTTCGTAAGATGTATGCATGCCAGG |
| | | GAGTGGGAGGACCTGGACACAAAGCAAGAGTGCTTATGGGAATTCCTCATC |
| | | CTGCTGGACTGAAAGCTAAGAAGCAGTACATCAAGGCCAACAGCAAGTTCAT |
| | | CGGCATCACCGAGCTGAAGAAGCTGGGAGGGGGCAAACGGGGAGGCGGCA |
| | | AAAAGATGACCAACAGCGTGGACGACGCCCTGATCAACAGCACCAAGATCTA |
| | | CAGCTACTTCCCCAGCGTGATCAGCAAAGTGAACCAGGGCGCTCAGGGCAA |
| | | GAAACTGTGATAA |
| 185 | String 2 non-GS aa | MIVTDSQYALYMAEQATQEVKNWMATSTLQEQIAWGIPHPAGLKRRYAVRM |
| | | YSPVSILDIRSYGLNKIVRMYRMGHQAAMQMLKETIAKNPEIVIYSYGQMVHQ |
| | | ALSPRTLNAWVKVIYMATPGIRYQYNVLAEQATQDVKNWMSFNCRGEFFAYT |
| | | PGPGVRFPLTFGWCFAAQNPEIVIYQYMDDLARQNYTPGPGVRYPLTFGWCF |
| | | KLAFKRKGGIGGYRSYNTQGYFPDWRYAHTQGFFPDWQNYTARYPIQKETW |
| | | ETWWTDYWGNMYGQMVHQPISPRTLNAWANAYGERIIDIIAKLIGPTPVNII |
| | | GRNMLYRYADCKTILRALRRAYVKVIEEKAFSPEVIPMFTALARPIQKEIWETW |
| | | WTEYWGNNFRDYVDRFFKTLRARAIEAQQHMARARTALSEGATPQDLNTML |
| | | NTDIYKRWIILGLNKRRAKLTPLCVTLSMACQGVGGPSHKARVLTAFTIPSTKK |
| | | QYIKANSKFIGITELKKLGGGKRGGGKKMTNSVDDALINSTKIYSYFPSVISKVN |
| | | QGAQGKKL |
| 186 | String 2 non-GS | ATGATAGTAACAGACTCACAGTATGCATTATACATGGCTGAACAAGCTACAC |
| | nt1 | AAGAGGTAAAAAATTGGATGGCGACTAGTACCCTTCAGGAACAAATAGCAT |
| | | GGGGAATACCACACCCAGCAGGGTTAAAAAGACGGTATGCAGTAAGAATGT |
| | | ATAGCCCTGTCAGCATTTTGGACATACGTTCTTACGGGTTAAATAAAATAGT |
| | | AAGAATGTATCGTATGGGACATCAAGCAGCCATGCAAATGTTAAAAGAGACC |
| | | ATCGCAAAAAATCCAGAAATAGTCATCTATTCCTACGGGCAAATGGTACACC |
| | | AGGCCCTATCACCTAGAACTTTGAATGCATGGGTAAAAGTAATATATATGGC |
| | | CACACCAGGGATTAGATATCAATATAATGTGCTTGCTGAACAAGCTACACAA |
| | | GATGTAAAAAATTGGATGAGCTTTAATTGTAGAGGAGAATTTTTCGCGTACA |
| | | CACCGGGACCAGGGGTCAGATTTCCACTGACCTTTGGATGGTGCTTCGCAG |
| | | CACAAAATCCAGAAATAGTCATCTATCAATATATGGATGACTTGGCCCGTCA |
| | | AAACTACACACCGGGACCAGGGGTCAGATATCCACTGACCTTTGGATGGTG |
| | | CTTCAAGCTAGCATTTAAAAGAAAAGGGGGGATTGGGGGGTACCGCTCTTA |
| | | TAACACACAAGGCTACTTCCCTGATTGGAGGTATGCCCACACACAAGGCTTC |
| | | TTCCCTGATTGGCAAAACTACACAGCTAGGTATCCCATCCAAAAAGAAACAT |
| | | GGGAGACATGGTGGACAGACTATTGGGGGAATATGTACGGGCAAATGGTAC |
| | | ACCAGCCCATATCACCTAGAACTTTGAATGCATGGGCTAATGCGTACGGGGA |
| | | AAGAATAATAGACATAATAGCAAAGTTAATAGGACCTACACCTGTCAACATA |
| | | ATTGGAAGAAATATGTTGTATAGATACGCGGATTGTAAGACCATTTTAAGAG |
| | | CATTACGACGTGCATATGTAAAAGTAATAGAGGAGAAGGCTTTTAGCCCAGA |
| | | GGTAATACCCATGTTTACAGCATTAGCACGTCCCATCCAAAAAGAAATATGG |
| | | GAGACATGGTGGACAGAGTATTGGGGAAACAATTTTAGAGACTATGTAGAC |
| | | CGGTTCTTTAAAACTTTAAGAGCTAGGGCTATAGAGGCGCAACAGCATATG |
| | | GCTCGTGCTCGAACAGCATTATCAGAAGGAGCCACCCCACAAGATTTAAACA |
| | | CCATGTTAAATACAGACATCTATAAAAGATGGATAATTCTGGGGTTAAATAA |
| | | AAGACGTGCCAAGTTGACCCCACTCTGTGTCACTTTATCAATGGCATGTCAG |
| | | GGAGTGGGAGGACCTTCCCACAAAGCAAGAGTGTTGACTGCATTCACCATA |
| | | CCTAGTACAAAGAAGCAGTACATCAAGGCCAACAGCAAGTTCATCGGCATCA |
| | | CCGAGCTGAAGAAGCTGGGAGGGGGCAAACGGGGAGGCGGCAAAAAGATG |
| | | ACCAACAGCGTGGACGACGCCCTGATCAACAGCACCAAGATCTACAGCTACT |
| | | TCCCCAGCGTGATCAGCAAAGTGAACCAGGGCGCTCAGGGCAAGAAACTG |
| 213 | String 2 non-GS | ATGATCGTGACCGATAGCCAGTACGCCCTGTACATGGCCGAACAGGCCACA |
| | nt2 | CAAGAAGTGAAGAACTGGATGGCGACCAGCACACTGCAAGAGCAGATCGCC |
| | | TGGGGCATTCCTCATCCTGCCGGACTGAAAAGACGGTATGCAGTGAGAATG |
| | | TACAGCCCCGTGTCCATCCTGGACATCCGTTCTTACGGCCTGAACAAGATCG |
| | | TGCGGATGTACCGTATGGGACATCAGGCCGCTATGCAGATGCTGAAAGAGA |
| | | CAATCGCCAAGAATCCCGAGATCGTGATCTACTCCTACGGCCAGATGGTTCA |
| | | CCAGGCTCTGAGCCCCAGAACACTGAACGCCTGGGTCAAAGTGATCTATAT |
| | | GGCCACCCCTGGCATCAGATACCAGTACAACGTGCTGGCCGAACAGGCTAC |
| | | CCAGGACGTGAAGAACTGGATGAGCTTCAATTGCAGGGGCGAGTTCTTCGC |
| | | GTACACACCTGGACCTGGCGTCAGATTCCCTCTGACCTTCGGCTGGTGCTTC |
| | | GCAGCCCAGAATCCTGAGATCGTGATCTACCAGTACATGGACGACCTGGCC |
| | | CGTCAGAACTACACACCTGGACCTGGCGTGCGGTATCCTCTGACCTTCGGCT |
| | | GGTGCTTCAAGCTGGCATTCAAGCGGAAAGGCGGCATCGGCGGCTATCGCT |
| | | CTTATAACACCCAGGGCTACTTCCCCGATTGGAGGTATGCCCACACCCAGGG |
| | | ATTCTTCCCAGACTGGCAGAACTACACCGCTAGGTATCCCATCCAGAAAGAG |
| | | ACATGGGAAACCTGGTGGACCGACTACTGGGGGAATATGTACGGCCAGATG |
| | | GTGCATCAGCCTATCAGCCCCAGAACACTGAACGCCTGGGCTAATGCGTAC |
| | | GGCGAGCGGATCATCGACATCATTGCCAAGCTGATCGGACCCACACCTGTG |
| | | AACATCATCGGCCGGAACATGCTGTATAGATACGCGGACTGCAAGACCATCC |
| | | TGAGAGCCCTGCGACGTGCATATGTGAAAGTGATCGAGGAAAAGGCTTTCA |
| | | GCCCCGAAGTGATCCCCATGTTCACAGCCCTTGCACGTCCCATCCAGAAAGA |
| | | GATCTGGGAAACCTGGTGGACCGAGTACTGGGGAAACAATTTCAGAGACTA |
| | | CGTGGACCGGTTCTTCAAGACCCTGAGAGCCAGAGCCATTGAAGCCCAGCA |
| | | GCACATGGCTCGTGCTCGAACAGCTCTGTCTGAAGGCGCCACACCTCAGGA |
| | | CCTGAACACCATGCTGAACACCGACATCTACAAGCGGTGGATCATCCTGGGC |
| | | CTGAACAAAAGACGTGCCAAGCTGACCCCTCTGTGTGTGACCCTGTCAATGG |
| | | CCTGTCAAGGTGTTGGCGGACCTTCTCACAAGGCCAGAGTTCTGACCGCCT |
| | | TCACAATCCCTAGCACCAAGAAGCAGTACATCAAGGCCAACAGCAAGTTCAT |
| | | CGGCATCACCGAGCTGAAGAAGCTGGGAGGGGGCAAACGGGGAGGCGGCA |
| | | AAAAGATGACCAACAGCGTGGACGACGCCCTGATCAACAGCACCAAGATCTA |
| | | CAGCTACTTCCCCAGCGTGATCAGCAAAGTGAACCAGGGCGCTCAGGGCAA |
| | | GAAACTGTGATAA |
| 214 | String 2 non-GS | ATGATTGTAACAGATTCTCAATATGCTCTGTACATGGCTGAACAAGCTACAC |
| | nt3 | AAGAAGTGAAAAATTGGATGGCGACATCAACACTTCAGGAACAGATTGCTTG |
| | | GGGAATTCCTCACCCTGCTGGATTGAAAAGACGGTATGCAGTGAGAATGTA |
| | | CTCTCCTGTGTCAATTCTGGATATTCGTTCTTACGGATTAAATAAAATTGTA |
| | | AGAATGTACCGTATGGGACACCAAGCAGCAATGCAAATGCTGAAAGAAACCA |
| | | TTGCTAAAAATCCTGAAATTGTGATTTACTCCTACGGACAGATGGTGCACCA |
| | | GGCTTTGTCTCCAAGAACATTGAATGCTTGGGTAAAAGTAATTTATATGGCC |
| | | ACACCTGGAATAAGATACCAATACAATGTGCTTGCAGAACAAGCAACACAAG |
| | | ATGTGAAAAATTGGATGTCTTTCAATTGCAGAGGAGAATTCTTCGCGTACAC |
| | | CCCTGGACCTGGAGTGAGATTTCCTCTGACCTTTGGATGGTGCTTTGCAGC |
| | | TCAAAATCCTGAAATTGTCATTTACCAATACATGGATGATCTTGCCCGTCAG |
| | | AATTACACCCCTGGACCTGGAGTGAGATACCCTCTGACCTTTGGATGGTGCT |
| | | TTAAACTGGCATTTAAAAGAAAAGGAGGAATTGGAGGATACCGCTCTTATAA |
| | | TACCCAGGGATACTTTCCTGATTGGAGGTATGCCCACACCCAAGGATTCTTC |
| | | CCTGATTGGCAAAATTACACCGCTAGGTATCCAATCCAGAAAGAAACATGGG |
| | | AAACATGGTGGACAGATTACTGGGGGAATATGTACGGACAGATGGTTCACC |
| | | AGCCAATTTCTCCAAGAACACTGAATGCTTGGGCTAATGCGTACGGAGAAAG |
| | | AATTATTGATATTATTGCTAAGTTGATTGGACCAACACCAGTGAATATTATT |
| | | GGAAGAAATATGTTGTATAGATACGCGGATTGCAAAACCATTITAAGAGCTT |
| | | TACGACGTGCATATGTGAAAGTGATTGAAGAAAAAGCTTTTTCTCCTGAAGT |
| | | GATTCCAATGTTTACAGCTCTGGCACGTCCAATCCAGAAAGAAATCTGGGAA |
| | | ACATGGTGGACAGAATACTGGGGAAACAATTTTAGAGATTATGTGGATAGA |
| | | TTCTTCAAAACCTTGAGAGCTAGAGCAATCGAAGCACAGCAGCACATGGCTC |
| | | GTGCTCGAACAGCATTGTCTGAAGGAGCAACACCTCAGGATTTGAATACAAT |
| | | GTTGAATACAGATATTTACAAAAGATGGATTATTCTGGGACTGAATAAAAGA |
| | | CGTGCCAAATTGACCCCTTTGTGTGTGACCTTGTCAATGGCATGCCAGGGA |
| | | GTGGGAGGACCTTCTCACAAAGCAAGAGTGCTTACAGCTTTTACAATCCCTT |
| | | CAACAAAGAAGCAGTACATCAAGGCCAACAGCAAGTTCATCGGCATCACCGA |
| | | GCTGAAGAAGCTGGGAGGGGGCAAACGGGGAGGCGGCAAAAAGATGACCA |
| | | ACAGCGTGGACGACGCCCTGATCAACAGCACCAAGATCTACAGCTACTTCCC |
| | | CAGCGTGATCAGCAAAGTGAACCAGGGCGCTCAGGGCAAGAAACTGTGATA |
| | | A |
| 205 | String 1.1 non-GS | MDCKTILKALRMYGERIVDIIARIVTDSQYALAEQASQEVKNWMKRNRSFNCG |
| | aa | GEFFAAAYGHQAAMQMLKETIAKQNPEIVIYRTAFTIPSIRMAKLTPLCVTLSM |
| | | MTAFTIPSVRQNYTPGPGIRYPLTFGWCFKLRKRAFKRKGGIGGYRKYNTQGF |
| | | FPDWKRMYSALSEGATPQDLNTMLNTYYLVGPTPVNIIGRNLLRYYVKVVEEK |
| | | AFSPEVIPMFSALKQNPDIVIYQYMDDLARGFRDYVDRFYKTLRARAEQASQD |
| | | VKNWMSGARAIEAQQHLRRAVRMYSPTSILDIAMAPIQKETWEAWWTEYWG |
| | | HTQGYFPDWQNYTRRYEIYKRWIILGLNKRAAGLNKIVRMYKMRTSTLQEQIG |
| | | WARMKPIQKETWEIWWTDYWGQMVHQPLSPRTLNAWYTPGPGTRYPLTFG |
| | | WAMYGQMVHQAISPRTLNAWVKVVYMATPGIRYQYNVLRKMYACQGVGGP |
| | | GHKARVLMGIPHPAGLKAKKQYIKANSKFIGITELKKLGGGKRGGGKKMTNSV |
| | | DDALINSTKIYSYFPSVISKVNQGAQGKKL |
| 206 | String 1.1 non-GS | ATGGACTGCAAGACCATCCTGAAGGCCCTGCGCATGTACGGCGAGAGAATC |
| | nt2 | GTGGACATCATTGCCAGGATCGTGACCGATAGCCAGTACGCCCTGGCCGAA |
| | | CAGGCCAGCCAAGAAGTGAAGAACTGGATGAAGCGGAACCGGAGCTTCAAT |
| | | TGTGGCGGCGAGTTCTTCGCTGCCGCGTATGGACATCAGGCCGCTATGCAG |
| | | ATGCTGAAAGAGACAATCGCCAAGCAGAACCCCGAGATCGTGATCTACCGG |
| | | ACCGCCTTCACAATCCCCAGCATCAGAATGGCGAAGCTGACCCCTCTGTGTG |
| | | TGACCCTGTCCATGATGACCGCCTTCACAATCCCTAGCGTGCGTCAGAACTA |
| | | CACACCCGGACCTGGCATCAGATACCCTCTGACCTTCGGCTGGTGCTTCAAG |
| | | CTGAGAAAACGGGCATTCAAGCGGAAAGGCGGCATCGGCGGCTATAGAAAA |
| | | TACAACACCCAGGGCTTCTTCCCCGATTGGAAGCGAATGTATTCTGCTCTGT |
| | | CTGAAGGCGCCACACCTCAGGACCTGAACACCATGCTGAACACCTACTACCT |
| | | CGTTGGACCCACACCTGTGAACATCATCGGCCGGAATCTGCTGAGGTATTAC |
| | | GTGAAGGTGGTGGAAGAAAAGGCTTTCAGCCCCGAAGTGATCCCCATGTTC |
| | | AGCGCTCTTAAGCAGAACCCCGACATCGTGATCTACCAGTACATGGACGACC |
| | | TGGCCAGAGGTTTCAGAGACTACGTGGACCGGTTCTACAAGACCCTGAGAG |
| | | CCCGAGCCGAACAGGCTTCCCAGGACGTGAAGAACTGGATGAGCGGCGCAA |
| | | GAGCCATTGAAGCCCAGCAGCACCTCCGACGGGCTGTGAGAATGTACAGCC |
| | | CCACCAGCATCCTGGACATCGCTATGGCACCCATCCAGAAAGAGACATGGGA |
| | | AGCCTGGTGGACCGAGTACTGGGGGCACACCCAGGGCTACTTCCCCGACTG |
| | | GCAGAATTACACCCGGCGCTATGAGATCTACAAGCGGTGGATCATCCTGGG |
| | | CCTGAACAAGCGAGCAGCTGGCCTGAACAAGATCGTGCGGATGTACAAAAT |
| | | GCGTACCAGCACACTGCAAGAGCAGATCGGCTGGGCAAGGATGAAACCCAT |
| | | CCAGAAAGAGACCTGGGAAATCTGGTGGACCGACTACTGGGGCCAGATGGT |
| | | TCACCAGCCTCTGAGCCCCAGAACACTGAATGCTTGGTACACACCTGGACCT |
| | | GGCACAAGATACCCTCTGACCTTTGGCTGGGCGATGTATGGACAGATGGTG |
| | | CATCAGGCCATCTCTCCCAGAACACTGAACGCCTGGGTCAAAGTGGTGTATA |
| | | TGGCAACCCCTGGCATCAGATACCAGTACAACGTGCTGCGTAAGATGTATGC |
| | | CTGTCAAGGTGTTGGAGGACCTGGACACAAGGCCAGAGTTCTGATGGGCAT |
| | | TCCTCATCCTGCCGGACTGAAAGCTAAGAAGCAGTACATCAAGGCCAACAGC |
| | | AAGTTCATCGGCATCACCGAGCTGAAGAAGCTGGGAGGGGGCAAACGGGGA |
| | | GGCGGCAAAAAGATGACCAACAGCGTGGACGACGCCCTGATCAACAGCACC |
| | | AAGATCTACAGCTACTTCCCCAGCGTGATCAGCAAAGTGAACCAGGGCGCTC |
| | | AGGGCAAGAAACTGTGATAA |
| 207 | String 1.1 non-GS | ATGGATTGCAAAACAATCTTAAAAGCTTTACGCATGTACGGAGAAAGAATTG |
| | nt3 | TGGATATTATTGCTAGGATTGTAACAGATTCTCAATATGCTCTGGCTGAACA |
| | | AGCTTCTCAGGAAGTGAAAAATTGGATGAAGCGGAACCGGTCTTTTAATTGT |
| | | GGAGGAGAATTCTTCGCTGCCGCGTATGGACACCAAGCAGCAATGCAAATG |
| | | TTGAAAGAAACCATCGCCAAACAAAATCCTGAAATTGTGATTTACCGGACCG |
| | | CTTTTACCATTCCATCAATTAGAATGGCGAAATTGACCCCTTTGTGTGTGAC |
| | | CTTGTCCATGATGACCGCTTTTACCATTCCAAGTGTTCGTCAGAATTACACC |
| | | CCTGGACCTGGAATCAGATACCCTCTGACCTTTGGATGGTGCTTTAAACTGA |
| | | GAAAACGGGCATTTAAAAGAAAAGGAGGAATTGGAGGATACAGAAAATACA |
| | | ACACCCAAGGATTCTTCCCTGATTGGAAGCGAATGTATTCTGCATTGTCTGA |
| | | AGGAGCAACCCCTCAAGATTTGAATACCATGTTGAATACCTACTACCTTGTA |
| | | GGACCAACACCTGTAAATATCATCGGAAGAAATCTTCTTAGGTATTACGTGA |
| | | AAGTGGTGGAAGAAAAAGCTTTTTCTCCTGAAGTGATTCCAATGTTTTCTGC |
| | | TCTTAAACAAAATCCTGATATTGTAATTTACCAATACATGGATGATCTGGCC |
| | | AGAGGTTTTAGAGATTACGTGGATAGATTTTACAAAACACTGAGAGCACGA |
| | | GCTGAACAAGCTTCTCAAGACGTTAAAAATTGGATGAGCGGCGCAAGAGCA |
| | | ATTGAAGCACAGCAGCACCTCCGACGGGCTGTGAGAATGTACTCACCAACCT |
| | | CAATTCTGGATATTGCTATGGCACCAATTCAGAAAGAAACATGGGAAGCTTG |
| | | GTGGACAGAATACTGGGGGCACACACAGGGATACTTTCCTGATTGGCAGAA |
| | | TTACACACGGCGCTATGAAATTTACAAAAGATGGATTATTCTGGGACTGAAT |
| | | AAACGAGCAGCTGGATTAAATAAAATTGTAAGAATGTACAAAATGCGTACAT |
| | | CAACACTTCAGGAACAGATTGGATGGGCAAGGATGAAACCAATCCAGAAAG |
| | | AAACCTGGGAAATATGGTGGACAGATTACTGGGGACAGATGGTGCACCAGC |
| | | CATTGTCTCCAAGAACATTGAATGCTTGGTACACACCTGGACCTGGAACAAG |
| | | ATACCCTCTGACATTTGGATGGGCGATGTATGGACAGATGGTTCACCAGGC |
| | | AATTTCTCCAAGAACATTAAATGCATGGGTTAAAGTTGTTTATATGGCAACA |
| | | CCTGGAATAAGATACCAATACAATGTGCTTCGTAAGATGTATGCATGCCAGG |
| | | GAGTGGGAGGACCTGGACACAAAGCAAGAGTGCTTATGGGAATTCCTCATC |
| | | CTGCTGGACTGAAAGCTAAGAAGCAGTACATCAAGGCCAACAGCAAGTTCAT |
| | | CGGCATCACCGAGCTGAAGAAGCTGGGAGGGGGCAAACGGGGAGGCGGCA |
| | | AAAAGATGACCAACAGCGTGGACGACGCCCTGATCAACAGCACCAAGATCTA |
| | | CAGCTACTTCCCCAGCGTGATCAGCAAAGTGAACCAGGGCGCTCAGGGCAA |
| | | GAAACTGTGATAA |
| 208 | String 2.1 non-GS | MIVTDSQYALYMAEQATQEVKNWMATSTLQEQIAWGIPHPAGLKRRYAVRM |
| | aa | YSPVSILDIRSYGLNKIVRMYRMGHQAAMQMLKETIAKNPEIVIYSYGQMVHQ |
| | | ALSPRTLNAWVKVIYMATPGIRYQYNVLAEQATQDVKNWMSFNCRGEFFAYT |
| | | PGPGVRFPLTFGWCFAAQNPEIVIYQYMDDLARQNYTPGPGVRYPLTFGWCF |
| | | KLAFKRKGGIGGYRSYNTQGYFPDWRYAHTQGFFPDWQNYTARYPIQKETW |
| | | ETWWTDYWGNMYGQMVHQPISPRTLNAWANAYGERIIDIIAKLIGPTPVNII |
| | | GRNMLYRYADCKTILRALRRAYVKVIEEKAFSPEVIPMFTALARPIQKETWETW |
| | | WTEYWGNNFRDYVDRFFKTLRARAIEAQQHMARARTALSEGATPQDLNTML |
| | | NTDIYKRWIILGLNKRRAKLTPLCVTLSMACQGVGGPSHKARVLTAFTIPSTKK |
| | | QYIKANSKFIGITELKKLGGGKRGGGKKMTNSVDDALINSTKIYSYFPSVISKVN |
| | | QGAQGKKL |
| 209 | String 2.1 non-GS | ATGATCGTGACCGATAGCCAGTACGCCCTGTACATGGCCGAACAGGCCACA |
| | nt2 | CAAGAAGTGAAGAACTGGATGGCGACCAGCACACTGCAAGAGCAGATCGCC |
| | | TGGGGCATTCCTCATCCTGCCGGACTGAAAAGACGGTATGCAGTGAGAATG |
| | | TACAGCCCCGTGTCCATCCTGGACATCCGTTCTTACGGCCTGAACAAGATCG |
| | | TGCGGATGTACCGTATGGGACATCAGGCCGCTATGCAGATGCTGAAAGAGA |
| | | CAATCGCCAAGAATCCCGAGATCGTGATCTACTCCTACGGCCAGATGGTTCA |
| | | CCAGGCTCTGAGCCCCAGAACACTGAACGCCTGGGTCAAAGTGATCTATAT |
| | | GGCCACCCCTGGCATCAGATACCAGTACAACGTGCTGGCCGAACAGGCTAC |
| | | CCAGGACGTGAAGAACTGGATGAGCTTCAATTGCAGGGGCGAGTTCTTCGC |
| | | GTACACACCTGGACCTGGCGTCAGATTCCCTCTGACCTTCGGCTGGTGCTTC |
| | | GCAGCCCAGAATCCTGAGATCGTGATCTACCAGTACATGGACGACCTGGCC |
| | | CGTCAGAACTACACACCTGGACCTGGCGTGCGGTATCCTCTGACCTTCGGCT |
| | | GGTGCTTCAAGCTGGCATTCAAGCGGAAAGGCGGCATCGGCGGCTATCGCT |
| | | CTTATAACACCCAGGGCTACTTCCCCGATTGGAGGTATGCCCACACCCAGGG |
| | | ATTCTTCCCAGACTGGCAGAACTACACCGCTAGGTATCCCATCCAGAAAGAG |
| | | ACATGGGAAACCTGGTGGACCGACTACTGGGGGAATATGTACGGCCAGATG |
| | | GTGCATCAGCCTATCAGCCCCAGAACACTGAACGCCTGGGCTAATGCGTAC |
| | | GGCGAGCGGATCATCGACATCATTGCCAAGCTGATCGGACCCACACCTGTG |
| | | AACATCATCGGCCGGAACATGCTGTATAGATACGCGGACTGCAAGACCATCC |
| | | TGAGAGCCCTGCGACGTGCATATGTGAAAGTGATCGAGGAAAAGGCTTTCA |
| | | GCCCCGAAGTGATCCCCATGTTCACAGCCCTTGCACGTCCCATCCAGAAAGA |
| | | GACTTGGGAAACCTGGTGGACCGAGTACTGGGGAAACAATTTCAGAGACTA |
| | | CGTGGACCGGTTCTTCAAGACCCTGAGAGCCAGAGCCATTGAAGCCCAGCA |
| | | GCACATGGCTCGTGCTCGAACAGCTCTGTCTGAAGGCGCCACACCTCAGGA |
| | | CCTGAACACCATGCTGAACACCGACATCTACAAGCGGTGGATCATCCTGGGC |
| | | CTGAACAAAAGACGTGCCAAGCTGACCCCTCTGTGTGTGACCCTGTCAATGG |
| | | CCTGTCAAGGTGTTGGCGGACCTTCTCACAAGGCCAGAGTTCTGACCGCCT |
| | | TCACAATCCCTAGCACCAAGAAGCAGTACATCAAGGCCAACAGCAAGTTCAT |
| | | CGGCATCACCGAGCTGAAGAAGCTGGGAGGGGGCAAACGGGGAGGCGGCA |
| | | AAAAGATGACCAACAGCGTGGACGACGCCCTGATCAACAGCACCAAGATCTA |
| | | CAGCTACTTCCCCAGCGTGATCAGCAAAGTGAACCAGGGCGCTCAGGGCAA |
| | | GAAACTGTGATAA |
| 210 | String 2.1 non-GS | ATGATTGTAACAGATTCTCAATATGCTCTGTACATGGCTGAACAAGCTACAC |
| | nt3 | AAGAAGTGAAAAATTGGATGGCGACATCAACACTTCAGGAACAGATTGCTTG |
| | | GGGAATTCCTCACCCTGCTGGATTGAAAAGACGGTATGCAGTGAGAATGTA |
| | | CTCTCCTGTGTCAATTCTGGATATTCGTTCTTACGGATTAAATAAAATTGTA |
| | | AGAATGTACCGTATGGGACACCAAGCAGCAATGCAAATGCTGAAAGAAACCA |
| | | TTGCTAAAAATCCTGAAATTGTGATTTACTCCTACGGACAGATGGTGCACCA |
| | | GGCTTTGTCTCCAAGAACATTGAATGCTTGGGTAAAAGTAATTTATATGGCC |
| | | ACACCTGGAATAAGATACCAATACAATGTGCTTGCAGAACAAGCAACACAAG |
| | | ATGTGAAAAATTGGATGTCTTTCAATTGCAGAGGAGAATTCTTCGCGTACAC |
| | | CCCTGGACCTGGAGTGAGATTTCCTCTGACCTTTGGATGGTGCTTTGCAGC |
| | | TCAAAATCCTGAAATTGTCATTTACCAATACATGGATGATCTTGCCCGTCAG |
| | | AATTACACCCCTGGACCTGGAGTGAGATACCCTCTGACCTTTGGATGGTGCT |
| | | TTAAACTGGCATTTAAAAGAAAAGGAGGAATTGGAGGATACCGCTCTTATAA |
| | | TACCCAGGGATACTTTCCTGATTGGAGGTATGCCCACACCCAAGGATTCTTC |
| | | CCTGATTGGCAAAATTACACCGCTAGGTATCCAATCCAGAAAGAAACATGGG |
| | | AAACATGGTGGACAGATTACTGGGGGAATATGTACGGACAGATGGTTCACC |
| | | AGCCAATTTCTCCAAGAACACTGAATGCTTGGGCTAATGCGTACGGAGAAAG |
| | | AATTATTGATATTATTGCTAAGTTGATTGGACCAACACCAGTGAATATTATT |
| | | GGAAGAAATATGTTGTATAGATACGCGGATTGCAAAACCATTITAAGAGCTT |
| | | TACGACGTGCATATGTGAAAGTGATTGAAGAAAAAGCTTTTTCTCCTGAAGT |
| | | GATTCCAATGTTTACAGCTCTGGCACGTCCAATCCAGAAAGAAACTTGGGAA |
| | | ACATGGTGGACAGAATACTGGGGAAACAATTTTAGAGATTATGTGGATAGA |
| | | TTCTTCAAAACCTTGAGAGCTAGAGCAATCGAAGCACAGCAGCACATGGCTC |
| | | GTGCTCGAACAGCATTGTCTGAAGGAGCAACACCTCAGGATTTGAATACAAT |
| | | GTTGAATACAGATATTTACAAAAGATGGATTATTCTGGGACTGAATAAAAGA |
| | | CGTGCCAAATTGACCCCTTTGTGTGTGACCTTGTCAATGGCATGCCAGGGA |
| | | GTGGGAGGACCTTCTCACAAAGCAAGAGTGCTTACAGCTITTACAATCCCTT |
| | | CAACAAAGAAGCAGTACATCAAGGCCAACAGCAAGTTCATCGGCATCACCGA |
| | | GCTGAAGAAGCTGGGAGGGGGCAAACGGGGAGGCGGCAAAAAGATGACCA |
| | | ACAGCGTGGACGACGCCCTGATCAACAGCACCAAGATCTACAGCTACTTCCC |
| | | CAGCGTGATCAGCAAAGTGAACCAGGGCGCTCAGGGCAAGAAACTGTGATAA |
| 187 | String 1 GS aa | MDCKTILKALRKYGGSGGGGSGGNAYGERIVDIIAKKGGSGGGGSGGRARIVT |
| | | DSQYALSMNGGSGGGGSGGYMAAEQASQEVKNWMKRYGGSGGGGSGGYNY |
| | | SFNCGGEFFSGWGGSGGGGSGGYAAGHQAAMQMLKETISREGGSGGGGSG |
| | | GMAKQNPEIVIYRMGGSGGGGSGGSATAFTIPSISGGSGGGGSGGRAAKLTPL |
| | | CVTLSMMGGSGGGGSGGTAFTIPSVRWGGSGGGGSGGMMRQNYTPGPGIRY |
| | | PLTFGWCFKLKKRGGSGGGGSGGYSAFKRKGGIGGYRRYGGSGGGGSGGYSY |
| | | NTQGYYPDWKRYGGSGGGGSGGQNDSALSEGATPQDLNTMLNTRRGGSGG |
| | | GGSGGRGRLVGPTPVNIIGRNLLFWRGGSGGGGSGGMYMVKVVEEKAFSPEV |
| | | IPMFSALKRGGSGGGGSGGFYYKQNPDIVIYQYMDDLKRYGGSGGGGSGGYA |
| | | AFRDYVDRFYKTLRADDDGGSGGGGSGGYMYAEQASQDVKNWMSMFGGSG |
| | | GGGSGGRAIEAQQHLKRGGSGGGGSGGNQDVRMYSPTSILDIGMGGGSGGG |
| | | GSGGIRYPIQKETWEAWWTEYWGRRGGSGGGGSGGYSFHTQGYFPDWQNY |
| | | TGRYGGSGGGGSGGNYEIYKRWIILGLNKRRGGSGGGGSGGRAAGLNKIVRM |
| | | YRKYGGSGGGGSGGRMFTSTLQEQIGWKIFGGSGGGGSGGNIYPIQKEIWET |
| | | WWTDYWGNNGGSGGGGSGGMGGGQMVHQPLSPRTLNAWYRYGGSGGGG |
| | | SGGYAYYTPGPGTRYPLTFGWAKGGGSGGGGSGGFFFGQMVHQAISPRTLNA |
| | | WVKVVYYYGGSGGGGSGGYMYTPGIRYQYNVLKKTGGSGGGGSGGKAAACQ |
| | | GVGGPGHKARVLKTQGGSGGGGSGGTFGIPHPAGLKRRGGSGGGGSGGKKQ |
| | | YIKANSKFIGITELKKLGGGKRGGGKKMTNSVDDALINSTKIYSYFPSVISKVNQ |
| | | GAQGKKL |
| 188 | String 1 GS nt1 | ATGGATTGTAAGACTATTTTAAAAGCATTGCGGAAGTACGGTGGCTCTGGA |
| | | GGCGGTGGATCAGGTGGAAATGCGTACGGGGAAAGAATAGTAGACATAATA |
| | | GCAAAGAAGGGTGGAAGCGGAGGCGGTGGATCGGGTGGCCGTGCGCGTAT |
| | | AGTAACAGACTCACAGTATGCATTATCGATGAATGGTGGCAGCGGAGGCGG |
| | | TGGATCCGGCGGTTACATGGCCGCCGAGCAAGCTTCACAGGAGGTAAAAAA |
| | | TTGGATGAAACGGTATGGAGGCAGTGGTGGCGGAGGTAGTGGTGGATATA |
| | | ATTACAGTTTTAATTGTGGAGGGGAATTTTTCAGCGGCTGGGGAGGTAGCG |
| | | GCGGTGGAGGCAGCGGAGGTTACGCGGCTGGACATCAAGCAGCCATGCAAA |
| | | TGTTAAAAGAGACCATCTCACGAGAGGGAGGTAGTGGAGGCGGTGGATCGG |
| | | GCGGTATGGCTAAACAAAATCCAGAGATAGTTATCTATAGGATGGGTGGCA |
| | | GTGGAGGTGGCGGAAGCGGAGGTTCTGCGACTGCATTCACCATACCTAGTA |
| | | TATCCGGTGGCAGTGGTGGCGGAGGTTCAGGTGGACGCGCGGCTAAGTTG |
| | | ACCCCACTCTGTGTCACTTTATCCATGATGGGTGGATCTGGTGGAGGCGGT |
| | | TCTGGCGGTACTGCATTCACCATACCTAGTGTACGGTGGGGAGGCTCGGGT |
| | | GGAGGCGGTTCCGGAGGCATGATGCGGCAGAACTACACACCAGGGCCAGG |
| | | GATCAGATATCCACTGACCTTTGGATGGTGCTTCAAGCTAAAAAAGCGAGGC |
| | | GGTTCCGGTGGAGGCGGTAGCGGAGGTTACTCAGCATTTAAAAGAAAAGGG |
| | | GGGATTGGGGGGTACAGGCGTTATGGTGGCTCTGGTGGAGGCGGTAGCGG |
| | | CGGTTACAGTTACAACACACAAGGCTACTACCCTGATTGGAAGCGATATGGT |
| | | GGATCGGGAGGCGGTGGAAGTGGTGGACAGAATGATTCAGCATTATCAGAA |
| | | GGAGCCACCCCACAAGATTTAAACACCATGCTAAACACAAGACGTGGTGGAT |
| | | CGGGCGGTGGAGGCTCAGGAGGCCGCGGGCGGTTAGTAGGACCTACACCT |
| | | GTCAACATAATTGGAAGAAATCTGTTGTTCTGGCGGGGCGGATCTGGTGGC |
| | | GGAGGTTCGGGCGGAATGTATATGGTAAAAGTAGTAGAAGAGAAGGCTTTC |
| | | AGCCCAGAAGTAATACCCATGTTTTCAGCATTAAAGAGGGGTGGCTCCGGC |
| | | GGAGGTGGCTCTGGCGGATTCTACTATAAACAAAATCCAGACATAGTTATCT |
| | | ATCAATACATGGATGATTTGAAACGTTATGGTGGCAGTGGAGGCGGTGGAT |
| | | CTGGAGGTTATGCTGCCTTTAGAGACTATGTAGACCGGTTCTATAAAACTCT |
| | | AAGAGCCGACGATGATGGCGGTAGCGGAGGCGGTGGATCAGGAGGCTACA |
| | | TGTACGCTGAACAAGCTTCACAAGATGTAAAAAATTGGATGTCTATGTTTGG |
| | | CGGTTCTGGCGGAGGTGGCAGTGGTGGCAGGGCTATTGAGGCGCAACAGC |
| | | ATCTGAAGAGAGGTGGAAGTGGCGGTGGAGGCTCCGGTGGCAATCAAGAT |
| | | GTAAGAATGTATAGCCCTACCAGCATTCTGGACATAGGCATGGGAGGAGGC |
| | | TCGGGAGGCGGTGGATCGGGAGGCATCAGGTACCCCATACAAAAAGAAACA |
| | | TGGGAAGCATGGTGGACAGAGTATTGGGGGAGAAGAGGTGGATCCGGTGG |
| | | AGGCGGTAGTGGTGGCTACAGTTTCCACACACAAGGCTACTTCCCTGATTG |
| | | GCAGAACTACACAGGTCGATACGGCGGTTCAGGTGGAGGCGGTTCAGGAGG |
| | | TAACTACGAAATCTATAAAAGATGGATAATCCTGGGATTAAATAAACGTAGA |
| | | GGAGGCTCCGGCGGAGGTGGCTCAGGTGGACGGGCTGCTGGGTTAAATAA |
| | | AATAGTAAGAATGTATCGAAAGTATGGCGGTTCAGGCGGAGGTGGCTCCGG |
| | | AGGCAGAATGTTCACTAGTACCCTTCAGGAACAAATAGGATGGAAAATATTT |
| | | GGTGGATCAGGCGGTGGAGGCTCTGGAGGTAACATCTATCCCATCCAAAAA |
| | | GAAATATGGGAGACATGGTGGACAGACTATTGGGGAAACAACGGOGGATCC |
| | | GGCGGTGGAGGCTCGGGAGGTATGGGTGGGGGGCAAATGGTACACCAGCC |
| | | CCTATCACCTAGAACTTTGAATGCATGGTACCGTTATGGCGGATCGGGCGG |
| | | AGGTGGCAGCGGAGGCTACGCTTACTACACACCGGGACCAGGGACTAGATA |
| | | TCCACTGACCTTTGGATGGGCCAAGGGTGGCGGTTCTGGAGGTGGOGGATC |
| | | CGGCGGTTTCTTTTTCGGGCAAATGGTACATCAGGCCATATCACCTAGAACT |
| | | TTAAATGCATGGGTAAAAGTAGTATATTACTATGGTGGCTCAGGTGGAGGC |
| | | GGTAGTGGTGGATACATGTATACACCAGGGATTAGATATCAATATAATGTGC |
| | | TTAAGAAGACTGGTGGCTCAGGCGGAGGTGGCAGCGGCGGTAAGGCGGCG |
| | | GCATGTCAGGGAGTGGGAGGACCTGGCCACAAAGCAAGAGTGTTGAAGACC |
| | | CAGGGCGGATCGGGCGGAGGTGGCTCTGGTGGAACATTCGGAATACCACAC |
| | | CCAGCAGGGTTAAAAAGACGTGGCGGATCAGGTGGCGGAGGTTCGGGAGG |
| | | CAAGAAGCAGTACATCAAGGCCAACAGCAAGTTCATCGGCATCACCGAGCTG |
| | | AAGAAGCTGGGAGGGGGCAAACGGGGAGGCGGCAAAAAGATGACCAACAG |
| | | CGTGGACGACGCCCTGATCAACAGCACCAAGATCTACAGCTACTTCCCCAGC |
| | | GTGATCAGCAAAGTGAACCAGGGCGCTCAGGGCAAGAAACTG |
| 215 | String 1 GS nt2 | ATGGACTGCAAGACCATCCTGAAGGCCCTGCGGAAGTACGGTGGCTCTGGA |
| | | GGCGGTGGATCAGGTGGAAATGCGTACGGCGAGAGAATCGTGGACATCATT |
| | | GCCAAGAAGGGTGGAAGCGGAGGCGGTGGATCGGGTGGCCGTGCGCGTAT |
| | | CGTGACCGATAGCCAGTACGCCCTGTCGATGAATGGAGGTAGCGGTGGCGG |
| | | AGGTTCTGGCGGATACATGGCCGCCGAACAGGCCAGCCAAGAAGTGAAGAA |
| | | CTGGATGAAACGGTATGGAGGCAGTGGTGGCGGAGGTAGTGGTGGATATA |
| | | ATTACAGCTTCAATTGTGGCGGCGAGTTCTTCAGCGGCTGGGGAGGTAGCG |
| | | GCGGTGGAGGCAGCGGAGGTTACGCGGCTGGACATCAGGCCGCTATGCAG |
| | | ATGCTGAAAGAGACAATCTCACGAGAGGGAGGTAGTGGAGGCGGTGGATCG |
| | | GGCGGTATGGCTAAGCAGAACCCCGAGATCGTGATCTACAGGATGGGTGGC |
| | | AGTGGAGGTGGCGGAAGCGGAGGTTCTGCGACCGCCTTCACAATCCCCAGC |
| | | ATCTCCGGTGGCAGTGGTGGCGGAGGTTCAGGTGGACGCGCGGCTAAGCT |
| | | GACCCCTCTGTGTGTGACCCTGTCCATGATGGGTGGATCTGGTGGAGGCGG |
| | | TTCTGGCGGTACCGCCTTCACAATCCCTAGCGTGCGGTGGGGTGGCAGTGG |
| | | CGGTGGAGGCAGTGGTGGCATGATGCGGCAGAACTACACACCCGGACCTGG |
| | | CATCAGATACCCTCTGACCTTCGGCTGGTGCTTCAAGCTGAAAAAGCGAGGC |
| | | GGTTCCGGTGGAGGCGGTAGCGGAGGTTACTCAGCATTCAAGCGGAAAGGC |
| | | GGCATCGGCGGCTATAGGCGTTATGGTGGCTCTGGTGGAGGCGGTAGCGG |
| | | CGGTTACAGTTACAACACCCAGGGCTACTACCCCGATTGGAAGCGATATGGT |
| | | GGATCGGGAGGCGGTGGAAGTGGTGGACAGAATGATTCTGCTCTGTCTGAA |
| | | GGCGCCACACCTCAGGACCTGAACACCATGCTGAACACCAGACGTGGTGGA |
| | | TCGGGCGGTGGAGGCTCAGGAGGCCGCGGGCGGCTCGTTGGACCCACACC |
| | | TGTGAACATCATCGGCCGGAATCTGCTGTTCTGGCGGGGCGGATCTGGTGG |
| | | CGGAGGTTCGGGCGGAATGTATATGGTGAAGGTGGTGGAAGAAAAGGCTTT |
| | | CAGCCCCGAAGTGATCCCCATGTTCAGCGCTCTTAAGAGGGGTGGCTCCGG |
| | | CGGAGGTGGCTCTGGCGGATTCTACTATAAGCAGAACCCCGACATCGTGAT |
| | | CTACCAGTACATGGACGACCTGAAACGTTATGGTGGCAGTGGAGGCGGTGG |
| | | ATCTGGAGGTTATGCTGCCTTCAGAGACTACGTGGACCGGTTCTACAAGAC |
| | | CCTGAGAGCCGACGATGATGGCGGTAGCGGAGGCGGTGGATCAGGAGGCT |
| | | ACATGTACGCCGAACAGGCTTCCCAGGACGTGAAGAACTGGATGTCTATGT |
| | | TTGGCGGTTCTGGCGGAGGTGGCAGTGGTGGCAGAGCCATTGAAGCCCAG |
| | | CAGCACCTCAAGAGAGGTGGAAGTGGCGGTGGAGGCTCCGGTGGCAATCAA |
| | | GATGTGAGAATGTACAGCCCCACCAGCATCCTGGACATCGGCATGGGAGGA |
| | | GGCTCGGGAGGCGGTGGATCGGGAGGCATCAGGTACCCCATCCAGAAAGA |
| | | GACATGGGAAGCCTGGTGGACCGAGTACTGGGGGAGGAGGGGCGGTAGCG |
| | | GCGGAGGTGGCAGTGGAGGTTACAGTTTCCACACCCAGGGCTACTTCCCCG |
| | | ACTGGCAGAATTACACCGGTCGATACGGCGGTTCAGGTGGAGGCGGTTCAG |
| | | GAGGTAACTACGAGATCTACAAGCGGTGGATCATCCTGGGCCTGAACAAGC |
| | | GTAGAGGAGGCTCCGGCGGAGGTGGCTCAGGTGGACGGGCTGCTGGCCTG |
| | | AACAAGATCGTGCGGATGTACCGAAAGTATGGCGGTAGCGGAGGTGGCGGA |
| | | AGCGGCGGTAGAATGTTCACCAGCACACTGCAAGAGCAGATCGGCTGGAAA |
| | | ATATTTGGTGGATCAGGCGGTGGAGGCTCTGGAGGTAACATCTATCCCATC |
| | | CAGAAAGAGATCTGGGAAACCTGGTGGACCGACTACTGGGGAAACAACGGA |
| | | GGTTCTGGTGGAGGCGGTTCAGGAGGTATGGGTGGGGGCCAGATGGTTCA |
| | | CCAGCCTCTGAGCCCCAGAACACTGAATGCTTGGTACCGTTATGGCGGATC |
| | | GGGCGGAGGTGGCAGCGGAGGCTACGCTTACTACACACCTGGACCTGGCAC |
| | | AAGATACCCTCTGACCTTTGGCTGGGCCAAGGGTGGAGGTAGCGGCGGAGG |
| | | TGGCTCGGGTGGCTTCTTTTTCGGACAGATGGTGCATCAGGCCATCTCTCC |
| | | CAGAACACTGAACGCCTGGGTCAAAGTGGTGTATTACTATGGTGGCTCAGG |
| | | TGGAGGCGGTAGTGGTGGATACATGTATACCCCTGGCATCAGATACCAGTA |
| | | CAACGTGCTGAAGAAGACTGGTGGCTCAGGCGGAGGTGGCAGCGGCGGTA |
| | | AGGCGGCGGCCTGTCAAGGTGTTGGAGGACCTGGACACAAGGCCAGAGTTC |
| | | TGAAGACCCAGGGCGGATCGGGCGGAGGTGGCTCTGGTGGAACATTCGGC |
| | | ATTCCTCATCCTGCCGGACTGAAAAGACGTGGCGGATCAGGTGGCGGAGGT |
| | | TCGGGAGGCAAGAAGCAGTACATCAAGGCCAACAGCAAGTTCATCGGCATC |
| | | ACCGAGCTGAAGAAGCTGGGAGGGGGCAAACGGGGAGGCGGCAAAAAGAT |
| | | GACCAACAGCGTGGACGACGCCCTGATCAACAGCACCAAGATCTACAGCTAC |
| | | TTCCCCAGCGTGATCAGCAAAGTGAACCAGGGCGCTCAGGGCAAGAAACTG |
| | | TGATAA |
| 216 | String 1 GS nt3 | ATGGATTGCAAAACAATCTTAAAAGCTTTACGGAAGTACGGTGGCTCTGGA |
| | | GGCGGTGGATCAGGTGGAAATGCGTACGGAGAAAGAATTGTGGATATTATT |
| | | GCTAAGAAGGGTGGAAGCGGAGGCGGTGGATCGGGTGGCCGTGCGCGTAT |
| | | TGTAACAGATTCTCAATATGCTCTGTCGATGAATGGAGGTAGCGGTGGCGG |
| | | AGGTTCTGGCGGATACATGGCCGCTGAACAAGCTTCTCAGGAAGTGAAAAA |
| | | TTGGATGAAACGGTATGGAGGCAGTGGTGGCGGAGGTAGTGGTGGATATA |
| | | ATTACTCTTTTAATTGTGGAGGAGAATTCTTCAGCGGCTGGGGAGGTAGCG |
| | | GCGGTGGAGGCAGCGGAGGTTACGCGGCTGGACACCAAGCAGCAATGCAAA |
| | | TGTTGAAAGAAACCATCTCACGAGAGGGAGGTAGTGGAGGCGGTGGATCGG |
| | | GCGGTATGGCTAAACAAAATCCTGAAATTGTGATTTACAGGATGGGTGGCA |
| | | GTGGAGGTGGCGGAAGCGGAGGTTCTGCGACCGCTTTTACCATTCCATCAA |
| | | TTTCCGGTGGCAGTGGTGGCGGAGGTTCAGGTGGACGCGCGGCTAAATTGA |
| | | CCCCTTTGTGTGTGACCTTGTCCATGATGGGTGGATCTGGTGGAGGCGGTT |
| | | CTGGCGGTACCGCTTTTACCATTCCAAGTGTTCGGTGGGGTGGCAGTGGCG |
| | | GTGGAGGCAGTGGTGGCATGATGCGGCAGAATTACACCCCTGGACCTGGAA |
| | | TCAGATACCCTCTGACCTTTGGATGGTGCTTTAAACTGAAAAAGCGAGGCG |
| | | GTTCCGGTGGAGGCGGTAGCGGAGGTTACTCAGCATTTAAAAGAAAAGGAG |
| | | GAATTGGAGGATACAGGCGTTATGGTGGCTCTGGTGGAGGCGGTAGCGGC |
| | | GGTTACAGTTACAACACCCAAGGATACTACCCTGATTGGAAGCGATATGGTG |
| | | GATCGGGAGGCGGTGGAAGTGGTGGACAGAATGATTCTGCATTGTCTGAAG |
| | | GAGCAACCCCTCAAGATTTGAATACCATGTTGAATACCAGACGTGGTGGATC |
| | | GGGCGGTGGAGGCTCAGGAGGCCGCGGGCGGCTTGTAGGACCAACACCTG |
| | | TAAATATCATCGGAAGAAATCTTCTTTTCTGGCGGGGCGGATCTGGTGGCG |
| | | GAGGTTCGGGCGGAATGTATATGGTGAAAGTGGTGGAAGAAAAAGCTTTTT |
| | | CTCCTGAAGTGATTCCAATGTTTTCTGCTCTTAAGAGGGGGGCTCCGGCG |
| | | GAGGTGGCTCTGGCGGATTCTACTATAAACAAAATCCTGATATTGTAATTTA |
| | | CCAATACATGGATGATCTGAAACGTTATGGTGGCAGTGGAGGCGGTGGATC |
| | | TGGAGGTTATGCTGCCTTTAGAGATTACGTGGATAGATTTTACAAAACACTG |
| | | AGAGCAGACGATGATGGCGGTAGCGGAGGCGGTGGATCAGGAGGCTACAT |
| | | GTACGCTGAACAAGCTTCTCAAGACGTTAAAAATTGGATGTCTATGTTTGGC |
| | | GGTTCTGGCGGAGGTGGCAGTGGTGGCAGAGCAATTGAAGCACAGCAGCAC |
| | | CTCAAGAGAGGTGGAAGTGGCGGTGGAGGCTCCGGTGGCAATCAAGATGT |
| | | GAGAATGTACTCACCAACCTCAATTCTGGATATTGGCATGGGAGGAGGCTC |
| | | GGGAGGCGGTGGATCGGGAGGCATCAGGTACCCAATTCAGAAAGAAACATG |
| | | GGAAGCTTGGTGGACAGAATACTGGGGGAGGAGGGGCGGTAGCGGCGGAG |
| | | GTGGCAGTGGAGGTTACAGTTTCCACACACAGGGATACTTTCCTGATTGGC |
| | | AGAATTACACAGGTCGATACGGCGGTTCAGGTGGAGGCGGTTCAGGAGGTA |
| | | ACTACGAAATTTACAAAAGATGGATTATTCTGGGACTGAATAAACGTAGAGG |
| | | AGGCTCCGGCGGAGGTGGCTCAGGTGGACGGGCTGCTGGATTAAATAAAAT |
| | | TGTAAGAATGTACCGAAAGTATGGCGGTAGCGGAGGTGGCGGAAGCGGCG |
| | | GTAGAATGTTCACATCAACACTTCAGGAACAGATTGGATGGAAAATATTTGG |
| | | TGGATCAGGCGGTGGAGGCTCTGGAGGTAACATCTATCCAATCCAGAAAGA |
| | | AATCTGGGAAACATGGTGGACAGATTACTGGGGAAACAACGGAGGTTCTGG |
| | | TGGAGGCGGTTCAGGAGGTATGGGTGGGGGACAGATGGTGCACCAGCCAT |
| | | TGTCTCCAAGAACATTGAATGCTTGGTACCGTTATGGCGGATCGGGCGGAG |
| | | GTGGCAGCGGAGGCTACGCTTACTACACACCTGGACCTGGAACAAGATACC |
| | | CTCTGACATTTGGATGGGCCAAGGGTGGAGGTAGCGGCGGAGGTGGCTCG |
| | | GGTGGCTTCTTTTTCGGACAGATGGTTCACCAGGCAATTTCTCCAAGAACAT |
| | | TAAATGCATGGGTTAAAGTTGTTTATTACTATGGTGGCTCAGGTGGAGGCG |
| | | GTAGTGGTGGATACATGTATACACCTGGAATAAGATACCAATACAATGTGCT |
| | | TAAGAAGACTGGTGGCTCAGGCGGAGGTGGCAGCGGCGGTAAGGCGGCGG |
| | | CATGCCAGGGAGTGGGAGGACCTGGACACAAAGCAAGAGTGCTTAAGACCC |
| | | AGGGCGGATCGGGCGGAGGTGGCTCTGGTGGAACATTCGGAATTCCTCATC |
| | | CTGCTGGACTGAAAAGACGTGGCGGATCAGGTGGCGGAGGTTCGGGAGGC |
| | | AAGAAGCAGTACATCAAGGCCAACAGCAAGTTCATCGGCATCACCGAGCTGA |
| | | AGAAGCTGGGAGGGGGCAAACGGGGAGGCGGCAAAAAGATGACCAACAGC |
| | | GTGGACGACGCCCTGATCAACAGCACCAAGATCTACAGCTACTTCCCCAGCG |
| | | TGATCAGCAAAGTGAACCAGGGCGCTCAGGGCAAGAAACTGTGATAA |
| 189 | String 2 GS aa | MIVTDSQYALSMNGGSGGGGSGGMMQAEQATQEVKNWMSGAGGSGGGGS |
| | | GGNMFTSTLQEQIAWKIFGGSGGGGSGGTFGIPHPAGLKRRGGSGGGGSGGL |
| | | QDVRMYSPVSILDIGMGGGSGGGGSGGRAAGLNKIVRMYRKYGGSGGGGSG |
| | | GYAAGHQAAMQMLKETISREGGSGGGGSGGMYAKNPEIVIYKMGGSGGGGS |
| | | GGYAFGQMVHQALSPRTLNAWVKVIGRRGGSGGGGSGGYMYTPGIRYQYNV |
| | | LKKTGGSGGGGSGGYMAAEQATQDVKNWMKRYGGSGGGGSGGQNRSFNCR |
| | | GEFFAGNGGSGGGGSGGMMMYTPGPGVRFPLTFGWCFMGNGGSGGGGSGG |
| | | AYAQNPEIVIYQYMDDLKRGGSGGGGSGGMMRQNYTPGPGVRYPLTFGWCF |
| | | KLKKRGGSGGGGSGGYSAFKRKGGIGGYRRYGGSGGGGSGGYNTQGYFPDW |
| | | KIGGSGGGGSGGTYAHTQGFFPDWQNYTKRGGSGGGGSGGNIYPIQKETWE |
| | | TWWTDYWGGNGGSGGGGSGGMMYGQMVHQPISPRTLNAWARNGGSGGG |
| | | GSGGMAYGERIIDIIAKKGGGSGGGGSGGRGGLIGPTPVNIIGRNMLFWRGGS |
| | | GGGGSGGRAADCKTILRALRMRGGSGGGGSGGAVKVIEEKAFSPEVIPMFTAL |
| | | AIGGSGGGGSGGIMYPIQKEIWETWWTEYWGAWGGSGGGGSGGWAFRDYV |
| | | DRFFKTLRAGKGGSGGGGSGGRAIEAQQHMKRGGSGGGGSGGNNYTALSEG |
| | | ATPQDLNTMLNTWGGGSGGGGSGGNYDIYKRWIILGLNKRRGGSGGGGSGG |
| | | RAAKLTPLCVTLSMMGGSGGGGSGGFAAACQGVGGPSHKARVLSGWGGSGG |
| | | GGSGGAFTAFTIPSTRWGGSGGGGSGGKKQYIKANSKFIGITELKKLGGGKRG |
| | | GGKKMTNSVDDALINSTKIYSYFPSVISKVNQGAQGKKL |
| 190 | String 2 GS nt1 | ATGATAGTAACAGACTCACAGTATGCATTATCAATGAACGGAGGTAGCGGT |
| | | GGCGGAGGTTCTGGCGGAATGATGCAGGCTGAACAAGCTACACAAGAGGTA |
| | | AAAAATTGGATGAGTGGAGCAGGCGGTAGCGGCGGAGGTGGCAGTGGAGG |
| | | TAATATGTTCACTAGTACCCTTCAGGAACAAATAGCATGGAAAATCTTTGGA |
| | | GGCTCCGGAGGCGGTGGATCCGGCGGAACTTTCGGAATACCACACCCAGCA |
| | | GGGTTAAAACGGCGTGGAGGTTCTGGTGGAGGCGGTTCAGGAGGTTTGCA |
| | | GGATGTAAGAATGTATAGCCCTGTCAGCATTTTGGACATAGGGATGGGTGG |
| | | AGGTAGCGGCGGAGGTGGCTCGGGTGGCAGAGCCGCAGGGTTAAATAAAA |
| | | TAGTAAGAATGTATCGTAAGTACGGTGGAAGCGGAGGTGGCGGATCCGGTG |
| | | GATACGCAGCGGGACATCAAGCAGCCATGCAAATGTTAAAAGAGACCATCTC |
| | | AAGGGAGGGTGGCAGTGGCGGTGGAGGCAGTGGTGGCATGTACGCAAAAA |
| | | ATCCAGAAATAGTCATCTATAAAATGGGGGGTAGCGGAGGTGGCGGAAGCG |
| | | GCGGTTATGCGTTCGGGCAAATGGTACACCAGGCCCTATCACCTAGAACTTT |
| | | GAATGCATGGGTAAAAGTAATAGGCAGGCGCGGAGGCAGTGGAGGTGGCG |
| | | GATCGGGAGGTTACATGTATACACCAGGGATTAGATATCAATATAATGTGCT |
| | | TAAAAAGACCGGTGGAAGTGGAGGCGGTGGAAGCGGAGGTTACATGGCCG |
| | | CTGAACAAGCTACACAAGATGTAAAAAATTGGATGAAACGATATGGAGGCA |
| | | GTGGAGGTGGCGGATCAGGTGGACAAAATAGGAGCTTTAATTGTAGAGGAG |
| | | AATTTTTCGCTGGGAACGGCGGATCTGGTGGCGGAGGTTCTGGCGGTATGA |
| | | TGATGTACACACCGGGACCAGGGGTCAGATTTCCACTGACCTTTGGATGGT |
| | | GCTTCATGGGCAACGGAGGCTCGGGTGGAGGCGGTTCCGGCGGAGCATAC |
| | | GCACAAAATCCAGAAATAGTCATCTATCAATATATGGATGACTTGAAACGCG |
| | | GAGGCTCCGGAGGTGGCGGAAGCGGCGGAATGATGAGACAAAACTACACAC |
| | | CGGGACCAGGGGTCAGATATCCACTGACCTTTGGATGGTGCTTCAAGCTAA |
| | | AGAAACGCGGCGGATCTGGCGGAGGTGGCAGCGGTGGATATAGCGCATTTA |
| | | AAAGAAAAGGGGGGATTGGGGGGTACCGCCGTTACGGAGGTTCGGGTGGA |
| | | GGCGGTAGTGGAGGCTATAACACACAAGGCTACTTCCCTGATTGGAAGATT |
| | | GGCGGATCGGGAGGCGGTGGATCAGGTGGCACGTATGCACACACACAAGG |
| | | CTTCTTCCCTGATTGGCAAAACTACACAAAGCGTGGCGGTTCTGGCGGTGG |
| | | AGGCTCGGGTGGAAATATCTACCCCATCCAAAAAGAAACATGGGAGACATG |
| | | GTGGACAGACTATTGGGGAGGGAACGGTGGAAGTGGTGGAGGCGGTTCTG |
| | | GCGGAATGATGTACGGGCAAATGGTACACCAGCCCATATCACCTAGAACTTT |
| | | GAATGCATGGGCAAGGAACGGCGGTAGCGGCGGTGGAGGCTCAGGTGGAA |
| | | TGGCCTATGGGGAAAGAATAATAGACATAATAGCAAAGAAGGGTGGAGGCT |
| | | CTGGCGGAGGTGGCAGTGGTGGCCGTGGAGGCTTAATAGGACCTACACCTG |
| | | TCAACATAATTGGAAGAAATATGTTGTTCTGGAGAGGAGGTAGTGGCGGTG |
| | | GAGGCTCCGGCGGTCGGGCAGCGGATTGTAAGACCATTITAAGAGCATTAC |
| | | GTATGAGGGGTGGATCGGGAGGTGGCGGATCGGGCGGAGCAGTAAAAGTA |
| | | ATAGAGGAGAAGGCTTTTAGCCCAGAGGTAATACCCATGTTTACAGCATTAG |
| | | CAATCGGTGGATCCGGAGGCGGTGGAAGTGGCGGTATAATGTATCCCATCC |
| | | AAAAAGAAATATGGGAGACATGGTGGACAGAGTATTGGGGCGCGTGGGGA |
| | | GGCTCAGGAGGTGGCGGATCAGGAGGTTGGGCCTTTAGAGACTATGTAGAC |
| | | CGGTTCTTTAAAACTTTAAGAGCTGGTAAAGGAGGTTCCGGTGGCGGAGGT |
| | | TCAGGTGGAAGGGCTATAGAGGCGCAACAGCATATGAAAAGAGGOGGATCA |
| | | GGTGGAGGCGGTTCCGGAGGTAACAACTACACAGCATTATCAGAAGGAGCC |
| | | ACCCCACAAGATTTAAACACCATGTTAAATACATGGGGAGGAGGTTCAGGTG |
| | | GAGGCGGTTCTGGTGGAAATTACGACATCTATAAAAGATGGATAATTCTGG |
| | | GGTTAAATAAACGACGCGGTGGATCCGGTGGAGGCGGTTCGGGTGGAAGA |
| | | GCAGCTAAGTTGACCCCACTCTGTGTCACTTTATCTATGATGGGOGGATCGG |
| | | GCGGAGGTGGCAGCGGTGGCTTCGCAGCGGCATGTCAGGGAGTGGGAGGA |
| | | CCTTCCCACAAAGCAAGAGTGTTGAGCGGTTGGGGCGGTTCTGGTGGAGGC |
| | | GGTTCCGGTGGCGCCTTTACTGCATTCACCATACCTAGTACACGTTGGGGT |
| | | GGCTCAGGCGGAGGTGGCAGTGGTGGCAAGAAGCAGTACATCAAGGCCAAC |
| | | AGCAAGTTCATCGGCATCACCGAGCTGAAGAAGCTGGGAGGGGGCAAACGG |
| | | GGAGGCGGCAAAAAGATGACCAACAGCGTGGACGACGCCCTGATCAACAGC |
| | | ACCAAGATCTACAGCTACTTCCCCAGCGTGATCAGCAAAGTGAACCAGGGCG |
| | | CTCAGGGCAAGAAACTG |
| 217 | String 2 GS nt2 | ATGATCGTGACCGATAGCCAGTACGCCCTGTCAATGAACGGAGGTAGCGGT |
| | | GGCGGAGGTTCTGGCGGAATGATGCAGGCCGAACAGGCCACACAAGAAGTG |
| | | AAGAACTGGATGAGTGGAGCAGGCGGTAGCGGCGGAGGTGGCAGTGGAGG |
| | | TAATATGTTCACCAGCACACTGCAAGAGCAGATCGCCTGGAAAATCTTTGGT |
| | | GGCTCTGGAGGCGGTGGATCAGGTGGAACTTTCGGCATTCCTCATCCTGCC |
| | | GGACTGAAACGGCGTGGAGGTTCTGGTGGAGGCGGTTCAGGAGGTTTGCA |
| | | GGATGTGAGAATGTACAGCCCCGTGTCCATCCTGGACATCGGGATGGGTGG |
| | | AGGTAGCGGCGGAGGTGGCTCGGGTGGCAGAGCCGCAGGCCTGAACAAGA |
| | | TCGTGCGGATGTACCGTAAGTACGGTGGAAGCGGAGGCGGTGGATCGGGT |
| | | GGCTACGCAGCGGGACATCAGGCCGCTATGCAGATGCTGAAAGAGACAATC |
| | | TCAAGGGAGGGTGGCAGTGGCGGTGGAGGCAGTGGTGGCATGTACGCCAA |
| | | GAATCCCGAGATCGTGATCTACAAAATGGGGGTAGCGGAGGTGGCGGAAG |
| | | CGGCGGTTATGCGTTCGGCCAGATGGTTCACCAGGCTCTGAGCCCCAGAAC |
| | | ACTGAACGCCTGGGTCAAAGTGATCGGCAGGCGCGGAGGCAGTGGAGGTG |
| | | GCGGATCGGGAGGTTACATGTATACCCCTGGCATCAGATACCAGTACAACG |
| | | TGCTGAAAAAGACCGGTGGAAGTGGAGGCGGTGGAAGCGGAGGTTACATG |
| | | GCCGCCGAACAGGCTACCCAGGACGTGAAGAACTGGATGAAACGATATGGA |
| | | GGCAGTGGAGGTGGCGGATCAGGTGGACAAAATAGGAGCTTCAATTGCAGG |
| | | GGCGAGTTCTTCGCTGGGAACGGCGGATCTGGTGGCGGAGGTTCTGGCGG |
| | | TATGATGATGTACACACCTGGACCTGGCGTCAGATTCCCTCTGACCTTCGGC |
| | | TGGTGCTTCATGGGCAACGGAGGCTCGGGTGGAGGCGGTTCCGGCGGAGC |
| | | ATACGCCCAGAATCCTGAGATCGTGATCTACCAGTACATGGACGACCTGAAA |
| | | CGCGGAGGTAGCGGCGGTGGAGGCAGCGGAGGTATGATGAGACAGAACTA |
| | | CACACCTGGACCTGGCGTGCGGTATCCTCTGACCTTCGGCTGGTGCTTCAA |
| | | GCTGAAGAAACGCGGCGGATCTGGCGGAGGTGGCAGCGGTGGATATAGCG |
| | | CATTCAAGCGGAAAGGCGGCATCGGCGGCTATCGCCGTTACGGAGGTTCGG |
| | | GTGGAGGCGGTAGTGGAGGCTATAACACCCAGGGCTACTTCCCCGATTGGA |
| | | AGATTGGCGGATCGGGAGGCGGTGGATCAGGTGGCACGTATGCACACACCC |
| | | AGGGATTCTTCCCAGACTGGCAGAACTACACCAAGCGTGGCGGTTCTGGCG |
| | | GTGGAGGCTCGGGTGGAAATATCTACCCCATCCAGAAAGAGACATGGGAAA |
| | | CCTGGTGGACCGACTACTGGGGAGGGAACGGTGGAAGTGGTGGAGGCGGT |
| | | TCTGGCGGAATGATGTACGGCCAGATGGTGCATCAGCCTATCAGCCCCAGA |
| | | ACACTGAACGCCTGGGCAAGGAACGGCGGTAGCGGCGGTGGAGGCTCAGG |
| | | TGGAATGGCCTATGGCGAGCGGATCATCGACATCATTGCCAAGAAGGGTGG |
| | | AGGCTCTGGCGGAGGTGGCAGTGGTGGCCGTGGAGGCCTGATCGGACCCA |
| | | CACCTGTGAACATCATCGGCCGGAACATGCTGTTCTGGAGAGGAGGTAGTG |
| | | GCGGTGGAGGCTCCGGCGGTCGGGCAGCGGACTGCAAGACCATCCTGAGA |
| | | GCCCTGCGTATGAGGGGTGGATCGGGAGGTGGCGGATCGGGCGGAGCAGT |
| | | GAAAGTGATCGAGGAAAAGGCTTTCAGCCCCGAAGTGATCCCCATGTTCAC |
| | | AGCCCTTGCAATCGGAGGCAGTGGTGGCGGAGGTAGTGGTGGAATAATGTA |
| | | TCCCATCCAGAAAGAGATCTGGGAAACCTGGTGGACCGAGTACTGGGGCGC |
| | | GTGGGGAGGCTCAGGAGGTGGCGGATCAGGAGGTTGGGCCTTCAGAGACT |
| | | ACGTGGACCGGTTCTTCAAGACCCTGAGAGCCGGTAAAGGAGGTAGTGGAG |
| | | GCGGTGGATCGGGCGGTAGAGCCATTGAAGCCCAGCAGCACATGAAAAGAG |
| | | GAGGTAGTGGAGGCGGTGGATCGGGCGGTAACAACTACACAGCTCTGTCTG |
| | | AAGGCGCCACACCTCAGGACCTGAACACCATGCTGAACACCTGGGGAGGAG |
| | | GTTCAGGTGGAGGCGGTTCTGGTGGAAATTACGACATCTACAAGCGGTGGA |
| | | TCATCCTGGGCCTGAACAAACGACGCGGAGGTAGCGGCGGTGGAGGCAGC |
| | | GGAGGTAGAGCAGCTAAGCTGACCCCTCTGTGTGTGACCCTGTCTATGATG |
| | | GGCGGATCGGGCGGAGGTGGCAGCGGTGGCTTCGCAGCGGCCTGTCAAGG |
| | | TGTTGGCGGACCTTCTCACAAGGCCAGAGTTCTGAGCGGTTGGGGCGGTTC |
| | | TGGTGGAGGCGGTTCCGGTGGCGCCTTTACCGCCTTCACAATCCCTAGCAC |
| | | CCGTTGGGGTGGCTCAGGCGGAGGTGGCAGTGGTGGCAAGAAGCAGTACA |
| | | TCAAGGCCAACAGCAAGTTCATCGGCATCACCGAGCTGAAGAAGCTGGGAG |
| | | GGGGCAAACGGGGAGGCGGCAAAAAGATGACCAACAGCGTGGACGACGCC |
| | | CTGATCAACAGCACCAAGATCTACAGCTACTTCCCCAGCGTGATCAGCAAAG |
| | | TGAACCAGGGCGCTCAGGGCAAGAAACTGTGATAA |
| 218 | String 2 GS nt3 | ATGATTGTAACAGATTCTCAATATGCTCTGTCAATGAACGGAGGTAGCGGTG |
| | | GCGGAGGTTCTGGCGGAATGATGCAGGCTGAACAAGCTACACAAGAAGTGA |
| | | AAAATTGGATGAGTGGAGCAGGCGGTAGCGGCGGAGGTGGCAGTGGAGGT |
| | | AATATGTTCACATCAACACTTCAGGAACAGATTGCTTGGAAAATCTTTGGTG |
| | | GCTCTGGAGGCGGTGGATCAGGTGGAACTTTCGGAATTCCTCACCCTGCTG |
| | | GATTGAAACGGCGTGGAGGTTCTGGTGGAGGCGGTTCAGGAGGTTTGCAG |
| | | GATGTGAGAATGTACTCTCCTGTGTCAATTCTGGATATTGGGATGGGTGGA |
| | | GGTAGCGGCGGAGGTGGCTCGGGTGGCAGAGCCGCAGGATTAAATAAAATT |
| | | GTAAGAATGTACCGTAAGTACGGTGGAAGCGGAGGCGGTGGATCGGGTGG |
| | | CTACGCAGCGGGACACCAAGCAGCAATGCAAATGCTGAAAGAAACCATTTCA |
| | | AGGGAGGGTGGCAGTGGCGGTGGAGGCAGTGGTGGCATGTACGCTAAAAA |
| | | TCCTGAAATTGTGATTTACAAAATGGGGGGTAGCGGAGGTGGCGGAAGCGG |
| | | CGGTTATGCGTTCGGACAGATGGTGCACCAGGCTTTGTCTCCAAGAACATT |
| | | GAATGCTTGGGTAAAAGTAATTGGCAGGCGCGGAGGCAGTGGAGGTGGCG |
| | | GATCGGGAGGTTACATGTATACACCTGGAATAAGATACCAATACAATGTGCT |
| | | TAAAAAGACCGGTGGAAGTGGAGGCGGTGGAAGCGGAGGTTACATGGCCG |
| | | CAGAACAAGCAACACAAGATGTGAAAAATTGGATGAAACGATATGGAGGCA |
| | | GTGGAGGTGGCGGATCAGGTGGACAAAATAGGTCTTTCAATTGCAGAGGAG |
| | | AATTCTTCGCTGGGAACGGCGGATCTGGTGGCGGAGGTTCTGGCGGTATGA |
| | | TGATGTACACCCCTGGACCTGGAGTGAGATTTCCTCTGACCTTTGGATGGT |
| | | GCTTTATGGGCAACGGAGGCTCGGGTGGAGGCGGTTCCGGCGGAGCATAC |
| | | GCTCAAAATCCTGAAATTGTCATTTACCAATACATGGATGATCTTAAACGCG |
| | | GAGGTAGCGGCGGTGGAGGCAGCGGAGGTATGATGAGACAGAATTACACCC |
| | | CTGGACCTGGAGTGAGATACCCTCTGACCTTTGGATGGTGCTTTAAACTGAA |
| | | GAAACGCGGCGGATCTGGCGGAGGTGGCAGCGGTGGATATAGCGCATTTAA |
| | | AAGAAAAGGAGGAATTGGAGGATACCGCCGTTACGGAGGTTCGGGTGGAG |
| | | GCGGTAGTGGAGGCTATAATACCCAGGGATACTTTCCTGATTGGAAGATTG |
| | | GCGGATCGGGAGGCGGTGGATCAGGTGGCACGTATGCACACACCCAAGGAT |
| | | TCTTCCCTGATTGGCAAAATTACACCAAGCGTGGCGGTTCTGGCGGTGGAG |
| | | GCTCGGGTGGAAATATCTACCCAATCCAGAAAGAAACATGGGAAACATGGT |
| | | GGACAGATTACTGGGGAGGGAACGGTGGAAGTGGTGGAGGCGGTTCTGGC |
| | | GGAATGATGTACGGACAGATGGTTCACCAGCCAATTTCTCCAAGAACACTGA |
| | | ATGCTTGGGCAAGGAACGGCGGTAGCGGCGGTGGAGGCTCAGGTGGAATG |
| | | GCCTATGGAGAAAGAATTATTGATATTATTGCTAAGAAGGGTGGAGGCTCT |
| | | GGCGGAGGTGGCAGTGGTGGCCGTGGAGGCTTGATTGGACCAACACCAGT |
| | | GAATATTATTGGAAGAAATATGTTGTTCTGGAGAGGAGGTAGTGGCGGTGG |
| | | AGGCTCCGGCGGTCGGGCAGCGGATTGCAAAACCATTITAAGAGCTTTACG |
| | | TATGAGGGGTGGATCGGGAGGTGGCGGATCGGGCGGAGCAGTGAAAGTGA |
| | | TTGAAGAAAAAGCTTTTTCTCCTGAAGTGATTCCAATGTTTACAGCTCTGGC |
| | | AATCGGAGGCAGTGGTGGCGGAGGTAGTGGTGGAATAATGTATCCAATCCA |
| | | GAAAGAAATCTGGGAAACATGGTGGACAGAATACTGGGGCGCGTGGGGAG |
| | | GCTCAGGAGGTGGCGGATCAGGAGGTTGGGCCTTTAGAGATTATGTGGATA |
| | | GATTCTTCAAAACCTTGAGAGCTGGTAAAGGAGGTAGTGGAGGCGGTGGAT |
| | | CGGGCGGTAGAGCAATCGAAGCACAGCAGCACATGAAAAGAGGAGGTAGTG |
| | | GAGGCGGTGGATCGGGCGGTAACAACTACACAGCATTGTCTGAAGGAGCAA |
| | | CACCTCAGGATTTGAATACAATGTTGAATACATGGGGAGGAGGTTCAGGTG |
| | | GAGGCGGTTCTGGTGGAAATTACGATATTTACAAAAGATGGATTATTCTGG |
| | | GACTGAATAAACGACGCGGAGGTAGCGGCGGTGGAGGCAGCGGAGGTAGA |
| | | GCAGCTAAATTGACCCCTTTGTGTGTGACCTTGTCTATGATGGGOGGATCG |
| | | GGCGGAGGTGGCAGCGGTGGCTTCGCAGCGGCATGCCAGGGAGTGGGAGG |
| | | ACCTTCTCACAAAGCAAGAGTGCTTAGCGGTTGGGGCGGTTCTGGTGGAGG |
| | | CGGTTCCGGTGGCGCCTTTACAGCTTTTACAATCCCTTCAACACGTTGGGGT |
| | | GGCTCAGGCGGAGGTGGCAGTGGTGGCAAGAAGCAGTACATCAAGGCCAAC |
| | | AGCAAGTTCATCGGCATCACCGAGCTGAAGAAGCTGGGAGGGGGCAAACGG |
| | | GGAGGCGGCAAAAAGATGACCAACAGCGTGGACGACGCCCTGATCAACAGC |
| | | ACCAAGATCTACAGCTACTTCCCCAGCGTGATCAGCAAAGTGAACCAGGGCG |
| | | CTCAGGGCAAGAAACTGTGATAA |
| 191 | P2P16 aa | KKQYIKANSKFIGITELKKLGGGKRGGGKKMTNSVDDALINSTKIYSYFPSVISK |
| | | VNQGAQGKKL |
| 192 | P2P16 nt | AAGAAGCAGTACATCAAGGCCAACAGCAAGTTCATCGGCATCACCGAGCTGA |
| | | AGAAGCTGGGAGGGGGCAAACGGGGAGGCGGCAAAAAGATGACCAACAGC |
| | | GTGGACGACGCCCTGATCAACAGCACCAAGATCTACAGCTACTTCCCCAGCG |
| | | TGATCAGCAAAGTGAACCAGGGCGCTCAGGGCAAGAAACTG |
| 193 | GS linker aa | GGSGGGGSGG |
| 194 | GS linker nt | GGCGGATCGGGTGGAGGCGGTTCAGGTGGC |
| 195 | 5′UTR | GGGCGAACTAGTATTCTTCTGGTCCCCACAGACTCAGAGAGAACCCGCCACC |
| 196 | 3′UTR | CTCGAGCTGGTACTGCATGCACGCAATGCTAGCTGCCCCTTTCCCGTCCTGG |
| | | GTACCCCGAGTCTCCCCCGACCTCGGGTCCCAGGTATGCTCCCACCTCCACC |
| | | TGCCCCACTCACCACCTCTGCTAGTTCCAGACACCTCCCAAGCACGCAGCAA |
| | | TGCAGCTCAAAACGCTTAGCCTAGCCACACCCCCACGGGAAACAGCAGTGAT |
| | | TAACCTTTAGCAATAAACGAAAGTTTAACTAAGCTATACTAACCCCAGGGTT |
| | | GGTCAATTTCGTGCCAGCCACACCGAGACCTGGTCCAGAGTCGCTAGCCGC |
| | | GTCGCT |
| 197 | Poly A structure | AAAAAAAAAAAAAAAAAAAAAAAAAAAAAAGCAUAUGACUAAAAAAAAAAAA |
| | | AAAAAAAAAAAAAAAAAAAAAAAAAAAAAAAAAAAAAAAAAAAAAAAAAAAAA |
| | | AAAAA |

### DESCRIPTION OF THE FIGURES

FIG. 1: FIG. 1 shows string representations of two RNA molecules. The first RNA molecule (String 1) encodes for an immunogenic peptide comprising 33 fragments and the second RNA molecule (String 2) encodes for an immunogenic peptide comprising 32 fragments. Every two fragments are separated by up to four cleavage enhancing amino acids. The length of the immunogenic peptides are around 550 amino acids.

FIG. 2: FIG. 2 shows string representations of two RNA molecules. The first RNA molecule (String 1) encodes for an immunogenic peptide comprising 33 fragments and the second RNA molecule (String 2) encodes for an immunogenic peptide comprising 32 fragments. Every two fragments are separated by a non-immunogenic linker of 10 amino acids, flanked by cleavage enhancing amino acids (up to four). The length of the immunogenic peptides are around 950 amino acids.

FIG. 3: Amino acid changes between RNA strings 1 and 2 and RNA strings 1.1 and 2.1.

FIG. 4: In vitro expression of mRNA-encoded polypeptides from 2 mRNA strings with or without flexible Glycine-Serin (GS) linker (string 1 non-GS, string 2 non-GS and string2 GS). For each string, 3 different nucleotide sequences were generated (nt1, nt2 and nt3), wherein nt1 corresponds to the wild-type sequence and nt2 and nt3 represent two different codon optimization methods. Each string was in vitro transcribed in the presence of either unmodified (uRNA) or methyl pseudouridine (modRNA). For string 2 GS only nt3 was tested. Each mRNA string, i.e. uRNA/modRNA, nt1-3, string 1-2, GS/non-GS, was transfected separately in a human cell line in the presence or absence of the proteasome inhibitor MG132. Relative abundance for each peptide is shown for (A) two tryptic peptides common to each string tested with modRNA; (B) tryptic peptide specific for string 1 non-GS, modRNA and uRNA; (C) tryptic peptide specific for strings 2 GS and non-GS, modRNA and uRNA. The tryptic peptides common to all strings, YPLTFGWCFK and IYSYFPSVISK, tested are located in the central part and the C-terminal region of the polypeptides encoded by mRNA string 1 non-GS (D) and String 2 non-GS and GS (E). String-specific tryptic peptides location tested for string 1 non-GS, TSTLQEQIGWAR, (D) and string 2 GS and non-GS, QNYTPGPGVR, (E) are also shown.

FIG. 5: In vitro processing and presentation by HLA-I alleles from mRNA-encoded polypeptides from 2 modRNA strings (string 1 and string 2) was assessed with immunopeptidomics. Each string was transfected in an A375 cell line encoding a single HLA class I allele and targeted mass spectrometry allowed to detect HIV specific epitopes in string 1 (A-B) and string 2 (C-D). The detected epitope locations are annotated under string 1 (A) and string 2 (C). The detailed information related to each detected epitope, such as the HLA class-I allele, location at the edge of a fragment, and a non-HIV junctional sequence (edge) and the sequence match for string 1 is shown in panel (B) and the corresponding information for string 2 is shown in panel (D). Legend: Epitope sequence perfectly matches to a 2021 consensus B=ConB; Epitope sequence perfectly matches to a 2021 consensus C=ConC; Epitopes sequence perfectly matches to Consensus B and C=ConB/C; epitope not a perfect match in either consensus B or C=Polymorph.

FIG. 6: TCR engagement from epitope processed and presented from different mRNA strings in the K562 cell line. A Jurkat cell line expressing a single TCR was co-incubated with mono HLA-I allele K562 cells to measure TCR downstream signaling expressed as log 2 fold signal of luciferase. K562 were pulsed with a peptide encoded by the tested string (PP) as positive control to control the TCR activity for the specific epitope tested or transfected with a mRNA encoding for a polypeptide (stripped bar). Two different mRNA strings (string 1 and string 2) were tested with different nucleotide optimization (nt2 and nt3). PHA-L was used as a positive control for TCR activity. % unique epitopes, AK11, DA9, +DA9+, KF11 and IW9, were tested to react with their cognate TCRs. Processing and presentation of the exact epitopes KF11 and IW9 were investigated from different mRNA strings. A polymorphic version of DA9, marked as +DA9+, encoded by string2 versions was cross-reactive with the non-cognate DA9-specific TCR. AK11 was tested using A*11:01, EL9 was tested using A*26:01, KF11 was tested with B*57:01, DA9 was tested using B*14:01 and IW9 was presented by both the cognate HLA-I allele B*57:03 and non-cognate B*57:01 allele, marked as +B*57:01+ for both string1 and string2. Legend: AK11:ACQGVGGPGHK; DA9 DRFYKTLRA; +DA9+:DRFFKTLRA; KF11:KAFSPEVIPMF; IW9: ISPRTLNAW

FIG. 7: TCR engagement from epitope processed and presented from different mRNA strings in iDCs generated from 2 donors. A Jurkat cell line expressing a single TCR was co-incubated with iDCs to measure TCR downstream signaling expressed as log 2 fold signal of luciferase. iDCs were pulsed with a peptide encoded by the tested string. To control for TCR activity the pulsed iDC were either previously co-transfected with a mRNA encoding an HLA class-I allele (PP) or without co-transfection and expressing only endogenous HLA-I from the donor (PP+eHLA) to control for potential endogenous HLA-I mediated background presentation. iDCs were co-transfected with an mRNA encoding for a polypeptide and the cognate HLA class I allele (stripped bar). Two different mRNA strings (string 1 and string 2) were tested with different nucleotide optimization (nt2 and nt3). PHA-L was used as a positive control for TCR activity. For donor 1 (A) and donor 2 (B) respectively 6 (AK11, DA9, +DA9+, KF11, EL9 and IW9) and 5 (AK11, DA9, +DA9+, EL9 and IW9) unique epitopes were tested to react with their cognate TCRs. Processing and presentation of epitope IW9 was investigated from two different mRNA strings (string1 and string2). A polymorphic version of DA9, marked as +DA9+, encoded by string2 versions was cross-reactive with the non-cognate DA9-specific TCR. AK11 was tested using A*11:01, EL9 was tested using A*26:01, KF11 was tested with B*57:01, DA9 was tested using B*14:01 and IW9 was presented by both the cognate HLA-I allele B*57:03 and non-cognate B*57:01 allele, marked as +B*57:01+ for both string1 and string2. Legend: AK11: ACQGVGGPGHK; DA9:DRFYKTLRA; +DA9+:DRFFKTLRA; KF11:KAFSPEVIPMF; IW9:ISPRTLNAW; EL9:EVIPMFSAL

FIG. 8: CD8+ and CD4+ T-cell responses to two different mRNA strings (string1 and string2): Elispot data for IFN-γ production by isolated CD8+ T and isolated CD4+ T cells stimulated with peptide pools. T-cells were isolated from HLA-A*02:01 CB6F1 transgenic mice and stimulated with peptide pools for 18 hours on Elispot plates coated with anti-IFN-γ antibody. CD8+ T cells from groups (n=5 mice per group) immunized with string1 (A) or string 2 (B) were incubated with 11mer peptide pools covering the vaccine fragments containing at least one known/predicted HIV-1 epitope (prctrk<1%) from A*02:01 (white box—black circle) or H2b/H2d (striped box—white square) or neither (diamond box—white triangle). CD8+ T cells from groups immunized with string1 (C) or string 2 (D) were incubated with heterologous minimal A*02:01 (white box—black circle) or H2b/H2b (striped box—white square) HIV epitope pools, i.e. minimal HIV epitopes (8-11-mers) containing at least one polymorphism compared to the immunizing string sequence. Pool I/K, tested on string1 immunized groups and Pool G/H, tested on string2 immunized groups are exact match of sequences found only in respective string2 and string1. Isolated CD4+ T and CD8+ T cells (E) were incubated with a 15-mer peptide pool spanning the entire tetanus toxin. CD8+ and CD4+ T isolated cells of indicated immunized groups were tested with an AH-1 irrelevant 15-mer peptide pool negative control (F) and concanavalin A positive control (G). Quantification of IFNg spot-forming units per 100.000 cells for each peptide pool. Panel (H) shows a summary of the experimental setup.

FIG. 9: CD8+ and CD4+ T-cell responses to two different mRNA strings (string1 and string2): Elispot data for IFN-γ production by isolated CD8+ T and isolated CD4+ T cells stimulated with peptide pools. T-cells were isolated from HLA-A*02:01 CB6F1 transgenic mice and stimulated with peptide pools for 18 hours on Elispot plates coated with anti-IFN-γ antibody. CD8+ T cells from groups (n=5 mice per group) immunized with either a single mRNA, string1 (10 μg) or string 2 (10 μg), or co-immunized with both mRNA string1+string2 in a 1:1 ratio (10 μg+10 μg; 5 μg+5 μg; 2.5 μg+2.5 μg). Animals immunized with string1, alone or in combination, (A, C) or with string2, alone or in combination (B, D) were incubated with 11mer peptide pools covering the vaccine fragments containing at least one known/predicted HIV epitope (prctrk<1%) from A*02:01 (white box—black circle) or H2b/H2d (striped box—white square) or neither (diamond box—white triangle), respectively pool A, B, C and D, E, F. CD8+ T cells from groups immunized with string1 (A) or string 2 (B) were also incubated with minimal A*02:01 (white box—black circle) or H2b/H2b (striped box—white square) HIV epitopes pools (8-11-mers) exclusively encoded by either string1 (Pool G and H) or string 2 (Pool I/K). Similarly, CD4+ T cells from groups immunized with string1 (C) or string 2 (D) were incubated with minimal A*02:01 (white box—black circle) or H2b/H2b (striped box—white square) HIV epitopes pools (8-11-mers). Isolated CD4+ T and CD8+ T cells (E) were incubated with a 15-mer peptide pool spanning the entire tetanus toxin. CD8+ and CD4+ T isolated cells of indicated immunized groups were tested with an AH-1 irrelevant 15-mer peptide pool negative control (F) and concanavalin A positive control (G). Quantification of IFNg spot-forming units per 100.000 cells for each peptide pool. Panel (H) shows a summary of the experimental setup.

FIG. 10: TCR engagement from epitope processed and presented from different mRNA strings in the K562 cell line: Jurkat cell line expressing a single TCR were co-incubated with mono HLA-I allele K562 to measure TCR downstream signaling expressed as log 2 fold signal of luciferase. K562 were pulsed with a peptide encoded by the tested string (PP) as positive control to control the TCR activity for the specific epitope tested or transfected with a mRNA string encoding for a polypeptide. PHA-L was used as a positive control for TCR activity. 5 unique epitopes were tested to react with their cognate TCRs. Four different mRNA strings, with distinct amino acid sequences have been tested each with 2 nucleotide sequences optimization (nt2 and nt3). String1 and its derivative string1.1 (4 point mutations) have been tested by transfecting cells with either 3 μg (A) or 0.3 μg (B) of methylpseudo-uridine mRNA in K562 cells. All epitopes displayed were a perfect match in the sequence of these mRNA strings. String2 and its derivative string2.1 (1 point mutation) have been tested by transfected cells with either 3 μg (C) or 0.3 μg (D) of methylpseudo-uridine mRNA in K562 cells. AK11 was tested using A*11:01, EL9 was tested using A*26:01, KF11 was tested with B*57:01, DA9 was tested using B*14:01 and IW9 tested using HLA-I allele B*57:03. Epitopes marked with a star (*) contain at least one polymorphism compared to the sequence of these mRNA strings.

FIG. 11: TCR engagement from epitopes processed and presented from different mRNA strings in the K562 cell line in iDCs generated from 2 donors: Effector Jurkat cell lines expressing a single TCR were co-incubated with Target mono HLA-I allele K562 or HLA-I transfected iDCs from donors to measure TCR downstream signaling expressed as log 2 fold signal of luciferase. Target cells were pulsed with a peptide encoded by the tested string (PP) as positive control to control the TCR activity for the specific epitope tested or transfected with an mRNA string encoding for a polypeptide. Target iDCs were also tested with pulsed peptide but without co-transfection of HLA alleles to control for background presentation by the endogenous HLA-I from the donor (PP 10 μM+eHLA). PHA-L was used as a positive control for TCR activity. 3 unique epitopes were tested to react with their cognate TCRs.

Four different mRNA strings, with distinct amino acid sequences, were tested each with 2 nucleotide sequence optimizations (nt2 and nt3). String1 and string2 were tested by transfecting cells with either 3 μg of methylpseudo-uridine mRNA in the Target K562 cells (A) or in Target iDCs from 2 donors (B,C). The tested TL9 and RI8 epitopes were both encoded by string1 and string2 with a perfect sequence match. In parallel, the epitope TW10 encoded by String2 contained a single mutation polymorphism, while being a perfect match for String1. TL9 was tested using the HLA allele B*39:10, E18 was tested using the HLA allele B*52:01 and TW10 was tested with the HLA alleles B*57:01, B*57:03 and B*58:01.

FIG. 12: CD8+ and CD4+ T-cell responses to two different mRNA strings (string1 and string2) in combination via intramuscular lipid nanoparticle (LNP) nanocarrier: Elispot data for IFN-γ production from CD8+ T cell-depleted and CD4+ T cell-depleted splenocytes stimulated with peptide pools. Splenocytes were isolated from HLA-A*02:01 CB6F1 transgenic mice and stimulated with peptide pools for 18 hours on Elispot plates coated with anti-IFN-γ antibody. CD4+ T cell-depleted and CD8+ T cell-depleted splenocytes were generated from animal groups (n=5 mice per group) co-immunized with a combination of single LNP-formulated mRNA string1+string2 in a 1:1 ratio (2.5 μg+2.5 μg) or receiving a saline placebo. mRNA combination were tested with string1+string2 as unmodified (uRNA) or as methyl pseudouridine (modRNA). Animals were immunized with a single intramuscular injection and splenocytes were harvested 7 days after injection. CD4+ T cell-depleted (FIG. 12A) or CD8+ T cell-depleted splenocytes (FIG. 12B) from animals immunized with a string1+string2 modRNA or uRNA combination were incubated with 11mer peptide pools covering the vaccine fragments containing at least one known/predicted HIV epitope (prctrk<1%) from A*02:01 (white box—black circle) or H2b/H2d (striped box—white square) or neither (diamond box—white triangle). CD4+ T cell-depleted and CD8+ T cell depleted-splenocytes were incubated with a 15-mer peptide pool spanning the entire tetanus toxin (FIG. 12C). The background IFN-γ signals from CD4+ T cell-depleted and CD8+ T cell depleted-splenocytes incubated with DMSO peptide diluent control is indicated as the median of signals measured across all groups tested in panel (A) or (B)—“Median DMSO”. Quantification of IFNg spot-forming units per 10e6 cells for each peptide pool. FIG. 12D shows a summary of the experimental outline.

### EXAMPLES

### Example 1

HLA-I restricted epitopes for improved coverage and escape mutational constraints have been selected. These panels of CD8+ T cell epitopes have been assembled into multi-epitopic cassettes to produce vaccines, as described below.

A list of CD8+ T cell highly networked epitopes, offering a broad coverage based on HLA class-I alleles prevalence, has been compiled and used to design a rational set of RNA vaccines with them. We used the highly networked scoring approach described in Gaiha et al. Structural topology defines protective CD8+ T cell epitopes in the HIV proteome. Science 364, 480-484 (2019) to generate an initial list of epitopes of interest, based on their potential high threshold for mutation and with published data indicating immunogenicity in human. We evaluated their binding with internal pMHC prediction tools and performed pMHC binding assays on epitope-MHC pairs that failed internal checks. We identified sequences for each epitope within HIV Clades B and C consensus 2021 and evaluated the conservation. For every amino acid within the epitopes where conservation in patients' sequences was below 75% (according to the Los Alamos HIV database) we considered adding epitopes with polymorphic sequences to achieve coverage across the majority of patients. The epitopes were organized and processed into fragments that were distributed on two distinct RNAs. Fragments were separated by supplementary residues with two linker approaches, generating 2 sets of 2 RNAs.

Each epitope has been matched to an HLA class-I allele they can bind, their amino acid sequence defined in the context of HIV Clades B and C consensus, and screened for the most frequent polymorphisms within the clades; see Table 1 below.

Epitopes were selected from Los Alamos HIV database's (LADB) “A”- and “Silver” CTL epitope lists (https://www.hiv.lanl.gov/content/immunology/tables/tables.html). Epitopes with expected high mutational constraints were selected. After multiple selection rounds, 70 epitopes were top-listed, for which the network score was above the selection threshold and had literature-defined proven human immunogenicity and their HLA class-I binding partners.

TABLE 1
CD8+ T-cell highly networked epitopes from HIV

| B_optimal_epi- | C_optimal_epi- | Epitope | | clade_B_C_iden- | |
|---|---|---|---|---|---|
| tope | tope | length | polymorphisms | tical | allele |
| KLTPLCVTL | KLTPLCVTL | 9 | | yes | A*02:01 |
| SFNCGGEFF | SFNCRGEFF | 9 | | no | C*04:01 |
| RAIEAQQHL | RAIEAQQHM | 9 | | no | B*15:01, B*15:03, |
| | | | | | B*49:01, C*03:04, |
| | | | | | C*08:01, C*15:02 |
| GQMVHQAISP | GQMVHQAISP | 10 | GQMVHQALSP, GQMVHQPLSP, | yes | B*58:02 |
| | | | GQMVHQPISP | | |
| GQMVHQAISPR | GQMVHQAISPR | 11 | GQMVHQALSPR, GQMVHQPLSPR, | yes | A*74:01 |
| | | | GQMVHQPISPR | | |
| VHQAISPRTL | VHQAISPRTL | 10 | VHQALSPRTL, VHQPLSPRTL, | yes | C*03:04 |
| | | | VHQPISPRTL | | |
| HQAISPRTL | HQAISPRTL | 9 | HQALSPRTL, HQPLSPRTL, | yes | B*15:10, B*42:01 |
| | | | HQPISPRTL | | |
| QAISPRTLNAW | QAISPRTLNAW | 11 | QALSPRTLNAW, QPLSPRTLNAW, | yes | A*25:01 |
| | | | QPISPRTLNAW | | |
| ISPRTLNAW | ISPRTLNAW | 9 | LSPRTLNAW, LSPRTLNAW | yes | B*53:01, B*57:01, |
| | | | | | B*57:03 |
| SPRTLNAWV | SPRTLNAWV | 9 | | yes | A*68:02, B*07:02, |
| | | | | | B*42:01, B*58:02 |
| VKVVEEKAF | VKVIEEKAF | 9 | | no | B*15:01, B*15:03 |
| EEKAFSPEV | EEKAFSPEV | 9 | | yes | B*42:01, B*44:15 |
| KAFSPEVIPM | KAFSPEVIPM | 10 | | yes | C*03:04 |
| KAFSPEVIPMF | KAFSPEVIPMF | 11 | | yes | B*57:01, B*57:03, |
| | | | | | B*58:02 |
| EVIPMFSAL | EVIPMFTAL | 9 | | no | A*25:01, A*26:01, |
| | | | | | A*26:02, A*26:03 |
| VIPMFSAL | VIPMFTAL | 8 | | no | C*01:02 |
| SALSEGATP | TALSEGATP | 9 | | no | E*01:03 |
| SEGATPQDL | SEGATPQDL | 9 | | yes | B*40:01, B*44:03, |
| | | | | | B*53:01 |
| ATPQDLNTMLNT | ATPQDLNTMLNT | 12 | | yes | B*58:02 |
| TPQDLNTML | TPQDLNTML | 9 | | yes | B*07:02, B*39:10, |
| | | | | | B*42:01, B*81:01, |
| | | | | | C*08:02 |
| GHQAAMQML | GHQAAMQML | 9 | | yes | B*15:10, B*39:01, |
| | | | | | B*53:01 |
| TSTLQEQIGW | TSTLQEQIAW | 10 | | no | B*57:01, B*57:03, |
| | | | | | B*58:01 |
| EIYKRWII | DIYKRWII | 8 | | no | B*08:01 |
| EIYKRWIIL | DIYKRWIIL | 9 | | no | A*24:02 |
| KRWIILGLNK | KRWIILGLNK | 10 | | yes | B*27:05 |
| GLNKIVRMY | GLNKIVRMY | 9 | | yes | B*15:01, B*53:01, |
| | | | | | C*01:02 |
| VRMYSPTSI | VRMYSPVSI | 9 | | no | B*14:02, B*73:01, |
| | | | | | C*06:02 |
| RMYSPTSI | RMYSPVSI | 8 | | no | B*52:01 |
| RMYSPTSIL | RMYSPVSIL | 9 | | no | E*01:03 |
| YSPTSILDI | YSPVSILDI | 9 | | no | C*01:02, C*12:02 |
| FRDYVDRFY | FRDYVDRFF | 9 | | no | C*18:01 |
| RDYVDRFYKTL | RDYVDRFFKTL | 11 | | no | A*24:02, A*68:02 |
| DYVDRFYKTLR | DYVDRFFKTLR | 11 | | no | A*33:03 |
| YVDRFYKTL | YVDRFFKTL | 9 | | no | A*01:01, A*02:07, |
| | | | | | A*26:01, B*07:02, |
| | | | | | B*15:03, B*15:10, |
| | | | | | C*02:02, C*03:04 |
| DRFYKTLRA | DRFFKTLRA | 9 | | no | B*14:01, B*14:02 |
| AEQASQEVKNW | AEQATQDVKNW | 11 | AEQASQDVKNW, AEQATQEVKNW | no | B*15:03, B*44:02, |
| | | | | | B*44:03 |
| AEQASQEVKNWM | AEQATQDVKNWM | 12 | AEQASQDVKNWM, AEQATQEVKNWM | no | C*05:01 |
| QASQEVKNW | QATQDVKNW | 9 | QASQDVKNWM, QATQEVKNWM | no | B*53:01, B*57:01, |
| | | | | | B*58:01 |
| DCKTILKAL | DCKTILRAL | 9 | | no | B*08:01, B*15:03 |
| ACQGVGGPGHK | ACQGVGGPGHK | 11 | ACQGVGGPSHK | yes | A*11:01 |
| GPGHKARVL | GPGHKARVL | 9 | GPSHKARVL | yes | B*07:02, B*42:01 |
| HTQGYFPDW | HTQGFFPDW | 9 | NTQGYYPDW, NTQGYFPDW, | no | B*15:03, B*57:03, |
| | | | NTQGFFPDW | | B*58:01, C*15:02 |
| TQGYFPDWQNY | TQGFFPDWQNY | 11 | | no | B*15:01, B*49:01 |
| YFPDWQNY | FFPDWQNY | 8 | | no | A*29:02 |
| YFPDWQNYT | FFPDWQNYT | 9 | | no | B*49:01, C*06:02 |
| YTPGPGIRY | YTPGPGVRY | 9 | YTPGPGTRY, YTPGPGVRF | no | B*58:01 |
| TPGPGIRYPL | TPGPGVRYPL | 10 | TPGPGTRYPL, TPGPGVRFPL | no | B*07:02, B*35:01, |
| | | | | | B*42:01 |
| IRYPLTFGW | VRYPLTFGW | 9 | TRYPLTFGW, VRFPLTFGW | no | A*33:01, B*35:01, |
| | | | | | B*53:01 |
| RYPLTFGW | RYPLTFGW | 8 | RFPLTFGW | yes | A*23:01, A*24:02, |
| | | | | | C*04:01 |
| RYPLTFGWCF | RYPLTFGWCF | 10 | | yes | A*23:01 |
| YPLTFGWCF | YPLTFGWCF | 9 | | yes | B*18:01, B*35:01, |
| | | | | | B*53:01, C*02:02, |
| | | | | | C*04:01 |
| LTFGWCFKL | LTFGWCFKL | 9 | | yes | A*25:01, A*68:02 |
| LVGPTPVNI | LVGPTPVNI | 9 | LIGPTPVNI | yes | A*02:01 |
| TPVNIIGRNLL | TPVNIIGRNML | 11 | | no | B*81:01 |
| GIPHPAGLK | GIPHPAGLK | 9 | | yes | A*03:01 |
| TAFTIPSI | TAFTIPSI | 8 | TAFTIPST, TAFTIPSV | yes | B*51:01 |
| TPGIRYQYNVL | TPGIRYQYNVL | 11 | | yes | B*42:01, B*42:02 |
| IRYQYNVL | IRYQYNVL | 8 | | yes | B*14:01, B*73:01 |
| KQNPDIVIY | AQNPEIVIY | 9 | KONPEIVIY, AKNPEIVIY | no | A*30:02 |
| VIYQYMDDL | VIYQYMDDL | 9 | | yes | A*02:02 |
| PIQKETWEAW | PIQKETWETW | 10 | PIQKEIWETW, PIQKETWEIW | no | A*25:01, A*32:01 |
| TWEAWWTEYW | TWETWWTDYW | 10 | IWETWWTDYW, IWETWWTEYW, | no | B*49:01 |
| | | | TWEIWWTDYW, TWETWWTEYW | | |
| WEAWWTEYW | WETWWTDYW | 9 | WETWWTDYW, WETWWTEYW, | no | B*44:03 |
| | | | WEIWWTDYW | | |
| IVTDSQYAL | IVTDSQYAL | 9 | | yes | C*08:02 |
| FKRKGGIGGY | FKRKGGIGGY | 10 | | yes | B*15:03, C*01:02 |
| KRKGGIGGY | KRKGGIGGY | 9 | | yes | B*27:05 |
| GERIVDII | GERIIDII | 8 | | no | B*40:02 |
| GERIVDIIA | GERIIDIIA | 9 | | no | B*40:06 |

Epitope-allele pairs reported in LADB were collected across many studies included for further evaluation, we found the corresponding sequences in Clade B and Clade C 2021 consensus sequences from LADB. If the LADB epitope sequence differed from Clades B/C, it was discarded, and only consensus sequences were retained. To further validate published information regarding epitope/HLA interactions, we used a peptide-MHC binding prediction tool. Our analysis returned that approximately 40% of published epitope-allele pairs were predicted as non-binders. Out of these pairs, we aimed to check the binding of epitopes where the HLA class-I partner allele was observed in >2% prevalence of either the Caucasian European or Southern African populations.

To check the binding, we used a pMHC stability assay. Not all desired epitope-allele pairs could be tested due to the unavailability of MHC monomers for some alleles. For available alleles, 18 epitope/allele pairs were confirmed as binders and retained, while 37 epitope/allele pairs were non-binders and excluded from the vaccine design. For epitope/allele pairs that could not be checked for binding, they were nevertheless retained in the vaccine, but were not used for coverage calculations.

The selected epitopes are structurally important and should be enriched in conserved regions of the HIV proteome. To check for this and ensure that relevant polymorphisms were not missing, we checked what percentage of patient sequences within Clade B and Clade C matched the consensus by comparing sequence data from LADB for proteins of interest (Gag/Pol/Env/Nef). Every amino acid within each epitope where the match was below 75% was added to the most prevalent polymorphic versions of the epitopes. In total, 17 polymorphic fragments were added to the vaccine.

Overlapping epitopes were combined and merged into peptide fragments to reduce the number of linkers needed and reduce the size of the overall RNA cassette. Fragments were designed to maximize the removal of predicted HIV-specific Class-II core epitopes to limit unwanted elicitation of HIV-specific CD4 response and to minimize the length of the coding sequence.

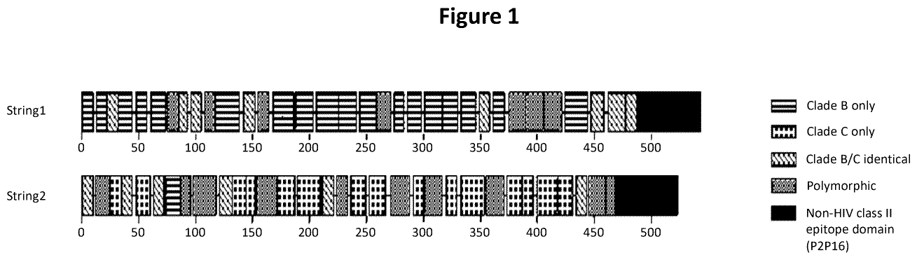

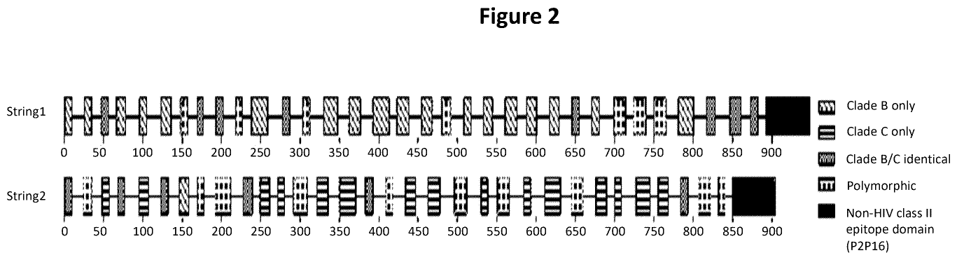

We distributed the epitopes and fragments between two encoding RNA molecules in which the epitopes were combined into 26 fragments represented in Clade B and Clade C consensus sequences (52 fragments total), to which the 17 polymorphic fragments were added. Four of the fragments containing a polymorphism were matched to identical Clade B/C consensus sequences. To reduce redundancy and limit the final RNA template size, we swapped the consensus fragments from Clade C with their polymorphic version. The vaccine is bivalent and contains two RNAs: one RNA is matched to “Clade B” consensus (26 fragments), and the second RNA is matched to “Clade C” consensus (26 fragments plus 4 polymorphic fragments). We distributed the 13 remaining polymorphic fragments across both RNAs strings, with 7 on the “Clade B RNA string” and 6 on the “Clade C RNA string”. In our final design, RNA string 1 (“Clade B string”) encompasses 33 fragments with a cumulative length of 411 amino acids, and string 2 (“Clade C string”) has 32 fragments of 405 amino acids total, as seen in FIGS. 1 and 2.

String 1 (“Clade B string”) (SEQ ID NOs: 183 and 187) comprises the following 33 fragments comprising 1, 2, 3, 4, 5, 6, 7 of the above epitopes from the pol, gag, env and nef proteins:

DCKTILKAL, GERIVDIIA, IVTDSQYAL, AEQASQEVKNWM,
SFNCGGEFF, GHQAAMQMLKETI, KONPEIVIY, TAFTIPSI,
KLTPLCVTL, TAFTIPSV, QNYTPGPGIRYPLTFGWCFKL,
FKRKGGIGGY, NTQGYYPDW, SALSEGATPQDLNTMLNT,
LVGPTPVNIIGRNLL, VKVVEEKAFSPEVIPMFSAL,
KQNPDIVIYQYMDDL, FRDYVDRFYKTLRA, AEQASQDVKNWM,
RAIEAQQHL, VRMYSPTSILDI, PIQKETWEAWWTEYW,
HTQGYFPDWQNYT, EIYKRWIILGLNK, GLNKIVRMY,
TSTLQEQIGW, PIQKEIWETWWTDYW, GQMVHQPLSPRTLNAW,
YTPGPGTRYPLTFGW, GQMVHQAISPRTLNAWVKVV,
TPGIRYQYNVL, ACQGVGGPGHKARVL,
and
GIPHPAGLK.

String 2 (“Clade C string”) (SEQ ID NOs: 185 and 189) comprises the following 32 fragments comprising 1, 2, 3, 4, 5, 6, 7 of the above epitopes from the pol, gag, env and nef proteins:

IVTDSQYAL, AEQATQEVKNWM, TSTLQEQIAW, GIPHPAGLK,
VRMYSPVSILDI, GLNKIVRMY, GHQAAMQMLKETI, AKNPEIVIY,
GQMVHQALSPRTLNAWVKVI, TPGIRYQYNVL, AEQATQDVKNWM,
SFNCRGEFF, YTPGPGVRFPLTFGWCF, AQNPEIVIYQYMDDL,
QNYTPGPGVRYPLTFGWCFKL, FKRKGGIGGY, NTQGYFPDW,
HTQGFFPDWQNYT, PIQKETWETWWTDYW, GQMVHQPISPRTLNAW,
GERIIDIIA, LIGPTPVNIIGRNML, DCKTILRAL,
VKVIEEKAFSPEVIPMFTAL, PIQKEIWETWWTEYW,
FRDYVDRFFKTLRA, RAIEAQQHM, TALSEGATPQDLNTMLNT,
DIYKRWITIGLNK, KLTPLCVTL, ACQGVGGPSHKARVL,
and
TAFTIPST.

String 1.1 (“Clade B string”) (SEQ ID NO: 205) comprises the following 33 fragments comprising 1, 2, 3, 4, 5, 6, 7 of the above epitopes from the pol, gag, env and nef proteins:

DCKTILKAL, GERIVDIIA, IVTDSQYAL, AEQASQEVKNWM,
SFNCGGEFF, GHQAAMQMLKETI, KQNPEIVIY, TAFTIPSI,
KLTPLCVTL, TAFTIPSV, QNYTPGPGIRYPLTFGWCFKL,
FKRKGGIGGY, NTQGFFPDW, SALSEGATPQDLNTMLNT,
LVGPTPVNIIGRNLL, VKVVEEKAFSPEVIPMFSAL,
KQNPDIVIYQYMDDL, FRDYVDRFYKTLRA, AEQASQDVKNWM,
RAIEAQQHL, VRMYSPTSILDI, PIQKETWEAWWTEYW,
HTQGYFPDWQNYT, EIYKRWIILGLNK, GLNKIVRMY,
TSTLQEQIGW, PIQKETWEIWWTDYW, GQMVHQPLSPRTLNAW,
YTPGPGTRYPLTFGW, GQMVHQAISPRTLNAWVKWV,
TPGIRYQYNVL, ACQGVGGPGHKARVL,
and
GIPHPAGLK.

String 2.1 (“Clade C string”) (SEQ ID NO: 208) comprises the following 32 fragments comprising 1, 2, 3, 4, 5, 6, 7 of the above epitopes from the pol, gag, env and nef proteins:

IVTDSQYAL, AEQATQEVKNWM, TSTLQEQIAW, GIPHPAGLK,
VRMYSPVSILDI, GLNKIVRMY, GHQAAMQMLKETI, AKNPEIVIY,
GQMVHQALSPRTLNAWVKVI, TPGIRYQYNVL, AEQATQDVKNWM,
SFNCRGEFF, YTPGPGVRFPLTFGWCF, AQNPEIVIYQYMDDL,
QNYTPGPGVRYPLTFGWCFKL, FKRKGGIGGY, NTQGYFPDW,
HTQGFFPDWQNYT, PIQKETWETWWTDYW, GQMVHQPISPRTLNAW,
GERIIDIIA, LIGPTPVNIIGRNML, DCKTILRAL,
VKVIEEKAFSPEVIPMFTAL, PIQKETWETWWTEYW,
FRDYVDRFFKTLRA, RAIEAQQHM, TALSEGATPQDLNTMLNT,
DIYKRWIILGLNK, KLTPLCVTL, ACQGVGGPSHKARVL,
and
TAFTIPST.

We supplemented each string with an additional non-human sequence domain enriched in universal non-HIV class-II epitopes to elicit CD4+ T cells that can help activate CD8+ T cells.

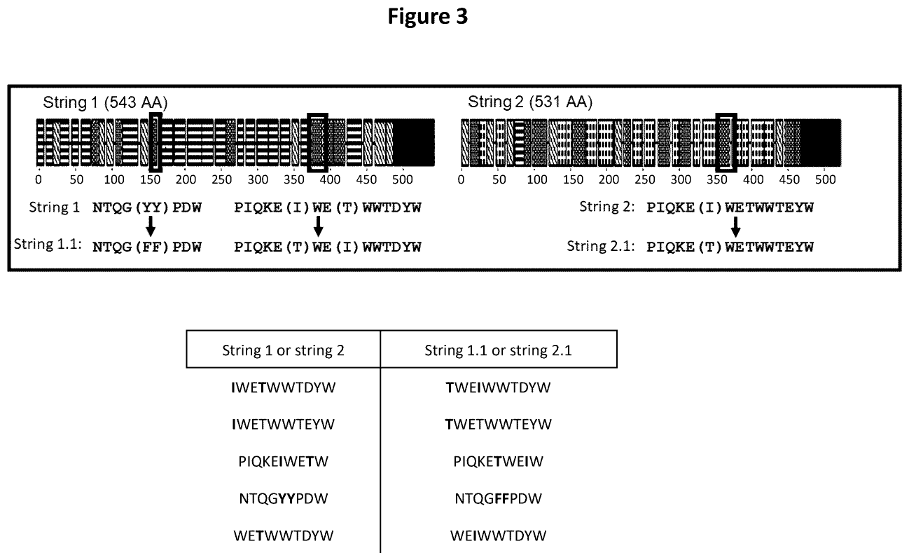

To link fragments in each string, two strategies were employed:
1) Direct addition of up to four cleavage-enhancing amino acids between every two fragments, as judged by the internal algorithm. In this strategy, the final length of the strings was ~550 amino acids. (FIG. 1)
2) Flexible non-immunogenic linker (10 amino acids), flanked by cleavage-enhancing amino acids (up to four), between every two fragments. In this strategy, the final length of the strings was ~950 amino acids. (FIG. 2) mRNA string 1 and 2 differ from mRNA strings 1.1 and 2.1 by very few amino acid changes as shown in FIG. 3.

Cleavage-enhancing supplementary residues were selected based on an algorithm, with the one additional parameter: the selected residues must not match the natural flanking residues present in either Consensus B or Consensus C sequences. This prevented the extension of our fragment to non-network residues, and avoided the addition of unwanted non-networked HIV class I epitopes or supplementary HIV class-II epitopes.

Fragments were randomized on each RNA, with a different order for string 1 and string 2 to improve downstream RNA analytics and repetition of junctional epitopes.

### Example 2—Mass Spectrometry Proteomics Analysis

Briefly, 5×10⁶ HEK293T cells were transfected with 1 μg of mRNA encoding strings 1 and 2 and MessengerMax Lipofectamine Reagent. Cells were incubated for 24 h in the presence or absence of 1 μM of proteasome inhibitor MG132. 24 h post-transfection, cells were harvested, washed in 1 mL PBS, snap frozen and stored until analysis. Frozen cell pellets were thawed and then lysed in cold lysis buffer. Approximately 100 μg of protein from each sample was normalized at a concentration of 1 μg/μL in lysis buffer. Lysates were diluted to reduce urea content in the lysis buffer to 1 M. Trypsin/Lys-C was added at a ratio of 1 μg per 50 μg total protein and the samples were incubated overnight. Synthetic tryptic peptides were labeled with heavy isotopes and injected for targeted MS/MS analysis. Data analysis was performed using Skyline-daily software. Retention times and peptide fragments were identified by matching with the spiked-in heavy isotope-labeled synthetic peptides. Relative abundance for each peptide was calculated by measuring the area under the curve (AUC) for the top ten most abundant fragment ions.

Loading normalizations were calculated using a panel of peptides derived from housekeeping proteins whose abundance within the cell is constant. AUCs for each housekeeping peptide were normalized to the mean AUC across samples, and the median of each mean AUC was calculated across the panel of housekeeping proteins. This median is the loading normalization factor for each sample. The Relative abundance is calculated by dividing the AUC of each peptide derived from each mRNA string by the loading normalization factor. AUCs for each target peptide were normalized to the mean AUC across samples.

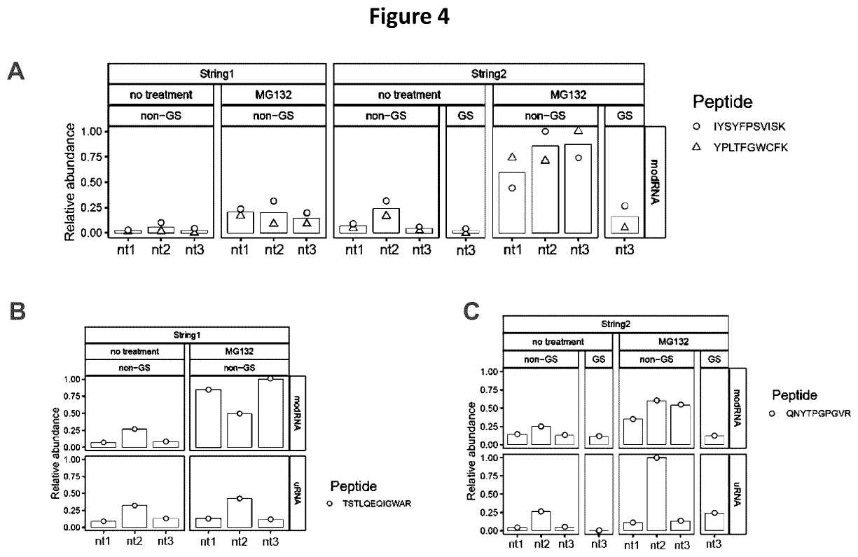

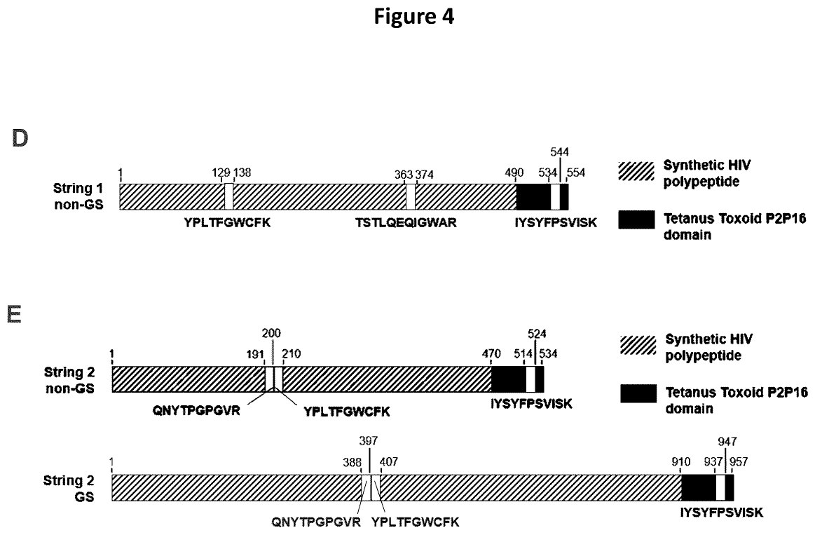

FIG. 4 shows the in vitro expression of mRNA-encoded polypeptides from 2 mRNA strings with or without flexible Glycine-Serin (GS) linker (string 1 non-GS, string 2 non-GS and string2 GS). For each string, 3 different nucleotide sequences were generated (nt1, nt2 and nt3), wherein nt1 corresponds to the wild-type sequence and nt2 and nt3 represent two different codon optimization methods. Each string was in vitro transcribed in the presence of either unmodified (uRNA) or methyl pseudouridine (modRNA). For string 2 GS only nt3 was tested. Each mRNA string, i.e. uRNA/modRNA, nt1-3, string 1-2, GS/non-GS, was transfected separately in a human cell line in the presence or absence of the proteasome inhibitor MG132. Relative abundance for each peptide is shown for (A) two tryptic peptides common to each string tested with modRNA; (B) tryptic peptide specific for string 1 non-GS, modRNA and uRNA; (C) tryptic peptide specific for strings 2 GS and non-GS, modRNA and uRNA. The tryptic peptides common to all strings, YPLTFGWCFK and IYSYFPSVISK, tested are located in the central part and the C-terminal region of the polypeptides encoded by mRNA string 1 non-GS (D) and String 2 non-GS and GS (E). String-specific tryptic peptides location tested for string 1 non-GS, TSTLQEQIGWAR, (D) and string 2 GS and non-GS, QNYTPGPGVR, (E) are also shown.

The experiments show overall that higher in vitro expression could be achieved using modRNA compared to uRNA. The detection of the IYSYFPSVISK tryptic peptide indicates that the different strings and codon optimized versions thereof were translated up to the C-terminal region of the encoded polypeptide. Codon optimization influences the level of translation from the different strings and appears to be sequence dependent. The presence of proteasome inhibitor MG132 led to an increase in detectable polypeptides, indicating that the translated polypeptide is targeted to proteasomal degradation pathway, required for the processing of epitopes.

### Example 3—Immunopeptidomics

A375 cells were either engineered to stably express BAP-tagged alleles of interest or the allele of interest was overexpressed within the cell and used for transfection. 5×107 engineered cells were transfected with 1 μg of mRNA encoding the modRNA strings 1 and 2 and Messenger Max Lipofectamine reagent prior to harvest. Transfected cells were lysed and cleared before processing. For BAP-tagged cell lines, the cleared lysate was biotinylated with biotin, ATP and BirA prior to incubation with NeutrAvidin beads to affinity-enrich biotinylated-HLA-peptide complexes. For overexpressed cell lines, sepharose beads were charged with a pan-class-I antibody and incubated with the cleared lysate to isolate all HLA-peptide complexes. Peptides were washed and eluted from antibody-bound HLA complexes and molecular weight filtration was performed to isolate peptides. Isolated peptides were then labeled with TMTzero, then reduced using TCEP, alkylated using iodoacetamide (IAA) and desalted prior to analysis by nLC-MS/MS.Samples were resuspended in a 3% acetonitrile, 5% formic acid supplemented with 150 femtomoles of each TMT-131C labeled heavy synthetic peptide per injection. Peptides were chromatographically separated using a Vanquish Neo uHPLC fitted with an Aurora Ultimate packed emitter column and heated at 60° C. during separation. Peptides were eluted into an Orbitrap Ascend Tribrid Mass Spectrometer equipped with a Nanospray Flex Ion source. Data were acquired using internal standard triggered parallel reaction monitoring. Fast, low-resolution precursor scans were used to look for m/z values in an inclusion list associated with the TMT-131C labeled heavy synthetic peptide internal standards. When an m/z value from the inclusion list was observed, a fast, low-resolution tandem mass spectrum (MS/MS) survey scan was performed, and characteristic fragment ions associated with the peptide were monitored. If five or more monitored ions were observed, a second MS/MS scan was performed with a mass offset equal to the difference between the TMT-131C labeled heavy synthetic peptide and the TMTzero labeled target HLA peptide.

Data analysis was performed using Skyline-daily software. Retention times and peptide fragments were identified by matching with the spiked-in heavy isotope-labeled synthetic peptides. Relative abundance for each peptide was calculated by measuring the AUC for the top ten most abundant fragment ions.

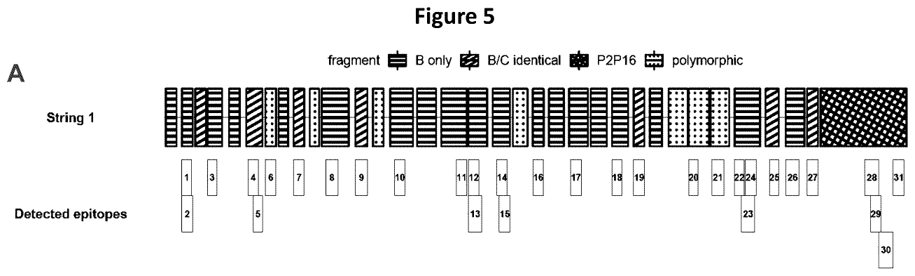

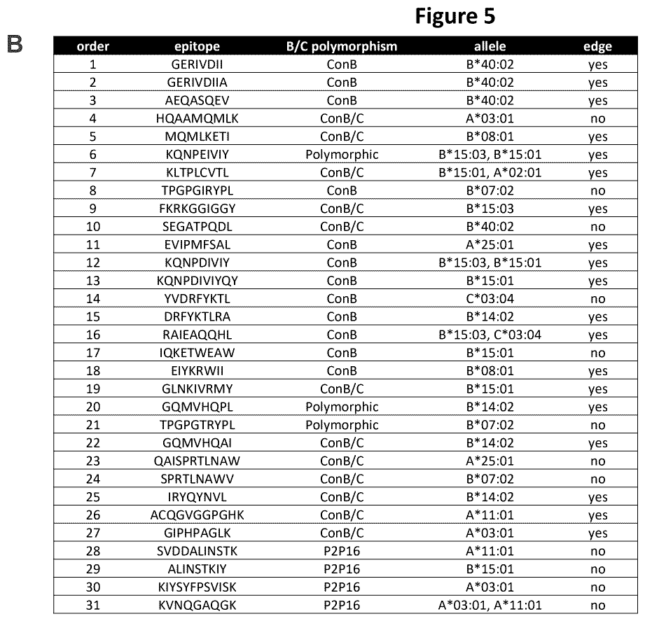

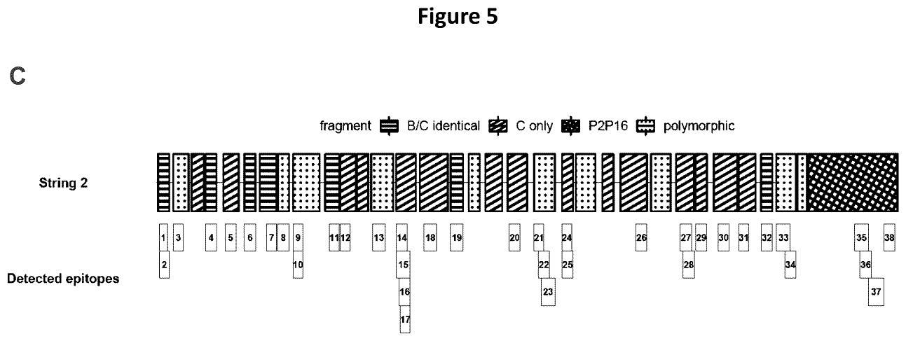

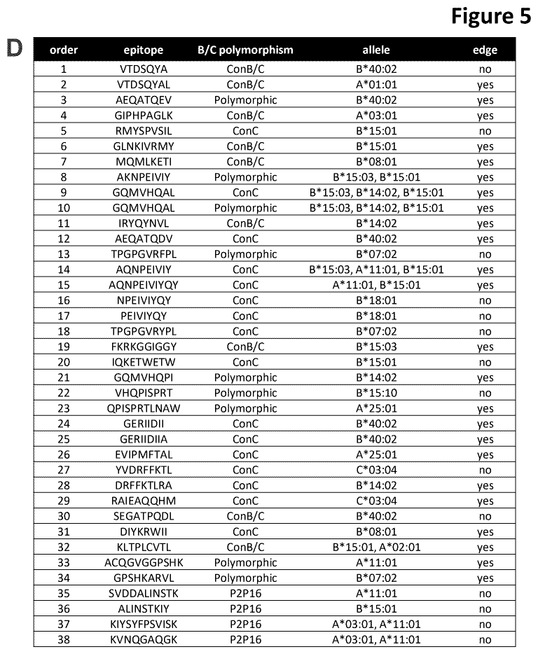

FIG. 5 shows the in vitro processing and presentation by HLA-I alleles from mRNA-encoded polypeptides from 2 modRNA strings (string 1 and string 2) was assessed with immunopeptidomics. Each string was transfected in an A375 cell line encoding a single HLA class I allele and targeted mass spectrometry allowed to detect HIV specific epitopes in string 1 (A-B) and string 2 (C-D). The detected epitope locations are annotated under string 1 (A) and string 2 (C). The detailed information related to each detected epitope, such as the HLA class-I allele, location at the edge of a fragment, and a non-HIV junctional sequence (edge) and the sequence match for string 1 is shown in panel (B) and the corresponding information for string 2 is shown in panel (D). Legend: Epitope sequence perfectly matches to a 2021 consensus B=ConB; Epitope sequence perfectly matches to a 2021 consensus C=ConC; Epitopes sequence perfectly matches to Consensus B and C=ConB/C; epitope not a perfect match in either consensus B or C=Polymorph.

These datasets confirm processing and presentation of epitopes from mRNA-encoded polypeptide onto human MHC class-I alleles. The detection via pull-down of epitope located at the edge of fragments confirm the cleavage of the polypeptide in non-HIV junctional area inter-fragments.

### Example 4—T-Cell Activation Assay with K562

NFAT-TCR/CD3 effector cells were purchased from Promega as cryopreserved cells. These Jurkat T-cells express luciferase as a reporter, driven by an NFAT-response element (NFAT-RE). The endogenous TCR and 132M-gene have been removed in the Jurkat reporter cells by CRISPR-Casp9-mediated knockout. The alpha- and beta-chain of the CD8-coreceptor were stably inserted in the Jurkat reporter cells by via transposon. Reporter NFAT-luciferase cells were co-electroporated with two mRNAs, encoding for a TCR clones alpha and beta chains. Post transfection, 2×10⁴ Jurkat cells were co-cultured with K562 cells at a 2:1 ratio, in a 384-well-plate with 25 μL medium (RPMI1640+10% non-heat inactivated FBS)/well. Prior to co-culture, the K562 cells were transfected with a mRNA encoding for an HIV-derived polypeptide (string 1 or string 2) and mRNAs encoding for an HLA class I alleles. As a positive control for the specificity of the used TCRs, K562 cells only transfected with mRNAs encoding the HLA-I pulsed with a minimal HIV-epitope peptide target, were co-cultured with each TCR-encoding mRNA transfected Jurkat reporter cells. Moreover, stimulation with 2 μg/ml Phytohemagglutinin-L (PHA-L) was used to corroborate TCR expression and downstream signaling. Transient expression of transfected HLA class-I was verified by flow cytometry after staining with HLA-A or HLA-B specific antibodies. After 16 h, an equal volume (15 μL) of luciferin (Bio-Glo, Promega) was added to each well and the luciferase activity was measured using a luminescence plate reader. The measured luminescence signal in the different wells corresponded to the level of TCR-mediated activation in the Jurkat cells. For each TCR, log 2 fold change of luminescence compared to the “effectors only control” was calculated and a cut-off of two-fold change was used to determine specific TCRs.

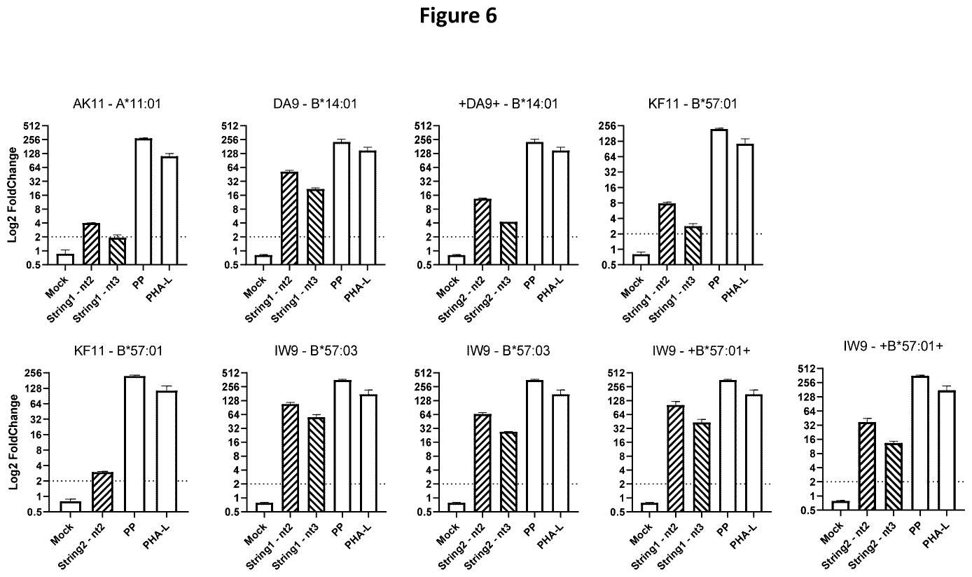

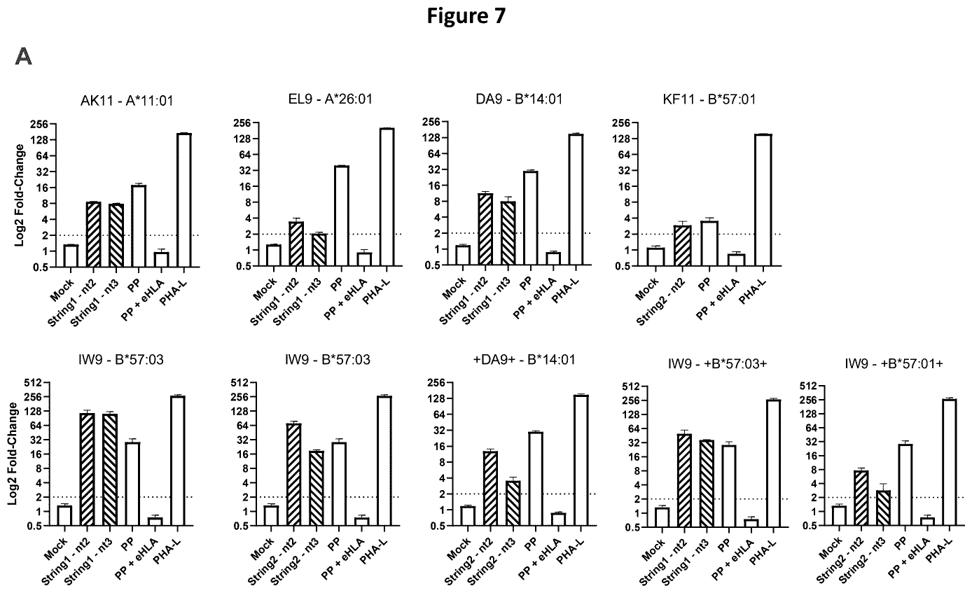

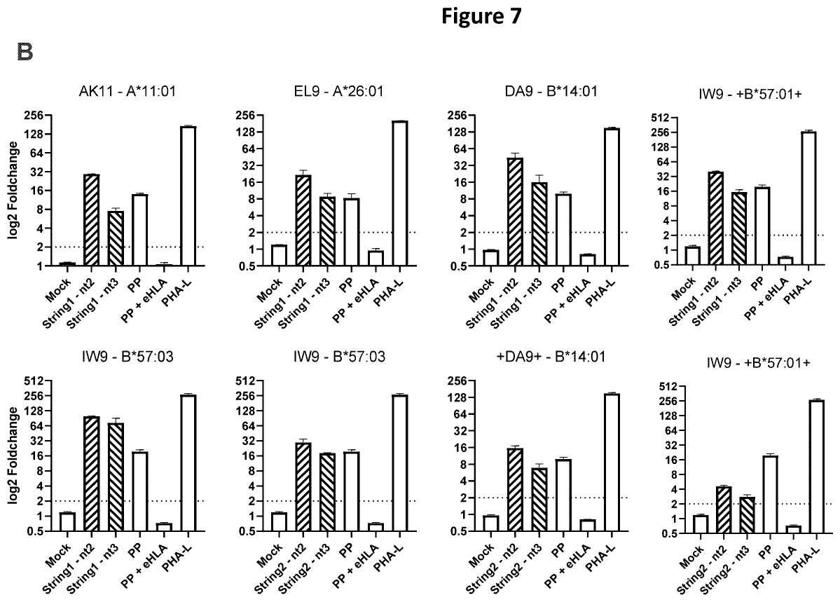

FIG. 6 shows the TCR engagement from epitope processed and presented from different mRNA strings in the K562 cell line. A Jurkat cell line expressing a single TCR was co-incubated with mono HLA-I allele K562 cells to measure TCR downstream signaling expressed as log 2 fold signal of luciferase. K562 were pulsed with a peptide encoded by the tested string (PP) as positive control to control the TCR activity for the specific epitope tested or transfected with a mRNA encoding for a polypeptide (stripped bar). Two different mRNA strings (string 1 and string 2) were tested with different nucleotide optimization (nt2 and nt3). PHA-L was used as a positive control for TCR activity. % unique epitopes, AK11, DA9, +DA9+, KF11 and IW9, were tested to react with their cognate TCRs. Processing and presentation of the exact epitopes KF11 and IW9 were investigated from different mRNA strings. A polymorphic version of DA9, marked as +DA9+, encoded by string2 versions was cross-reactive with the non-cognate DA9-specific TCR. Similarly, IW9 was presented by both the cognate HLA-I allele B*57:03 and non-cognate B*57:01 allele, marked as +B*57:01+ for both string1 and string2. Legend: AK11:ACQGVGGPGHK; DA9 DRFYKTLRA; +DA9+:DRFFKTLRA; KF11:KAFSPEVIPMF; IW9:ISPRTLNAW.

These datasets confirm that all mRNA-string encoded polypeptides can be enzymatically processed to generate epitopes that are then presented on their cognate HLA class I alleles. The epitope:HLA-I complexes are able, in vitro, to engage TCR downstream signaling. These data also show the impact of the nucleotide sequence optimization on the TCR-mediated response magnitude.

### Example 5—T-Cell Activation Assay with iDCs

Cells were used and prepared as described in example 4. Post transfection, 2×104 Jurkat cells were co-cultured with immature Dendritic cells (iDCs) cells at a 2:1 ratio, in a 384-well-plate with 25 μL medium (RPMI1640+10% non-heat inactivated FBS)/well. Prior to co-culture, iDCs were generated from donor PBMCs. Briefly, CD14+ monocytes were positively isolated from human PBMCs and cultivated for 5 days at 1×106 cells/mL in RPMI16407/5% pooled Human Serum (PHS)/1% Sodium pyruvate/0.5% Penicillin-Streptomycin supplemented with 1000U/mL IL-4 and 1600 U7 mL GM-CSF to generate iDCs. iDCs were then transfected with mRNAs encoding for an HIV-derived polypeptide (string 1 and 2) and mRNAs encoding for HLA class I alleles before co-incubation with the Jurkat reporter cells. As a positive control for the specificity of the used TCRs, iDCs only transfected with mRNAs encoding the HLA-I pulsed with a minimal HIV-epitope peptide target, were co-cultured with each TCR-encoding mRNA transfected Jurkat reporter cells. To evaluate the impact of endogenous HLA-I alleles from the PBMCs donor, a control including iDCs only pulsed with peptide was included. Moreover, stimulation with 2 μg/ml Phytohemagglutinin-L (PHA-L) was used to corroborate TCR expression and downstream signaling. After 16 h, an equal volume (15 μL) of luciferin (Bio-Glo, Promega) was added to each well and the luciferase activity was measured using a luminescence plate reader. The measured luminescence signal in the different wells corresponded to the level of TCR-mediated activation in the Jurkat cells. For each TCR, log 2 fold change of luminescence compared to the “effectors only control” was calculated and a cut-off of two-fold change was used to determine specific TCRs. FIG. 7 shows the TCR engagement from epitope processed and presented from different mRNA strings in iDCs generated from 2 donors. A Jurkat cell line expressing a single TCR was co-incubated with iDCs to measure TCR downstream signaling expressed as log 2 fold signal of luciferase. iDCs were pulsed with a peptide encoded by the tested string. To control for TCR activity the pulsed iDC were either previously co-transfected with a mRNA encoding an HLA class-I allele (PP) or without co-transfection and expressing only endogenous HLA-I from the donor (PP+eHLA) to control for potential endogenous HLA-I mediated background presentation. iDCs were co-transfected with an mRNA encoding for a polypeptide and the cognate HLA class I allele (stripped bar). Two different mRNA strings (string 1 and string 2) were tested with different nucleotide optimization (nt2 and nt3). PHA-L was used as a positive control for TCR activity. For donor 1 (A) and donor 2 (B) respectively 6 (AK11, DA9, +DA9+, KF11, EL9 and IW9) and 5 (AK11, DA9, +DA9+, EL9 and IW9) unique epitopes were tested to react with their cognate TCRs. Processing and presentation of epitope IW9 was investigated from two different mRNA strings (string1 and string2). A polymorphic version of DA9, marked as +DA9+, encoded by string2 versions was cross-reactive with the non-cognate DA9-specific TCR. Similarly, IW9 was presented by both the cognate HLA-I allele B*57:03 and non-cognate B*57:01 allele, marked as +B*57:01+ for both string1 and string2. Legend: AK11:ACQGVGGPGHK; DA9 DRFYKTLRA; +DA9+:DRFFKTLRA; KF11:KAFSPEVIPMF; IW9:ISPRTLNAW; EL9:EVIPMFSAL.

These datasets confirm that all mRNA-string encoded polypeptides can be enzymatically processed to generate epitopes that are then presented on their cognate HLA class I alleles in PBMCs derived human iDCs. The epitope:HLA-I complexes are able, in vitro, to engage TCR downstream signaling. These data also show the impact of the nucleotide sequence optimization on the TCR-mediated response magnitude.

### Example 6

Immunogenicity studies in BALB-C (wildtype) and HLA Knock-In mice with the RNA molecules depicted in FIGS. 1 and 2 are performed. For this, compositions with lipids for intra-muscular (i.m.) or intravenous (i.v.) delivery are combined with the RNA molecules.

### Vaccination of Mice

Wild type mice: Wild type mice are vaccinated i.m. with lipid nanoparticle formulations or i.v. with lipoplex formulations. Wild type mice receive the vaccine, i.e., 2-4 RNAs co-formulated or 2-4 RNA single formulated, with up to 3 doses (low mid high) following different prime/boost regimen—i.e., time between injection and number of boosts. Vaccine-induced CD8+ T cells are quantified from splenocytes and whole blood via peptide pool ELISPOT upon culling. Drained lymph nodes are harvested for immunostaining of relevant markers. Cytokine levels in blood are measured by multiplex ELISA during the course of the experiment. The magnitude of HIV-specific CD8+ T cells are compared across regimen, dosing, formulation (if applicable) and RNA platforms (if applicable), to define optimal vaccination schedule for both routes of injection.

HLA knock-in (HLA KI) mice: HLA KI mice are vaccinated i.m. with lipid nanoparticle formulations or i.v. with lipoplex formulations. The HLA panel is designed to cover relevant HLA-I alleles, i.e., highly prevalent in the population or linked to negative/positive outcome during the course of HIV infection. HLA KI mice receive the vaccines according to the optimal regimen defined for wildtype mice for each RNA platform/lipid formulation. Vaccine-induced CD8+ T cells are quantified from splenocytes and whole blood via ELISPOT with specific peptide for the HLA-I investigated. Drained lymph nodes are harvested for immunostaining of relevant markers. These experiments confirm the proper presentation and immunogenicity of the epitopes selected.

### Example 7

### Sample Preparation and Immunization

mRNAs produced by in vitro transcription were tested for endotoxin content and formulated in a lipoplex lipid nanocarrier. Each mRNA was formulated alone at concentration of either 0.1, 0.2 or 0.25 mg/mL 24 h prior injection and kept at 4° C. On the day of immunization, particles were lightly resuspended and, when applicable, diluted in PBS and or co-mixed to a final injection volume of 100 μL. CB6F1-Tg(HLA-A*0201/H2-Kb)A*0201 (CB6F1) mice were immunized by tail vein injection (29G needle) at d0 (prime), d7 (boost) and d14 (boost). 6 h post immunization, a blood sample from each animal was recovered and stored at −80° C. On d21, 1 week after the last boost, animals were sacrificed and their spleens recovered for immediate CD4+ and CD8+ T cell isolation. Animals were followed for health monitoring during the entire experiment until sacrifice.

### T Cell Isolation and ELISpot

Freshly recovered splenocyte were subjected to either a negative CD4- or CD8-isolation kit (Miltenyi biotec Cat 130-104-454 and 130-104-075). Briefly, 10e7 cells were incubated with a biotin antibody cocktail and incubated at 4° C. for 5 min. The antibody/cell mix was then incubated with antibiotin microbeads (2 μl/10e6 cells) for 10 min at 4° C. Unlabelled CD4+ or CD8+ T cells were separated from the labelled cells via magnetic separation, by recovering the flowthrough of the cell suspension. Cells were immediately used for IFNg-ELISpot assay. 5.10e4 BMDCs from CB6F1 were pulsed with 2-6 μg/mL peptides test or control for 2 h at 37° C. and then co-incubated overnight with 1.10e5 isolated T-cells on anti-IFNg coated plate (Mabtech 321-4HPW-2). Spots were detected using a biotinylated anti mouse IFNg detection antibody, followed by streptavidin-HRP incubation.

CD8+ and CD4+ T-Cell Responses to Two Different mRNA Strings (String1 and String2)

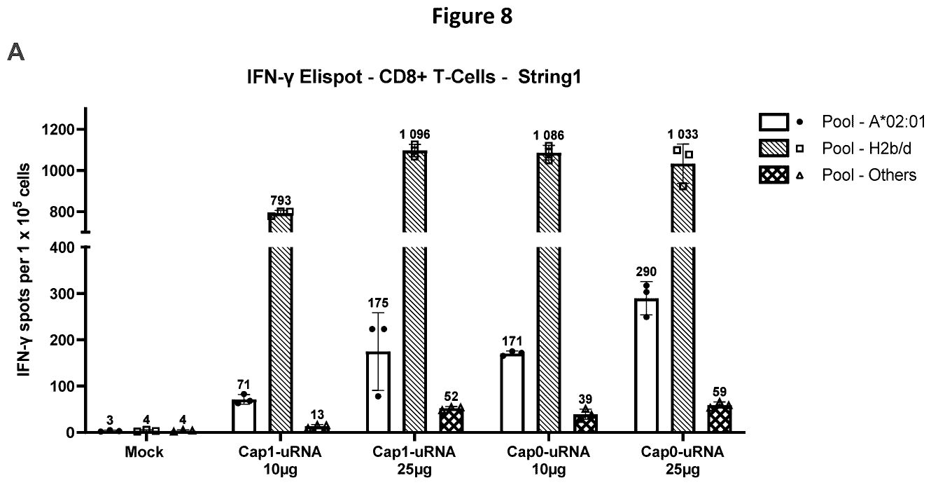

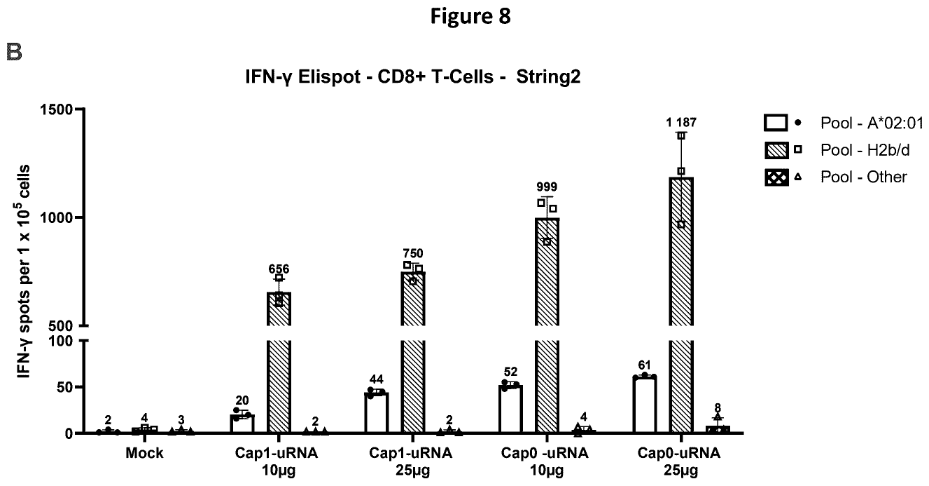

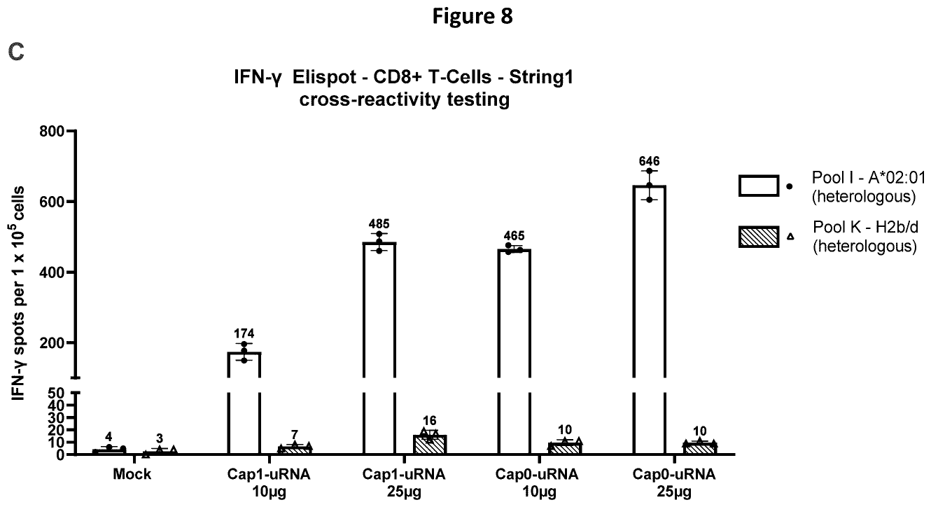

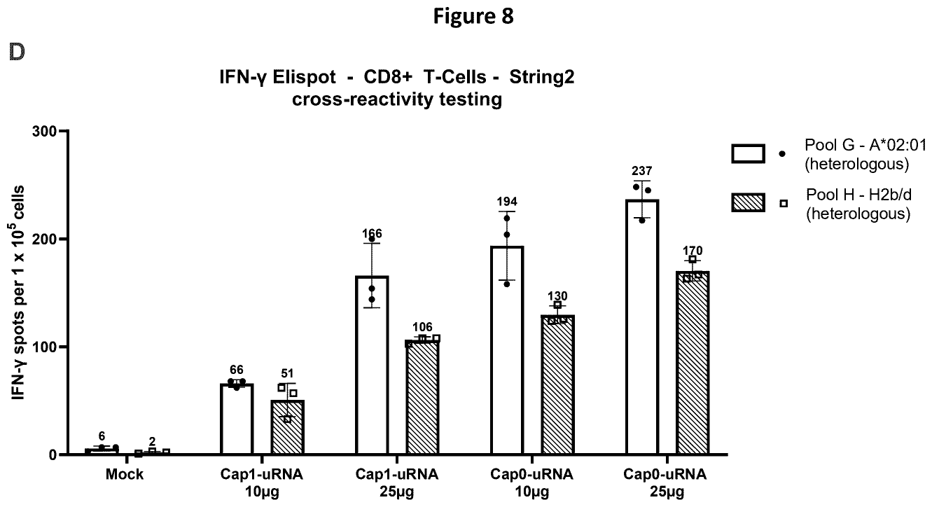

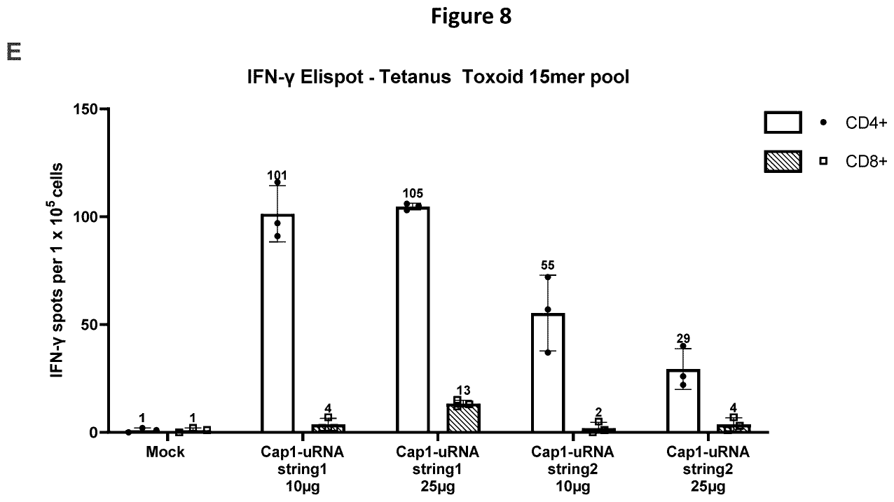

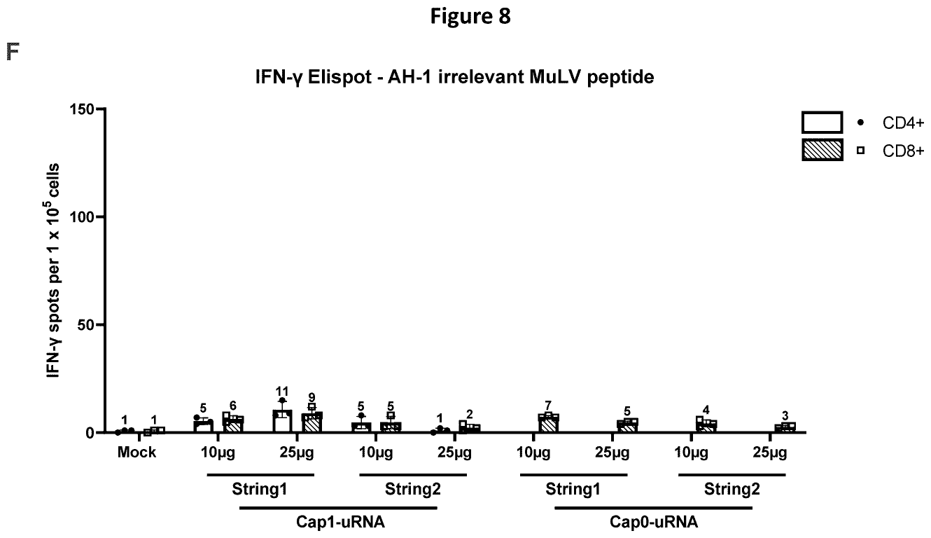

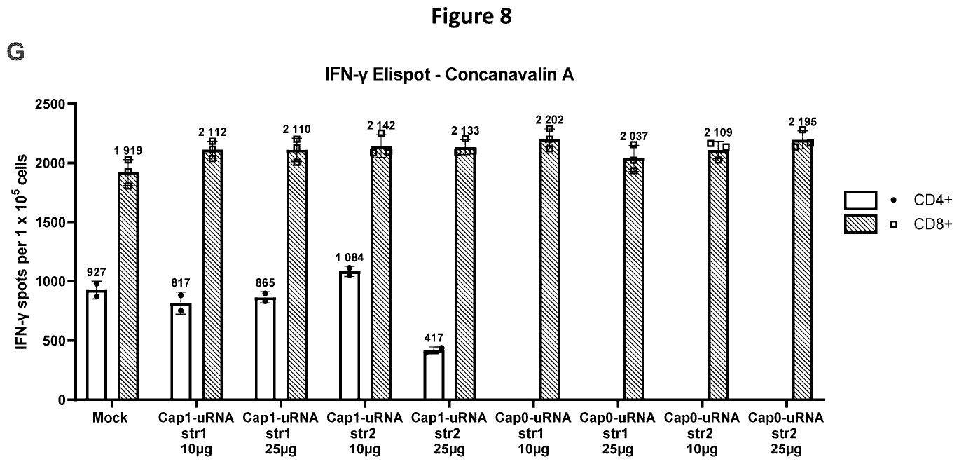

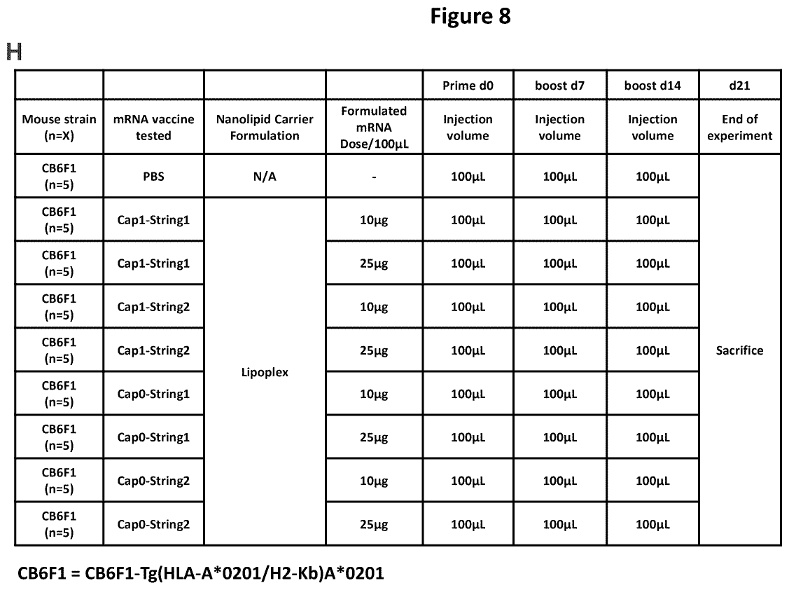

Elispot data for IFN-γ production by isolated CD8+ T and isolated CD4+ T cells stimulated with peptide pools. T-cells were isolated from HLA-A*02:01 CB6F1 transgenic mice and stimulated with peptide pools for 18 hours on Elispot plates coated with anti-IFN-γ antibody. CD8+ T cells from groups (n=5 mice per group) immunized with string1 (FIG. 8A) or string 2 (FIG. 8B) were incubated with 11mer peptide pools covering the vaccine fragments containing at least one known/predicted HIV-1 epitopes (prctrk<1%) from A*02:01 (white box—black circle) or H2b/H2d (striped box—white square) or neither (diamond box—white triangle). CD8+ T cells from groups immunized with string1 (FIG. 8C) or string 2 (FIG. 8D) were incubated with heterologous minimal A*02:01 (white box—black circle) or H2b/H2b (striped box—white square) HIV epitope pools, i.e. minimal HIV epitopes (8-11-mers) containing at least one polymorphism compared to the immunizing string sequence. Pool I/K, tested on string1 immunized groups and Pool G/H, tested on string2 immunized groups are exact match of sequences found only in respective string2 and string1. Isolated CD4+ T and CD8+ T cells (FIG. 8E) were incubated with a 15-mer peptide pool spanning the entire tetanus toxin. CD8+ and CD4+ T isolated cells of indicated immunized groups were tested with an AH-1 irrelevant 15-mer peptide pool negative control (FIG. 8F) and concanavalin A positive control (FIG. 8G). Quantification of IFNg spot-forming units per 100.000 cells for each peptide pool. FIG. 8H shows a summary of the experimental setup.

All strings tested, irrespective of the capping or sequence are immunogenic in the CB6F1 mouse model. The mRNA strings used in immunization elicits HIV-specific CD8+ T cell responses restricted by both Mouse MHC-I and human HLA-1 Allele A*02:01 (FIGS. 8A and B). Using a heterologous minimal peptide pool for CD8+ T cell stimulation indicates that the T cells generated by the mRNA strings are cross-reactive to polymorphic versions of their encoded epitopes (FIGS. 8C and D). The use of a P2P16 domain in the sequence to support the HIV CD8+ T cell by engaging non-HIV-specific CD4+ T cells has been validated using a specific 15-mer tetanus toxin peptide pool (FIG. 8E). The tetanus toxin (TT) peptide pool did not appear to elicit detectable TT-specific CD8+ T cell responses, limiting the competition between HIV- and TT-specific CD8+ T cells (FIG. 8E).

### Example 8

Sample preparation and immunization as well as T cell isolation and ELISpot methods were performed as in Example 7.

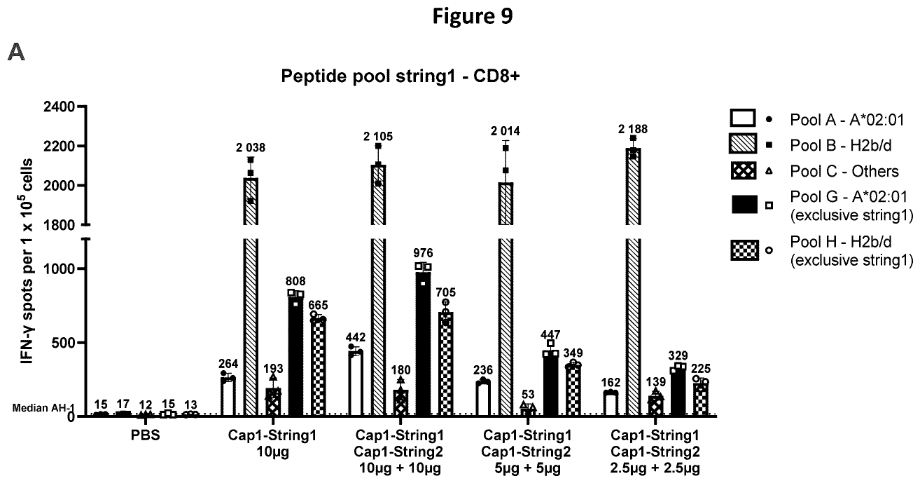

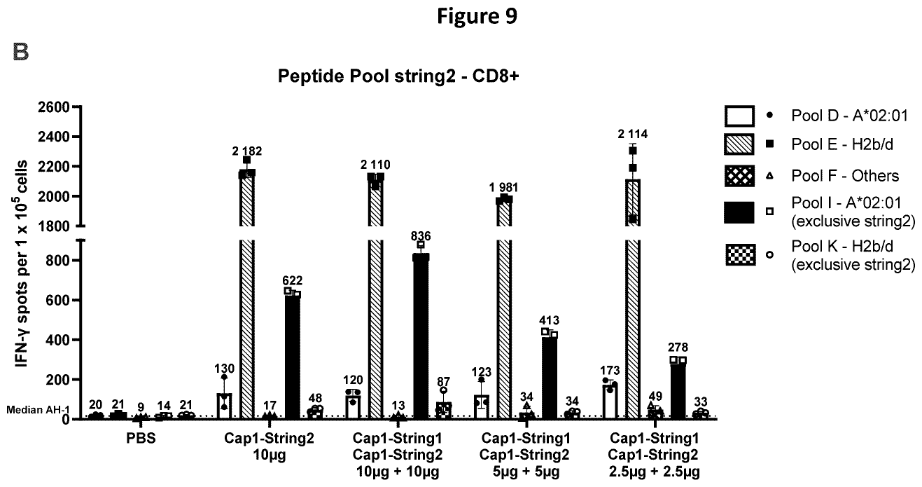

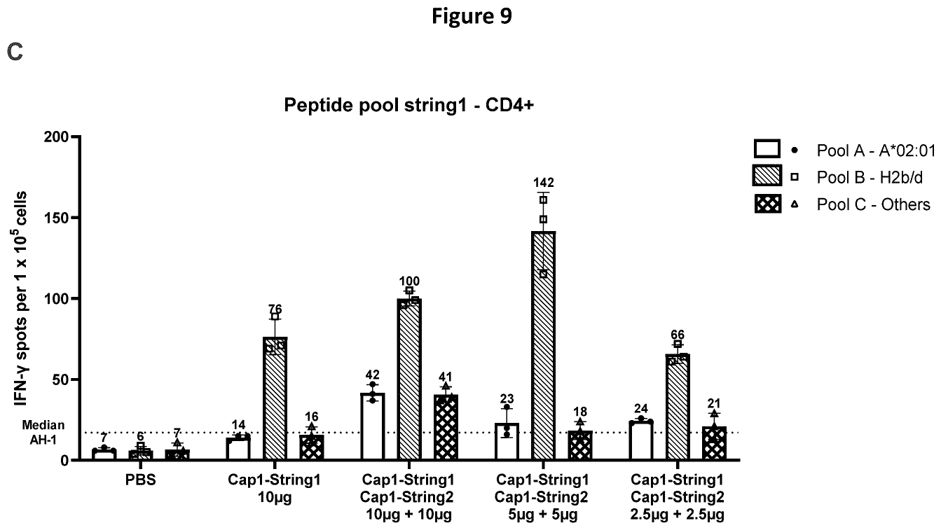

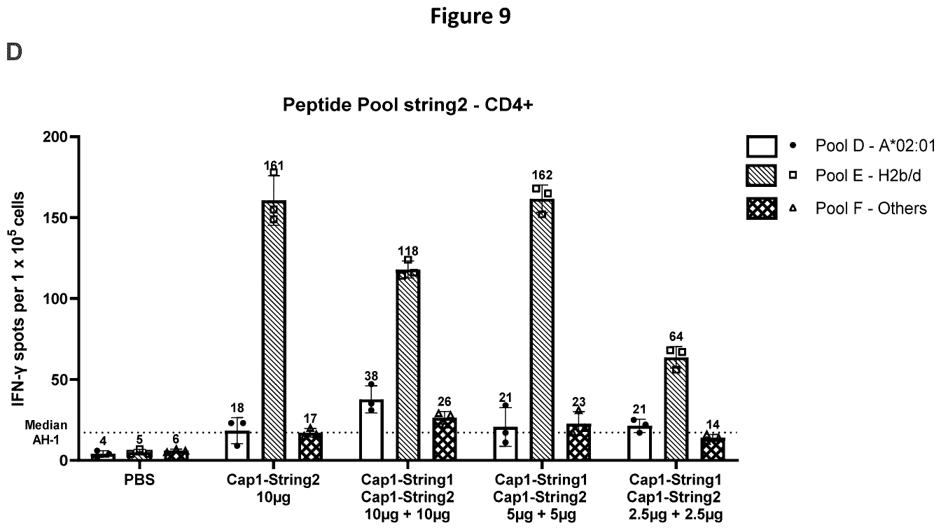

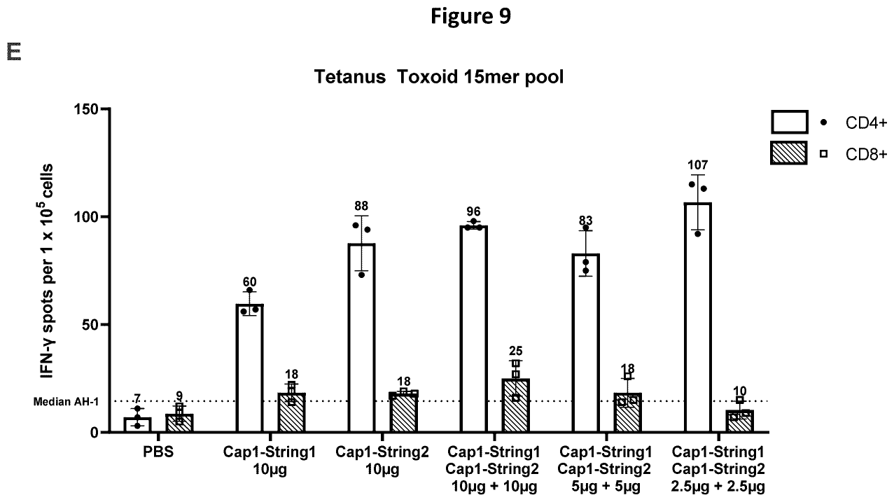

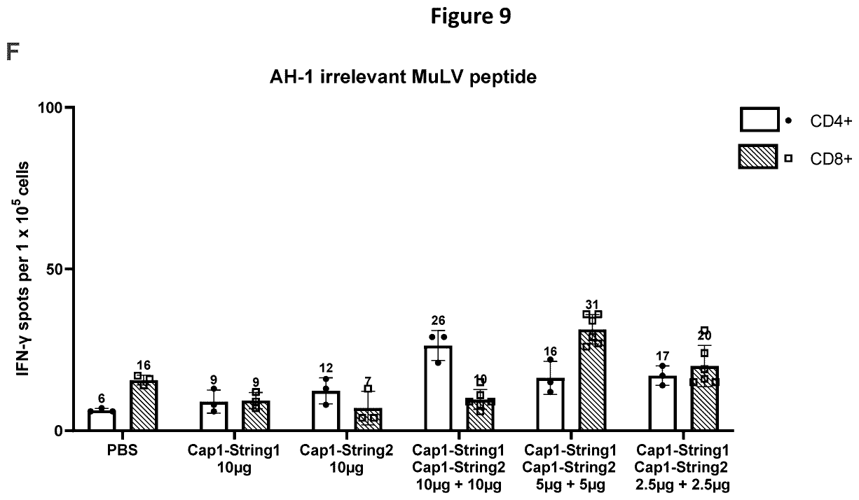

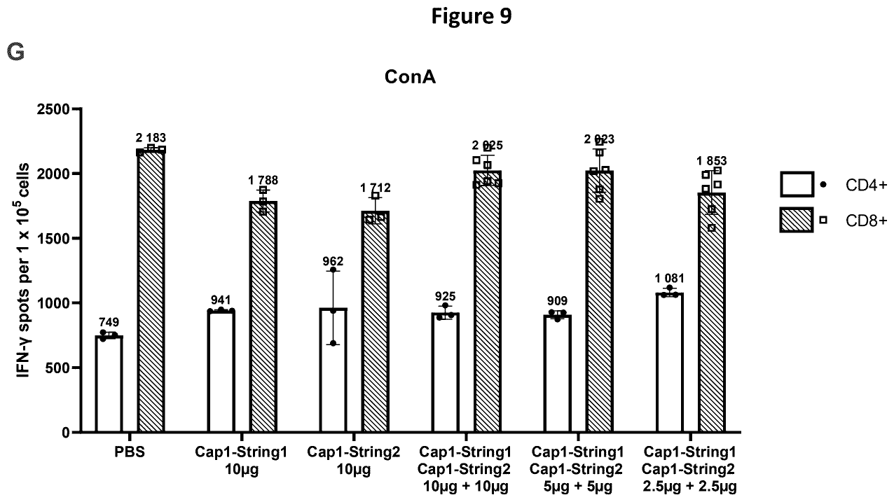

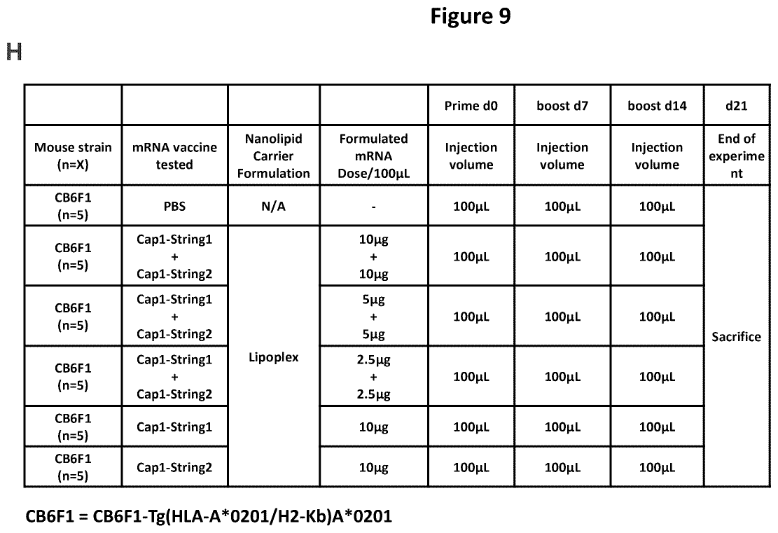

CD8+ and CD4+ T-cell responses to two different mRNA strings (string1 and string2): Elispot data for IFN-γ production by isolated CD8+ T and isolated CD4+ T cells stimulated with peptide pools. T-cells were isolated from HLA-A*02:01 CB6F1 transgenic mice and stimulated with peptide pools for 18 hours on Elispot plates coated with anti-IFN-γ antibody. CD8+ T cells from groups (n=5 mice per group) immunized with either a single mRNA, string1 (10 μg) or string 2 (10 μg), or co-immunized with both mRNA string1+string2 in a 1:1 ratio (10 μg+10 μg; 5 μg+5 μg; 2.5 μg+2.5 μg). Animals immunized with string1, alone or in combination, (FIG. 9A, 9C) or with string2, alone or in combination (FIG. 9B, 9D) were incubated with 11mer peptide pools covering the vaccine fragments containing at least one known/predicted HIV epitope (prctrk<1%) from A*02:01 (white box—black circle) or H2b/H2d (striped box—white square) or neither (diamond box—white triangle), respectively pool A, B, C and D, E, F. CD8+ T cells from groups immunized with string1 (FIG. 9C) or string 2 (FIG. 9D) were also incubated with minimal A*02:01 (white box—black circle) or H2b/H2b (striped box—white square) HIV epitopes pools (8-11-mers) exclusively encoded by either string1 (Pool G and H) or string 2 (Pool I/K). Similarly, CD4+ T cells from groups immunized with string1 (C) or string 2 (D) were incubated with minimal A*02:01 (white box—black circle) or H2b/H2b (striped box—white square) HIV epitopes pools (8-11-mers). Isolated CD4+ T and CD8+ T cells (FIG. 9E) were incubated with a 15-mer peptide pool spanning the entire tetanus toxin. CD8+ and CD4+ T isolated cells of indicated immunized groups were tested with an AH-1 irrelevant 15-mer peptide pool negative control (FIG. 9F) and concanavalin A positive control (FIG. 9G). Quantification of IFNg spot-forming units per 100.000 cells for each peptide pool. FIG. 9H shows a summary of the experimental setup.

Single mRNA string and bivalent combination vaccine (string1+string2) immunizations are immunogenic in the CB6F1 models. The mRNA strings, alone or in combination, elicit HIV-specific CD8+ T cell responses restricted by both Mouse MHC-I and human HLA-1 Allele A*02:01 (FIGS. 9C and D). Responses against non-saturating HIV minimal epitopes peptides pools (Pool G, H, I and K), show a dose response to the bivalent vaccine. In comparison, low/modest levels of HIV-specific CD4+ T cell responses restricted by A*02:01 and H2b/d MHC-I alleles (FIGS. 9C and D) were elicited by the vaccine, warranting the addition of the P2P6 tetanus toxin helper epitopes. The use of a P2P16 domain in the sequence to support the HIV CD8+ T cell by engaging non-HIV-specific CD4+ T cells has been validated using a specific 15-mer tetanus toxin peptide pool (FIG. 9E). The tetanus toxin (TT) peptide pool did not appear to elicit detectable TT-specific CD8+ T cell responses, limiting the competition between HIV- and TT-specific CD8+ T cells (FIG. 9E).

### Example 9

TCR Engagement from Epitope Processed and Presented from Different mRNA Strings in the K562 Cell Line

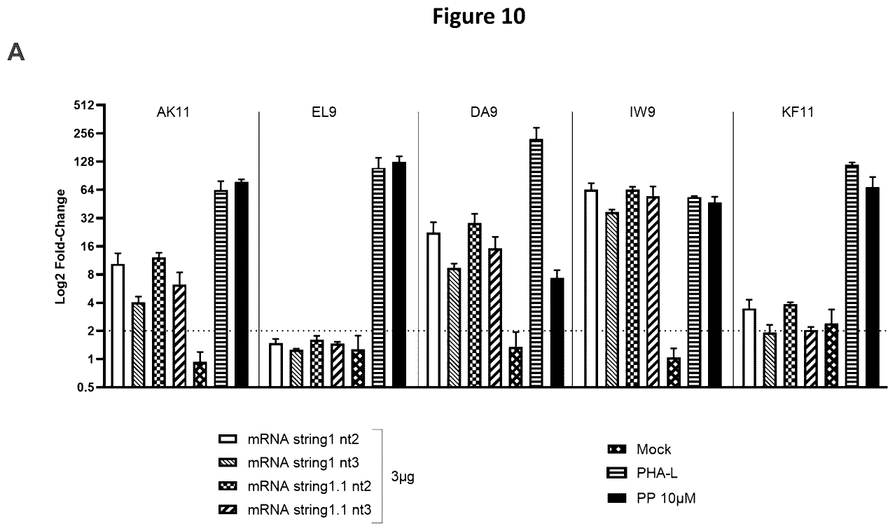

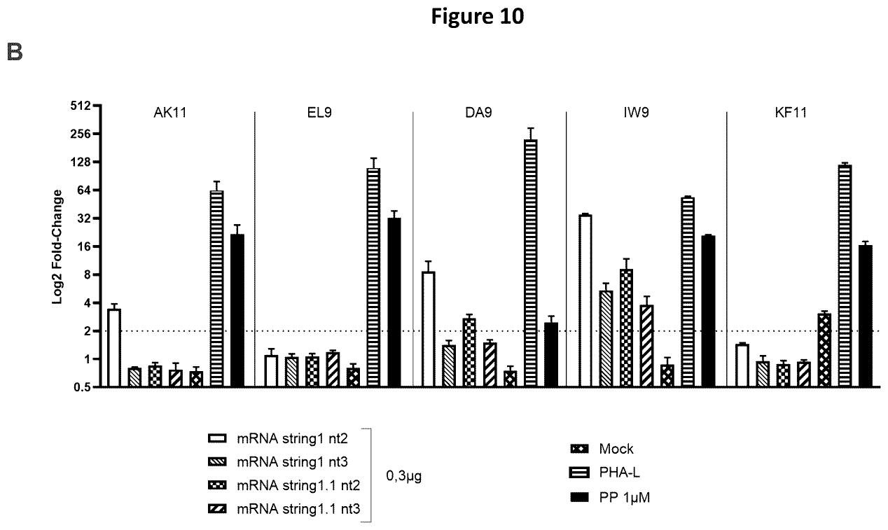

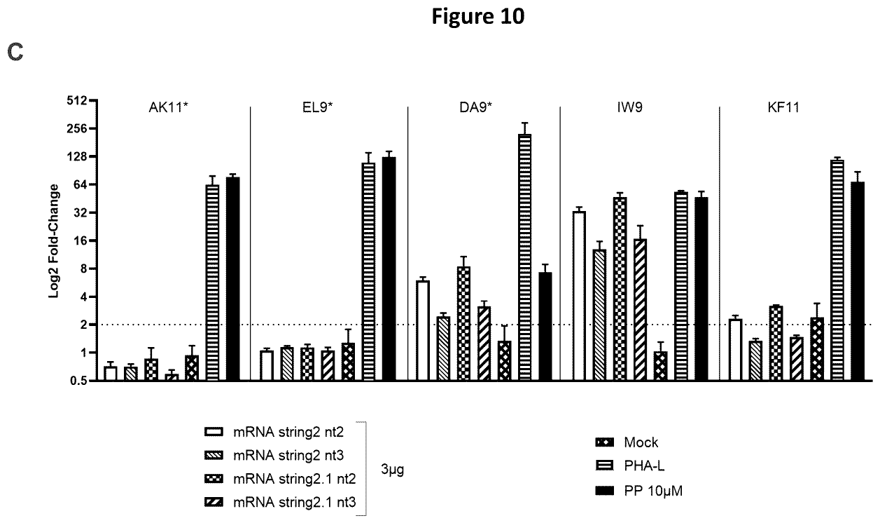

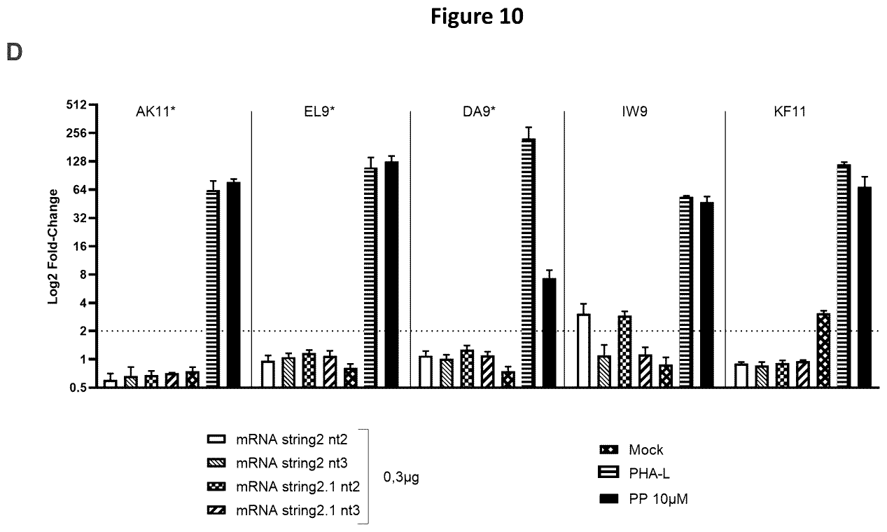

Jurkat cell line expressing a single TCR were co-incubated with mono HLA-I allele K562 to measure TCR downstream signaling expressed as log 2 fold signal of luciferase. K562 were pulsed with a peptide encoded by the tested string (PP) as positive control to control the TCR activity for the specific epitope tested or transfected with a mRNA string encoding for a polypeptide. PHA-L was used as a positive control for TCR activity. 5 unique epitopes were tested to react with their cognate TCRs. Four different mRNA strings, with distinct amino acid sequences have been tested each with 2 nucleotide sequences optimization (nt2 and nt3). String1 and its derivative string1.1 (4 point mutations) have been tested by transfecting cells with either 3 μg (FIG. 10A) or 0.3 μg (FIG. 10B) of methylpseudo-uridine mRNA in K562 cells. All epitopes displayed were a perfect match in the sequence of these mRNA strings. String2 and its derivative string2.1 (1 point mutation) have been tested by transfected cells with either 3 μg (FIG. 10C) or 0.3 μg (FIG. 10D) of methylpseudo-uridine mRNA in K562 cells. Epitopes marked with a star (*) contain at least one polymorphism compared to the sequence of these mRNA strings.

String1 and its derivative string1.1 (including 4 point mutations), encodes all five tested epitopes with a perfect sequence match. The epitopes AK11, DA9 and IW9 could be detected with 3 μg of transfected mRNA for each string (FIG. 10A). Sequence optimization of string1a and 2a allowed the supplementary engagement of the TCR detecting the KF11 epitopes, despite a high untreated background. The EL9 epitope was consistently not detected in that assay, which is not due to a lack of TCR reactivity, as functionality can be observer in both a specific PHA-L and specific peptide pulsed (PP) conditions. Using a suboptimal mRNA transfection of 0.3 μg lead to the loss of signal for AK11, KF11 and DA9 for all or some of the strings. This observation is expected to be linked to lower total encoded string polyprotein translation. As such, strongest responses with 3 μg (FIG. 10A—IW9, DA9) could still be detected with 0.3 μg transfection. String2 and its derivative string2.1 (1 point mutation), encodes for polymorphic versions of 3 of the epitopes tested. Only IW9 and KF11 epitopes have a perfect sequence match with the strings 2 and 2.1. Hence, AK11, EL9 are not detected in this assay (FIGS. 10C and D). However, the polymorphic version of DA9 encoded in these set of mRNA strings appears to be cross-reactive and engages the TCR efficiently with 3 μg of mRNA (FIG. 10C) but not sufficiently to be detected with only 0.3 μg (FIG. 10D). Consistently with the previous observation with String1 and 2, IW9 triggers a strong responses at 3 μg (FIG. 10C) than can still be detected with 0.3 μg (FIG. 10D). While only some codon-optimized sequences, String2 nt2 and string2.1 nt3, appear to produce a detectable response at 3 μg with the TCR detecting KF11.

Legend:
AK11: ACQGVGGPGHK;
DA9: DRFYKTLRA;
KF11: KAFSPEVIPMF;
IW9: ISPRTLNAW;
EL9: EVIPMFSAL,
Polymorphic DA9 in string2 and string2.1:
DRFFKTLRA
(referenced as +DA9+ in FIG. 6).

### Example 10

TCR Engagement from Epitopes Processed and Presented from Different mRNA Strings in the K562 Cell Line in iDCs Generated from 2 Donors:

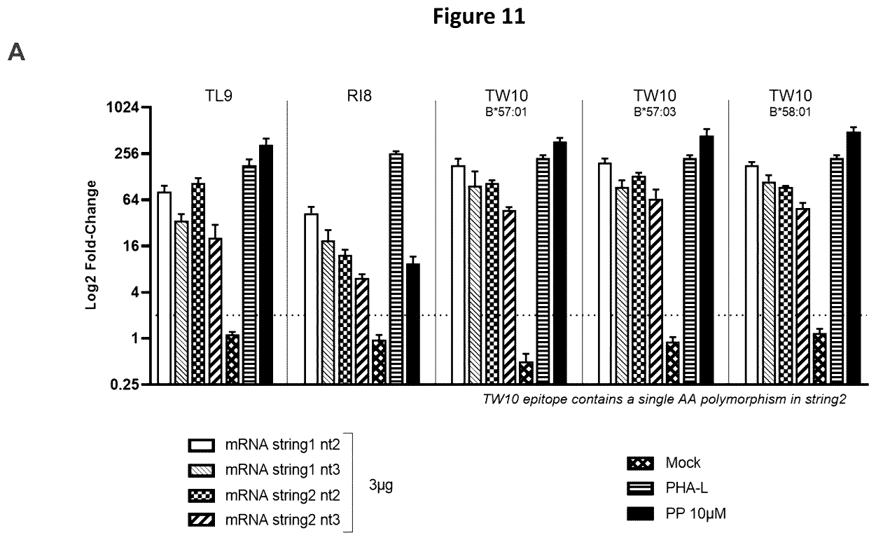

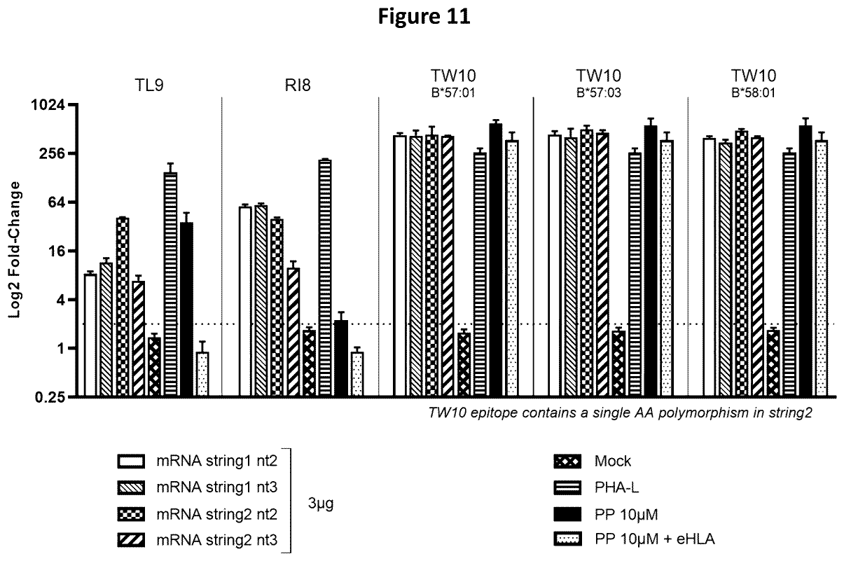

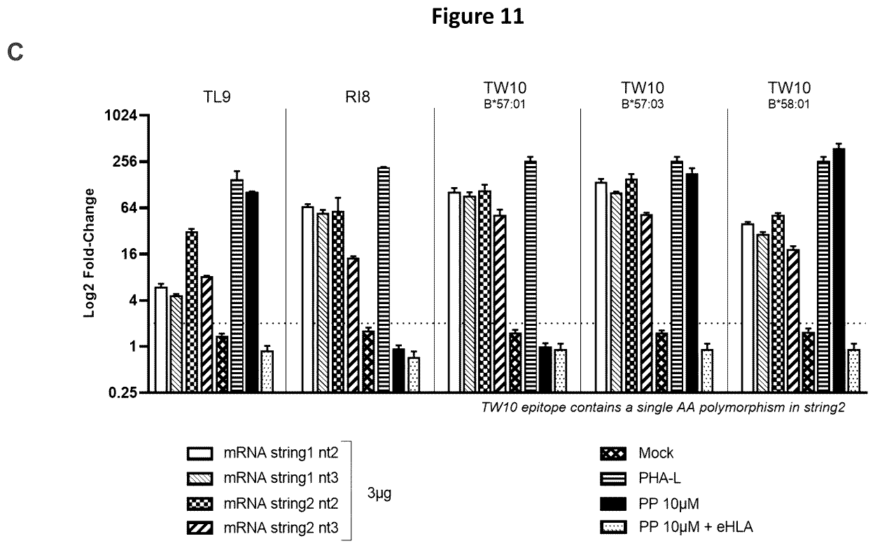

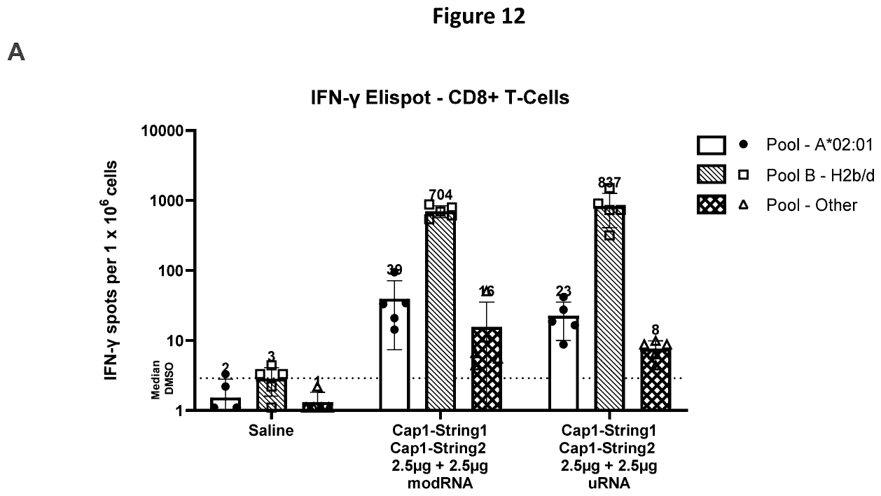

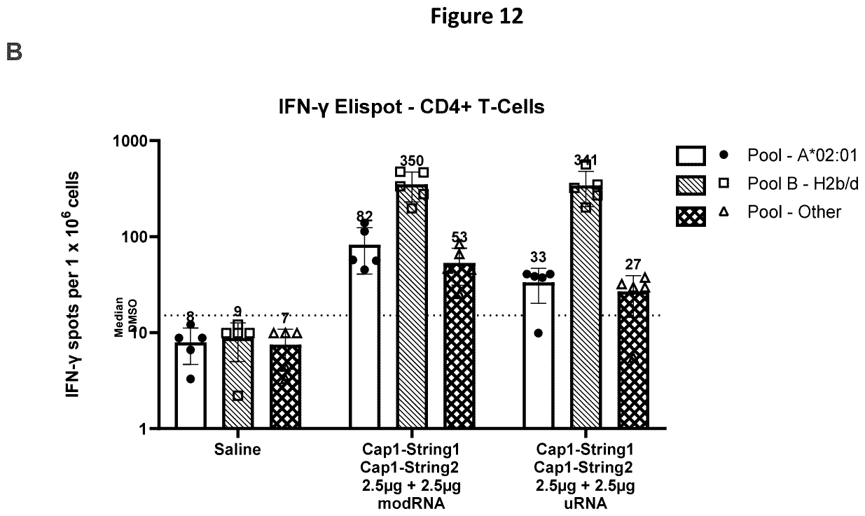

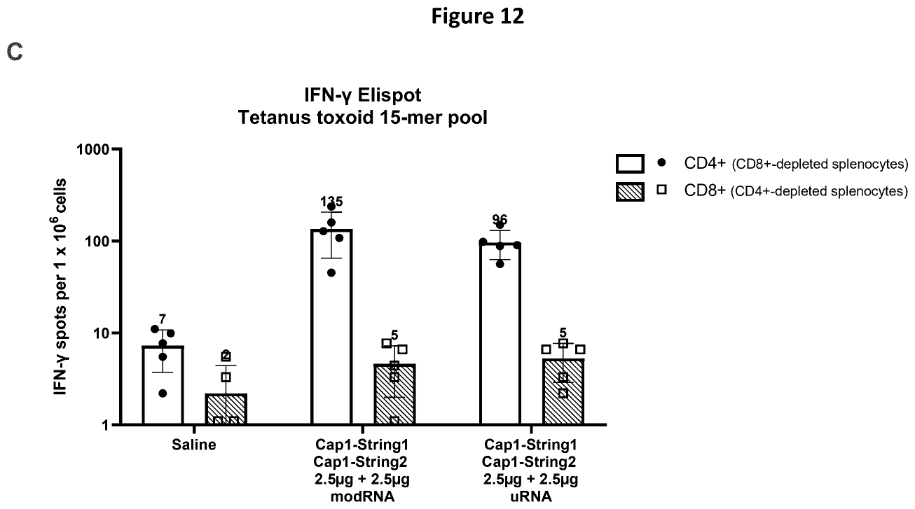

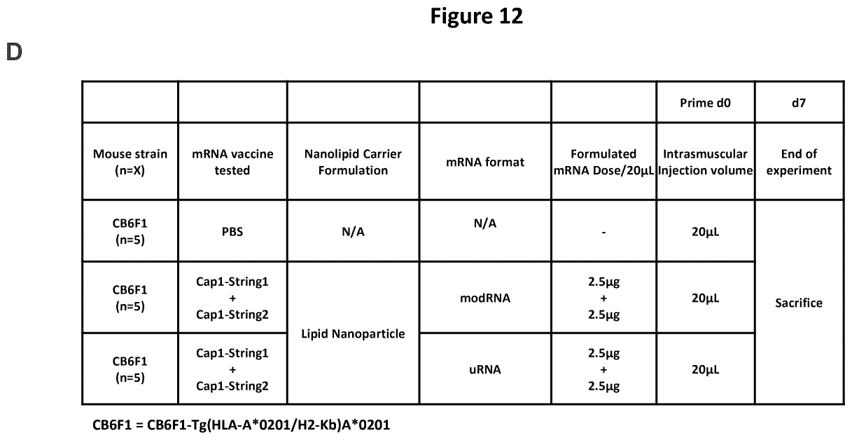

Effector Jurkat cell lines expressing a single TCR were co-incubated with Target mono HLA-I allele K562 or HLA-I transfected iDCs from donors to measure TCR downstream signaling expressed as log 2 fold signal of luciferase. Target cells were pulsed with a peptide encoded by the tested string (PP) as positive control to control the TCR activity for the specific epitope tested or transfected with an mRNA string encoding for a polypeptide. Target iDCs were also tested with pulsed peptide but without co-transfection of HLA alleles to control for background presentation by the endogenous HLA-I from the donor (PP 10 μM+eHLA). PHA-L was used as a positive control for TCR activity. 3 unique epitopes were tested to react with their cognate TCRs. Four different mRNA strings, with distinct amino acid sequences, were tested each with 2 nucleotide sequence optimizations (nt2 and nt3). String1 and string2 were tested by transfecting cells with either 3 μg of methylpseudo-uridine mRNA in the Target K562 cells (FIG. 11A) or in Target iDCs from 2 donors (FIGS. 11B and C). The tested TL9 and RI8 epitopes were both encoded by string1 and string2 with a perfect sequence match. In parallel, the epitope TW10 encoded by String2 contained a single mutation polymorphism, while being a perfect match for String1. TL9 was tested using the HLA allele B*39:10, E18 was tested using the HLA allele B*52:01 and TW10 was tested with the HLA alleles B*57:01, B*57:03 and B*58:01.

String1 encodes all three tested epitopes with a perfect sequence match. In comparison, string2 only encodes the epitopes TL9 and RI8 with a perfect match, but contains a single amino acid mutation in the epitope TW10. Both String1 and String2 were tested with different nucleotide optimization (nt2 and nt3). Each of the epitopes tested were assessed for cleavage, processing and presentation on their cognate HLA from the polypeptide translated from string1 and string2 mRNA templates. Functionality of all tested TCR clones was tested with a selective T-cell mitogen (PHA-L) (FIGS. 11A, B and C). Specificity of the TCR clones for their epitope:HLA-I complex was validated using single expressing HLA-I allele and peptide pulsing (PP) (FIG. 11A). Of note, the epitope TW10 was able to engage the TCR clone when presented by HLA-I alleles B*57:01, B*57:03 and B*58:01. String1 and 2 consistently engaged the cognate TCR of TL9 and RI8 in K562 cell line and iDCs generated from two independent donors (FIGS. 11A, B and C). The HLA-I haplotype background of the two donors did not contain a HLA-I allele able to present and engage these two epitopes when iDCs were tested with pulsed peptide without supplementary exogenous expression of HLA-I (PP 10 μM+eHLA) (FIGS. 11B and C). Similarly, string1 and 2 consistently engaged the cognate TCR of TW10 in the K562 cell line and iDCs generated from two independent donors with all three tested HLA-I alleles, with no interferences from the donor HLA-I haplotype (FIGS. 11A and C). However, the second donor appeared to express at least one HLA-I allele in its haplotype able to present TW10 and engage the tested TCR clone.

These datasets confirm that all mRNA-string encoded polypeptides can be enzymatically processed to generate epitopes that are then presented on their cognate HLA class I alleles. The epitope:HLA-I complexes are able, in vitro, to engage TCR downstream signaling. The pre-existing HLA-I able to present TW10 in a donor prevents singling out the HLA-I allele triggering the downstream cascade signaling from the TCR engagement. The data nonetheless confirms that the polypeptide translated from our mRNA string1 and string2 allows for presentation of epitopes to engage T-cells activation. These data also show the impact of the nucleotide sequence optimization on the TCR-mediated response magnitude.

### Example 11

### Methods

### Sample Preparation and Immunization

mRNAs produced by in vitro transcription with an integrity>80% were tested for endotoxin content and formulated in lipid nanoparticles. Each mRNA was formulated alone at a final concentration of 0.5 mg/mL and stored at −80° C. On the day of immunization, particles were thawed and mixed at a 1:1 ratio (string1:string2) to a final injection volume of 20 μL for intramuscular application. CB6F1-Tg(HLA-A*0201/H2-Kb)A*0201 (CB6F1) mice were immunized with a single injection in the left hind gastrocnemius muscle at d0 (using a 1 mL TB syringe from Becton Dickinson 309659). On d7 animals were sacrificed and their spleens recovered for immediate splenocyte processing. CD4+ or CD8+ T cell were depleted from splenocytes to investigate, respectively, CD8+ and CD4+ T cells IFN-γ release via ELISpot. Animals were followed for health monitoring during the entire experiment until sacrifice.

### T Cell Isolation and ELISpot

Freshly recovered splenocytes were subjected to either CD4+ T cell- or CD8+ T cell−depletion (EasySep™ Mouse CD8a Positive Selection Kit II (#18953) and EasySep™ Mouse CD4 Positive Selection Kit II (#18952) from STEM CELL Technologies). Briefly, 20-40 millions splenocytes were incubated with antibody cocktail targeting CD4+ T cells or CD8+ Tcells and incubated at room temperature for 5 mins. The antibody/cell mix was then incubated with magnetic particles (20-25 μl/per sample) for 3 mins at room temperature. Unlabelled splenocytes were separated from the labelled cells via magnetic separation for 10 mins at room temperature. Cells were immediately used for the IFNg-ELISpot assay. 96-well ELISpot plates were blocked with serum-free assay media (X-VIVO+1% Pen-Strep+1% Glutamax) for at least 1 h at 37° C. 100 μL containing 300,000 splenocytes from CB6F1 were pulsed with 100 μL 0.3 uM per peptide or DMSO control for 20 h at 37° C. and incubated overnight on anti-IFNg coated plates (R&D Systems #XEL485). Spots were detected using a biotinylated anti mouse IFNg detection antibody, followed by Streptavidin-Alkaline Phosphatase overnight incubation at 4° C. After plate drying for several days, an ELISpot plate reader (ImmunoSpot® S6 Core Analyzer, CTL) was used to count and analyze spot numbers per well.

CD8+ and CD4+ T-cell responses to two different mRNA strings (string1 and string2) in combination via intramuscular lipid nanoparticle (LNP) nanocarrier.

Elispot data for IFN-γ production from CD8+ T cell-depleted and CD4+ T cell-depleted splenocytes stimulated with peptide pools. Splenocytes were isolated from HLA-A*02:01 CB6F1 transgenic mice and stimulated with peptide pools for 18 hours on Elispot plates coated with anti-IFN-γ antibody. CD4+ T cell-depleted and CD8+ T cell-depleted splenocytes were generated from animal groups (n=5 mice per group) co-immunized with a combination of single LNP-formulated mRNA string1+string2 in a 1:1 ratio (2.5 μg+2.5 μg) or receiving a saline placebo. mRNA combination were tested with string1+string2 as unmodified (uRNA) or as methyl pseudouridine (modRNA). Animals were immunized with a single intramuscular injection and splenocytes were harvested 7 days after injection. CD4+ T cell-depleted (FIG. 12A) or CD8+ T cell-depleted splenocytes (FIG. 12B) from animals immunized with a string1+string2 modRNA or uRNA combination were incubated with 11mer peptide pools covering the vaccine fragments containing at least one known/predicted HIV epitope (prctrk<1%) from A*02:01 (white box—black circle) or H2b/H2d (striped box—white square) or neither (diamond box—white triangle). CD4+ T cell-depleted and CD8+ T cell depleted-splenocytes were incubated with a 15-mer peptide pool spanning the entire tetanus toxin (FIG. 12C). The background IFN-γ signals from CD4+ T cell-depleted and CD8+ T cell depleted-splenocytes incubated with DMSO peptide diluent control is indicated as the median of signals measured across all groups tested in panel (A) or (B)—“Median DMSO”. Quantification of IFNg spot-forming units per 10e6 cells for each peptide pool. FIG. 12D shows a summary of the experimental outline.

LNP intramuscular immunizations of a bivalent combination vaccine (string1+string2) are immunogenic in the CB6F1 models after a single injection. The combination of mRNA string1 and string2, as uRNA or modRNA, elicits robust HIV-specific CD8+ T cell responses restricted by Mouse MHC-I, and a low response for epitope restricted by the human HLA-1 Allele A*02:01 in that mouse model (FIG. 12. A). uRNA and modRNA combinations display similar levels of immunogenicity, indicating that the vaccine strategy is applicable for both mRNA formats using LNP nanocarriers. Similarly, HIV-specific CD4+ T cell responses from CD8+ T cell-depleted splenocytes showed similar low/modest responses restricted by A*02:01 and H2b/d MHC-I alleles for both uRNA and modRNA string combination immunizations (FIG. 12B) further indicating immunogenic properties of the vaccine, but still warranting the addition of the P2P6 tetanus toxin helper epitopes. The use of a P2P16 domain in the sequence to support the HIV CD8+ T cell by engaging non-HIV-specific CD4+ T cells has been validated using a specific 15-mer tetanus toxin peptide pool (FIG. 12C). IFN-γ production from CD8+ T cell-depleted splenocytes indicates a P2P16-specific CD4+ T cells response after immunization. The tetanus toxin (TT) peptide pool did not elicit a detectable level of TT-specific CD8+ T cell responses, limiting the competition between HIV- and TT-specific CD8+ T cells (FIG. 12C).

We claim:

1. A composition comprising an RNA molecule, wherein the RNA molecule comprises an expression cassette encoding an immunogenic peptide, which peptide comprises at least two fragments, wherein each fragment comprises at least one epitope, wherein the epitope is derived from an amino acid sequence encoded by the human immunodeficiency virus (HIV) and wherein the epitope is comprised within or comprises a sequence selected from any one of SEQ ID NOs: 1 to 148 and 198 to 202 or a variant thereof.

2. The composition according to claim 1, wherein at least one of the fragments comprises at least 2, 3, 4, 5, 6, 7, 8, 9, or 10 epitopes.

3. The composition according to claim 1 or 2, wherein the epitopes in one fragment are different from the epitopes in at least one other fragment or in all other fragments.

4. The composition according to any one of claims 1 to 3, wherein at least 60%, 65%, 70% 75%, 80%, 85%, 90%, 95%, 97%, 98%, 99% or all of the fragments do not comprise any epitope that is comprised in another fragment.

5. The composition according to any one of claims 1 to 4, wherein the sequences of the epitopes overlap in at least one of the fragments.

6. The composition according to any one of claims 1 to 5, wherein the sequences of the epitopes do not overlap in at least one of the fragments.

7. The composition according to any one of claims 1 to 6, wherein the peptide comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 35 or 40 fragments in total.

8. The composition according to any one of claims 1 to 7, wherein the peptide comprises at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 66, 67, 68, 69, or 70 epitopes of those depicted in SEQ ID NOs: 1 to 148 and 198 to 202 in total.

9. The composition according to any one of claims 1 to 8, wherein each fragment comprises the same or different number of epitopes.

10. The composition according to any one of claims 1 to 9, wherein the epitopes are combined within the expression cassette so as to minimize the length of the RNA molecule.

11. The composition according to any one of claims 1 to 10, wherein the epitopes are derived from the gp41, gp120, p31, p24, nef, p51, protease, tat, rev, vif, vpr, vpx, or vpu protein of HIV.

12. The composition according to any one of claims 1 to 11, wherein the peptide comprises at least one epitope derived from each of the gp41, gp120 or nef proteins of HIV.

13. The composition according to any one of claims 1 to 12, wherein at least one amino acid separates the sequences of the non-overlapping epitopes and/or wherein at least one amino acid separates at least two fragments.

14. The composition according to any one of claims 1 to 13, wherein two or more non-overlapping epitopes comprised within a fragment or two or more fragments are not flanked 5′ and 3′ by the consensus flanking amino acid sequence of the HIV amino acid sequence for the clade from which the epitopes are derived.

15. The composition according to any one of claims 1 to 14, wherein a linker separates at least two fragments.

16. The composition according to any one of claims 1 to 15, wherein the variant of an epitope is a polymorph of the epitope, wherein the epitope is a consensus sequence from at least two different clones of HIV and/or wherein the variant differs from the epitope in one, two, three, four or five amino acids.

17. The composition according to any one of claims 1 to 16, wherein the composition further comprises a second RNA molecule, wherein the second RNA molecule comprises a second expression cassette encoding a second immunogenic peptide, which peptide comprises at least two fragments, wherein each fragment comprises at least one epitope, wherein the epitope is derived from an amino acid sequence encoded by the human immunodeficiency virus (HIV) and wherein the epitope is comprised within or comprises a sequence selected from any one of SEQ ID NOs: 1 to 148 and 198 to 202 or a variant thereof.

18. The composition according to claim 17, wherein at least one of the fragments of the second immunogenic peptide comprises at least 2, 3, 4, 5, 6, 7, 8, 9, or 10 epitopes.

19. The composition according to claim 17 or 18, wherein the epitopes in one fragment of the second immunogenic peptide are different from the epitopes in at least one other fragment of the second immunogenic peptide or in all other fragments of the second immunogenic peptide.

20. The composition according to any one of claims 17 to 19, wherein at least 60%, 65%, 70%, 75%, 80%, 85%, 90%, 95%, 97%, 98%, 99% or all of the fragments of the second immunogenic peptide do not comprise any epitope that is comprised in another fragment of the second immunogenic peptide.

21. The composition according to any one of claims 17 to 20, wherein the sequences of the epitopes overlap in at least one of the fragments of the second immunogenic peptide.

22. The composition according to any one of claims 17 to 21, wherein the sequences of the epitopes do not overlap in at least one of the fragments of the second immunogenic peptide.

23. The composition according to any one of claims 17 to 22, wherein the of the second immunogenic peptide comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 35 or 40 fragments in total.

24. The composition according to any one of claims 17 to 23, wherein the of the second immunogenic peptide comprises at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 66, 67, 68, 69, or 70 epitopes of those depicted in SEQ ID Nos: 1 to 148 and 198 to 202 in total.

25. The composition according to any one of claims 17 to 24, wherein each fragment of the second immunogenic peptide comprises the same or different number of epitopes.

26. The composition according to any one of claims 17 to 25, wherein the epitopes are combined within the expression cassette of the second immunogenic RNA molecule so as to minimize the length of the RNA molecule.

27. The composition according to any one of claims 17 to 26, wherein the epitopes are derived from the gp41, gp120, p31, p24, nef, p51, protease, tat, rev, vif, vpr, vpx, or vpu protein of HIV.

28. The composition according to any one of claims 17 to 27, wherein the of the second immunogenic peptide comprises at least one epitope derived from each of the gp41, gp120 or nef proteins of HIV.

29. The composition according to any one of claims 17 to 28, wherein two or more non-overlapping epitopes comprised within a fragment of the second immunogenic peptide or two or more fragments of the second immunogenic peptide are not flanked 5′ and 3′ by the consensus flanking amino acid sequence of the HIV amino acid sequence for the clade from which the epitopes are derived.

30. The composition according to any one of claims 17 to 29, wherein at least one amino acid separates the sequences of the non-overlapping epitopes and/or wherein at least one amino acid separates at least two fragments of the second immunogenic peptide.

31. The composition according to any one of claims 17 to 30, wherein a linker separates at least two fragments of the second immunogenic peptide.

32. The composition according to any one of claims 17 to 31, wherein the variant of an epitope is a polymorph of the epitope, wherein the epitope is a consensus sequence from at least two different clones of HIV and/or wherein the variant differs from the epitope in one, two, three, four or five amino acids.

33. The composition according to any one of claims 1 to 32, wherein the immunogenic peptide comprises epitopes derived from one clade of HIV and the second immunogenic peptide comprises epitopes derived from another clade of HIV.

34. The composition according to claim 33, wherein the one clade is clade B and the another clade is clade C.

35. The composition according to claim 33 or 34, wherein each immunogenic peptide comprises epitopes which are identical in both clades.

36. The composition according to any one of claims 33 to 35, wherein each immunogenic peptide comprises polymorphic epitopes from each respective clade.

37. The composition according to any one of claims 1 to 36, wherein the epitope is a T cell epitope.

38. The composition according to any one of claims 1 to 37, wherein the epitope is CD8 minimal epitope.

39. The composition according to any one of claims 1 to 38, wherein the epitope is 9 to 21 amino acids in length.

40. The composition according to any one of claims 1 to 39, wherein the epitope is 9 or more amino acids in length.

41. The composition according to any one of claims 1 to 40, wherein the epitope is 10 or more amino acids in length.

42. The composition according to any one of claims 1 to 41, wherein the epitope is 11 or more amino acids in length.

43. The composition according to any one of claims 1 to 42, wherein the epitope is 9 to 14 amino acids in length.

44. The composition according to any one of claims 1 to 43, wherein the epitope is 9 to 13 amino acids in length.

45. The composition according to any one of claims 1 to 44, wherein the epitope is 9 to 12 amino acids in length.

46. The composition according to any one of claims 1 to 45, wherein the epitope is 9 to 11 amino acids in length.

47. The composition according to any one of claims 1 to 46, wherein the epitope is 9 or 10 amino acids in length.

48. The composition according to any one of claims 1 to 47, wherein the epitope is 9 amino acids in length.

49. The composition according to any one of claims 1 to 48, wherein the fragments range from 9 to 21 amino acids in length.

50. The composition according to any one of claims 1 to 49, wherein the peptide comprises the amino acid sequence of
a) SEQ ID NOs: 60, 103, 98, 149, 2, 150, 142, 86, 1, 141, 151, 152, 130, 153, 154, 155, 156, 157, 158, 4, 159, 160, 161, 162, 35, 28, 163, 164, 165, 166, 87, 167, and 85 or
b) SEQ ID NOs: 60, 103, 98, 149, 2, 150, 142, 86, 1, 141, 151, 152, 199, 153, 154, 155, 156, 157, 158, 4, 159, 160, 161, 162, 35, 28, 203, 164, 165, 166, 87, 167, and 85.

51. The composition according to any one of claims 1 to 49, wherein the peptide comprises the amino acid sequence of
a) SEQ ID NOs: 98, 125, 29, 85, 168, 35, 150, 149, 169, 87, 57, 3, 170, 171, 172, 99, 131, 173, 174, 175, 104, 176, 61, 177, 178, 179, 5, 180, 181, 1, 182, and 140 or
b) SEQ ID NOs: 98, 125, 29, 85, 168, 35, 150, 149, 169, 87, 57, 3, 170, 171, 172, 99, 131, 173, 174, 175, 104, 176, 61, 177, 204, 179, 5, 180, 181, 1, 182, and 140.

52. The composition according to any one of claims 1 to 49, wherein the peptide comprises the amino acid sequence of SEQ ID NO: 183, 185, 187, 189, 205 or 208.

53. The composition according to any one of claims 17 to 49, wherein
the first peptide comprises the amino acid sequence of
a) SEQ ID NOs: 60, 103, 98, 149, 2, 150, 142, 86, 1, 141, 151, 152, 130, 153, 154, 155, 156, 157, 158, 4, 159, 160, 161, 162, 35, 28, 163, 164, 165, 166, 87, 167, and 85 or
b) SEQ ID NOs: 60, 103, 98, 149, 2, 150, 142, 86, 1, 141, 151, 152, 199, 153, 154, 155, 156, 157, 158, 4, 159, 160, 161, 162, 35, 28, 203, 164, 165, 166, 87, 167, and 85,
and the second peptide comprises the amino acid sequence of
c) SEQ ID NOs: 98, 125, 29, 85, 168, 35, 150, 149, 169, 87, 57, 3, 170, 171, 172, 99, 131, 173, 174, 175, 104, 176, 61, 177, 178, 179, 5, 180, 181, 1, 182, and 140 or
d) SEQ ID NOs: 98, 125, 29, 85, 168, 35, 150, 149, 169, 87, 57, 3, 170, 171, 172, 99, 131, 173, 174, 175, 104, 176, 61, 177, 204, 179, 5, 180, 181, 1, 182, and 140.

54. The composition according to any one of claims 17 to 49, wherein
a) the first peptide comprises the amino acid sequence of SEQ ID NO: 183 or 205 and the second peptide comprises the amino acid sequence of 185 or 208, or
b) the first peptide comprises the amino acid sequence of SEQ ID NO: 187 and the second peptide comprises the amino acid sequence of SEQ ID NO: 189,
wherein optionally the first peptide comprises the amino acid of SEQ ID 205 and the second peptide comprises the amino acid of SEQ ID NO: 208.

55. The composition according to any one of claims 1 to 54, wherein the RNA molecule comprises the nucleotide sequence of SEQ ID NO: 184, 186, 188, 190, 206, 207, 209, 210 or 211 to 218.

56. The composition according to any one of claims 1 to 55, wherein the epitope is a non-protective epitope which can lead to CD8+ T cell protection collapse.

57. The composition according to claim 56, wherein protection collapse is T cell escape or T cell exhaustion or loss of CD4+ helper T cells.

58. The composition according to any one of claims 1 to 57, wherein the peptide further comprises sequences which enhance epitope presentation on the surface of a cell.

59. The composition according to claim 58, wherein the cell is an immune cell.

60. The composition according to claim 59, wherein the immune cell is an antigen presenting cell (APC).

61. The composition according to any one of claims 1 to 60, wherein the peptide further comprises a signal peptide.

62. The composition according to any one of claims 1 to 61, wherein the peptide further comprises a MHC class I trafficking signal (MITD).

63. The composition according to any one of claims 1 to 62, wherein the peptide further comprises a non-HIV HLA-II helper epitope.

64. The composition according to claim 63, wherein the helper epitope is the P2 and/or P16 amino acid sequences derived from the tetanus toxoid (TT) of Clostridium tetani.

65. The composition according to any one of claims 1 to 64, wherein the RNA molecule is linear or circular.

66. The composition according to any one of claims 1 to 65, wherein the RNA molecule comprises a 5′ cap.

67. The composition according to claim 66, wherein the 5′ cap is a modified or artificial cap or a cap analog.

68. The composition according to any one of claims 1 to 67, wherein the expression cassette further comprises a 5′ untranslated region (5′ UTR) and/or a 3′ untranslated region (3′ UTR).

69. The composition according to claim 68, wherein the 5′ UTR comprises the nucleotide sequence of SEQ ID NO: 195, or a nucleotide sequence having at least 99%, 98%, 97%, 96%, 95%, 90%, 85%, or 80% identity to the nucleotide sequence of SEQ ID NO: 195.

70. The composition according to claim 69, wherein the 3′ UTR comprises the nucleotide sequence of SEQ ID NO: 196, or a nucleotide sequence having at least 99%, 98%, 97%, 96%, 95%, 90%, 85%, or 80% identity to the nucleotide sequence of SEQ ID NO: 196.

71. The composition according to any one of claims 1 to 70, wherein the expression cassette further comprises a poly A structure.

72. The composition according to claim 71, wherein the poly A structure is an interrupted poly A structure.

73. The composition according to claim 72, wherein the poly A structure comprises the nucleotide sequence of SEQ ID NO: 197, or a nucleotide sequence having at least 99%, 98%, 97%, 96%, 95%, 90%, 85%, or 80% identity to the nucleotide sequence of SEQ ID NO: 197.

74. The composition according to any one of claims 1 to 73, wherein the RNA molecule is a replicable RNA molecule.

75. The composition according to claim 74, wherein the replicable RNA molecule further encodes an RNA-dependent RNA polymerase (replicase) which is able to replicate the replicable RNA molecule.

76. The composition according to claim 74, wherein the replicable RNA molecule does not encode an RNA-dependent RNA polymerase (replicase).

77. The composition according to any one of claims 74 to 76, wherein the composition further comprises a non-replicable RNA molecule that encodes an RNA-dependent RNA polymerase (replicase) which is able to replicate the replicable RNA molecule.

78. The composition according to any one of claims 1 to 77, wherein the RNA molecule is non-immunogenic.

79. The composition according to claim 78, wherein the RNA molecule is made non-immunogenic by removal of double-stranded RNA.

80. The composition according to any one of claims 1 to 79, wherein the RNA molecule comprises a nucleotide modification.

81. The composition according to claim 80, wherein the modification is the substitution of one or more U residues with pseudouridine, N1-methylpseudoruridine or 5-methyluridine.

82. The composition according to claim 81, wherein the one or more substituted U residues is N1-methylpseudouridine.

83. The composition according to claim 80 or 81, wherein at least 50%, at least 70%, at least 90% at least 99% or 100% of the U residues in the RNA molecule are substituted.

84. The composition according to any one of claims 1 to 83, wherein the RNA molecule is formulated in the composition with at least one lipid.

85. The composition according to claim 84, wherein the RNA molecule and at least one lipid form particles.

86. The composition according to claim 85, wherein the particles are lipid nanoparticles (LNP), or lipoplexes (LPX) or liposomes.

87. The composition according to claim 85 or 86, wherein the particles are nanoparticles, in which:
(i) the number of positive charges in the nanoparticles does not exceed the number of negative charges in the nanoparticles, and/or
(ii) the nanoparticles have a net negative charge, and/or
(iii) the zeta potential of the nanoparticles is 0 or less.

88. The composition according to claim 87, wherein the charge ratio of positive charges to negative charges in the nanoparticles is between 1:1 and 1:8, preferably between 1:1 and 1:4.

89. The composition according to any one of claims 84 to 88, wherein the at least one lipid is a cationic lipid.

90. The composition according to claim 89, wherein the lipid comprises a cationic head group.

91. The composition according to claim 84 to 90, wherein the lipid is a pH responsive lipid.

92. The composition according to any one of claims 84 to 91, wherein the at least one lipid is a PEGylated-lipid.

93. The composition according to any one of claims 84 to 92, wherein the composition further comprises at least one helper lipid.

94. The composition according to claim 93, wherein the helper lipid is a neutral lipid.

95. The composition according to any one of claim 89 or 90, wherein the at least one cationic lipid comprises 1,2-di-O-octadecenyl-3-trimethylammonium propane (DOTMA), 1,2-dioleyloxy-3-dimethylaminopropane (DODMA), and/or 1,2-dioleoyl-3-trimethylammonium-propane (DOTAP).

96. The composition according to any one of claim 93 or 94, wherein the at least one helper lipid comprises 1,2-di-(9Z-octadecenoyl)-sn-glycero-3-phosphoethanolamine (DOPE), cholesterol (Chol), 1,2-dioleoyl-sn-glycero-3-phosphocholine (DOPC), and/or 1,2-distearoyl-sn-glycero-3-phosphocholine (DSPC).

97. The composition according to claim 93 or 94, wherein the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1.

98. The composition according to any one of claims 87 to 97, wherein the nanoparticles are lipoplexes comprising DODMA and DOPE in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2.

99. The composition according to any one of claims 87 to 97, wherein the nanoparticles are lipoplexes comprising DODMA and Cholesterol in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2.

100. The composition according to any one of claims 87 to 97, wherein the nanoparticles are lipoplexes comprising DODMA and DSPC in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2.

101. The composition according to any one of claims 87 to 97, wherein the nanoparticles are lipoplexes comprising DODMA:Cholesterol:DOPE:PEGcerC16 in a molar ratio of 40:48:10:2.

102. The composition according to any one of claims 87 to 97, wherein the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

103. The composition according to any one of claims 87 to 97, wherein the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

104. The composition according to any one of claims 87 to 97, wherein the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

105. The composition according to claim 85 or 86, wherein the particles are LNPs, which are complexed with and/or encapsulate the RNA molecule.

106. The composition according to claim 85 or 86, wherein the particles are vesicles encapsulating the RNA molecule, preferably unilamellar liposomes.

107. The composition according to any one of claims 1 to 84, wherein the RNA molecule(s) is formulated in a composition comprising a polyalkyleneimine, preferably is a polyalkyleneimine.

108. The composition according to claim 107, wherein the molar ratio of the number of nitrogen atoms (N) in the polyalkyleneimine to the number of phosphor atoms (P) in the RNA molecules (N:P ratio) is 2.0 to 15.0, preferably 6.0 to 12.0.

109. The composition according to claim 107 or 108, wherein the ionic strength of the composition is 50 mM or less, preferably wherein the concentration of monovalent cationic ions is 25 mM or less and the concentration of divalent cationic ions is 20 μM or less.

110. The composition according to claim 107 to 109, wherein the particles formed are polyplexes.

111. The composition according to any one of claims 107 to 110, wherein the polyalkyleneimine comprises the following general formula (I): 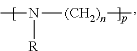
wherein
R is H, an acyl group or a group comprising the following general formula (II): 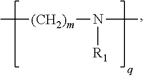
wherein R1 is H or a group comprising the following general formula (III): 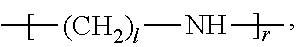
n, m, and l are independently selected from integers from 2 to 10; and
p, q, and r are integers, wherein the sum of p, q, and r is such that the average molecular weight of the polymer is 1.5.102 to 107 Da, preferably 5000 to 105 Da, more preferably 10000 to 40000 Da, more preferably 15000 to 30000 Da, even more preferably 20000 to 25000 Da.

112. The composition according to any one of claims 107 to 111, wherein the polyalkyleneimine comprises polyethylenimine and/or polypropylenimine, preferably polyethyleneimine.

113. The composition according to any one of claims 107 to 112, wherein at least 92% of the N atoms in the polyalkyleneimine are protonatable.

114. The composition according to any one of claims 1 to 113, wherein the composition is a pharmaceutical composition.

115. The composition according to claim 114, wherein the composition further comprises a pharmaceutically acceptable carrier or excipient.

116. The composition according to any one of claims 1 to 115, wherein the composition is in the form of a dry powder.

117. The composition according to any one of claims 1 to 115, wherein the composition is lyophilized.

118. The composition according to any one of claims 1 to 115, wherein the composition is frozen.

119. The composition according to claim 118, wherein the composition has a temperature of −20° C. or lower.

120. The composition according to any one of claims 1 to 119 further comprising one or more additives, wherein the additives optionally are selected from the group consisting of buffering substances, saccharides, stabilizers, cryoprotectants, lyoprotectants, and chelating agents.

121. The composition according to claim 120, wherein the buffering substances comprise at least one selected from the group consisting of 4-(2-hydroxyethyl)-1-piperazineethanesulfonic acid (HEPES), 2-(N-morpholino)ethanesulfonic acid (MES), 3-morpholino-2-hydroxypropanesulfonic acid (MOPSO), acetic acid, acetate buffers and analogues, phosphoric acid and phosphate buffers, and citric acid and citrate buffers.

122. The composition according to claim 120 or 121, wherein the saccharides comprise at least one selected from the group consisting of monosaccharides, disaccharides, trisaccharides, oligosaccharides, and polysaccharides preferably from glucose, trehalose, and saccharose.

123. The composition according to any one of claims 120 to 122, wherein the cryoprotectants comprise at least one selected from the group consisting of glycols, such as ethylene glycol, propylene glycol, and glycerol.

124. The composition according to any one of claims 120 to 123, wherein the chelating agent comprises EDTA.

125. The composition according to any one of claims 1 to 124, wherein the composition is a vaccine.

126. A medical preparation comprising the composition according to any one of claims 1 to 125.

127. A kit comprising one or more RNA molecules defined in any one of claims 1 to 58.

128. The kit according to claim 127, wherein the RNA molecule is in the form of a dry powder composition.

129. The kit according to claim 127 or 128, wherein the RNA molecules are lyophilized.

130. The kit according to any one of claims 127 to 129, wherein the kit further comprises instructions for administering the RNA molecules.

131. A method for preventing HIV infection in a subject, said method comprising administering the composition according to any one of claims 1 to 125 to the subject.

132. A method for preventing HIV infection in a subject, said method comprising dissolving the composition according to 116 into a suitable liquid pharmaceutical solution forming a solution for administration and administering the solution for administration to the subject.

133. A method for treating HIV infection in a HIV-positive subject, said method comprising administering the composition according to any one of claims 1 to 125 to the subject.

134. A method for treating HIV infection in a HIV-positive subject, said method comprising dissolving the composition according to 116 into a suitable liquid pharmaceutical solution forming a solution for administration and administering the solution for administration to the subject.

135. The method according to any one of claims 131 to 134, wherein the severity of one or more symptoms of the HIV infection is reduced.

136. The method according to any one of claims 131 to 135, wherein the method involves only a single administration of the composition.

137. The method according to any one of claims 131 to 135, wherein the method comprises multiple administrations of the composition.

138. The method according to anyone of claims 131 to 137, further comprising administering a booster dose of the composition.

139. The method according to any one of claims 131 to 138, wherein administering the composition comprises intradermal, subcutaneous, or intramuscular administration, such as by intradermal, subcutaneous or intramuscular injection.

140. The method according to claim 139, wherein the injection is by use of a needle or is by use of a needleless injection device.

141. The method according to any one of claims 131 to 139, wherein administering comprises administration by intramuscular injection, preferably with a needle.

142. A composition according to any one of claims 1 to 125 for use in a method for preventing or treating HIV infection in a subject, said method comprising administering the composition to the subject.

143. The composition for use according to claim 142, wherein the subject is HIV-positive.

144. A composition according to any one of claims 1 to 125 for use in the manufacture of a medicament to prevent or treat HIV infection in a subject.

145. A method for treating or preventing HIV infection comprising administering a first RNA molecule and a second RNA molecule to a subject,
wherein the first RNA molecule comprises a nucleotide sequence encoding a first peptide, which first peptide comprises the amino acid sequences of
a) SEQ ID NOs: 60, 103, 98, 149, 2, 150, 142, 86, 1, 141, 151, 152, 130, 153, 154, 155, 156, 157, 158, 4, 159, 160, 161, 162, 35, 28, 163, 164, 165, 166, 87, 167, and 85 or
b) SEQ ID NOs: 60, 103, 98, 149, 2, 150, 142, 86, 1, 141, 151, 152, 199, 153, 154, 155, 156, 157, 158, 4, 159, 160, 161, 162, 35, 28, 203, 164, 165, 166, 87, 167, and 85; and
wherein the second RNA molecule comprises a nucleotide sequence encoding a second peptide, which second peptide comprises the amino acid sequences of
c) SEQ ID NOs: 98, 125, 29, 85, 168, 35, 150, 149, 169, 87, 57, 3, 170, 171, 172, 99, 131, 173, 174, 175, 104, 176, 61, 177, 178, 179, 5, 180, 181, 1, 182, and 140 or
d) SEQ ID NOs: 98, 125, 29, 85, 168, 35, 150, 149, 169, 87, 57, 3, 170, 171, 172, 99, 131, 173, 174, 175, 104, 176, 61, 177, 204, 179, 5, 180, 181, 1, 182, and 140.

146. The method according to claim 145, wherein
a) the first RNA molecule comprises the nucleotide sequence of SEQ ID NO: 184, 211, 212, 206 or 207 and the second RNA molecule comprises the nucleotide sequence of SEQ ID NO: 186, 213, 214, 209 or 210, or
b) the first RNA molecule comprises the nucleotide sequence of SEQ ID NO: 188, 215 or 216, and the second RNA molecule comprises the nucleotide sequence of SEQ ID NO: 190, 217 or 218.

147. The method according to claim 146, wherein the first RNA molecule comprises the nucleotide sequence of SEQ ID NO: 206 or 207, and the second RNA molecule comprises the nucleotide sequence of SEQ ID NO: 209 or 210.

148. The method according to claim 145-147, wherein the first and second RNA molecules are administered at least 2 weeks apart.

149. The method according to any one of claims 145 to 148, wherein the 5′ to 3′ order of the amino acid sequences in the first and/or second peptide is the order given or wherein the 5′ to 3′ order of the amino acid sequences in the first and/or second peptides is different from the order given.


SEQUENCE LISTING (246 sequences)

SEQ ID NO: 1 - protein, 9 aa, Human immunodeficiency virus 1
KLTPLCVTL

SEQ ID NO: 2 - protein, 9 aa, Human immunodeficiency virus 1
SFNCGGEFF

SEQ ID NO: 3 - protein, 9 aa, Human immunodeficiency virus 1
SFNCRGEFF

SEQ ID NO: 4 - protein, 9 aa, Human immunodeficiency virus 1
RAIEAQQHL

SEQ ID NO: 5 - protein, 9 aa, Human immunodeficiency virus 1
RAIEAQQHM

SEQ ID NO: 6 - protein, 10 aa, Human immunodeficiency virus 1
GQMVHQAISP

SEQ ID NO: 7 - protein, 11 aa, Human immunodeficiency virus 1
GQMVHQAISPR

SEQ ID NO: 8 - protein, 10 aa, Human immunodeficiency virus 1
VHQAISPRTL

SEQ ID NO: 9 - protein, 9 aa, Human immunodeficiency virus 1
HQAISPRTL

SEQ ID NO: 10 - protein, 11 aa, Human immunodeficiency virus 1
QAISPRTLNAW

SEQ ID NO: 11 - protein, 9 aa, Human immunodeficiency virus 1
ISPRTLNAW

SEQ ID NO: 12 - protein, 9 aa, Human immunodeficiency virus 1
SPRTLNAWV

SEQ ID NO: 13 - protein, 9 aa, Human immunodeficiency virus 1
VKVVEEKAF

SEQ ID NO: 14 - protein, 9 aa, Human immunodeficiency virus 1
VKVIEEKAF

SEQ ID NO: 15 - protein, 9 aa, Human immunodeficiency virus 1
EEKAFSPEV

SEQ ID NO: 16 - protein, 10 aa, Human immunodeficiency virus 1
KAFSPEVIPM

SEQ ID NO: 17 - protein, 11 aa, Human immunodeficiency virus 1
KAFSPEVIPMF

SEQ ID NO: 18 - protein, 9 aa, Human immunodeficiency virus 1
EVIPMFSAL

SEQ ID NO: 19 - protein, 9 aa, Human immunodeficiency virus 1
EVIPMFTAL

SEQ ID NO: 20 - protein, 8 aa, Human immunodeficiency virus 1
VIPMFSAL

SEQ ID NO: 21 - protein, 8 aa, Human immunodeficiency virus 1
VIPMFTAL

SEQ ID NO: 22 - protein, 9 aa, Human immunodeficiency virus 1
SALSEGATP

SEQ ID NO: 23 - protein, 9 aa, Human immunodeficiency virus 1
TALSEGATP

SEQ ID NO: 24 - protein, 9 aa, Human immunodeficiency virus 1
SEGATPQDL

SEQ ID NO: 25 - protein, 12 aa, Human immunodeficiency virus 1
ATPQDLNTMLNT

SEQ ID NO: 26 - protein, 9 aa, Human immunodeficiency virus 1
TPQDLNTML

SEQ ID NO: 27 - protein, 9 aa, Human immunodeficiency virus 1
GHQAAMQML

SEQ ID NO: 28 - protein, 10 aa, Human immunodeficiency virus 1
TSTLQEQIGW

SEQ ID NO: 29 - protein, 10 aa, Human immunodeficiency virus 1
TSTLQEQIAW

SEQ ID NO: 30 - protein, 8 aa, Human immunodeficiency virus 1
EIYKRWII

SEQ ID NO: 31 - protein, 9 aa, Human immunodeficiency virus 1
DIYKRWIIL

SEQ ID NO: 32 - protein, 9 aa, Human immunodeficiency virus 1
EIYKRWIIL

SEQ ID NO: 33 - protein, 9 aa, Human immunodeficiency virus 1
DIYKRWIIL

SEQ ID NO: 34 - protein, 10 aa, Human immunodeficiency virus 1
KRWIILGLNK

SEQ ID NO: 35 - protein, 9 aa, Human immunodeficiency virus 1
GLNKIVRMY

SEQ ID NO: 36 - protein, 9 aa, Human immunodeficiency virus 1
VRMYSPTSI

SEQ ID NO: 37 - protein, 9 aa, Human immunodeficiency virus 1
VRMYSPVSI

SEQ ID NO: 38 - protein, 8 aa, Human immunodeficiency virus 1
RMYSPTSI

SEQ ID NO: 39 - protein, 8 aa, Human immunodeficiency virus 1
RMYSPVSI

SEQ ID NO: 40 - protein, 9 aa, Human immunodeficiency virus 1
RMYSPTSIL

SEQ ID NO: 41 - protein, 9 aa, Human immunodeficiency virus 1
RMYSPVSIL

SEQ ID NO: 42 - protein, 9 aa, Human immunodeficiency virus 1
YSPTSILDI

SEQ ID NO: 43 - protein, 9 aa, Human immunodeficiency virus 1
YSPVSILDI

SEQ ID NO: 44 - protein, 9 aa, Human immunodeficiency virus 1
FRDYVDRFY

SEQ ID NO: 45 - protein, 9 aa, Human immunodeficiency virus 1
FRDYVDRFF

SEQ ID NO: 46 - protein, 11 aa, Human immunodeficiency virus 1
RDYVDRFYKTL

SEQ ID NO: 47 - protein, 11 aa, Human immunodeficiency virus 1
RDYVDRFFKTL

SEQ ID NO: 48 - protein, 11 aa, Human immunodeficiency virus 1
DYVDRFYKTLR

SEQ ID NO: 49 - protein, 11 aa, Human immunodeficiency virus 1
DYVDRFFKTLR

SEQ ID NO: 50 - protein, 9 aa, Human immunodeficiency virus 1
YVDRFYKTL

SEQ ID NO: 51 - protein, 9 aa, Human immunodeficiency virus 1
YVDRFFKTL

SEQ ID NO: 52 - protein, 9 aa, Human immunodeficiency virus 1
DRFYKTLRA

SEQ ID NO: 53 - protein, 9 aa, Human immunodeficiency virus 1
DRFFKTLRA

SEQ ID NO: 54 - protein, 11 aa, Human immunodeficiency virus 1
AEQASQEVKNW

SEQ ID NO: 55 - protein, 11 aa, Human immunodeficiency virus 1
AEQATQDVKNW

SEQ ID NO: 56 - protein, 12 aa, Human immunodeficiency virus 1
AEQASQEVKNWM

SEQ ID NO: 57 - protein, 12 aa, Human immunodeficiency virus 1
AEQATQDVKNWM

SEQ ID NO: 58 - protein, 9 aa, Human immunodeficiency virus 1
QASQEVKNW

SEQ ID NO: 59 - protein, 9 aa, Human immunodeficiency virus 1
QATQDVKNW

SEQ ID NO: 60 - protein, 9 aa, Human immunodeficiency virus 1
DCKTILKAL

SEQ ID NO: 61 - protein, 9 aa, Human immunodeficiency virus 1
DCKTILRAL

SEQ ID NO: 62 - protein, 11 aa, Human immunodeficiency virus 1
ACQGVGGPGHK

SEQ ID NO: 63 - protein, 9 aa, Human immunodeficiency virus 1
GPGHKARVL

SEQ ID NO: 64 - protein, 9 aa, Human immunodeficiency virus 1
HTQGYFPDW

SEQ ID NO: 65 - protein, 9 aa, Human immunodeficiency virus 1
HTQGFFPDW

SEQ ID NO: 66 - protein, 11 aa, Human immunodeficiency virus 1
TQGYFPDWQNY

SEQ ID NO: 67 - protein, 11 aa, Human immunodeficiency virus 1
TQGFFPDWQNY

SEQ ID NO: 68 - protein, 8 aa, Human immunodeficiency virus 1
YFPDWQNY

SEQ ID NO: 69 - protein, 8 aa, Human immunodeficiency virus 1
FFPDWQNY

SEQ ID NO: 70 - protein, 9 aa, Human immunodeficiency virus 1
YFPDWQNYT

SEQ ID NO: 71 - protein, 9 aa, Human immunodeficiency virus 1
FFPDWQNYT

SEQ ID NO: 72 - protein, 9 aa, Human immunodeficiency virus 1
YTPGPGIRY

SEQ ID NO: 73 - protein, 9 aa, Human immunodeficiency virus 1
YTPGPGVRY

SEQ ID NO: 74 - protein, 10 aa, Human immunodeficiency virus 1
TPGPGIRYPL

SEQ ID NO: 75 - protein, 10 aa, Human immunodeficiency virus 1
TPGPGVRYPL

SEQ ID NO: 76 - protein, 9 aa, Human immunodeficiency virus 1
IRYPLTFGW

SEQ ID NO: 77 - protein, 9 aa, Human immunodeficiency virus 1
VRYPLTFGW

SEQ ID NO: 78 - protein, 8 aa, Human immunodeficiency virus 1
RYPLTFGW

SEQ ID NO: 79 - protein, 10 aa, Human immunodeficiency virus 1
RYPLTFGWCF

SEQ ID NO: 80 - protein, 9 aa, Human immunodeficiency virus 1
YPLTFGWCF

SEQ ID NO: 81 - protein, 9 aa, Human immunodeficiency virus 1
LTFGWCFKL

SEQ ID NO: 82 - protein, 9 aa, Human immunodeficiency virus 1
LVGPTPVNI

SEQ ID NO: 83 - protein, 11 aa, Human immunodeficiency virus 1
TPVNIIGRNLL

SEQ ID NO: 84 - protein, 11 aa, Human immunodeficiency virus 1
TPVNIIGRNML

SEQ ID NO: 85 - protein, 9 aa, Human immunodeficiency virus 1
GIPHPAGLK

SEQ ID NO: 86 - protein, 8 aa, Human immunodeficiency virus 1
TAFTIPSI

SEQ ID NO: 87 - protein, 11 aa, Human immunodeficiency virus 1
TPGIRYQYNVL

SEQ ID NO: 88 - protein, 8 aa, Human immunodeficiency virus 1
IRYQYNVL

SEQ ID NO: 89 - protein, 9 aa, Human immunodeficiency virus 1
KQNPDIVIY

SEQ ID NO: 90 - protein, 9 aa, Human immunodeficiency virus 1
AQNPEIVIY

SEQ ID NO: 91 - protein, 9 aa, Human immunodeficiency virus 1
VIYQYMDDL

SEQ ID NO: 92 - protein, 10 aa, Human immunodeficiency virus 1
PIQKETWEAW

SEQ ID NO: 93 - protein, 10 aa, Human immunodeficiency virus 1
PIQKETWETW

SEQ ID NO: 94 - protein, 10 aa, Human immunodeficiency virus 1
TWEAWWTEYW

SEQ ID NO: 95 - protein, 10 aa, Human immunodeficiency virus 1
TWETWWTDYW

SEQ ID NO: 96 - protein, 9 aa, Human immunodeficiency virus 1
WEAWWTEYW

SEQ ID NO: 97 - protein, 9 aa, Human immunodeficiency virus 1
WETWWTDYW

SEQ ID NO: 98 - protein, 9 aa, Human immunodeficiency virus 1
IVTDSQYAL

SEQ ID NO: 99 - protein, 10 aa, Human immunodeficiency virus 1
FKRKGGIGGY

SEQ ID NO: 100 - protein, 9 aa, Human immunodeficiency virus 1
KRKGGIGGY

SEQ ID NO: 101 - protein, 8 aa, Human immunodeficiency virus 1
GERIVDII

SEQ ID NO: 102 - protein, 8 aa, Human immunodeficiency virus 1
GERIIDII

SEQ ID NO: 103 - protein, 9 aa, Human immunodeficiency virus 1
GERIVDIIA

SEQ ID NO: 104 - protein, 9 aa, Human immunodeficiency virus 1
GERIIDIIA

SEQ ID NO: 105 - protein, 10 aa, Human immunodeficiency virus 1
GQMVHQALSP

SEQ ID NO: 106 - protein, 10 aa, Human immunodeficiency virus 1
GQMVHQPLSP

SEQ ID NO: 107 - protein, 10 aa, Human immunodeficiency virus 1
GQMVHQPISP

SEQ ID NO: 108 - protein, 11 aa, Human immunodeficiency virus 1
GQMVHQALSPR

SEQ ID NO: 109 - protein, 11 aa, Human immunodeficiency virus 1
GQMVHQPLSPR

SEQ ID NO: 110 - protein, 11 aa, Human immunodeficiency virus 1
GQMVHQPISPR

SEQ ID NO: 111 - protein, 10 aa, Human immunodeficiency virus 1
VHQALSPRTL

SEQ ID NO: 112 - protein, 10 aa, Human immunodeficiency virus 1
VHQPLSPRTL

SEQ ID NO: 113 - protein, 10 aa, Human immunodeficiency virus 1
VHQPISPRTL

SEQ ID NO: 114 - protein, 9 aa, Human immunodeficiency virus 1
HQALSPRTL

SEQ ID NO: 115 - protein, 9 aa, Human immunodeficiency virus 1
HQPLSPRTL

SEQ ID NO: 116 - protein, 9 aa, Human immunodeficiency virus 1
HQPISPRTL

SEQ ID NO: 117 - protein, 11 aa, Human immunodeficiency virus 1
QALSPRTLNAW

SEQ ID NO: 118 - protein, 11 aa, Human immunodeficiency virus 1
QPLSPRTLNAW

SEQ ID NO: 119 - protein, 11 aa, Human immunodeficiency virus 1
QPISPRTLNAW

SEQ ID NO: 120 - protein, 9 aa, Human immunodeficiency virus 1
LSPRTLNAW

SEQ ID NO: 121 - protein, 9 aa, Human immunodeficiency virus 1
LSPRTLNAW

SEQ ID NO: 122 - protein, 11 aa, Human immunodeficiency virus 1
AEQASQDVKNW

SEQ ID NO: 123 - protein, 11 aa, Human immunodeficiency virus 1
AEQATQEVKNW

SEQ ID NO: 124 - protein, 12 aa, Human immunodeficiency virus 1
AEQASQDVKNWM

SEQ ID NO: 125 - protein, 12 aa, Human immunodeficiency virus 1
AEQATQEVKNWM

SEQ ID NO: 126 - protein, 10 aa, Human immunodeficiency virus 1
QASQDVKNWM

SEQ ID NO: 127 - protein, 10 aa, Human immunodeficiency virus 1
QATQEVKNWM

SEQ ID NO: 128 - protein, 11 aa, Human immunodeficiency virus 1
ACQGVGGPSHK

SEQ ID NO: 129 - protein, 9 aa, Human immunodeficiency virus 1
GPSHKARVL

SEQ ID NO: 130 - protein, 9 aa, Human immunodeficiency virus 1
NTQGYYPDW

SEQ ID NO: 131 - protein, 9 aa, Human immunodeficiency virus 1
NTQGYFPDW

SEQ ID NO: 132 - protein, 9 aa, Human immunodeficiency virus 1
YTPGPGTRY

SEQ ID NO: 133 - protein, 9 aa, Human immunodeficiency virus 1
YTPGPGVRF

SEQ ID NO: 134 - protein, 10 aa, Human immunodeficiency virus 1
TPGPGTRYPL

SEQ ID NO: 135 - protein, 10 aa, Human immunodeficiency virus 1
TPGPGVRFPL

SEQ ID NO: 136 - protein, 9 aa, Human immunodeficiency virus 1
TRYPLTFGW

SEQ ID NO: 137 - protein, 9 aa, Human immunodeficiency virus 1
VRFPLTFGW

SEQ ID NO: 138 - protein, 8 aa, Human immunodeficiency virus 1
RFPLTFGW

SEQ ID NO: 139 - protein, 9 aa, Human immunodeficiency virus 1
LIGPTPVNI

SEQ ID NO: 140 - protein, 8 aa, Human immunodeficiency virus 1
TAFTIPST

SEQ ID NO: 141 - protein, 8 aa, Human immunodeficiency virus 1
TAFTIPSV

SEQ ID NO: 142 - protein, 9 aa, Human immunodeficiency virus 1
KQNPEIVIY

SEQ ID NO: 143 - protein, 9 aa, Human immunodeficiency virus 1
AKNPEIVIY

SEQ ID NO: 144 - protein, 10 aa, Human immunodeficiency virus 1
PIQKEIWETW

SEQ ID NO: 145 - protein, 10 aa, Human immunodeficiency virus 1
IWETWWTDYW

SEQ ID NO: 146 - protein, 10 aa, Human immunodeficiency virus 1
IWETWWTEYW

SEQ ID NO: 147 - protein, 9 aa, Human immunodeficiency virus 1
WETWWTDYW

SEQ ID NO: 148 - protein, 9 aa, Human immunodeficiency virus 1
WETWWTEYW

SEQ ID NO: 149 - protein, 12 aa, Human immunodeficiency virus 1
AEQASQEVKNWM

SEQ ID NO: 150 - protein, 13 aa, Human immunodeficiency virus 1
GHQAAMQMLKETI

SEQ ID NO: 151 - protein, 21 aa, Human immunodeficiency virus 1
QNYTPGPGIRYPLTFGWCFKL

SEQ ID NO: 152 - protein, 10 aa, Human immunodeficiency virus 1
FKRKGGIGGY

SEQ ID NO: 153 - protein, 18 aa, Human immunodeficiency virus 1
SALSEGATPQDLNTMLNT

SEQ ID NO: 154 - protein, 15 aa, Human immunodeficiency virus 1
LVGPTPVNIIGRNLL

SEQ ID NO: 155 - protein, 20 aa, Human immunodeficiency virus 1
VKVVEEKAFSPEVIPMFSAL

SEQ ID NO: 156 - protein, 15 aa, Human immunodeficiency virus 1
KQNPDIVIYQYMDDL

SEQ ID NO: 157 - protein, 14 aa, Human immunodeficiency virus 1
FRDYVDRFYKTLRA

SEQ ID NO: 158 - protein, 12 aa, Human immunodeficiency virus 1
AEQASQDVKNWM

SEQ ID NO: 159 - protein, 12 aa, Human immunodeficiency virus 1
VRMYSPTSILDI

SEQ ID NO: 160 - protein, 15 aa, Human immunodeficiency virus 1
PIQKETWEAWWTEYW

SEQ ID NO: 161 - protein, 13 aa, Human immunodeficiency virus 1
HTQGYFPDWQNYT

SEQ ID NO: 162 - protein, 13 aa, Human immunodeficiency virus 1
EIYKRWIILGLNK

SEQ ID NO: 163 - protein, 15 aa, Human immunodeficiency virus 1
PIQKEIWETWWTDYW

SEQ ID NO: 164 - protein, 16 aa, Human immunodeficiency virus 1
GQMVHQPLSPRTLNAW

SEQ ID NO: 165 - protein, 15 aa, Human immunodeficiency virus 1
YTPGPGTRYPLTFGW

SEQ ID NO: 166 - protein, 20 aa, Human immunodeficiency virus 1
GQMVHQAISPRTLNAWVKVV

SEQ ID NO: 167 - protein, 15 aa, Human immunodeficiency virus 1
ACQGVGGPGHKARVL

SEQ ID NO: 168 - protein, 12 aa, Human immunodeficiency virus 1
VRMYSPVSILDI

SEQ ID NO: 169 - protein, 20 aa, Human immunodeficiency virus 1
GQMVHQALSPRTLNAWVKVI

SEQ ID NO: 170 - protein, 17 aa, Human immunodeficiency virus 1
YTPGPGVRFPLTFGWCF

SEQ ID NO: 171 - protein, 15 aa, Human immunodeficiency virus 1
AQNPEIVIYQYMDDL

SEQ ID NO: 172 - protein, 21 aa, Human immunodeficiency virus 1
QNYTPGPGVRYPLTFGWCFKL

SEQ ID NO: 173 - protein, 13 aa, Human immunodeficiency virus 1
HTQGFFPDWQNYT

SEQ ID NO: 174 - protein, 15 aa, Human immunodeficiency virus 1
PIQKETWETWWTDYW

SEQ ID NO: 175 - protein, 16 aa, Human immunodeficiency virus 1
GQMVHQPISPRTLNAW

SEQ ID NO: 176 - protein, 15 aa, Human immunodeficiency virus 1
LIGPTPVNIIGRNML

SEQ ID NO: 177 - protein, 20 aa, Human immunodeficiency virus 1
VKVIEEKAFSPEVIPMFTAL

SEQ ID NO: 178 - protein, 15 aa, Human immunodeficiency virus 1
PIQKEIWETWWTEYW

SEQ ID NO: 179 - protein, 14 aa, Human immunodeficiency virus 1
FRDYVDRFFKTLRA

SEQ ID NO: 180 - protein, 18 aa, Human immunodeficiency virus 1
TALSEGATPQDLNTMLNT

SEQ ID NO: 181 - protein, 13 aa, Human immunodeficiency virus 1
DIYKRWIILGLNK

SEQ ID NO: 182 - protein, 15 aa, Human immunodeficiency virus 1
ACQGVGGPSHKARVL

SEQ ID NO: 183 - protein, 553 aa, synthetic construct
MDCKTILKALRMYGERIVDIIARIVTDSQYALAEQASQEVKNWMKRNRSFNCGGEFFAAAYGHQAAMQMLKETIAKQNPEIVIYRTAFTIPSIRMAKLTPLCVTLSMMTAFTIPSVRQNYTPGPGIRYPLTFGWCFKLRKRAFKRKGGIGGYRKYNTQGYYPDWKRMYSALSEGATPQDLNTMLNTYYLVGPTPVNIIGRNLLRYYVKVVEEKAFSPEVIPMFSALKQNPDIVIYQYMDDLARGFRDYVDRFYKTLRARAEQASQDVKNWMSGARAIEAQQHLRRAVRMYSPTSILDIAMAPIQKETWEAWWTEYWGHTQGYFPDWQNYTRRYEIYKRWIILGLNKRAAGLNKIVRMYKMRTSTLQEQIGWARMKPIQKEIWETWWTDYWGQMVHQPLSPRTLNAWYTPGPGTRYPLTFGWAMYGQMVHQAISPRTLNAWVKVVYMATPGIRYQYNVLRKMYACQGVGGPGHKARVLMGIPHPAGLKAKKQYIKANSKFIGITELKKLGGGKRGGGKKMTNSVDDALINSTKIYSYFPSVISKVNQGAQGKKL

SEQ ID NO: 184 - RNA, 1659 nt, synthetic construct
  source 1..1659: mol_type = other RNA
atggattgtaagactattttaaaagcattgcgcatgtacggggaaagaatagtagacataatagcaaggatagtaacagactcacagtatgcattagccgagcaagcttcacaggaggtaaaaaattggatgaagaggaacaggagttttaattgtggaggggaatttttcgctgccgcgtatggacatcaagcagccatgcaaatgttaaaagagaccatcgccaaacaaaatccagagatagttatctatcggactgcattcaccatacctagtataagaatggcgaagttgaccccactctgtgtcactttatccatgatgactgcattcaccatacctagtgtacgtcagaactacacaccagggccagggatcagatatccactgacctttggatggtgcttcaagctaagaaaacgggcatttaaaagaaaaggggggattggggggtacagaaaatacaacacacaaggctactaccctgattggaagcgaatgtattcagcattatcagaaggagccaccccacaagatttaaacaccatgctaaacacatactacttagtaggacctacacctgtcaacataattggaagaaatctgttgaggtattacgtaaaagtagtagaagagaaggctttcagcccagaagtaatacccatgttttcagcattaaaacaaaatccagacatagttatctatcaatacatggatgatttggccagaggttttagagactatgtagaccggttctataaaactctaagagcccgagctgaacaagcttcacaagatgtaaaaaattggatgagcggcgcaagggctattgaggcgcaacagcatctgcgacgggctgtaagaatgtatagccctaccagcattctggacatagctatggcacccatacaaaaagaaacatgggaagcatggtggacagagtattgggggcacacacaaggctacttccctgattggcagaactacacacggcgctatgaaatctataaaagatggataatcctgggattaaataaacgagcagctgggttaaataaaatagtaagaatgtataaaatgcgtactagtacccttcaggaacaaataggatgggcaaggatgaaacccatccaaaaagaaatatgggagacatggtggacagactattgggggcaaatggtacaccagcccctatcacctagaactttgaatgcatggtacacaccgggaccagggactagatatccactgacctttggatgggcgatgtatgggcaaatggtacatcaggccatatcacctagaactttaaatgcatgggtaaaagtagtatatatggcaacaccagggattagatatcaatataatgtgcttcgtaagatgtatgcatgtcagggagtgggaggacctggccacaaagcaagagtgttgatgggaataccacacccagcagggttaaaagctaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactg

SEQ ID NO: 185 - protein, 533 aa, synthetic construct
MIVTDSQYALYMAEQATQEVKNWMATSTLQEQIAWGIPHPAGLKRRYAVRMYSPVSILDIRSYGLNKIVRMYRMGHQAAMQMLKETIAKNPEIVIYSYGQMVHQALSPRTLNAWVKVIYMATPGIRYQYNVLAEQATQDVKNWMSFNCRGEFFAYTPGPGVRFPLTFGWCFAAQNPEIVIYQYMDDLARQNYTPGPGVRYPLTFGWCFKLAFKRKGGIGGYRSYNTQGYFPDWRYAHTQGFFPDWQNYTARYPIQKETWETWWTDYWGNMYGQMVHQPISPRTLNAWANAYGERIIDIIAKLIGPTPVNIIGRNMLYRYADCKTILRALRRAYVKVIEEKAFSPEVIPMFTALARPIQKEIWETWWTEYWGNNFRDYVDRFFKTLRARAIEAQQHMARARTALSEGATPQDLNTMLNTDIYKRWIILGLNKRRAKLTPLCVTLSMACQGVGGPSHKARVLTAFTIPSTKKQYIKANSKFIGITELKKLGGGKRGGGKKMTNSVDDALINSTKIYSYFPSVISKVNQGAQGKKL

SEQ ID NO: 186 - RNA, 1599 nt, synthetic construct
  source 1..1599: mol_type = other RNA
atgatagtaacagactcacagtatgcattatacatggctgaacaagctacacaagaggtaaaaaattggatggcgactagtacccttcaggaacaaatagcatggggaataccacacccagcagggttaaaaagacggtatgcagtaagaatgtatagccctgtcagcattttggacatacgttcttacgggttaaataaaatagtaagaatgtatcgtatgggacatcaagcagccatgcaaatgttaaaagagaccatcgcaaaaaatccagaaatagtcatctattcctacgggcaaatggtacaccaggccctatcacctagaactttgaatgcatgggtaaaagtaatatatatggccacaccagggattagatatcaatataatgtgcttgctgaacaagctacacaagatgtaaaaaattggatgagctttaattgtagaggagaatttttcgcgtacacaccgggaccaggggtcagatttccactgacctttggatggtgcttcgcagcacaaaatccagaaatagtcatctatcaatatatggatgacttggcccgtcaaaactacacaccgggaccaggggtcagatatccactgacctttggatggtgcttcaagctagcatttaaaagaaaaggggggattggggggtaccgctcttataacacacaaggctacttccctgattggaggtatgcccacacacaaggcttcttccctgattggcaaaactacacagctaggtatcccatccaaaaagaaacatgggagacatggtggacagactattgggggaatatgtacgggcaaatggtacaccagcccatatcacctagaactttgaatgcatgggctaatgcgtacggggaaagaataatagacataatagcaaagttaataggacctacacctgtcaacataattggaagaaatatgttgtatagatacgcggattgtaagaccattttaagagcattacgacgtgcatatgtaaaagtaatagaggagaaggcttttagcccagaggtaatacccatgtttacagcattagcacgtcccatccaaaaagaaatatgggagacatggtggacagagtattggggaaacaattttagagactatgtagaccggttctttaaaactttaagagctagggctatagaggcgcaacagcatatggctcgtgctcgaacagcattatcagaaggagccaccccacaagatttaaacaccatgttaaatacagacatctataaaagatggataattctggggttaaataaaagacgtgccaagttgaccccactctgtgtcactttatcaatggcatgtcagggagtgggaggaccttcccacaaagcaagagtgttgactgcattcaccatacctagtacaaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactg

SEQ ID NO: 187 - protein, 982 aa, synthetic construct
MDCKTILKALRKYGGSGGGGSGGNAYGERIVDIIAKKGGSGGGGSGGRARIVTDSQYALSMNGGSGGGGSGGYMAAEQASQEVKNWMKRYGGSGGGGSGGYNYSFNCGGEFFSGWGGSGGGGSGGYAAGHQAAMQMLKETISREGGSGGGGSGGMAKQNPEIVIYRMGGSGGGGSGGSATAFTIPSISGGSGGGGSGGRAAKLTPLCVTLSMMGGSGGGGSGGTAFTIPSVRWGGSGGGGSGGMMRQNYTPGPGIRYPLTFGWCFKLKKRGGSGGGGSGGYSAFKRKGGIGGYRRYGGSGGGGSGGYSYNTQGYYPDWKRYGGSGGGGSGGQNDSALSEGATPQDLNTMLNTRRGGSGGGGSGGRGRLVGPTPVNIIGRNLLFWRGGSGGGGSGGMYMVKVVEEKAFSPEVIPMFSALKRGGSGGGGSGGFYYKQNPDIVIYQYMDDLKRYGGSGGGGSGGYAAFRDYVDRFYKTLRADDDGGSGGGGSGGYMYAEQASQDVKNWMSMFGGSGGGGSGGRAIEAQQHLKRGGSGGGGSGGNQDVRMYSPTSILDIGMGGGSGGGGSGGIRYPIQKETWEAWWTEYWGRRGGSGGGGSGGYSFHTQGYFPDWQNYTGRYGGSGGGGSGGNYEIYKRWIILGLNKRRGGSGGGGSGGRAAGLNKIVRMYRKYGGSGGGGSGGRMFTSTLQEQIGWKIFGGSGGGGSGGNIYPIQKEIWETWWTDYWGNNGGSGGGGSGGMGGGQMVHQPLSPRTLNAWYRYGGSGGGGSGGYAYYTPGPGTRYPLTFGWAKGGGSGGGGSGGFFFGQMVHQAISPRTLNAWVKVVYYYGGSGGGGSGGYMYTPGIRYQYNVLKKTGGSGGGGSGGKAAACQGVGGPGHKARVLKTQGGSGGGGSGGTFGIPHPAGLKRRGGSGGGGSGGKKQYIKANSKFIGITELKKLGGGKRGGGKKMTNSVDDALINSTKIYSYFPSVISKVNQGAQGKKL

SEQ ID NO: 188 - RNA, 2946 nt, synthetic construct
  source 1..2946: mol_type = other RNA
atggattgtaagactattttaaaagcattgcggaagtacggtggctctggaggcggtggatcaggtggaaatgcgtacggggaaagaatagtagacataatagcaaagaagggtggaagcggaggcggtggatcgggtggccgtgcgcgtatagtaacagactcacagtatgcattatcgatgaatggtggcagcggaggcggtggatccggcggttacatggccgccgagcaagcttcacaggaggtaaaaaattggatgaaacggtatggaggcagtggtggcggaggtagtggtggatataattacagttttaattgtggaggggaatttttcagcggctggggaggtagcggcggtggaggcagcggaggttacgcggctggacatcaagcagccatgcaaatgttaaaagagaccatctcacgagagggaggtagtggaggcggtggatcgggcggtatggctaaacaaaatccagagatagttatctataggatgggtggcagtggaggtggcggaagcggaggttctgcgactgcattcaccatacctagtatatccggtggcagtggtggcggaggttcaggtggacgcgcggctaagttgaccccactctgtgtcactttatccatgatgggtggatctggtggaggcggttctggcggtactgcattcaccatacctagtgtacggtggggaggctcgggtggaggcggttccggaggcatgatgcggcagaactacacaccagggccagggatcagatatccactgacctttggatggtgcttcaagctaaaaaagcgaggcggttccggtggaggcggtagcggaggttactcagcatttaaaagaaaaggggggattggggggtacaggcgttatggtggctctggtggaggcggtagcggcggttacagttacaacacacaaggctactaccctgattggaagcgatatggtggatcgggaggcggtggaagtggtggacagaatgattcagcattatcagaaggagccaccccacaagatttaaacaccatgctaaacacaagacgtggtggatcgggcggtggaggctcaggaggccgcgggcggttagtaggacctacacctgtcaacataattggaagaaatctgttgttctggcggggcggatctggtggcggaggttcgggcggaatgtatatggtaaaagtagtagaagagaaggctttcagcccagaagtaatacccatgttttcagcattaaagaggggtggctccggcggaggtggctctggcggattctactataaacaaaatccagacatagttatctatcaatacatggatgatttgaaacgttatggtggcagtggaggcggtggatctggaggttatgctgcctttagagactatgtagaccggttctataaaactctaagagccgacgatgatggcggtagcggaggcggtggatcaggaggctacatgtacgctgaacaagcttcacaagatgtaaaaaattggatgtctatgtttggcggttctggcggaggtggcagtggtggcagggctattgaggcgcaacagcatctgaagagaggtggaagtggcggtggaggctccggtggcaatcaagatgtaagaatgtatagccctaccagcattctggacataggcatgggaggaggctcgggaggcggtggatcgggaggcatcaggtaccccatacaaaaagaaacatgggaagcatggtggacagagtattgggggagaagaggtggatccggtggaggcggtagtggtggctacagtttccacacacaaggctacttccctgattggcagaactacacaggtcgatacggcggttcaggtggaggcggttcaggaggtaactacgaaatctataaaagatggataatcctgggattaaataaacgtagaggaggctccggcggaggtggctcaggtggacgggctgctgggttaaataaaatagtaagaatgtatcgaaagtatggcggttcaggcggaggtggctccggaggcagaatgttcactagtacccttcaggaacaaataggatggaaaatatttggtggatcaggcggtggaggctctggaggtaacatctatcccatccaaaaagaaatatgggagacatggtggacagactattggggaaacaacggcggatccggcggtggaggctcgggaggtatgggtggggggcaaatggtacaccagcccctatcacctagaactttgaatgcatggtaccgttatggcggatcgggcggaggtggcagcggaggctacgcttactacacaccgggaccagggactagatatccactgacctttggatgggccaagggtggcggttctggaggtggcggatccggcggtttctttttcgggcaaatggtacatcaggccatatcacctagaactttaaatgcatgggtaaaagtagtatattactatggtggctcaggtggaggcggtagtggtggatacatgtatacaccagggattagatatcaatataatgtgcttaagaagactggtggctcaggcggaggtggcagcggcggtaaggcggcggcatgtcagggagtgggaggacctggccacaaagcaagagtgttgaagacccagggcggatcgggcggaggtggctctggtggaacattcggaataccacacccagcagggttaaaaagacgtggcggatcaggtggcggaggttcgggaggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactg

SEQ ID NO: 189 - protein, 956 aa, synthetic construct
MIVTDSQYALSMNGGSGGGGSGGMMQAEQATQEVKNWMSGAGGSGGGGSGGNMFTSTLQEQIAWKIFGGSGGGGSGGTFGIPHPAGLKRRGGSGGGGSGGLQDVRMYSPVSILDIGMGGGSGGGGSGGRAAGLNKIVRMYRKYGGSGGGGSGGYAAGHQAAMQMLKETISREGGSGGGGSGGMYAKNPEIVIYKMGGSGGGGSGGYAFGQMVHQALSPRTLNAWVKVIGRRGGSGGGGSGGYMYTPGIRYQYNVLKKTGGSGGGGSGGYMAAEQATQDVKNWMKRYGGSGGGGSGGQNRSFNCRGEFFAGNGGSGGGGSGGMMMYTPGPGVRFPLTFGWCFMGNGGSGGGGSGGAYAQNPEIVIYQYMDDLKRGGSGGGGSGGMMRQNYTPGPGVRYPLTFGWCFKLKKRGGSGGGGSGGYSAFKRKGGIGGYRRYGGSGGGGSGGYNTQGYFPDWKIGGSGGGGSGGTYAHTQGFFPDWQNYTKRGGSGGGGSGGNIYPIQKETWETWWTDYWGGNGGSGGGGSGGMMYGQMVHQPISPRTLNAWARNGGSGGGGSGGMAYGERIIDIIAKKGGGSGGGGSGGRGGLIGPTPVNIIGRNMLFWRGGSGGGGSGGRAADCKTILRALRMRGGSGGGGSGGAVKVIEEKAFSPEVIPMFTALAIGGSGGGGSGGIMYPIQKEIWETWWTEYWGAWGGSGGGGSGGWAFRDYVDRFFKTLRAGKGGSGGGGSGGRAIEAQQHMKRGGSGGGGSGGNNYTALSEGATPQDLNTMLNTWGGGSGGGGSGGNYDIYKRWIILGLNKRRGGSGGGGSGGRAAKLTPLCVTLSMMGGSGGGGSGGFAAACQGVGGPSHKARVLSGWGGSGGGGSGGAFTAFTIPSTRWGGSGGGGSGGKKQYIKANSKFIGITELKKLGGGKRGGGKKMTNSVDDALINSTKIYSYFPSVISKVNQGAQGKKL

SEQ ID NO: 190 - RNA, 2868 nt, synthetic construct
  source 1..2868: mol_type = other RNA
atgatagtaacagactcacagtatgcattatcaatgaacggaggtagcggtggcggaggttctggcggaatgatgcaggctgaacaagctacacaagaggtaaaaaattggatgagtggagcaggcggtagcggcggaggtggcagtggaggtaatatgttcactagtacccttcaggaacaaatagcatggaaaatctttggaggctccggaggcggtggatccggcggaactttcggaataccacacccagcagggttaaaacggcgtggaggttctggtggaggcggttcaggaggtttgcaggatgtaagaatgtatagccctgtcagcattttggacatagggatgggtggaggtagcggcggaggtggctcgggtggcagagccgcagggttaaataaaatagtaagaatgtatcgtaagtacggtggaagcggaggtggcggatccggtggatacgcagcgggacatcaagcagccatgcaaatgttaaaagagaccatctcaagggagggtggcagtggcggtggaggcagtggtggcatgtacgcaaaaaatccagaaatagtcatctataaaatgggcggtagcggaggtggcggaagcggcggttatgcgttcgggcaaatggtacaccaggccctatcacctagaactttgaatgcatgggtaaaagtaataggcaggcgcggaggcagtggaggtggcggatcgggaggttacatgtatacaccagggattagatatcaatataatgtgcttaaaaagaccggtggaagtggaggcggtggaagcggaggttacatggccgctgaacaagctacacaagatgtaaaaaattggatgaaacgatatggaggcagtggaggtggcggatcaggtggacaaaataggagctttaattgtagaggagaatttttcgctgggaacggcggatctggtggcggaggttctggcggtatgatgatgtacacaccgggaccaggggtcagatttccactgacctttggatggtgcttcatgggcaacggaggctcgggtggaggcggttccggcggagcatacgcacaaaatccagaaatagtcatctatcaatatatggatgacttgaaacgcggaggctccggaggtggcggaagcggcggaatgatgagacaaaactacacaccgggaccaggggtcagatatccactgacctttggatggtgcttcaagctaaagaaacgcggcggatctggcggaggtggcagcggtggatatagcgcatttaaaagaaaaggggggattggggggtaccgccgttacggaggttcgggtggaggcggtagtggaggctataacacacaaggctacttccctgattggaagattggcggatcgggaggcggtggatcaggtggcacgtatgcacacacacaaggcttcttccctgattggcaaaactacacaaagcgtggcggttctggcggtggaggctcgggtggaaatatctaccccatccaaaaagaaacatgggagacatggtggacagactattggggagggaacggtggaagtggtggaggcggttctggcggaatgatgtacgggcaaatggtacaccagcccatatcacctagaactttgaatgcatgggcaaggaacggcggtagcggcggtggaggctcaggtggaatggcctatggggaaagaataatagacataatagcaaagaagggtggaggctctggcggaggtggcagtggtggccgtggaggcttaataggacctacacctgtcaacataattggaagaaatatgttgttctggagaggaggtagtggcggtggaggctccggcggtcgggcagcggattgtaagaccattttaagagcattacgtatgaggggtggatcgggaggtggcggatcgggcggagcagtaaaagtaatagaggagaaggcttttagcccagaggtaatacccatgtttacagcattagcaatcggtggatccggaggcggtggaagtggcggtataatgtatcccatccaaaaagaaatatgggagacatggtggacagagtattggggcgcgtggggaggctcaggaggtggcggatcaggaggttgggcctttagagactatgtagaccggttctttaaaactttaagagctggtaaaggaggttccggtggcggaggttcaggtggaagggctatagaggcgcaacagcatatgaaaagaggcggatcaggtggaggcggttccggaggtaacaactacacagcattatcagaaggagccaccccacaagatttaaacaccatgttaaatacatggggaggaggttcaggtggaggcggttctggtggaaattacgacatctataaaagatggataattctggggttaaataaacgacgcggtggatccggtggaggcggttcgggtggaagagcagctaagttgaccccactctgtgtcactttatctatgatgggcggatcgggcggaggtggcagcggtggcttcgcagcggcatgtcagggagtgggaggaccttcccacaaagcaagagtgttgagcggttggggcggttctggtggaggcggttccggtggcgcctttactgcattcaccatacctagtacacgttggggtggctcaggcggaggtggcagtggtggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactg

SEQ ID NO: 191 - protein, 65 aa, synthetic construct
KKQYIKANSKFIGITELKKLGGGKRGGGKKMTNSVDDALINSTKIYSYFPSVISKVNQGAQGKKL

SEQ ID NO: 192 - RNA, 195 nt, synthetic construct
  source 1..195: mol_type = other RNA
aagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactg

SEQ ID NO: 193 - protein, 10 aa, synthetic construct
GGSGGGGSGG

SEQ ID NO: 194 - RNA, 30 nt, synthetic construct
  source 1..30: mol_type = other RNA
ggcggatcgggtggaggcggttcaggtggc

SEQ ID NO: 195 - RNA, 52 nt, synthetic construct
  source 1..52: mol_type = other RNA
gggcgaactagtattcttctggtccccacagactcagagagaacccgccacc

SEQ ID NO: 196 - RNA, 317 nt, synthetic construct
  source 1..317: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 197 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 198 - protein, 9 aa, Human immunodeficiency virus 1
WEIWWTDYW

SEQ ID NO: 199 - protein, 9 aa, Human immunodeficiency virus 1
NTQGFFPDW

SEQ ID NO: 200 - protein, 10 aa, Human immunodeficiency virus 1
PIQKETWEIW

SEQ ID NO: 201 - protein, 10 aa, Human immunodeficiency virus 1
TWEIWWTDYW

SEQ ID NO: 202 - protein, 10 aa, Human immunodeficiency virus 1
TWETWWTEYW

SEQ ID NO: 203 - protein, 15 aa, Human immunodeficiency virus 1
PIQKETWEIWWTDYW

SEQ ID NO: 204 - protein, 15 aa, Human immunodeficiency virus 1
PIQKETWETWWTEYW

SEQ ID NO: 205 - protein, 553 aa, synthetic construct
MDCKTILKALRMYGERIVDIIARIVTDSQYALAEQASQEVKNWMKRNRSFNCGGEFFAAAYGHQAAMQMLKETIAKQNPEIVIYRTAFTIPSIRMAKLTPLCVTLSMMTAFTIPSVRQNYTPGPGIRYPLTFGWCFKLRKRAFKRKGGIGGYRKYNTQGFFPDWKRMYSALSEGATPQDLNTMLNTYYLVGPTPVNIIGRNLLRYYVKVVEEKAFSPEVIPMFSALKQNPDIVIYQYMDDLARGFRDYVDRFYKTLRARAEQASQDVKNWMSGARAIEAQQHLRRAVRMYSPTSILDIAMAPIQKETWEAWWTEYWGHTQGYFPDWQNYTRRYEIYKRWIILGLNKRAAGLNKIVRMYKMRTSTLQEQIGWARMKPIQKETWEIWWTDYWGQMVHQPLSPRTLNAWYTPGPGTRYPLTFGWAMYGQMVHQAISPRTLNAWVKVVYMATPGIRYQYNVLRKMYACQGVGGPGHKARVLMGIPHPAGLKAKKQYIKANSKFIGITELKKLGGGKRGGGKKMTNSVDDALINSTKIYSYFPSVISKVNQGAQGKKL

SEQ ID NO: 206 - RNA, 1665 nt, synthetic construct
  source 1..1665: mol_type = other RNA
atggactgcaagaccatcctgaaggccctgcgcatgtacggcgagagaatcgtggacatcattgccaggatcgtgaccgatagccagtacgccctggccgaacaggccagccaagaagtgaagaactggatgaagcggaaccggagcttcaattgtggcggcgagttcttcgctgccgcgtatggacatcaggccgctatgcagatgctgaaagagacaatcgccaagcagaaccccgagatcgtgatctaccggaccgccttcacaatccccagcatcagaatggcgaagctgacccctctgtgtgtgaccctgtccatgatgaccgccttcacaatccctagcgtgcgtcagaactacacacccggacctggcatcagataccctctgaccttcggctggtgcttcaagctgagaaaacgggcattcaagcggaaaggcggcatcggcggctatagaaaatacaacacccagggcttcttccccgattggaagcgaatgtattctgctctgtctgaaggcgccacacctcaggacctgaacaccatgctgaacacctactacctcgttggacccacacctgtgaacatcatcggccggaatctgctgaggtattacgtgaaggtggtggaagaaaaggctttcagccccgaagtgatccccatgttcagcgctcttaagcagaaccccgacatcgtgatctaccagtacatggacgacctggccagaggtttcagagactacgtggaccggttctacaagaccctgagagcccgagccgaacaggcttcccaggacgtgaagaactggatgagcggcgcaagagccattgaagcccagcagcacctccgacgggctgtgagaatgtacagccccaccagcatcctggacatcgctatggcacccatccagaaagagacatgggaagcctggtggaccgagtactgggggcacacccagggctacttccccgactggcagaattacacccggcgctatgagatctacaagcggtggatcatcctgggcctgaacaagcgagcagctggcctgaacaagatcgtgcggatgtacaaaatgcgtaccagcacactgcaagagcagatcggctgggcaaggatgaaacccatccagaaagagacctgggaaatctggtggaccgactactggggccagatggttcaccagcctctgagccccagaacactgaatgcttggtacacacctggacctggcacaagataccctctgacctttggctgggcgatgtatggacagatggtgcatcaggccatctctcccagaacactgaacgcctgggtcaaagtggtgtatatggcaacccctggcatcagataccagtacaacgtgctgcgtaagatgtatgcctgtcaaggtgttggaggacctggacacaaggccagagttctgatgggcattcctcatcctgccggactgaaagctaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 207 - RNA, 1665 nt, synthetic construct
  source 1..1665: mol_type = other RNA
atggattgcaaaacaatcttaaaagctttacgcatgtacggagaaagaattgtggatattattgctaggattgtaacagattctcaatatgctctggctgaacaagcttctcaggaagtgaaaaattggatgaagcggaaccggtcttttaattgtggaggagaattcttcgctgccgcgtatggacaccaagcagcaatgcaaatgttgaaagaaaccatcgccaaacaaaatcctgaaattgtgatttaccggaccgcttttaccattccatcaattagaatggcgaaattgacccctttgtgtgtgaccttgtccatgatgaccgcttttaccattccaagtgttcgtcagaattacacccctggacctggaatcagataccctctgacctttggatggtgctttaaactgagaaaacgggcatttaaaagaaaaggaggaattggaggatacagaaaatacaacacccaaggattcttccctgattggaagcgaatgtattctgcattgtctgaaggagcaacccctcaagatttgaataccatgttgaatacctactaccttgtaggaccaacacctgtaaatatcatcggaagaaatcttcttaggtattacgtgaaagtggtggaagaaaaagctttttctcctgaagtgattccaatgttttctgctcttaaacaaaatcctgatattgtaatttaccaatacatggatgatctggccagaggttttagagattacgtggatagattttacaaaacactgagagcacgagctgaacaagcttctcaagacgttaaaaattggatgagcggcgcaagagcaattgaagcacagcagcacctccgacgggctgtgagaatgtactcaccaacctcaattctggatattgctatggcaccaattcagaaagaaacatgggaagcttggtggacagaatactgggggcacacacagggatactttcctgattggcagaattacacacggcgctatgaaatttacaaaagatggattattctgggactgaataaacgagcagctggattaaataaaattgtaagaatgtacaaaatgcgtacatcaacacttcaggaacagattggatgggcaaggatgaaaccaatccagaaagaaacctgggaaatatggtggacagattactggggacagatggtgcaccagccattgtctccaagaacattgaatgcttggtacacacctggacctggaacaagataccctctgacatttggatgggcgatgtatggacagatggttcaccaggcaatttctccaagaacattaaatgcatgggttaaagttgtttatatggcaacacctggaataagataccaatacaatgtgcttcgtaagatgtatgcatgccagggagtgggaggacctggacacaaagcaagagtgcttatgggaattcctcatcctgctggactgaaagctaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 208 - protein, 533 aa, synthetic construct
MIVTDSQYALYMAEQATQEVKNWMATSTLQEQIAWGIPHPAGLKRRYAVRMYSPVSILDIRSYGLNKIVRMYRMGHQAAMQMLKETIAKNPEIVIYSYGQMVHQALSPRTLNAWVKVIYMATPGIRYQYNVLAEQATQDVKNWMSFNCRGEFFAYTPGPGVRFPLTFGWCFAAQNPEIVIYQYMDDLARQNYTPGPGVRYPLTFGWCFKLAFKRKGGIGGYRSYNTQGYFPDWRYAHTQGFFPDWQNYTARYPIQKETWETWWTDYWGNMYGQMVHQPISPRTLNAWANAYGERIIDIIAKLIGPTPVNIIGRNMLYRYADCKTILRALRRAYVKVIEEKAFSPEVIPMFTALARPIQKETWETWWTEYWGNNFRDYVDRFFKTLRARAIEAQQHMARARTALSEGATPQDLNTMLNTDIYKRWIILGLNKRRAKLTPLCVTLSMACQGVGGPSHKARVLTAFTIPSTKKQYIKANSKFIGITELKKLGGGKRGGGKKMTNSVDDALINSTKIYSYFPSVISKVNQGAQGKKL

SEQ ID NO: 209 - RNA, 1605 nt, synthetic construct
  source 1..1605: mol_type = other RNA
atgatcgtgaccgatagccagtacgccctgtacatggccgaacaggccacacaagaagtgaagaactggatggcgaccagcacactgcaagagcagatcgcctggggcattcctcatcctgccggactgaaaagacggtatgcagtgagaatgtacagccccgtgtccatcctggacatccgttcttacggcctgaacaagatcgtgcggatgtaccgtatgggacatcaggccgctatgcagatgctgaaagagacaatcgccaagaatcccgagatcgtgatctactcctacggccagatggttcaccaggctctgagccccagaacactgaacgcctgggtcaaagtgatctatatggccacccctggcatcagataccagtacaacgtgctggccgaacaggctacccaggacgtgaagaactggatgagcttcaattgcaggggcgagttcttcgcgtacacacctggacctggcgtcagattccctctgaccttcggctggtgcttcgcagcccagaatcctgagatcgtgatctaccagtacatggacgacctggcccgtcagaactacacacctggacctggcgtgcggtatcctctgaccttcggctggtgcttcaagctggcattcaagcggaaaggcggcatcggcggctatcgctcttataacacccagggctacttccccgattggaggtatgcccacacccagggattcttcccagactggcagaactacaccgctaggtatcccatccagaaagagacatgggaaacctggtggaccgactactgggggaatatgtacggccagatggtgcatcagcctatcagccccagaacactgaacgcctgggctaatgcgtacggcgagcggatcatcgacatcattgccaagctgatcggacccacacctgtgaacatcatcggccggaacatgctgtatagatacgcggactgcaagaccatcctgagagccctgcgacgtgcatatgtgaaagtgatcgaggaaaaggctttcagccccgaagtgatccccatgttcacagcccttgcacgtcccatccagaaagagacttgggaaacctggtggaccgagtactggggaaacaatttcagagactacgtggaccggttcttcaagaccctgagagccagagccattgaagcccagcagcacatggctcgtgctcgaacagctctgtctgaaggcgccacacctcaggacctgaacaccatgctgaacaccgacatctacaagcggtggatcatcctgggcctgaacaaaagacgtgccaagctgacccctctgtgtgtgaccctgtcaatggcctgtcaaggtgttggcggaccttctcacaaggccagagttctgaccgccttcacaatccctagcaccaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 210 - RNA, 1605 nt, synthetic construct
  source 1..1605: mol_type = other RNA
atgattgtaacagattctcaatatgctctgtacatggctgaacaagctacacaagaagtgaaaaattggatggcgacatcaacacttcaggaacagattgcttggggaattcctcaccctgctggattgaaaagacggtatgcagtgagaatgtactctcctgtgtcaattctggatattcgttcttacggattaaataaaattgtaagaatgtaccgtatgggacaccaagcagcaatgcaaatgctgaaagaaaccattgctaaaaatcctgaaattgtgatttactcctacggacagatggtgcaccaggctttgtctccaagaacattgaatgcttgggtaaaagtaatttatatggccacacctggaataagataccaatacaatgtgcttgcagaacaagcaacacaagatgtgaaaaattggatgtctttcaattgcagaggagaattcttcgcgtacacccctggacctggagtgagatttcctctgacctttggatggtgctttgcagctcaaaatcctgaaattgtcatttaccaatacatggatgatcttgcccgtcagaattacacccctggacctggagtgagataccctctgacctttggatggtgctttaaactggcatttaaaagaaaaggaggaattggaggataccgctcttataatacccagggatactttcctgattggaggtatgcccacacccaaggattcttccctgattggcaaaattacaccgctaggtatccaatccagaaagaaacatgggaaacatggtggacagattactgggggaatatgtacggacagatggttcaccagccaatttctccaagaacactgaatgcttgggctaatgcgtacggagaaagaattattgatattattgctaagttgattggaccaacaccagtgaatattattggaagaaatatgttgtatagatacgcggattgcaaaaccattttaagagctttacgacgtgcatatgtgaaagtgattgaagaaaaagctttttctcctgaagtgattccaatgtttacagctctggcacgtccaatccagaaagaaacttgggaaacatggtggacagaatactggggaaacaattttagagattatgtggatagattcttcaaaaccttgagagctagagcaatcgaagcacagcagcacatggctcgtgctcgaacagcattgtctgaaggagcaacacctcaggatttgaatacaatgttgaatacagatatttacaaaagatggattattctgggactgaataaaagacgtgccaaattgacccctttgtgtgtgaccttgtcaatggcatgccagggagtgggaggaccttctcacaaagcaagagtgcttacagcttttacaatcccttcaacaaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 211 - RNA, 1665 nt, synthetic construct
  source 1..1665: mol_type = other RNA
atggactgcaagaccatcctgaaggccctgcgcatgtacggcgagagaatcgtggacatcattgccaggatcgtgaccgatagccagtacgccctggccgaacaggccagccaagaagtgaagaactggatgaagcggaaccggagcttcaattgtggcggcgagttcttcgctgccgcgtatggacatcaggccgctatgcagatgctgaaagagacaatcgccaagcagaaccccgagatcgtgatctaccggaccgccttcacaatccccagcatcagaatggcgaagctgacccctctgtgtgtgaccctgtccatgatgaccgccttcacaatccctagcgtgcgtcagaactacacacccggacctggcatcagataccctctgaccttcggctggtgcttcaagctgagaaaacgggcattcaagcggaaaggcggcatcggcggctatagaaaatacaacacccagggctactaccccgattggaagcgaatgtattctgctctgtctgaaggcgccacacctcaggacctgaacaccatgctgaacacctactacctcgttggacccacacctgtgaacatcatcggccggaatctgctgaggtattacgtgaaggtggtggaagaaaaggctttcagccccgaagtgatccccatgttcagcgctcttaagcagaaccccgacatcgtgatctaccagtacatggacgacctggccagaggtttcagagactacgtggaccggttctacaagaccctgagagcccgagccgaacaggcttcccaggacgtgaagaactggatgagcggcgcaagagccattgaagcccagcagcacctccgacgggctgtgagaatgtacagccccaccagcatcctggacatcgctatggcacccatccagaaagagacatgggaagcctggtggaccgagtactgggggcacacccagggctacttccccgactggcagaattacacccggcgctatgagatctacaagcggtggatcatcctgggcctgaacaagcgagcagctggcctgaacaagatcgtgcggatgtacaaaatgcgtaccagcacactgcaagagcagatcggctgggcaaggatgaaacccatccagaaagagatctgggaaacctggtggaccgactactggggccagatggttcaccagcctctgagccccagaacactgaatgcttggtacacacctggacctggcacaagataccctctgacctttggctgggcgatgtatggacagatggtgcatcaggccatctctcccagaacactgaacgcctgggtcaaagtggtgtatatggcaacccctggcatcagataccagtacaacgtgctgcgtaagatgtatgcctgtcaaggtgttggaggacctggacacaaggccagagttctgatgggcattcctcatcctgccggactgaaagctaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 212 - RNA, 1665 nt, synthetic construct
  source 1..1665: mol_type = other RNA
atggattgcaaaacaatcttaaaagctttacgcatgtacggagaaagaattgtggatattattgctaggattgtaacagattctcaatatgctctggctgaacaagcttctcaggaagtgaaaaattggatgaagcggaaccggtcttttaattgtggaggagaattcttcgctgccgcgtatggacaccaagcagcaatgcaaatgttgaaagaaaccatcgccaaacaaaatcctgaaattgtgatttaccggaccgcttttaccattccatcaattagaatggcgaaattgacccctttgtgtgtgaccttgtccatgatgaccgcttttaccattccaagtgttcgtcagaattacacccctggacctggaatcagataccctctgacctttggatggtgctttaaactgagaaaacgggcatttaaaagaaaaggaggaattggaggatacagaaaatacaacacccaaggatactaccctgattggaagcgaatgtattctgcattgtctgaaggagcaacccctcaagatttgaataccatgttgaatacctactaccttgtaggaccaacacctgtaaatatcatcggaagaaatcttcttaggtattacgtgaaagtggtggaagaaaaagctttttctcctgaagtgattccaatgttttctgctcttaaacaaaatcctgatattgtaatttaccaatacatggatgatctggccagaggttttagagattacgtggatagattttacaaaacactgagagcacgagctgaacaagcttctcaagacgttaaaaattggatgagcggcgcaagagcaattgaagcacagcagcacctccgacgggctgtgagaatgtactcaccaacctcaattctggatattgctatggcaccaattcagaaagaaacatgggaagcttggtggacagaatactgggggcacacacagggatactttcctgattggcagaattacacacggcgctatgaaatttacaaaagatggattattctgggactgaataaacgagcagctggattaaataaaattgtaagaatgtacaaaatgcgtacatcaacacttcaggaacagattggatgggcaaggatgaaaccaatccagaaagaaatctgggaaacatggtggacagattactggggacagatggtgcaccagccattgtctccaagaacattgaatgcttggtacacacctggacctggaacaagataccctctgacatttggatgggcgatgtatggacagatggttcaccaggcaatttctccaagaacattaaatgcatgggttaaagttgtttatatggcaacacctggaataagataccaatacaatgtgcttcgtaagatgtatgcatgccagggagtgggaggacctggacacaaagcaagagtgcttatgggaattcctcatcctgctggactgaaagctaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 213 - RNA, 1605 nt, synthetic construct
  source 1..1605: mol_type = other RNA
atgatcgtgaccgatagccagtacgccctgtacatggccgaacaggccacacaagaagtgaagaactggatggcgaccagcacactgcaagagcagatcgcctggggcattcctcatcctgccggactgaaaagacggtatgcagtgagaatgtacagccccgtgtccatcctggacatccgttcttacggcctgaacaagatcgtgcggatgtaccgtatgggacatcaggccgctatgcagatgctgaaagagacaatcgccaagaatcccgagatcgtgatctactcctacggccagatggttcaccaggctctgagccccagaacactgaacgcctgggtcaaagtgatctatatggccacccctggcatcagataccagtacaacgtgctggccgaacaggctacccaggacgtgaagaactggatgagcttcaattgcaggggcgagttcttcgcgtacacacctggacctggcgtcagattccctctgaccttcggctggtgcttcgcagcccagaatcctgagatcgtgatctaccagtacatggacgacctggcccgtcagaactacacacctggacctggcgtgcggtatcctctgaccttcggctggtgcttcaagctggcattcaagcggaaaggcggcatcggcggctatcgctcttataacacccagggctacttccccgattggaggtatgcccacacccagggattcttcccagactggcagaactacaccgctaggtatcccatccagaaagagacatgggaaacctggtggaccgactactgggggaatatgtacggccagatggtgcatcagcctatcagccccagaacactgaacgcctgggctaatgcgtacggcgagcggatcatcgacatcattgccaagctgatcggacccacacctgtgaacatcatcggccggaacatgctgtatagatacgcggactgcaagaccatcctgagagccctgcgacgtgcatatgtgaaagtgatcgaggaaaaggctttcagccccgaagtgatccccatgttcacagcccttgcacgtcccatccagaaagagatctgggaaacctggtggaccgagtactggggaaacaatttcagagactacgtggaccggttcttcaagaccctgagagccagagccattgaagcccagcagcacatggctcgtgctcgaacagctctgtctgaaggcgccacacctcaggacctgaacaccatgctgaacaccgacatctacaagcggtggatcatcctgggcctgaacaaaagacgtgccaagctgacccctctgtgtgtgaccctgtcaatggcctgtcaaggtgttggcggaccttctcacaaggccagagttctgaccgccttcacaatccctagcaccaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 214 - RNA, 1605 nt, synthetic construct
  source 1..1605: mol_type = other RNA
atgattgtaacagattctcaatatgctctgtacatggctgaacaagctacacaagaagtgaaaaattggatggcgacatcaacacttcaggaacagattgcttggggaattcctcaccctgctggattgaaaagacggtatgcagtgagaatgtactctcctgtgtcaattctggatattcgttcttacggattaaataaaattgtaagaatgtaccgtatgggacaccaagcagcaatgcaaatgctgaaagaaaccattgctaaaaatcctgaaattgtgatttactcctacggacagatggtgcaccaggctttgtctccaagaacattgaatgcttgggtaaaagtaatttatatggccacacctggaataagataccaatacaatgtgcttgcagaacaagcaacacaagatgtgaaaaattggatgtctttcaattgcagaggagaattcttcgcgtacacccctggacctggagtgagatttcctctgacctttggatggtgctttgcagctcaaaatcctgaaattgtcatttaccaatacatggatgatcttgcccgtcagaattacacccctggacctggagtgagataccctctgacctttggatggtgctttaaactggcatttaaaagaaaaggaggaattggaggataccgctcttataatacccagggatactttcctgattggaggtatgcccacacccaaggattcttccctgattggcaaaattacaccgctaggtatccaatccagaaagaaacatgggaaacatggtggacagattactgggggaatatgtacggacagatggttcaccagccaatttctccaagaacactgaatgcttgggctaatgcgtacggagaaagaattattgatattattgctaagttgattggaccaacaccagtgaatattattggaagaaatatgttgtatagatacgcggattgcaaaaccattttaagagctttacgacgtgcatatgtgaaagtgattgaagaaaaagctttttctcctgaagtgattccaatgtttacagctctggcacgtccaatccagaaagaaatctgggaaacatggtggacagaatactggggaaacaattttagagattatgtggatagattcttcaaaaccttgagagctagagcaatcgaagcacagcagcacatggctcgtgctcgaacagcattgtctgaaggagcaacacctcaggatttgaatacaatgttgaatacagatatttacaaaagatggattattctgggactgaataaaagacgtgccaaattgacccctttgtgtgtgaccttgtcaatggcatgccagggagtgggaggaccttctcacaaagcaagagtgcttacagcttttacaatcccttcaacaaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 215 - RNA, 2952 nt, synthetic construct
  source 1..2952: mol_type = other RNA
atggactgcaagaccatcctgaaggccctgcggaagtacggtggctctggaggcggtggatcaggtggaaatgcgtacggcgagagaatcgtggacatcattgccaagaagggtggaagcggaggcggtggatcgggtggccgtgcgcgtatcgtgaccgatagccagtacgccctgtcgatgaatggaggtagcggtggcggaggttctggcggatacatggccgccgaacaggccagccaagaagtgaagaactggatgaaacggtatggaggcagtggtggcggaggtagtggtggatataattacagcttcaattgtggcggcgagttcttcagcggctggggaggtagcggcggtggaggcagcggaggttacgcggctggacatcaggccgctatgcagatgctgaaagagacaatctcacgagagggaggtagtggaggcggtggatcgggcggtatggctaagcagaaccccgagatcgtgatctacaggatgggtggcagtggaggtggcggaagcggaggttctgcgaccgccttcacaatccccagcatctccggtggcagtggtggcggaggttcaggtggacgcgcggctaagctgacccctctgtgtgtgaccctgtccatgatgggtggatctggtggaggcggttctggcggtaccgccttcacaatccctagcgtgcggtggggtggcagtggcggtggaggcagtggtggcatgatgcggcagaactacacacccggacctggcatcagataccctctgaccttcggctggtgcttcaagctgaaaaagcgaggcggttccggtggaggcggtagcggaggttactcagcattcaagcggaaaggcggcatcggcggctataggcgttatggtggctctggtggaggcggtagcggcggttacagttacaacacccagggctactaccccgattggaagcgatatggtggatcgggaggcggtggaagtggtggacagaatgattctgctctgtctgaaggcgccacacctcaggacctgaacaccatgctgaacaccagacgtggtggatcgggcggtggaggctcaggaggccgcgggcggctcgttggacccacacctgtgaacatcatcggccggaatctgctgttctggcggggcggatctggtggcggaggttcgggcggaatgtatatggtgaaggtggtggaagaaaaggctttcagccccgaagtgatccccatgttcagcgctcttaagaggggtggctccggcggaggtggctctggcggattctactataagcagaaccccgacatcgtgatctaccagtacatggacgacctgaaacgttatggtggcagtggaggcggtggatctggaggttatgctgccttcagagactacgtggaccggttctacaagaccctgagagccgacgatgatggcggtagcggaggcggtggatcaggaggctacatgtacgccgaacaggcttcccaggacgtgaagaactggatgtctatgtttggcggttctggcggaggtggcagtggtggcagagccattgaagcccagcagcacctcaagagaggtggaagtggcggtggaggctccggtggcaatcaagatgtgagaatgtacagccccaccagcatcctggacatcggcatgggaggaggctcgggaggcggtggatcgggaggcatcaggtaccccatccagaaagagacatgggaagcctggtggaccgagtactgggggaggaggggcggtagcggcggaggtggcagtggaggttacagtttccacacccagggctacttccccgactggcagaattacaccggtcgatacggcggttcaggtggaggcggttcaggaggtaactacgagatctacaagcggtggatcatcctgggcctgaacaagcgtagaggaggctccggcggaggtggctcaggtggacgggctgctggcctgaacaagatcgtgcggatgtaccgaaagtatggcggtagcggaggtggcggaagcggcggtagaatgttcaccagcacactgcaagagcagatcggctggaaaatatttggtggatcaggcggtggaggctctggaggtaacatctatcccatccagaaagagatctgggaaacctggtggaccgactactggggaaacaacggaggttctggtggaggcggttcaggaggtatgggtgggggccagatggttcaccagcctctgagccccagaacactgaatgcttggtaccgttatggcggatcgggcggaggtggcagcggaggctacgcttactacacacctggacctggcacaagataccctctgacctttggctgggccaagggtggaggtagcggcggaggtggctcgggtggcttctttttcggacagatggtgcatcaggccatctctcccagaacactgaacgcctgggtcaaagtggtgtattactatggtggctcaggtggaggcggtagtggtggatacatgtatacccctggcatcagataccagtacaacgtgctgaagaagactggtggctcaggcggaggtggcagcggcggtaaggcggcggcctgtcaaggtgttggaggacctggacacaaggccagagttctgaagacccagggcggatcgggcggaggtggctctggtggaacattcggcattcctcatcctgccggactgaaaagacgtggcggatcaggtggcggaggttcgggaggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 216 - RNA, 2952 nt, synthetic construct
  source 1..2952: mol_type = other RNA
atggattgcaaaacaatcttaaaagctttacggaagtacggtggctctggaggcggtggatcaggtggaaatgcgtacggagaaagaattgtggatattattgctaagaagggtggaagcggaggcggtggatcgggtggccgtgcgcgtattgtaacagattctcaatatgctctgtcgatgaatggaggtagcggtggcggaggttctggcggatacatggccgctgaacaagcttctcaggaagtgaaaaattggatgaaacggtatggaggcagtggtggcggaggtagtggtggatataattactcttttaattgtggaggagaattcttcagcggctggggaggtagcggcggtggaggcagcggaggttacgcggctggacaccaagcagcaatgcaaatgttgaaagaaaccatctcacgagagggaggtagtggaggcggtggatcgggcggtatggctaaacaaaatcctgaaattgtgatttacaggatgggtggcagtggaggtggcggaagcggaggttctgcgaccgcttttaccattccatcaatttccggtggcagtggtggcggaggttcaggtggacgcgcggctaaattgacccctttgtgtgtgaccttgtccatgatgggtggatctggtggaggcggttctggcggtaccgcttttaccattccaagtgttcggtggggtggcagtggcggtggaggcagtggtggcatgatgcggcagaattacacccctggacctggaatcagataccctctgacctttggatggtgctttaaactgaaaaagcgaggcggttccggtggaggcggtagcggaggttactcagcatttaaaagaaaaggaggaattggaggatacaggcgttatggtggctctggtggaggcggtagcggcggttacagttacaacacccaaggatactaccctgattggaagcgatatggtggatcgggaggcggtggaagtggtggacagaatgattctgcattgtctgaaggagcaacccctcaagatttgaataccatgttgaataccagacgtggtggatcgggcggtggaggctcaggaggccgcgggcggcttgtaggaccaacacctgtaaatatcatcggaagaaatcttcttttctggcggggcggatctggtggcggaggttcgggcggaatgtatatggtgaaagtggtggaagaaaaagctttttctcctgaagtgattccaatgttttctgctcttaagaggggtggctccggcggaggtggctctggcggattctactataaacaaaatcctgatattgtaatttaccaatacatggatgatctgaaacgttatggtggcagtggaggcggtggatctggaggttatgctgcctttagagattacgtggatagattttacaaaacactgagagcagacgatgatggcggtagcggaggcggtggatcaggaggctacatgtacgctgaacaagcttctcaagacgttaaaaattggatgtctatgtttggcggttctggcggaggtggcagtggtggcagagcaattgaagcacagcagcacctcaagagaggtggaagtggcggtggaggctccggtggcaatcaagatgtgagaatgtactcaccaacctcaattctggatattggcatgggaggaggctcgggaggcggtggatcgggaggcatcaggtacccaattcagaaagaaacatgggaagcttggtggacagaatactgggggaggaggggcggtagcggcggaggtggcagtggaggttacagtttccacacacagggatactttcctgattggcagaattacacaggtcgatacggcggttcaggtggaggcggttcaggaggtaactacgaaatttacaaaagatggattattctgggactgaataaacgtagaggaggctccggcggaggtggctcaggtggacgggctgctggattaaataaaattgtaagaatgtaccgaaagtatggcggtagcggaggtggcggaagcggcggtagaatgttcacatcaacacttcaggaacagattggatggaaaatatttggtggatcaggcggtggaggctctggaggtaacatctatccaatccagaaagaaatctgggaaacatggtggacagattactggggaaacaacggaggttctggtggaggcggttcaggaggtatgggtgggggacagatggtgcaccagccattgtctccaagaacattgaatgcttggtaccgttatggcggatcgggcggaggtggcagcggaggctacgcttactacacacctggacctggaacaagataccctctgacatttggatgggccaagggtggaggtagcggcggaggtggctcgggtggcttctttttcggacagatggttcaccaggcaatttctccaagaacattaaatgcatgggttaaagttgtttattactatggtggctcaggtggaggcggtagtggtggatacatgtatacacctggaataagataccaatacaatgtgcttaagaagactggtggctcaggcggaggtggcagcggcggtaaggcggcggcatgccagggagtgggaggacctggacacaaagcaagagtgcttaagacccagggcggatcgggcggaggtggctctggtggaacattcggaattcctcatcctgctggactgaaaagacgtggcggatcaggtggcggaggttcgggaggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 217 - RNA, 2874 nt, synthetic construct
  source 1..2874: mol_type = other RNA
atgatcgtgaccgatagccagtacgccctgtcaatgaacggaggtagcggtggcggaggttctggcggaatgatgcaggccgaacaggccacacaagaagtgaagaactggatgagtggagcaggcggtagcggcggaggtggcagtggaggtaatatgttcaccagcacactgcaagagcagatcgcctggaaaatctttggtggctctggaggcggtggatcaggtggaactttcggcattcctcatcctgccggactgaaacggcgtggaggttctggtggaggcggttcaggaggtttgcaggatgtgagaatgtacagccccgtgtccatcctggacatcgggatgggtggaggtagcggcggaggtggctcgggtggcagagccgcaggcctgaacaagatcgtgcggatgtaccgtaagtacggtggaagcggaggcggtggatcgggtggctacgcagcgggacatcaggccgctatgcagatgctgaaagagacaatctcaagggagggtggcagtggcggtggaggcagtggtggcatgtacgccaagaatcccgagatcgtgatctacaaaatgggcggtagcggaggtggcggaagcggcggttatgcgttcggccagatggttcaccaggctctgagccccagaacactgaacgcctgggtcaaagtgatcggcaggcgcggaggcagtggaggtggcggatcgggaggttacatgtatacccctggcatcagataccagtacaacgtgctgaaaaagaccggtggaagtggaggcggtggaagcggaggttacatggccgccgaacaggctacccaggacgtgaagaactggatgaaacgatatggaggcagtggaggtggcggatcaggtggacaaaataggagcttcaattgcaggggcgagttcttcgctgggaacggcggatctggtggcggaggttctggcggtatgatgatgtacacacctggacctggcgtcagattccctctgaccttcggctggtgcttcatgggcaacggaggctcgggtggaggcggttccggcggagcatacgcccagaatcctgagatcgtgatctaccagtacatggacgacctgaaacgcggaggtagcggcggtggaggcagcggaggtatgatgagacagaactacacacctggacctggcgtgcggtatcctctgaccttcggctggtgcttcaagctgaagaaacgcggcggatctggcggaggtggcagcggtggatatagcgcattcaagcggaaaggcggcatcggcggctatcgccgttacggaggttcgggtggaggcggtagtggaggctataacacccagggctacttccccgattggaagattggcggatcgggaggcggtggatcaggtggcacgtatgcacacacccagggattcttcccagactggcagaactacaccaagcgtggcggttctggcggtggaggctcgggtggaaatatctaccccatccagaaagagacatgggaaacctggtggaccgactactggggagggaacggtggaagtggtggaggcggttctggcggaatgatgtacggccagatggtgcatcagcctatcagccccagaacactgaacgcctgggcaaggaacggcggtagcggcggtggaggctcaggtggaatggcctatggcgagcggatcatcgacatcattgccaagaagggtggaggctctggcggaggtggcagtggtggccgtggaggcctgatcggacccacacctgtgaacatcatcggccggaacatgctgttctggagaggaggtagtggcggtggaggctccggcggtcgggcagcggactgcaagaccatcctgagagccctgcgtatgaggggtggatcgggaggtggcggatcgggcggagcagtgaaagtgatcgaggaaaaggctttcagccccgaagtgatccccatgttcacagcccttgcaatcggaggcagtggtggcggaggtagtggtggaataatgtatcccatccagaaagagatctgggaaacctggtggaccgagtactggggcgcgtggggaggctcaggaggtggcggatcaggaggttgggccttcagagactacgtggaccggttcttcaagaccctgagagccggtaaaggaggtagtggaggcggtggatcgggcggtagagccattgaagcccagcagcacatgaaaagaggaggtagtggaggcggtggatcgggcggtaacaactacacagctctgtctgaaggcgccacacctcaggacctgaacaccatgctgaacacctggggaggaggttcaggtggaggcggttctggtggaaattacgacatctacaagcggtggatcatcctgggcctgaacaaacgacgcggaggtagcggcggtggaggcagcggaggtagagcagctaagctgacccctctgtgtgtgaccctgtctatgatgggcggatcgggcggaggtggcagcggtggcttcgcagcggcctgtcaaggtgttggcggaccttctcacaaggccagagttctgagcggttggggcggttctggtggaggcggttccggtggcgcctttaccgccttcacaatccctagcacccgttggggtggctcaggcggaggtggcagtggtggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 218 - RNA, 2874 nt, synthetic construct
  source 1..2874: mol_type = other RNA
atgattgtaacagattctcaatatgctctgtcaatgaacggaggtagcggtggcggaggttctggcggaatgatgcaggctgaacaagctacacaagaagtgaaaaattggatgagtggagcaggcggtagcggcggaggtggcagtggaggtaatatgttcacatcaacacttcaggaacagattgcttggaaaatctttggtggctctggaggcggtggatcaggtggaactttcggaattcctcaccctgctggattgaaacggcgtggaggttctggtggaggcggttcaggaggtttgcaggatgtgagaatgtactctcctgtgtcaattctggatattgggatgggtggaggtagcggcggaggtggctcgggtggcagagccgcaggattaaataaaattgtaagaatgtaccgtaagtacggtggaagcggaggcggtggatcgggtggctacgcagcgggacaccaagcagcaatgcaaatgctgaaagaaaccatttcaagggagggtggcagtggcggtggaggcagtggtggcatgtacgctaaaaatcctgaaattgtgatttacaaaatgggcggtagcggaggtggcggaagcggcggttatgcgttcggacagatggtgcaccaggctttgtctccaagaacattgaatgcttgggtaaaagtaattggcaggcgcggaggcagtggaggtggcggatcgggaggttacatgtatacacctggaataagataccaatacaatgtgcttaaaaagaccggtggaagtggaggcggtggaagcggaggttacatggccgcagaacaagcaacacaagatgtgaaaaattggatgaaacgatatggaggcagtggaggtggcggatcaggtggacaaaataggtctttcaattgcagaggagaattcttcgctgggaacggcggatctggtggcggaggttctggcggtatgatgatgtacacccctggacctggagtgagatttcctctgacctttggatggtgctttatgggcaacggaggctcgggtggaggcggttccggcggagcatacgctcaaaatcctgaaattgtcatttaccaatacatggatgatcttaaacgcggaggtagcggcggtggaggcagcggaggtatgatgagacagaattacacccctggacctggagtgagataccctctgacctttggatggtgctttaaactgaagaaacgcggcggatctggcggaggtggcagcggtggatatagcgcatttaaaagaaaaggaggaattggaggataccgccgttacggaggttcgggtggaggcggtagtggaggctataatacccagggatactttcctgattggaagattggcggatcgggaggcggtggatcaggtggcacgtatgcacacacccaaggattcttccctgattggcaaaattacaccaagcgtggcggttctggcggtggaggctcgggtggaaatatctacccaatccagaaagaaacatgggaaacatggtggacagattactggggagggaacggtggaagtggtggaggcggttctggcggaatgatgtacggacagatggttcaccagccaatttctccaagaacactgaatgcttgggcaaggaacggcggtagcggcggtggaggctcaggtggaatggcctatggagaaagaattattgatattattgctaagaagggtggaggctctggcggaggtggcagtggtggccgtggaggcttgattggaccaacaccagtgaatattattggaagaaatatgttgttctggagaggaggtagtggcggtggaggctccggcggtcgggcagcggattgcaaaaccattttaagagctttacgtatgaggggtggatcgggaggtggcggatcgggcggagcagtgaaagtgattgaagaaaaagctttttctcctgaagtgattccaatgtttacagctctggcaatcggaggcagtggtggcggaggtagtggtggaataatgtatccaatccagaaagaaatctgggaaacatggtggacagaatactggggcgcgtggggaggctcaggaggtggcggatcaggaggttgggcctttagagattatgtggatagattcttcaaaaccttgagagctggtaaaggaggtagtggaggcggtggatcgggcggtagagcaatcgaagcacagcagcacatgaaaagaggaggtagtggaggcggtggatcgggcggtaacaactacacagcattgtctgaaggagcaacacctcaggatttgaatacaatgttgaatacatggggaggaggttcaggtggaggcggttctggtggaaattacgatatttacaaaagatggattattctgggactgaataaacgacgcggaggtagcggcggtggaggcagcggaggtagagcagctaaattgacccctttgtgtgtgaccttgtctatgatgggcggatcgggcggaggtggcagcggtggcttcgcagcggcatgccagggagtgggaggaccttctcacaaagcaagagtgcttagcggttggggcggttctggtggaggcggttccggtggcgcctttacagcttttacaatcccttcaacacgttggggtggctcaggcggaggtggcagtggtggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgataa

SEQ ID NO: 219 - protein, 8 aa, synthetic construct
AEQASQEV

SEQ ID NO: 220 - protein, 9 aa, synthetic construct
HQAAMQMLK

SEQ ID NO: 221 - protein, 8 aa, synthetic construct
MQMLKETI

SEQ ID NO: 222 - protein, 11 aa, synthetic construct
KQNPDIVIYQY

SEQ ID NO: 223 - protein, 9 aa, synthetic construct
IQKETWEAW

SEQ ID NO: 224 - protein, 8 aa, synthetic construct
GQMVHQPL

SEQ ID NO: 225 - protein, 8 aa, synthetic construct
GQMVHQAI

SEQ ID NO: 226 - protein, 11 aa, synthetic construct
SVDDALINSTK

SEQ ID NO: 227 - protein, 9 aa, synthetic construct
ALINSTKIY

SEQ ID NO: 228 - protein, 12 aa, synthetic construct
KIYSYFPSVISK

SEQ ID NO: 229 - protein, 9 aa, synthetic construct
KVNQGAQGK

SEQ ID NO: 230 - protein, 7 aa, synthetic construct
VTDSQYA

SEQ ID NO: 231 - protein, 8 aa, synthetic construct
VTDSQYAL

SEQ ID NO: 232 - protein, 8 aa, synthetic construct
AEQATQEV

SEQ ID NO: 233 - protein, 8 aa, synthetic construct
MQMLKETI

SEQ ID NO: 234 - protein, 8 aa, synthetic construct
GQMVHQAL

SEQ ID NO: 235 - protein, 8 aa, synthetic construct
AEQATQDV

SEQ ID NO: 236 - protein, 11 aa, synthetic construct
AQNPEIVIYQY

SEQ ID NO: 237 - protein, 9 aa, synthetic construct
NPEIVIYQY

SEQ ID NO: 238 - protein, 8 aa, synthetic construct
PEIVIYQY

SEQ ID NO: 239 - protein, 9 aa, synthetic construct
IQKETWETW

SEQ ID NO: 240 - protein, 8 aa, synthetic construct
GQMVHQPI

SEQ ID NO: 241 - protein, 9 aa, synthetic construct
VHQPISPRT

SEQ ID NO: 242 - protein, 8 aa, synthetic construct
DIYKRWII

SEQ ID NO: 243 - protein, 11 aa, synthetic construct
SVDDALINSTK

SEQ ID NO: 244 - protein, 9 aa, synthetic construct
ALINSTKIY

SEQ ID NO: 245 - protein, 12 aa, synthetic construct
KIYSYFPSVISK

SEQ ID NO: 246 - protein, 9 aa, synthetic construct
KVNQGAQGK